(19) United States
(12) Patent Application Publication
(10) Pub. No.: US 2026/0028338 A1
(43) Pub. Date: Jan. 29, 2026

(54) HETEROBIFUNCTIONAL COMPOUNDS AND METHODS OF TREATING DISEASE

(71) Applicant: Halda Therapeutics OpCo, Inc., New Haven, CT (US)

(72) Inventors: Kyle J. Eastman, Killingworth, CT (US); Katherine J. Kayser-Bricker, Branford, CT (US); James John Mousseau, Salem, CT (US); Taavi Neklesa, Orange, CT (US); David E. Puleo, Fairfield, CT (US); Kanak Shail Raina, Branford, CT (US); Nilesh K. Zaware, Hamden, CT (US)

(21) Appl. No.: 19/349,384

(22) Filed: Oct. 3, 2025

Related U.S. Application Data
(63) Continuation of application No. PCT/US2024/023239, filed on Apr. 5, 2024.
(60) Provisional application No. 63/494,692, filed on Apr. 6, 2023.

(51) Int. Cl. C07D 471/04; A61K 47/54; A61K 47/55; A61P 35/00; C07D 495/14

(52) U.S. Cl. CPC C07D 471/04; A61K 47/545; A61K 47/55; A61P 35/00; C07D 495/14

(57) ABSTRACT

The invention provides heterobifunctional compounds, pharmaceutical compositions, and their use treating disease, such as cancer.

### CROSS-REFERENCE TO RELATED APPLICATIONS

This application is a continuation of International (PCT) Patent Application Serial No. PCT/US2024/023239, filed Apr. 5, 2024, which claims the benefit of and priority to U.S. Provisional Patent Application Ser. No. 63/494,692, filed Apr. 6, 2023; the contents of each of which are hereby incorporated by reference in their entirety.

### FIELD OF THE INVENTION

The invention provides heterobifunctional compounds, pharmaceutical compositions, and their use treating disease, such as cancer.

### BACKGROUND

Cancer continues to be a significant health problem despite the substantial research efforts and scientific advances reported in the literature for treating this disease. Solid tumors, including prostate cancer, breast cancer, and lung cancer remain highly prevalent among the world population. The incidence of prostate cancer increases with age, and with increasing longevity of human subjects, there continues to be a corresponding rise in the number of patients suffering from prostate cancer. Breast cancer is one of the most common cancers among women and is a leading cause of death for women between ages 50-55. Lung cancer is a leading cause of death among cancer patients, where over 85% of lung cancers are non-small cell lung cancer (NSCLC). Many lung cancers are attributed to tobacco smoking. Current treatment options for these cancers are not effective for all patients and/or can have substantial adverse side effects.

New therapies are needed to address this unmet need in cancer therapy. In particular, new therapies are needed that achieve an anti-cancer effect through a different mechanism than commonly available therapies. Exemplary mechanisms for common anti-cancer therapies include (a) alkylation of DNA which limits ability of the cell to reproduce, (b) topoisomerase inhibition, in which the therapeutic agent inhibits the activity of a topoisomerases thereby limiting separation of strands of DNA, and (c) mitotic inhibition, where the therapeutic agent reduces ability of the cell to divide. New therapies that achieve an anti-cancer effect through a different mechanism present an opportunity to treat cancers more effectively and/or to treat cancers that have become resistant to currently available medicines.

The present invention addresses the foregoing needs and provides other related advantages.

### SUMMARY

The invention provides heterobifunctional compounds, pharmaceutical compositions, and their use treating disease, such as cancer. In particular, one aspect of the invention provides a collection of heterobifunctional compounds, such as a compound represented by Formula I:

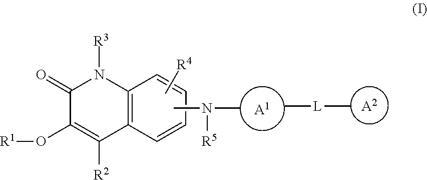

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a collection of heterobifunctional compounds, such as a compound represented by Formula II:

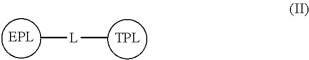

or a pharmaceutically acceptable salt thereof, where the variables are as defined in the detailed description. Further description of additional collections of heterobifunctional compounds are described in the detailed description. The compounds may be part of a pharmaceutical composition comprising a pharmaceutically acceptable carrier.

Another aspect of the invention provides a method of treating cancer. The method comprises administering to a patient in need thereof a therapeutically effective amount of a compound described herein, such as a compound of Formula I or II, to treat the cancer.

Another aspect of the invention provides a method of causing death of a cancer cell. The method comprises contacting a cancer cell with an effective amount of a compound described herein, such as a compound of Formula I or II, to cause death of the cancer cell.

### DETAILED DESCRIPTION

The invention provides heterobifunctional compounds, pharmaceutical compositions, and their use treating disease, such as cancer. The practice of the present invention employs, unless otherwise indicated, conventional techniques of organic chemistry, pharmacology, molecular biology (including recombinant techniques), cell biology, biochemistry, and immunology. Such techniques are explained in the literature, such as in “Comprehensive Organic Synthesis” (B. M. Trost & I. Fleming, eds., 1991-1992); “Handbook of experimental immunology” (D. M. Weir & C. C. Blackwell, eds.); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety.

Various aspects of the invention are set forth below in sections; however, aspects of the invention described in one particular section are not to be limited to any particular section. Further, when a variable is not accompanied by a definition, the previous definition of the variable controls.

### Definitions

Compounds of the present invention include those described generally herein, and are further illustrated by the classes, subclasses, and species disclosed herein. As used herein, the following definitions shall apply unless otherwise indicated. These definitions apply regardless of whether a term is used by itself or in combination with other terms, unless otherwise indicated. Hence, the definition of “alkyl” applies to “alkyl” as well as the “alkyl” portions of “—O-alkyl” etc. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

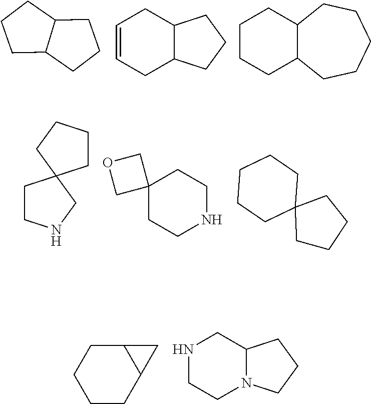

Exemplary bridged bicyclics include:

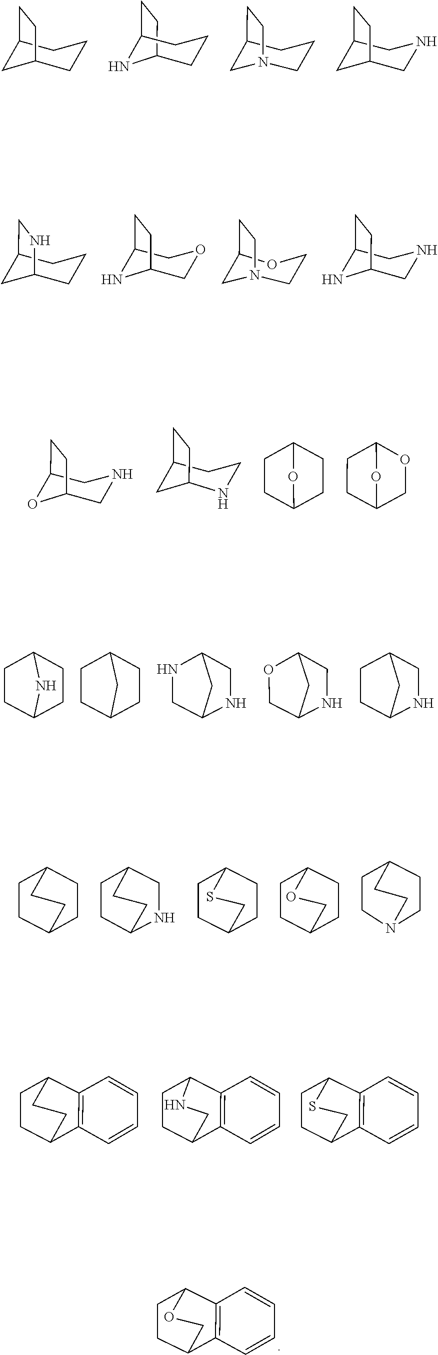

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “—(C₀ alkylene)-” refers to a bond. Accordingly, the term “—(C₀₋₃ alkylene)-” encompasses a bond (i.e., C₀) and a —(C₁₋₃ alkylene)- group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like. The term “haloaryl” refers to an aryl group that is substituted with at least one halogen. Exemplary haloaryl groups include chlorophenyl (e.g., 3-chlorophenyl, 4-chlorophenyl), fluorophenyl, and the like. The term “phenylene” refers to a bivalent phenyl group.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where unless otherwise specified, the radical or point of attachment is on the heteroaromatic ring or on one of the rings to which the heteroaromatic ring is fused. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, and tetrahydroisoquinolinyl. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted. The term “haloheteroaryl” refers to a heteroaryl group that is substituted with at least one halogen. Exemplary haloheteroaryl groups include chloropyridine, fluoropyridine, chloropyrazole, fluoropyrazole, and the like. The term “heteroarylene” refers to a bivalent heteroaryl group. Similarly, the terms “pyrazolylene”, “imidazolylene”, and “pyrrolylene”, respectively refer to bivalent pyrazolyl, imidazolyl, and pyrrolyl groups. Similarly, the terms “pyridazinylene,” “pyrimidinylene,” “pyrazinylene,” and “pyridinylene,” respectively refer to bivalent pyridazinyl, pyrimidinyl, pyrazinyl, and pyridinyl groups.

As used herein, the terms “heterocycle,” “heterocyclyl,” “heterocyclic radical,” and “heterocyclic ring” are used interchangeably and refer to a stable 5- to 7-membered monocyclic or 7-11-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted. The term “heterocyclylene” refers to a bivalent heterocyclyl group. The terms “piperidinylene,” “piperazinylene,” and “azetidinylene”, respectively refer to bivalent piperidinyl, piperazinyl, and azetidinyl groups.

As used herein, the term “heterocycloalkyl” refers to a saturated heterocyclyl. The term “heterocycloalkylene” refers to a bivalent heterocycloalkyl group.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N═C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂.

Each R^{∘} is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of R^{∘} selected from ═O and ═S; or each R^{∘} is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR*₂, =NNHC(O)R*, =NNHC(O)OR*, =NNHS(O)₂R*, =NR*, =NOR*, —O(C(R*₂))₂₋₃₀—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, R^{†} is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66 (1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. The invention includes compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

Chemical names, common names, and chemical structures may be used interchangeably to describe the same structure. If a chemical compound is referred to using both a chemical structure and a chemical name, and an ambiguity exists between the structure and the name, the structure predominates. It should also be noted that any carbon as well as heteroatom with unsatisfied valences in the text, schemes, examples and tables herein is assumed to have the sufficient number of hydrogen atom(s) to satisfy the valences.

The terms “a” and “an” as used herein mean “one or more” and include the plural unless the context is inappropriate.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl. The term “cycloalkylene” refers to a bivalent cycloalkyl group.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like. The term “chloroalkyl” refers to an alkyl group that is substituted with at least one chloro. The term “bromoalkyl” refers to an alkyl group that is substituted with at least one bromo. The term “haloalkylene” refers to a bivalent haloalkyl group.

The term “hydroxyalkyl” refers to an alkyl group that is substituted with at least one hydroxyl. Exemplary hydroxyalkyl groups include —CH₂CH₂OH, —C(H)(OH)CH₃, —CH₂C(H)(OH)CH₂CH₂OH, and the like.

The term “heteroalkyl” refers to an alkyl group in which one or more carbon atoms has been replaced by a heteroatom (e.g., N, O, or S). Exemplary heteroalkyl groups include —OCH₃, —CH₂OCH₃, —CH₂CH₂N(CH₃)₂, and —CH₂CH₂OH. The heteroalkyl group may contain, for example, from 2-4, 2-6, or 2-8 atoms selected from the group consisting of carbon and a heteroatom (e.g., N, O, or S). The phrase 3-8 membered heteroalkyl refers to a heteroalkyl group having from 3 to 8 atoms selected from the group consisting of carbon and a heteroatom. The term “heteroalkylene” refers to a bivalent heteroalkyl group.

The terms “alkenyl” and “alkynyl” are art-recognized and refer to unsaturated aliphatic groups analogous in length and possible substitution to the alkyls described above, but that contain at least one double or triple bond respectively. The term “haloalkenyl” refers to an alkenyl group that is substituted with at least one halogen. The term “fluoroalkenyl” refers to an alkenyl group that is substituted with at least one fluoro. The term “nitroalkenyl” refers to an alkenyl group that is substituted with at least one nitro.

The term “carbocyclylene” refers to a bivalent cycloaliphatic group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. The term “haloalkoxyl” refers to an alkoxyl group that is substituted with at least one halogen. Exemplary haloalkoxyl groups include —OCH₂F, —OCHF₂, —OCF₃, —OCH₂CF₃, —OCF₂CF₃, and the like.

The term “oxo” is art-recognized and refers to a “=O” substituent. For example, a cyclopentane substituted with an oxo group is cyclopentanone.

The term “amino” is art-recognized and refers to both unsubstituted and substituted amines, e.g., a moiety that may be represented by the general formulas:

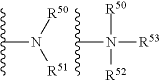

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8.

The term “amido” is art-recognized and refers to both unsubstituted and substituted amides, e.g., a moiety that may be represented by the general formulas:

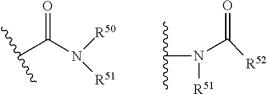

wherein R⁵⁰ and R⁵¹ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a 3-7 membered cycloalkyl, a 4-7 membered cycloalkenyl, 5-10 membered heteroaryl, or 3-10 membered heterocyclyl; and m is zero or an integer in the range of 1 to 8; and R⁵² is an alkyl, an alkenyl, or —(CH₂)ₘ—R⁶¹.

The symbol “ [symbol] ” indicates a point of attachment.

When any substituent or variable occurs more than one time in any constituent or the compound of the invention, its definition on each occurrence is independent of its definition at every other occurrence, unless otherwise indicated.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “IC₅₀” is art-recognized and refers to the concentration of a compound that is required to achieve 50% inhibition of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA [1975].

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

In addition, when a compound of the invention contains both a basic moiety (such as, but not limited to, a pyridine or imidazole) and an acidic moiety (such as, but not limited to, a carboxylic acid) zwitterions (“inner salts”) may be formed. Such acidic and basic salts used within the scope of the invention are pharmaceutically acceptable (i.e., non-toxic, physiologically acceptable) salts. Such salts of the compounds of the invention may be formed, for example, by reacting a compound of the invention with an amount of acid or base, such as an equivalent amount, in a medium such as one in which the salt precipitates or in an aqueous medium followed by lyophilization.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified.

### I. Heterobifunctional Compounds

One aspect of the invention provides heterobifunctional compounds. The compounds may be used in the pharmaceutical compositions and therapeutic methods described herein. Exemplary compounds are described in the following sections, along with exemplary procedures for making the compounds. Without being bound by theory, the compounds can facilitate therapeutic effects by binding to both BCL6 (B-cell lymphoma 6 protein) and BRD4 (bromodomain-containing protein 4).

### Part A: Compound of Formula I

One aspect of the invention provides a compound represented by Formula I:

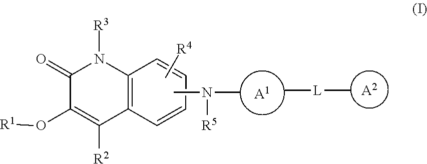

or a pharmaceutically acceptable salt thereof, wherein:
R¹ is —(C₁₋₃ alkylene)-C(O)N(R⁶)₂;
R², R³, R⁵, and R⁶ are independently hydrogen or C₁₋₄ alkyl;
R⁴ is hydrogen, C₁₋₄ alkyl, or halo;
R⁷ represents independently for each occurrence halo or C₁₋₄ alkyl;
A¹ is a pyrimidinylene, pyridazinylene, pyrazinylene, pyridinylene, or phenylene, each of which is substituted with n occurrences of R⁷;
L is a linker; and
A² is one of the following:

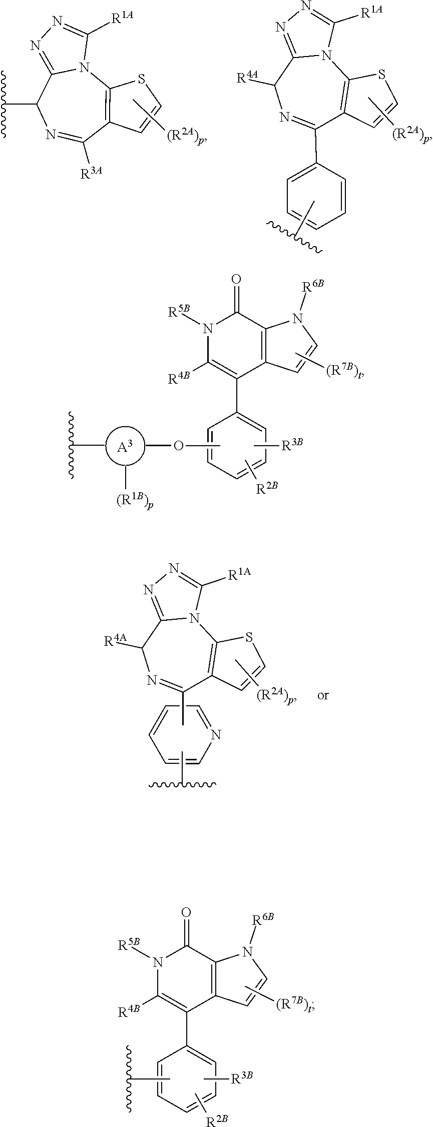

R^{1A} is C₁₋₄ alkyl or C₃₋₄ cycloalkyl;
R^{2A} represents independently for each occurrence C₁₋₄ alkyl or C₃₋₄ cycloalkyl;
R^{3A} is phenyl substituted with 1, 2, or 3 substituents independently selected from halo, C₁-C₄ alkyl, or C₁-C₄ haloalkyl;
R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, C₁₋₆ alkyl, or —(C₀₋₆ alkylene)-(5-6 membered heteroaryl containing 1, 2, or 3 heteroatoms independently selected from nitrogen, oxygen, and sulfur);
R^{5A} and R^{6A} are independently hydrogen, C₁₋₆ alkyl, or C₃₋₆ cycloalkyl; or R^{5A} and R^{6A} are taken together with the nitrogen atom to which they are attached to form a 3-7 membered ring containing 1 nitrogen atom;
R^{7A} is C₁₋₆ alkyl, —(C₁₋₆ alkylene)-(C₃₋₆ cycloalkyl), or C₃₋₆ cycloalkyl;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
A³ is phenylene, a 3-10 membered saturated monocyclic, bicyclic or spirocyclic carbocycylene, or C₁₋₆ alkylene; and
n, p, and t are independently 0, 1, or 2.

The definitions of variables in Formula I above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula I.

As defined generally above, R¹ is —(C₁₋₃ alkylene)-C(O)N(R⁶)₂. In certain embodiments, R¹ is —CH₂—C(O)N(H)CH₃. In certain embodiments, R¹ is selected from the groups depicted in the compounds in Table 1 below.

As defined generally above, R², R³, R⁵, and R⁶ are independently hydrogen or C₁₋₄ alkyl. In certain embodiments, R² is hydrogen or C₁₋₄ alkyl. In certain embodiments, R² is hydrogen. In certain embodiments, R² is C₁₋₄ alkyl. In certain embodiments, R³ is hydrogen or C₁₋₄ alkyl. In certain embodiments, R³ is hydrogen. In certain embodiments, R³ is C₁₋₄ alkyl. In certain embodiments, R³ is methyl. In certain embodiments, R⁵ is hydrogen or C₁₋₄ alkyl. In certain embodiments, R⁵ is hydrogen. In certain embodiments, R⁵ is C₁₋₄ alkyl. In certain embodiments, R⁶ is independently hydrogen or C₁₋₄ alkyl. In certain embodiments, R⁶ is hydrogen. In certain embodiments, R⁶ is C₁₋₄ alkyl. In certain embodiments, R², R³, R⁵, and R⁶ are each independently selected from the groups depicted in the compounds in Table 1 below.

As define generally above, R⁴ is hydrogen, C₁₋₄ alkyl, or halo. In certain embodiments, R⁴ is hydrogen. In certain embodiments, R⁴ is C₁₋₄ alkyl. In certain embodiments, R⁴ is halo. In certain embodiments, R⁴ and R⁵ are hydrogen. In certain embodiments, R⁴ is selected from the groups depicted in the compounds in Table 1 below.

As defined generally above, R⁷ represents independently for each occurrence halo or C₁₋₄ alkyl. In certain embodiments, R⁷ is halo. In certain embodiments, R⁷ is C₁₋₄ alkyl. In certain embodiments, R⁷ is chloro or fluoro. In certain embodiments, R⁷ is selected from the groups depicted in the compounds in Table 1 below.

As defined generally above, A¹ is a pyrimidinylene, pyridazinylene, pyrazinylene, pyridinylene, or phenylene, each of which is substituted with n occurrences of R⁷.

In certain embodiments, A¹ is pyrimidinylene substituted with n occurrences of R⁵. In certain embodiments, A¹ is

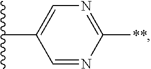

where ** is the point of attachment to L. In certain embodiments, A¹ is

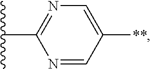

where ** is the point of attachment to L. In certain embodiments, A¹ is

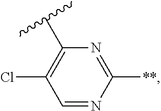

where ** is the point of attachment to L.

In certain embodiments, A¹ is pyridazinylene substituted with n occurrences of R⁵. In certain embodiments, A¹ is

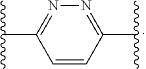 [SMILES: *c1ccc(*)nn1]

In certain embodiments, A¹ is pyrazinylene substituted with n occurrences of R⁵. In certain embodiments, A¹ is

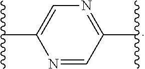 [SMILES: *c1cnc(*)cn1]

In certain embodiments, A¹ is pyridinylene substituted with n occurrences of R⁵. In certain embodiments, A¹ is

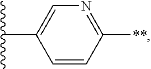

where ** is the point of attachment to L. In certain embodiments, A¹ is phenylene substituted with n occurrences of R⁵. In certain embodiments, A¹ is

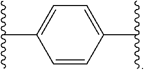 [SMILES: *c1ccc(*)cc1]

In certain embodiments, A¹ is a pyridazinylene substituted with 0 occurrences of R⁵. In certain embodiments, A¹ is a pyrimidinylene substituted with 0 occurrences of R⁵. In certain embodiments, A¹ is a pyrazinylene substituted with 0 occurrences of R⁵. In certain embodiments, A¹ is a pyridinylene, substituted with 0 occurrences of R⁵. In certain embodiments, A¹ is a phenylene substituted with 0 occurrences of R⁵. In certain embodiments, A¹ is selected from the groups depicted in the compounds in Table 1 below.

As defined generally above, A² is

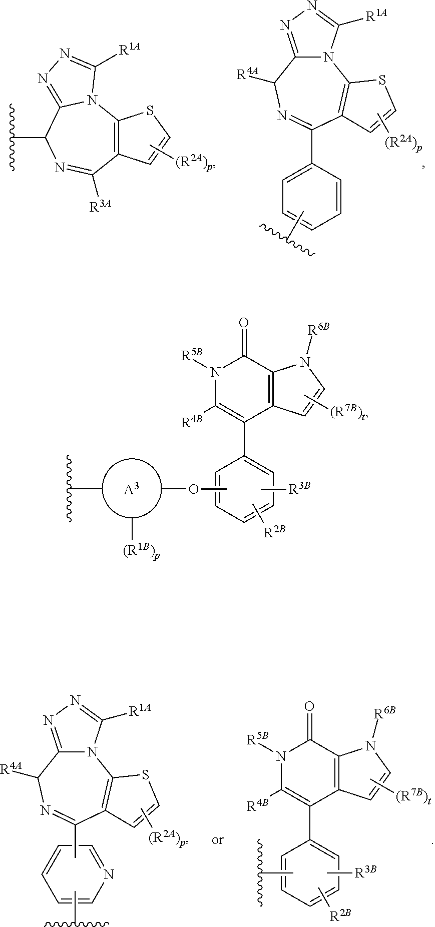

In certain embodiments, A² is

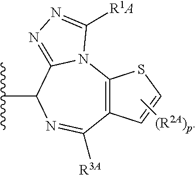

In certain embodiments, A² is

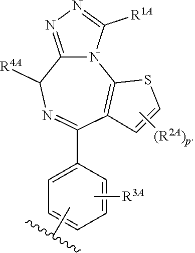

In certain embodiments, A² is

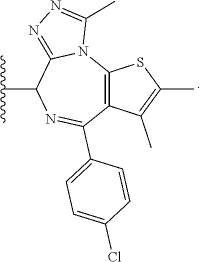 [SMILES: *C1N=C(c2ccc(Cl)cc2)c2c(sc(C)c2C)-n2c(C)nnc21]

In certain embodiments, A² is

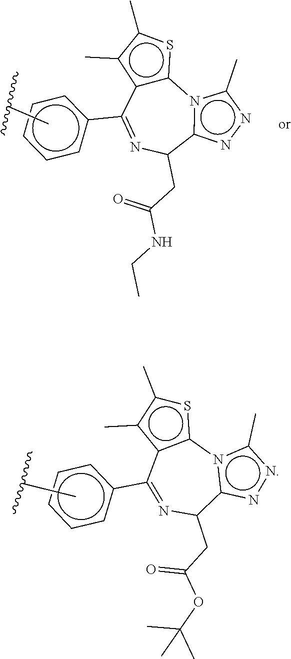

In certain embodiments, A² is

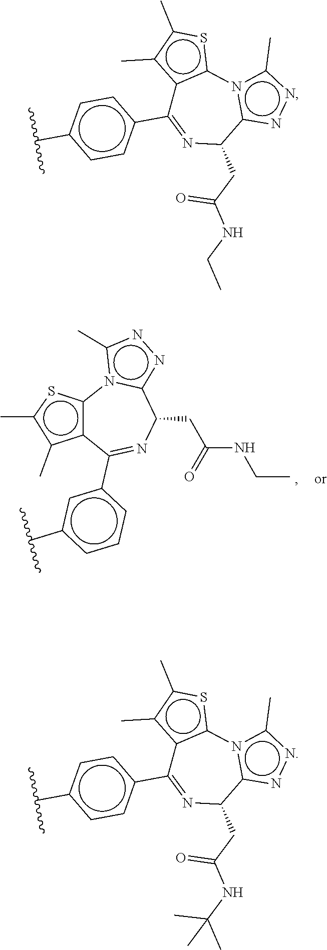

In certain embodiments, A² is

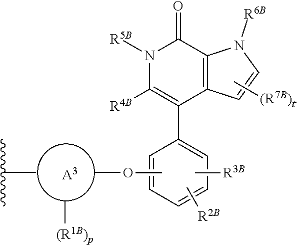

In certain embodiments, A² is

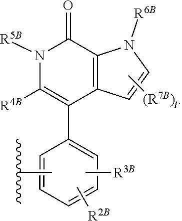

In certain embodiments, A² is

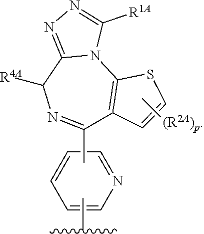

In certain embodiments, A² is

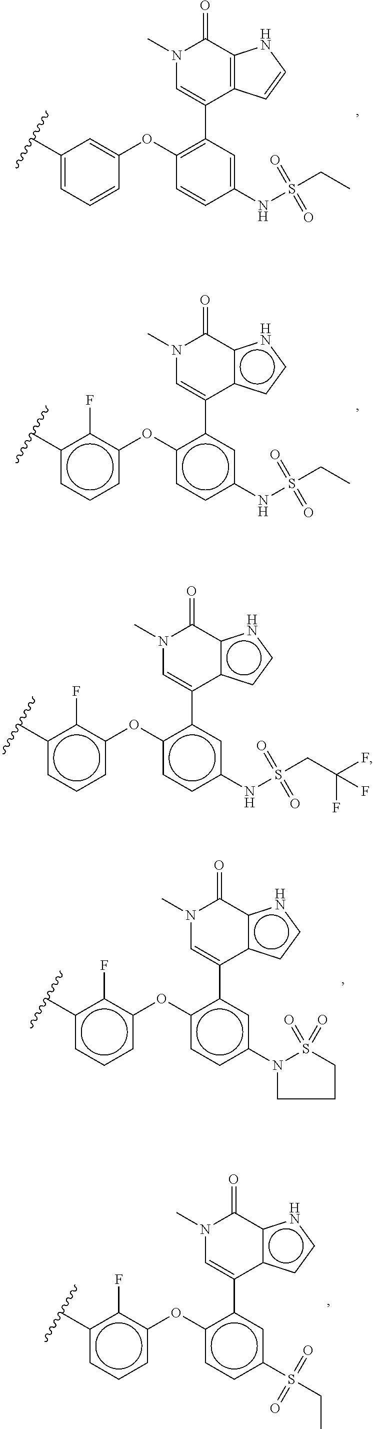

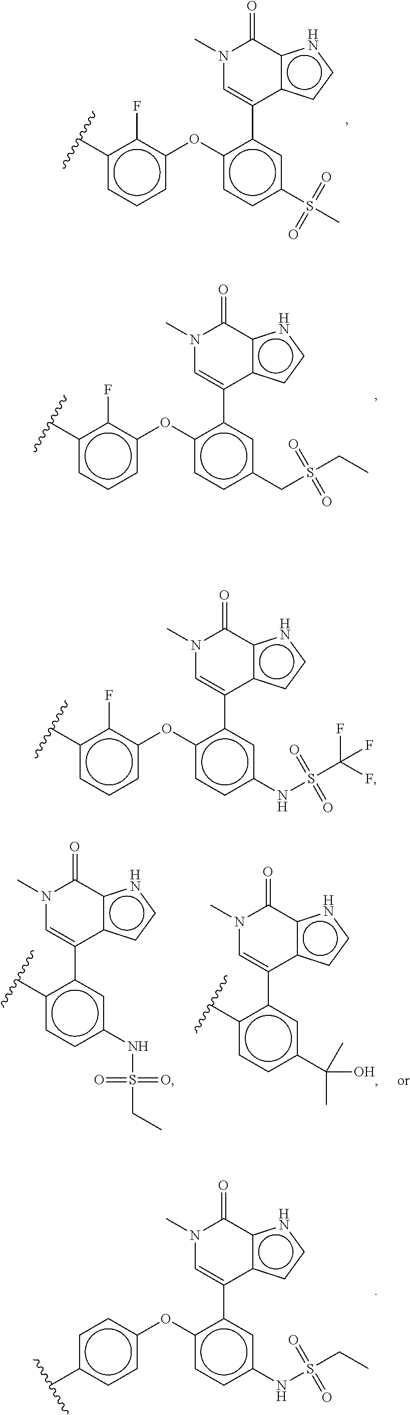

In certain embodiments, A² is

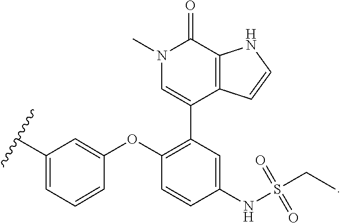 [SMILES: *c1cccc(Oc2ccc(NS(=O)(=O)CC)cc2-c2cn(C)c(=O)c3[nH]ccc23)c1]

In certain embodiments, A² is selected from the groups depicted in the compounds in Table 1 below.

As defined generally above, R^{1A} is C₁₋₄ alkyl or C₃₋₄ cycloalkyl. In certain embodiments, R^{1A} is C₁₋₄ alkyl. In certain embodiments, is methyl. In certain embodiments, R^{1A} is C₃₋₄ cycloalkyl. In certain embodiments, R^{1A} is selected from the groups depicted in the compounds in Table 1 below.

As defined generally above, R^{2A} represents independently for each occurrence C₁₋₄ alkyl or C₃₋₄ cycloalkyl. In certain embodiments, R^{2A} is C₁₋₄ alkyl. In certain embodiments, R^{2A} is methyl. In certain embodiments, R^{2A} is C₃₋₄ cycloalkyl. In certain embodiments, R^{2A} is selected from the groups depicted in the compounds in Table 1 below.

As defined generally above, R^{3A} is phenyl substituted with 1, 2, or 3 substituents independently selected from halo, C₁-C₄ alkyl, or C₁-C₄ haloalkyl. In certain embodiments, R^{3A} is phenyl substituted with halo. In certain embodiments, R^{3A} is phenyl substituted with 1 substituent selected from halo, C₁-C₄ alkyl, or C₁-C₄ haloalkyl. In certain embodiments, R^{3A} is phenyl substituted with 2 substituents independently selected from halo, C₁-C₄ alkyl, or C₁-C₄ haloalkyl. In certain embodiments, R^{3A} is phenyl substituted with 3 substituents independently selected from halo, C₁-C₄ alkyl, or C₁-C₄ haloalkyl. In certain embodiments, R^{3A} is selected from the groups depicted in the compounds in Table 1 below.

As defined generally above, R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, C₁₋₆ alkyl, or —(C₀₋₆ alkylene)-(5-6 membered heteroaryl containing 1, 2, or 3 heteroatoms independently selected from nitrogen, oxygen, and sulfur). In certain embodiments, R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, or —(C₁₋₆ alkylene)-OC(O)R^{7A}. In certain embodiments, R^{4A} is —(C₁₋₆ alkylene)-C(O)N(R^{5A})(R^{6A}). In certain embodiments, R^{4A} is —(C₁₋₆ alkylene)-CO₂R^{7A}. In certain embodiments, R^{4A} is —(C₁₋₆ alkylene)-N(R^{5A})C(O)R^{7A}. In certain embodiments, R^{4A} is —(C₁₋₆ alkylene)-OC(O)R^{7A}. In certain embodiments, R^{4A} is C₁₋₆ alkyl. In certain embodiments, R^{4A} is —(C₀₋₆ alkylene)-(5-6 membered heteroaryl containing 1, 2, or 3 heteroatoms independently selected from nitrogen, oxygen, and sulfur). In certain embodiments, R^{4A} is selected from the groups depicted in the compounds in Table 1 below.

As defined generally above, R^{5A} and R^{6A} are independently hydrogen, C₁₋₆ alkyl, or C₃₋₆ cycloalkyl; or R^{5A} and R^{6A} are taken together with the nitrogen atom to which they are attached to form a 3-7 membered ring containing 1 nitrogen atom. In certain embodiments, R^{5A} and R^{6A} are independently hydrogen, C₁₋₆ alkyl, or C₃₋₆ cycloalkyl. In certain embodiments, R^{5A} is hydrogen. In certain embodiments, R^{5A} is C₁₋₆ alkyl. In certain embodiments, R^{5A} is C₃₋₆ cycloalkyl. In certain embodiments, R^{6A} is hydrogen. In certain embodiments, R^{6A} is C₁₋₆ alkyl. In certain embodiments, R^{6A} is C₃₋₆ cycloalkyl. In certain embodiments, R^{5A} and R^{6A} are taken together with the nitrogen atom to which they are attached to form a 3-7 membered ring containing 1 nitrogen atom. In certain embodiments, R^{5A} and R^{6A} are taken together with the nitrogen atom to which they are attached to form a 3-membered ring containing 1 nitrogen atom. In certain embodiments, R^{5A} and R^{6A} are taken together with the nitrogen atom to which they are attached to form a 4-membered ring containing 1 nitrogen atom. In certain embodiments, R^{5A} and R^{6A} are taken together with the nitrogen atom to which they are attached to form a 5-membered ring containing 1 nitrogen atom. In certain embodiments, R^{5A} and R^{6A} are taken together with the nitrogen atom to which they are attached to form a 6-membered ring containing 1 nitrogen atom. In certain embodiments, R^{5A} and R^{6A} are taken together with the nitrogen atom to which they are attached to form a 7-membered ring containing 1 nitrogen atom. In certain embodiments, R^{5A} is selected from the groups depicted in the compounds in Table 1 below. In certain embodiments, R^{6A} is selected from the groups depicted in the compounds in Table 1 below.

As defined generally above, R^{7A} is C₁₋₆ alkyl, —(C₁₋₆ alkylene)-(C₃₋₆ cycloalkyl), or C₃₋₆ cycloalkyl. In certain embodiments, R^{7A} is C₁₋₆ alkyl. In certain embodiments, R^{7A} is —(C₁₋₆ alkylene)-(C₃₋₆ cycloalkyl). In certain embodiments, R^{7A} is C₃₋₆ cycloalkyl. In certain embodiments, R^{7A} is selected from the groups depicted in the compounds in Table 1 below.

As defined generally above, A³ is phenylene, a 3-10 membered saturated monocyclic, bicyclic or spirocyclic carbocycylene, or C₁₋₆ alkylene. In certain embodiments, A³ is phenylene. In certain embodiments, A³ is a 3-10 membered saturated monocyclic, bicyclic or spirocyclic carbocycylene. In certain embodiments, A³ is C₁₋₆ alkylene. In certain embodiments, A³ is methylene or ethylene. In certain embodiments, A³ is a 3-6 membered saturated monocyclic carbocycylene. In certain embodiments, A³ is a 6-10 membered saturated bicyclic or spirocyclic carbocycylene. In certain embodiments, A³ is selected from the groups depicted in the compounds in Table 1 below.

As defined generally above, R^{1B} represents independently for each occurrence halo, C₁₋₄ alkyl, or C₁₋₄ haloalkyl. In certain embodiments, R^{1B} is halo. In certain embodiments, RB is fluoro. In certain embodiments, R^{1B} is C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R^{2B} is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂. In certain embodiments, R^{3B} is —N(R^{8B})SO₂R^{9B}, —SO₂N(R^{8B})₂—SO₂R^{9B}, or —(C₁₋₆ alkylene)-SO₂R^{9B}. In certain embodiments, R^{3B} is —N(R^{8B})SO₂R^{9B}. In certain embodiments, R^{3B} is SO₂R^{9B}. In certain embodiments, R^{3B} is —(C₁₋₆ alkylene)-SO₂R^{9B}. In certain embodiments, R^{3B} is —SO₂N(R^{8B})₂. In certain embodiments, R^{3B} is C₁₋₆ hydroxyalkyl. In certain embodiments, R^{3B} is —C(OH)(CH₃)₂. In certain embodiments, R^{3B} is or a 4-7 membered saturated carbocyclic ring in which one CH₂ is replaced with SO₂. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{5B} is methyl. In certain embodiments, R^{5B} is C₃₋₄ cycloalkyl. In certain embodiments, R^{5B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{6B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} represents independently for each occurrence hydrogen or C₁₋₄ alkyl. In certain embodiments, R^{8B} is hydrogen. In certain embodiments, R^{8B} is C₁₋₄ alkyl. In certain embodiments, two occurrences of R^{8B} are taken together with the nitrogen atom to which they are attached to form a 3-7 membered heterocyclic ring containing 1 or 2 heteroatoms independently selected from nitrogen and oxygen. In certain embodiments, R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9B} is C₃₋₆cycloalkyl. In certain embodiments, R^{9B} is selected from the groups depicted in the compounds in Table 1 below.

As defined generally above, n and p are independently 0, 1, or 2. In certain embodiments, p is 2. In certain embodiments, p is 1. In certain embodiments, p is 0. In certain embodiments, t is 2. In certain embodiments, n is 0. In certain embodiments, n is 1. In certain embodiments, p is selected from the corresponding value in the groups depicted in the compounds in Table 1 below. In certain embodiments, n is selected from the corresponding value in the groups depicted in the compounds in Table 1 below.

In certain embodiments, the compound of Formula I is further defined by Formula Ia or a pharmaceutically acceptable salt thereof:

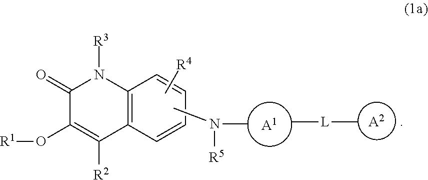

In certain embodiments, the definition of variables R¹, R², R³, R⁴, R⁵, A¹, and A² is one of the embodiments described above in connection with Formula I.

In certain embodiments, the compound of Formula I is further defined by Formula 1b or a pharmaceutically acceptable salt thereof:

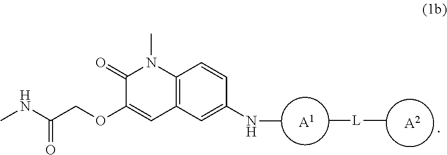

In certain embodiments, the definition of variables A¹ and A² is one of the embodiments described above in connection with Formula I.

The compounds may be further characterized according to, for example, the identity of L. Exemplary further embodiments for L are provided in Part C below.

### Part B: Compound of Formula II

Another aspect of the invention provides a compound represented by Formula II:

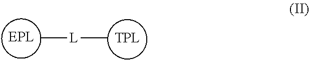

or a pharmaceutically acceptable salt thereof, wherein:
TPL is a group defined by Formula II-1 that is substituted by one occurrence of R^{II-1A}, wherein Formula II-1 is represented by:

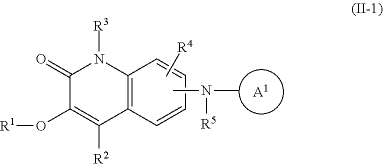

or a pharmaceutically acceptable salt thereof, wherein:
R^{II-1A} is a bond to L;
R¹ is —(C₁₋₃ alkylene)-C(O)N(R⁶)₂;
R², R³, R⁵, and R⁶ are independently hydrogen or C₁₋₄ alkyl;
R⁴ is hydrogen, C₁₋₄ alkyl, or halo;
R⁷ represents independently for each occurrence halo or C₁₋₄ alkyl;
A¹ is a pyrimidinyl, pyridazinyl, pyrazinyl, pyridinyl, or phenyl, each of which is substituted with n occurrences of R⁷;
L is a linker;
EPL is a moiety that binds to BRD4; and
n is 0, 1, or 2.

The definitions of variables in Formula II above encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a compound of Formula II.

As defined generally above, R¹ is —(C₁₋₃ alkylene)-C(O)N(R⁶)₂. In certain embodiments, R¹ is —CH₂—C(O)N(H)CH₃. In certain embodiments, R¹ is selected from the groups depicted in the compounds in Table 1 below.

As defined generally above, R², R³, R⁵, and R⁶ are independently hydrogen or C₁₋₄ alkyl. In certain embodiments, R² is hydrogen or C₁₋₄ alkyl. In certain embodiments, R² is hydrogen. In certain embodiments, R² is C₁₋₄ alkyl. In certain embodiments, R³ is hydrogen or C₁₋₄ alkyl. In certain embodiments, R³ is hydrogen. In certain embodiments, R³ is C₁₋₄ alkyl. In certain embodiments, R³ is methyl. In certain embodiments, R⁵ is hydrogen or C₁₋₄ alkyl. In certain embodiments, R⁵ is hydrogen. In certain embodiments, R⁵ is C₁₋₄ alkyl. In certain embodiments, R⁶ is independently hydrogen or C₁₋₄ alkyl. In certain embodiments, R⁶ is hydrogen. In certain embodiments, R⁶ is C₁₋₄ alkyl. In certain embodiments, R², R³, R⁵, and R⁶ are each independently selected from the groups depicted in the compounds in Table 1 below.

As define generally above, R⁴ is hydrogen, C₁₋₄ alkyl, or halo. In certain embodiments, R⁴ is hydrogen. In certain embodiments, R⁴ is C₁₋₄ alkyl. In certain embodiments, R⁴ is halo. In certain embodiments, R⁴ and R⁵ are hydrogen. In certain embodiments, R⁴ is selected from the groups depicted in the compounds in Table 1 below.

As defined generally above, R⁷ represents independently for each occurrence halo or C₁₋₄ alkyl. In certain embodiments, R⁷ is halo. In certain embodiments, R⁷ is C₁₋₄ alkyl. In certain embodiments, R⁷ is chloro or fluoro. In certain embodiments, R⁷ is selected from the groups depicted in the compounds in Table 1 below.

As defined generally above, A¹ is a pyrimidinyl, pyridazinyl, pyrazinyl, pyridinyl, or phenyl, each of which is substituted with n occurrences of R⁵. In certain embodiments, A¹ is pyridazinyl substituted with n occurrences of R⁵. In certain embodiments, A¹ is pyrimidinyl substituted with n occurrences of R⁵. In certain embodiments, A¹ is pyrazinyl substituted with n occurrences of R⁵. In certain embodiments, A¹ is pyridinyl substituted with n occurrences of R⁵. In In certain embodiments, A¹ is phenyl substituted with n occurrences of R⁵. In certain embodiments, A¹ is selected from the groups depicted in the compounds in Table 1 below.

In certain embodiments, n is 0. In certain embodiments, n is 1. In certain embodiments, n is 2. In certain embodiments, n is selected from the corresponding value in the groups depicted in the compounds in Table 1 below.

In certain embodiments, the TPL is

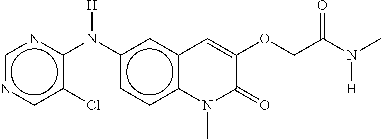

that is substituted by one occurrence of R^{II-1A}.

In certain embodiments, the TPL is

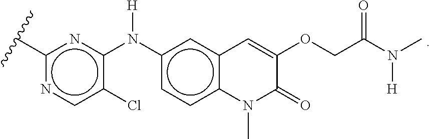

In certain embodiments, the EPL is defined by Formula II-2 that is substituted by one occurrence of R^{II-2A}, wherein Formula II-2 is represented by:

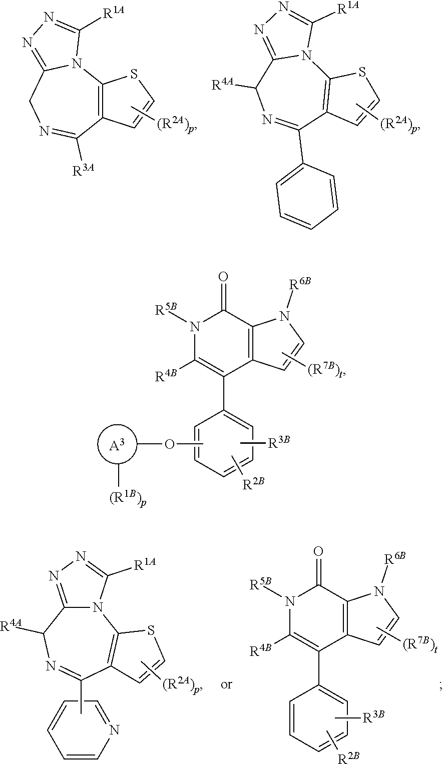

wherein
R^{II-2A} is a bond to L;
R^{1A} is C₁₋₄ alkyl or C₃₋₄ cycloalkyl;
R^{2A} represents independently for each occurrence C₁₋₄ alkyl or C₃₋₄ cycloalkyl;
R^{3A} is phenyl substituted with 1, 2, or 3 substituents independently selected from halo, C₁-C₄ alkyl, or C₁-C₄ haloalkyl;
R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, C₁₋₆ alkyl, or —(C₀₋₆ alkylene)-(5-6 membered heteroaryl containing 1, 2, or 3 heteroatoms independently selected from nitrogen, oxygen, and sulfur);
R^{5A} and R^{6A} are independently hydrogen, C₁₋₆ alkyl, or C₃₋₆ cycloalkyl; or R^{5A} and R^{6a} are taken together with the nitrogen atom to which they are attached to form a 3-7 membered ring containing 1 nitrogen atom;
R^{7A} is C₁₋₆ alkyl, —(C₁₋₆ alkylene)-(C₃₋₆ cycloalkyl), or C₃₋₆ cycloalkyl;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
A³ is phenylene, a 3-10 membered saturated monocyclic, bicyclic or spirocyclic carbocycylene, or C₁₋₆ alkylene; and
p and t are independently 0, 1, or 2.

In certain embodiments, the EPL is defined by Formula II-2 that is substituted by one occurrence of R^{II-2A}, wherein Formula II-2 is represented by:

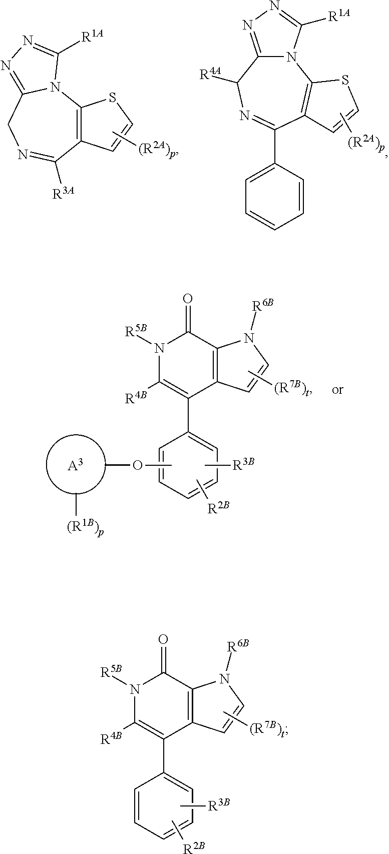

wherein
R^{II-2A} is a bond to L;
R^{1A} is C₁₋₄ alkyl or C₃₋₄ cycloalkyl;
R^{2A} represents independently for each occurrence C₁₋₄ alkyl or C₃₋₄ cycloalkyl;
R^{3A} is phenyl substituted with 1, 2, or 3 substituents independently selected from halo, C₁-C₄ alkyl, or C₁-C₄ haloalkyl;
R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, or —(C₀₋₆ alkylene)-(5-6 membered heteroaryl containing 1, 2, or 3 heteroatoms independently selected from nitrogen, oxygen, and sulfur);
R^{5A} and R^{6A} are independently hydrogen, C₁₋₆ alkyl, or C₃₋₆ cycloalkyl; or R^{5A} and R^{6a} are taken together with the nitrogen atom to which they are attached to form a 3-7 membered ring containing 1 nitrogen atom;
R^{7A} is C₁₋₆ alkyl, —(C₁₋₆ alkylene)-(C₃₋₆ cycloalkyl), or C₃₋₆ cycloalkyl;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
A³ is phenylene, a 3-10 membered saturated monocyclic, bicyclic or spirocyclic carbocycylene, or C₁₋₆ alkylene; and
p and t are independently 0, 1, or 2.

In certain embodiments, the EPL is

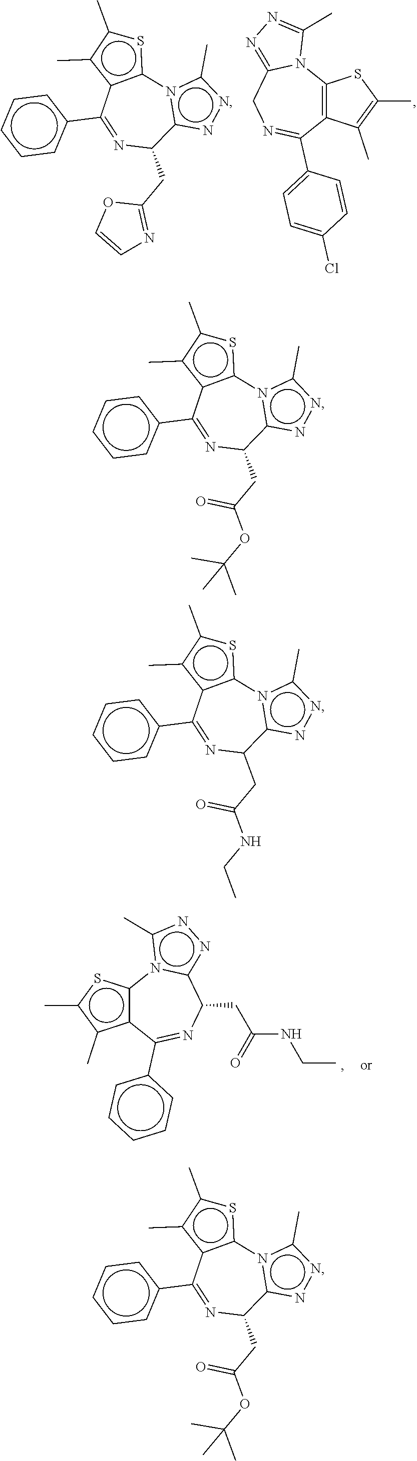

each of which is substituted by one occurrence of R^{II-2A}, wherein R^{II-2A} is a bond to L.

In certain embodiments, the EPL is one of the following:

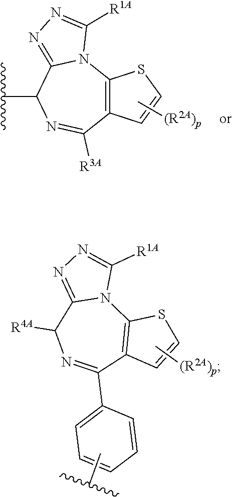

R^{1A} is C₁₋₄ alkyl or C₃₋₄ cycloalkyl;
R^{2A} represents independently for each occurrence C₁₋₄ alkyl or C₃₋₄ cycloalkyl;
R^{3A} is phenyl substituted with 1, 2, or 3 substituents independently selected from halo, C₁-C₄ alkyl, or C₁-C₄ haloalkyl;
R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, or —(C₁₋₆ alkylene)-OC(O)R^{7A}.
R^{5A} and R^{6A} are independently hydrogen, C₁₋₆ alkyl, or C₃₋₆ cycloalkyl; or R^{5A} and R^{6A} are taken together with the nitrogen atom to which they are attached to form a 3-7 membered ring containing 1 nitrogen atom;
R^{7A} is C₁₋₆ alkyl, —(C₁₋₆ alkylene)-(C₃₋₆ cycloalkyl), or C₃₋₆ cycloalkyl; and
p is 0, 1, or 2

In certain embodiments, the EPL is

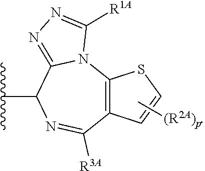

In certain embodiments, the EPL is

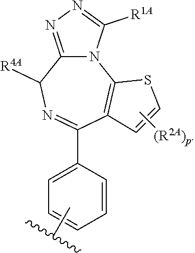

In certain embodiments, the EPL is

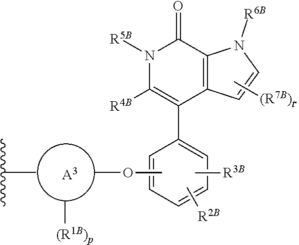

In certain embodiments, the EPL is

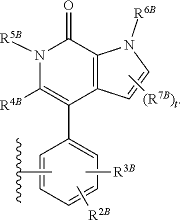

In certain embodiments, the EPL is

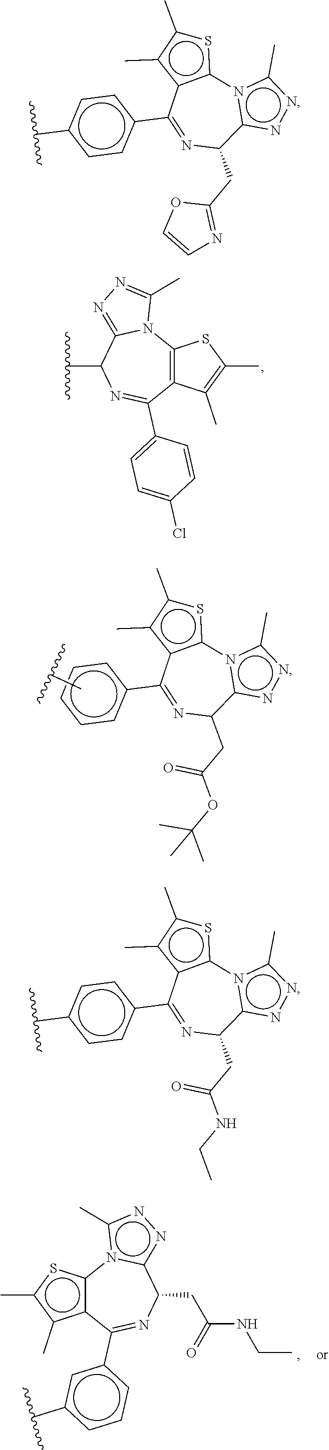

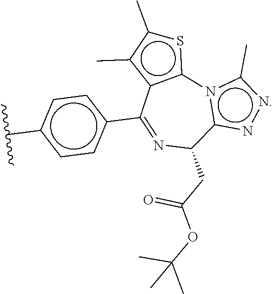

As defined generally above, R^{1A} is C₁₋₄ alkyl or C₃₋₄ cycloalkyl. In certain embodiments, R^{1A} is C₁₋₄ alkyl. In certain embodiments, is methyl. In certain embodiments, R^{1A} is C₃₋₄ cycloalkyl. In certain embodiments, R^{1A} is selected from the groups depicted in the compounds in Table 1 below.

As defined generally above, R^{2A} represents independently for each occurrence C₁₋₄ alkyl or C₃₋₄ cycloalkyl. In certain embodiments, R^{2A} is C₁₋₄ alkyl. In certain embodiments, R^{2A} is methyl. In certain embodiments, R^{2A} is C₃₋₄ cycloalkyl. In certain embodiments, R^{2A} is selected from the groups depicted in the compounds in Table 1 below.

As defined generally above, R^{3A} is phenyl substituted with 1, 2, or 3 substituents independently selected from halo, C₁-C₄ alkyl, or C₁-C₄ haloalkyl. In certain embodiments, R^{3A} is phenyl substituted with halo. In certain embodiments, R^{3A} is phenyl substituted with 1 substituent selected from halo, C₁-C₄ alkyl, or C₁-C₄ haloalkyl. In certain embodiments, R^{3A} is phenyl substituted with 2 substituents independently selected from halo, C₁-C₄ alkyl, or C₁-C₄ haloalkyl. In certain embodiments, R^{3A} is phenyl substituted with 3 substituents independently selected from halo, C₁-C₄ alkyl, or C₁-C₄ haloalkyl. In certain embodiments, R^{3A} is selected from the groups depicted in the compounds in Table 1 below.

As defined generally above, R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, C₁₋₆ alkyl, or —(C₀₋₆ alkylene)-(5-6 membered heteroaryl containing 1, 2, or 3 heteroatoms independently selected from nitrogen, oxygen, and sulfur). In certain embodiments, R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, or —(C₁₋₆ alkylene)-OC(O)R^{7A}. In certain embodiments, R^{4A} is —(C₁₋₆ alkylene)-C(O)N(R^{5A})(R^{6A}). In certain embodiments, R^{4A} is —(C₁₋₆ alkylene)-CO₂R^{7A}. In certain embodiments, R^{4A} is —(C₁₋₆ alkylene)-N(R^{5A})C(O)R^{7A}. In certain embodiments, R^{4A} is —(C₁₋₆ alkylene)-OC(O)R^{7A}. In certain embodiments, R^{4A} is C₁₋₆ alkyl. In certain embodiments, R^{4A} is —(C₀₋₆ alkylene)-(5-6 membered heteroaryl containing 1, 2, or 3 heteroatoms independently selected from nitrogen, oxygen, and sulfur). In certain embodiments, R^{4A} is selected from the groups depicted in the compounds in Table 1 below.

As defined generally above, R^{5A} and R^{6A} are independently hydrogen, C₁₋₆ alkyl, or C₃₋₆ cycloalkyl; or R^{5A} and R^{6A} are taken together with the nitrogen atom to which they are attached to form a 3-7 membered ring containing 1 nitrogen atom. In certain embodiments, R^{5A} and R^{6A} are independently hydrogen, C₁₋₆ alkyl, or C₃₋₆ cycloalkyl. In certain embodiments, R^{5A} is hydrogen. In certain embodiments, R^{5A} is C₁₋₆ alkyl. In certain embodiments, R^{5A} is C₃₋₆ cycloalkyl. In certain embodiments, R^{6A} is hydrogen. In certain embodiments, R^{6A} is C₁₋₆ alkyl. In certain embodiments, R^{6A} is C₃₋₆ cycloalkyl. In certain embodiments, R^{5A} and R^{6A} are taken together with the nitrogen atom to which they are attached to form a 3-7 membered ring containing 1 nitrogen atom. In certain embodiments, R^{5A} and R^{6A} are taken together with the nitrogen atom to which they are attached to form a 3-membered ring containing 1 nitrogen atom. In certain embodiments, R^{5A} and R^{6A} are taken together with the nitrogen atom to which they are attached to form a 4-membered ring containing 1 nitrogen atom. In certain embodiments, R^{5A} and R^{6A} are taken together with the nitrogen atom to which they are attached to form a 5-membered ring containing 1 nitrogen atom. In certain embodiments, R^{5A} and R^{6A} are taken together with the nitrogen atom to which they are attached to form a 6-membered ring containing 1 nitrogen atom. In certain embodiments, R^{5A} and R^{6A} are taken together with the nitrogen atom to which they are attached to form a 7-membered ring containing 1 nitrogen atom. In certain embodiments, R^{5A} is selected from the groups depicted in the compounds in Table 1 below. In certain embodiments, R^{6A} is selected from the groups depicted in the compounds in Table 1 below.

As defined generally above, R^{7A} is C₁₋₆ alkyl, —(C₁₋₆ alkylene)-(C₃₋₆ cycloalkyl), or C₃₋₆ cycloalkyl. In certain embodiments, R^{7A} is C₁₋₆ alkyl. In certain embodiments, R^{7A} is —(C₁₋₆ alkylene)-(C₃₋₆ cycloalkyl). In certain embodiments, R^{7A} is C₃₋₆ cycloalkyl. In certain embodiments, R^{7A} is selected from the groups depicted in the compounds in Table 1 below.

In certain embodiments, p is 2. In certain embodiments, p is 1. In certain embodiments, p is 0. In certain embodiments, t is 2. In certain embodiments, p is selected from the corresponding value in the groups depicted in the compounds in Table 1 below.

As defined generally above, R^{1B} represents independently for each occurrence halo, C₁₋₄ alkyl, or C₁₋₄ haloalkyl. In certain embodiments, R^{1B} is halo. In certain embodiments, RB is fluoro. In certain embodiments, R^{1B} is C₁₋₄ alkyl. In certain embodiments, R^{1B} is C₁₋₄ haloalkyl. In certain embodiments, R^{1B} is selected from the groups depicted in the compounds in Table 1 below.

As defined generally above, R^{2B} is hydrogen, halo, C₁₋₄ alkyl, or C₁₋₄ haloalkyl. In certain embodiments, R^{2B} is hydrogen. In certain embodiments, R^{2B} is halo. In certain embodiments, R^{2B} is C₁₋₄ alkyl. In certain embodiments, R^{2B} is C₁₋₄ haloalkyl. In certain embodiments, R^{2B} is selected from the groups depicted in the compounds in Table 1 below.

As defined generally above, R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂. In certain embodiments, R^{3B} is —N(R^{8B})SO₂R^{9B}, —SO₂N(R^{8B})₂—SO₂R^{9B}, or —(C₁₋₆ alkylene)-SO₂R^{9B}. In certain embodiments, R^{3B} is —N(R^{8B})SO₂R^{9B}. In certain embodiments, R^{3B} is SO₂R^{9B}. In certain embodiments, R^{3B} is —(C₁₋₆ alkylene)-SO₂R^{9B}. In certain embodiments, R^{3B} is —SO₂N(R^{8B})₂. In certain embodiments, R^{3B} is C₁₋₆ hydroxyalkyl. In certain embodiments, R^{3B} is —C(OH)(CH₃)₂. In certain embodiments, R^{3B} is or a 4-7 membered saturated carbocyclic ring in which one CH₂ is replaced with SO₂. In certain embodiments, R^{3B} is selected from the groups depicted in the compounds in Table 1 below.

As defined generally above, R^{4B} is hydrogen, halo, or C₁₋₄ alkyl. In certain embodiments, R^{4B} is hydrogen. In certain embodiments, R^{4B} is halo. In certain embodiments, R^{4B} is C₁₋₄ alkyl. In certain embodiments, R^{4B} is selected from the groups depicted in the compounds in Table 1 below.

As defined generally above, R^{5B} is C₁₋₄ alkyl or C₃₋₄ cycloalkyl. In certain embodiments, R^{5B} is C₁₋₄ alkyl. In certain embodiments, R^{5B} is methyl. In certain embodiments, R^{5B} is C₃₋₄ cycloalkyl. In certain embodiments, R^{5B} is selected from the groups depicted in the compounds in Table 1 below.

As defined generally above, R^{6B} is hydrogen, C₁₋₄ alkyl, or C₃₋₄ cycloalkyl. In certain embodiments, R^{6B} is hydrogen. In certain embodiments, R^{6B} is C₁₋₄ alkyl. In certain embodiments, R^{6B} is C₃₋₄ cycloalkyl. In certain embodiments, R^{6B} is selected from the groups depicted in the compounds in Table 1 below.

As defined generally above, R^{7B} represents independently for each occurrence C₁₋₄ alkyl or C₃₋₄ cycloalkyl. In certain embodiments, R^{7B} is C₁₋₄ alkyl. In certain embodiments, R^{7B} is methyl. In certain embodiments, R^{7B} is C₃₋₄ cycloalkyl. In certain embodiments, R^{7B} is selected from the groups depicted in the compounds in Table 1 below.

As defined generally above, R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} represents independently for each occurrence hydrogen or C₁₋₄ alkyl. In certain embodiments, R^{8B} is hydrogen. In certain embodiments, R^{8B} is C₁₋₄ alkyl. In certain embodiments, two occurrences of R^{8B} are taken together with the nitrogen atom to which they are attached to form a 3-7 membered heterocyclic ring containing 1 or 2 heteroatoms independently selected from nitrogen and oxygen. In certain embodiments, R^{8B} and R^{9B} are taken together with their intervening atoms to a form a 5-7 membered ring containing 1, 2, or 3 heteroatoms independently selected from nitrogen, oxygen, and sulfur. In certain embodiments, R^{8B} is selected from the groups depicted in the compounds in Table 1 below.

As defined generally above, R^{9B} is C₁₋₆ alkyl, C₁₋₆ haloalkyl, —(C₁₋₆ alkylene)-(C₃₋₆ cycloalkyl), or C₃₋₆ cycloalkyl. In certain embodiments, R^{9B} is C₁₋₆ alkyl or C₁₋₆ haloalkyl. In certain embodiments, R^{9B} is C₁₋₆ alkyl. In certain embodiments, R^{9B} is C₁₋₆ haloalkyl. In certain embodiments, R^{9B} is —(C₁₋₆ alkylene)-(C₃₋₆ cycloalkyl). In certain embodiments, R^{9B} is C₃₋₆cycloalkyl. In certain embodiments, R^{9B} is selected from the groups depicted in the compounds in Table 1 below.

In certain embodiments, p is 0. In certain embodiments, p is 1. In certain embodiments, p is 2. In certain embodiments, p is selected from the corresponding value in the groups depicted in the compounds in Table 1 below.

In certain embodiments, the EPL is

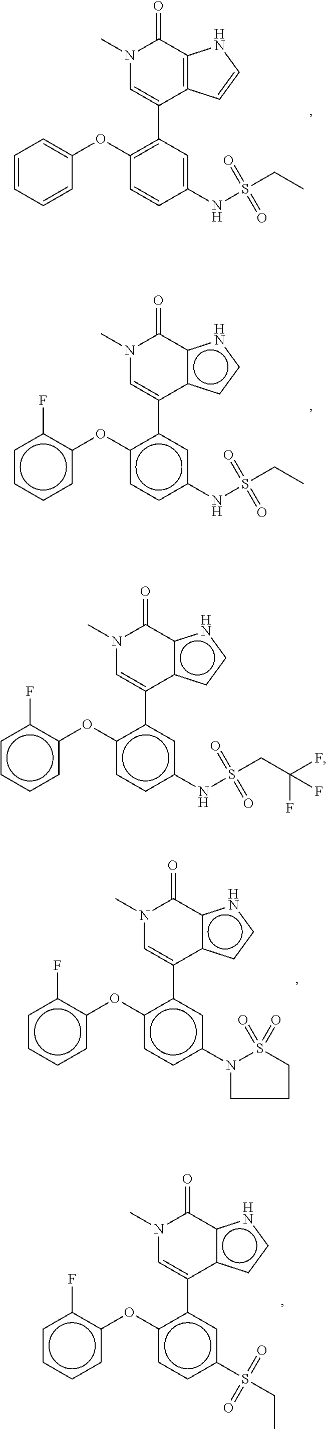

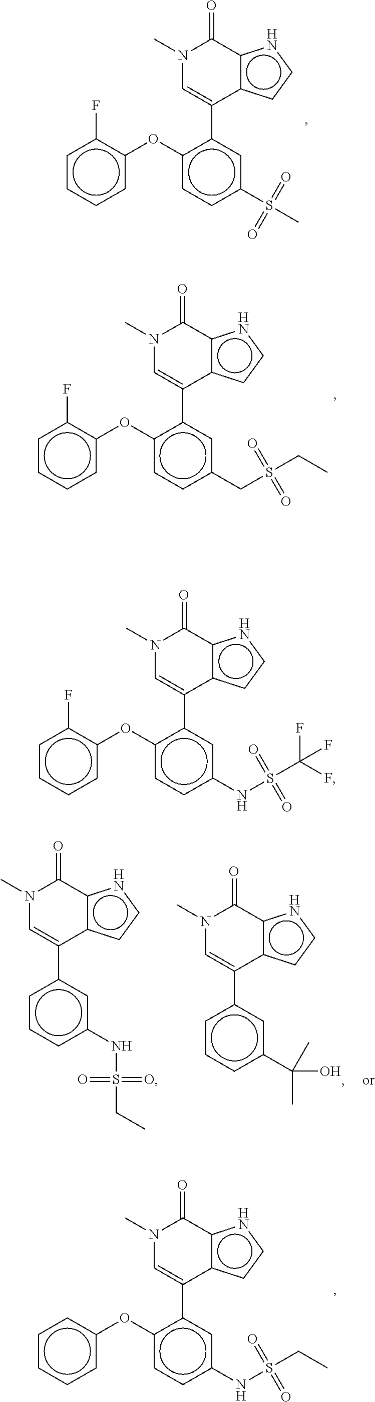

each of which is substituted by one occurrence of R^{II-2A} Wherein R^{II-2A} is a bond to L.

In certain embodiments, the EPL is

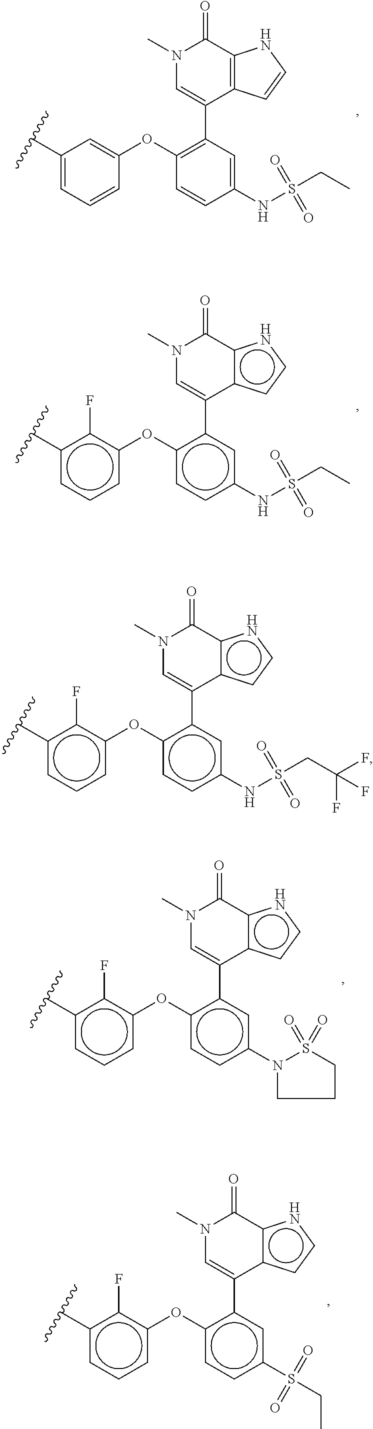

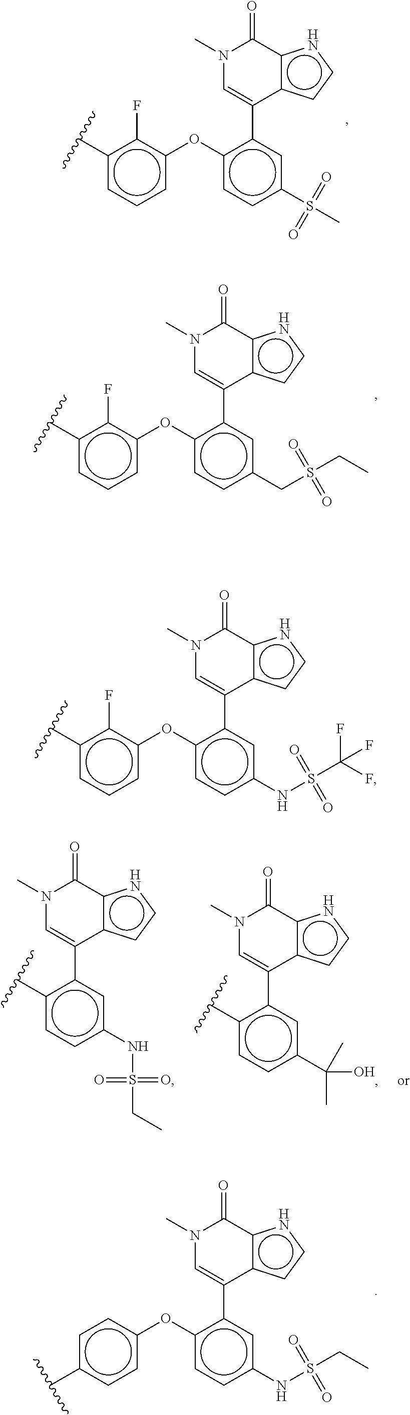

In certain embodiments, the EPL is defined by variable A² set forth above in connection with Formula I. In certain embodiments, the EPL is defined by one or more of the embodiments for variable A² set forth in connection with Formula I.

The compounds may be further characterized according to, for example, the identity of L. Exemplary further embodiments for L are provided in Part C below.

### Part C: Exemplary Further Description of Linker (L) Component of Compounds of Formula I and II

Compounds of Formula I and II may be further characterized according to, for example, the identity of the linker (L) component. A variety of linkers are known to one of skill in the art and may be used in the heterobifunctional compounds described herein. For example, in certain embodiments, L comprises one or more optionally substituted groups selected from amino acids, polyether chains, aliphatic groups, and any combinations thereof. In certain embodiments, L consists of one or more optionally substituted groups selected from amino acids, polyether chains, aliphatic groups, and any combinations thereof. In certain embodiments, L consists of one or more groups selected from amino acids, polyether chains, aliphatic groups, and any combinations thereof.

In some embodiments, L is symmetrical. In some embodiments, L is asymmetric. In certain embodiments, L is a bond.

In certain embodiments, L is a covalent bond or a bivalent C₁₋₃₀ saturated or unsaturated, straight or branched, hydrocarbon chain, wherein 1-15 methylene units of L are optionally and independently replaced by cyclopropylene, —N(H)—, —N(C₁₋₄ alkyl)-, —N(C₃₋₅ cycloalkyl)-, —O—, —C(O)—, —OC(O)—, —C(O)O—, —S—, —S(O)—, —S(O)₂—, —S(O)₂N(H)—, —S(O)₂N(C₁₋₄ alkyl)-, —S(O)₂N(C₃₋₅ cycloalkyl)-, —N(H)C(O)—, —N(C₁₋₄ alkyl)C(O)—, —N(C₃₋₅ cycloalkyl)C(O)—, —C(O)N(H)—, —C(O)N(C₁₋₄ alkyl)-, —C(O)N(C₃₋₅ cycloalkyl)-, phenylene, an 8-10 membered bicyclic arylene, a 4-7 membered saturated or partially unsaturated carbocyclylene, an 8-10 membered bicyclic saturated or partially unsaturated carbocyclylene, a 3-7 membered saturated or partially unsaturated heterocyclylene having 1-2 heteroatoms independently selected from nitrogen, oxygen, and sulfur, an 8-10 membered bicyclic saturated or partially unsaturated heterocyclylene having 1-2 heteroatoms independently selected from nitrogen, oxygen, and sulfur, a 5-6 membered heteroarylene having 1-4 heteroatoms independently selected from nitrogen, oxygen, and sulfur, or an 8-10 membered bicyclic heteroarylene having 1-5 heteroatoms independently selected from nitrogen, oxygen, or sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-11 membered heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-11 membered bicyclic heterocyclyl containing 1, 2, 3, or 4 heteroatoms independently selected from nitrogen, oxygen, and sulfur.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-11 membered spirocyclic heterocyclyl containing 1, 2, 3, or 4 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In yet other embodiments, L comprises a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units. In yet other embodiments, L is a diradical of a polyethylene glycol chain ranging in size from about 1 to about 12 ethylene glycol units, from about 1 to about 10 ethylene glycol units, from about 2 to about 6 ethylene glycol units, from about 2 to about 5 ethylene glycol units, or from about 2 to about 4 ethylene glycol units.

In certain embodiments, L is a heteroalkylene having from 4 to 30 atoms selected from carbon, oxygen, nitrogen, and sulfur. In certain embodiments, L is a heteroalkylene having from 4 to 20 atoms selected from carbon, oxygen, nitrogen, and sulfur. In certain embodiments, L is a heteroalkylene having from 4 to 10 atoms selected from carbon, oxygen, nitrogen, and sulfur. In certain embodiments, L is a heteroalkylene having from 4 to 30 atoms selected from carbon, oxygen, and nitrogen. In certain embodiments, L is a heteroalkylene having from 4 to 20 atoms selected from carbon, oxygen, and nitrogen. In certain embodiments, L is a heteroalkylene having from 4 to 10 atoms selected from carbon, oxygen, and nitrogen. In certain embodiments, L is a heteroalkylene having from 4 to 30 atoms selected from carbon and oxygen. In certain embodiments, L is a heteroalkylene having from 4 to 20 atoms selected from carbon and oxygen. In certain embodiments, L is a heteroalkylene having from 4 to 10 atoms selected from carbon and oxygen.

In additional embodiments, the L is an optionally substituted (poly)ethyleneglycol having between 1 and about 100 ethylene glycol units, between about 1 and about 50 ethylene glycol units, between 1 and about 25 ethylene glycol units, between about 1 and about 10 ethylene glycol units, between 1 and about 8 ethylene glycol units, between 1 and about 6 ethylene glycol units, between 2 and about 4 ethylene glycol units, or optionally substituted alkyl groups interdispersed with optionally substituted, O, N, S, P or Si atoms. In certain embodiments, L is substituted with an aryl, phenyl, benzyl, alkyl, alkylene, or heterocycle group.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted carbocyclyl, or optionally substituted heterocyclyl, wherein R** represents independently for each occurrence hydrogen, C₁₋₆ alkyl, or C₃₋₆ cycloalkyl.

In certain embodiments, L is a bivalent, saturated or unsaturated, straight or branched C₁₋₄₅ hydrocarbon chain, wherein 0-10 methylene units of the hydrocarbon are independently replaced with —O—, —S—, —N(R**)—, —OC(O)—, —C(O)O—, —S(O)—, —S(O)₂—, —N(R**)S(O)₂—, —S(O)₂N(R**)—, —N(R**)C(O)—, —C(O)N(R**)—, —OC(O)N(R**)—, —N(R**)C(O)O—, optionally substituted 3-10 membered carbocyclyl, or optionally substituted 3-10 membered heterocyclyl containing 1, 2, 3, or 4 heteroatoms selected from nitrogen, oxygen, and sulfur, wherein R** represents independently for each occurrence hydrogen, C₁₋₆ alkyl, or C₃₋₆ cycloalkyl.

In certain embodiments, L has the formula —N(R)-(optionally substituted 3-20 membered heteroalkylene)ₚ-CH₂—C(O)—, wherein R is hydrogen or optionally substituted C₁-C₆ alkyl, and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, 3, or 4 substituents independently selected from halogen, C₁-C₆ haloalkyl, C₃-C₆ cycloalkyl, hydroxyl, and cyano; R is hydrogen or optionally substituted C₁-C₆ alkyl; and p is 0 or 1.

In certain embodiments, L has the formula —N(R)-(3-20 membered heteroalkylene)ₚ-CH₂—C(O)—; wherein the 3-20 membered heteroalkylene is optionally substituted with 1, 2, or 3 substituents independently selected from halogen and C₁-C₆ haloalkyl; R is hydrogen or C₁-C₆ alkyl; and p is 0 or 1.

In certain embodiments, L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

In certain embodiments, L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

In certain embodiments, L is a bivalent, saturated or unsaturated, straight or branched C₅₋₄₀ hydrocarbon chain, wherein 1-20 methylene units of the hydrocarbon are independently replaced with —O—, —N(H)—, —N(C₁₋₆ alkyl)-, —N(H)C(O)—, —N(C₁₋₆ alkyl)C(O)—, —C(O)N(H)—, —C(O)N(C₁₋₆ alkyl)-, optionally substituted 3-10 membered carbocyclyl, or optionally substituted 3-10 membered heterocyclyl containing 1, 2, 3, or 4 heteroatoms independently selected from nitrogen, oxygen, and sulfur.

In certain embodiments, L is -(3-7 membered, monocyclic, saturated heterocyclic ring containing 1 or 2 heteroatoms selected from nitrogen)-(OCH₂CH₂)₁₋₁₅—O—***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₅—O—***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₆₋₁₀—O—***, wherein *** is the point of attachment to A².

In certain embodiments, L is -piperidinylene-(OCH₂CH₂)₁₋₁₅—O—***, wherein *** is the point of attachment to A².

In certain embodiments, L is

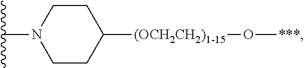

wherein *** is the point of attachment to A². In certain embodiments, L is

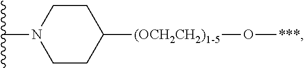

wherein *** is the point of attachment to A². In certain embodiments, L is

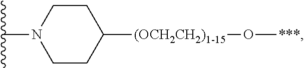

wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₁₅—N(H)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—N(C₁₋₄ alkyl)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—C(O)N(H)—C₁₋₁₀ alkylene-***, or -(3-7 membered, monocyclic, saturated heterocyclic ring containing 1 or 2 heteroatoms selected from nitrogen)-(OCH₂CH₂)₁₋₁₅—C(O)N(C₁₋₄ alkyl)-C₁₋₁₀ alkylene-***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₁₀—N(H)C(O)—C₁₋₅ alkylene-***, -(3-7 membered, monocyclic, saturated heterocyclic ring containing 1 or 2 heteroatoms selected from nitrogen)-(OCH₂CH₂)₁₋₁₀—N(C₁₋₄ alkyl)C(O)—C₁₋₅ alkylene-***, -(3-7 membered, monocyclic, saturated heterocyclic ring containing 1 or 2 heteroatoms selected from nitrogen)-(OCH₂CH₂)₁₋₁₀—C(O)N(H)—C₁₋₅ alkylene-***, or -(3-7 membered, monocyclic, saturated heterocyclic ring containing 1 or 2 heteroatoms selected from nitrogen)-OCH₂CH₂)₁₋₁₀—C(O)N(C₁₋₄ alkyl)-C₁₋₅ alkylene-***, wherein *** is the point of attachment to A².

In certain embodiments, L is -piperidinylene-(OCH₂CH₂)₁₋₅—N(H)C(O)—C₁₋₅ alkylene-***, -piperidinylene-(OCH₂CH₂)₁₋₅—N(C₁₋₄ alkyl)C(O)—C₁₋₅ alkylene-***, -piperidinylene-(OCH₂CH₂)₁₋₅—C(O)N(H)—C₁₋₅ alkylene-***, or -piperidinylene-(OCH₂CH₂)₁₋₅—C(O)N(C₁₋₄ alkyl)-C₁₋₅ alkylene-***, wherein *** is the point of attachment to A².

In certain embodiments, L is -(3-7 membered, monocyclic, saturated heterocyclic ring containing 1 or 2 heteroatoms selected from nitrogen)-(OCH₂CH₂)₁₋₁₀—***, -(3-7 membered, monocyclic, saturated heterocyclic ring containing 1 or 2 heteroatoms selected from nitrogen)-(C₀₋₁₀ alkylene)-O—***, or -(3-7 membered, monocyclic, saturated heterocyclic ring containing 1 or 2 heteroatoms selected from nitrogen)-C₁₋₁₀ alkylene, wherein *** is the point of attachment to A².

In certain embodiments, L is -piperidinylene-(OCH₂CH₂)₁₋₅—***, -piperidinylene-(C₀₋₅ alkylene)-O—***, or -piperidinylene-(C₁₋₅ alkylene)-***, wherein *** is the point of attachment to A².

In certain embodiments, L is -(3-7 membered monocyclic saturated heterocyclic ring containing 1 or 2 heteroatoms selected from nitrogen)-X¹—***, wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

In certain embodiments, L is -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

In certain embodiments, L is

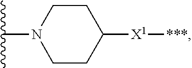

wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

In certain embodiments, L is -(piperazinylene)-X¹—***, wherein *** is the point of attachment to A², and X¹ is (i) C₁₋₅ alkylene where 1 or 2 methylene groups are optionally replaced by —O—, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

In certain embodiments, L is

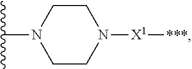

wherein *** is the point of attachment to A², and X¹ is (i) C₁₋₁₀ alkylene where 1 or 2 methylene groups are optionally replaced by —O—, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

In certain embodiments, L is -(3-7 membered monocyclic saturated heterocyclic ring containing 1 or 2 heteroatoms selected from nitrogen)-X²—(C₁₋₁₀ alkylene)-***, wherein *** is the point of attachment to A², and X² is —O—, —N(H)—, or —N(C₁₋₆ alkyl)-. In certain embodiments, L is -(3-7 membered monocyclic saturated heterocyclic ring containing 1 or 2 heteroatoms selected from nitrogen)-X²—(C₁₋₁₀ alkylene)-***, wherein *** is the point of attachment to A², and X² is —O—.

In certain embodiments, L is -(piperidinylene)-X²—(C₁₋₁₀ alkylene)-***, wherein *** is the point of attachment to A², and X² is —O—, —N(H)—, or —N(C₁₋₆ alkyl)-. In certain embodiments, L is -(piperidinylene)-X²—(C₁₋₁₀ alkylene)-***, wherein *** is the point of attachment to A², and X² is —O—.

In certain embodiments, L is -(piperidinylene)-X²-(a 3-7 membered monocyclic saturated heterocyclic ring containing 1 or 2 heteroatoms selected from nitrogen)-***, wherein *** is the point of attachment to A², and X² is —O—, —N(H)—, or —N(C₁₋₆ alkyl)-. In certain embodiments, L is -(piperidinylene)-X²-(a 3-7 membered monocyclic saturated heterocyclic ring containing 1 or 2 heteroatoms selected from nitrogen)-***, wherein *** is the point of attachment to A², and X² is —O—.

In certain embodiments, L is

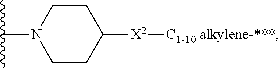

wherein *** is the point of attachment to A², and X² is —O—, —N(H)—, or —N(C₁₋₆ alkyl)-. In certain embodiments, L is

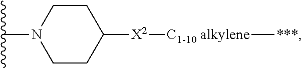

wherein *** is the point of attachment to A², and X² is —O—.

In certain embodiments, L is

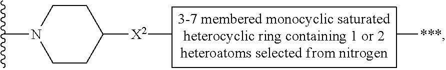

wherein *** is the point of attachment to A², and X² is —O—, —N(H)—, or —N(C₁₋₆ alkyl)-. In certain embodiments, L is

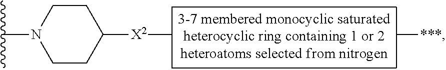

wherein *** is the point of attachment to A², and X² is —O—.

In certain embodiments, L is -(3-7 membered monocyclic saturated heterocyclic ring containing 1 or 2 heteroatoms selected from nitrogen)-X¹—***, wherein *** is the point of attachment to A², and X¹ is —(OCH₂CH₂)₁₋₁₀ where 1 CH₂ group is optionally replaced with —C(H)(C₃₋₆ cycloalkyl)-.

In certain embodiments, L is a 7-11 membered spirocyclic or fused bicyclic saturated heterocyclic ring containing 1, 2, or 3 heteroatoms selected from nitrogen and oxygen. In certain embodiments, L is a 7-8 membered spirocyclic or fused bicyclic saturated heterocyclic ring containing 2 heteroatoms selected from nitrogen.

In certain embodiments, L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond.

In certain embodiments, L is -(piperidinylene)-(C₁₋₅ alkylene)-(piperazinylene)-***, wherein *** is the point of attachment to A².

In certain embodiments, L is -(piperazinylene)-(azetidinylene)-*** or (azetidinylene)-(piperazinylene)-***, wherein *** is the point of attachment to A².

In certain embodiments, L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-(C₀₋₆ alkylene)-O—***, wherein X³ is C₁₋₁₀ alkylene, —O—, —N(H)—, —N(C₁₋₄ alkyl)-, or a bond, and *** is the point of attachment to A².

In certain embodiments, L is -(3-7 membered monocyclic saturated heterocyclic ring containing 1 or 2 heteroatoms selected from nitrogen)-(C₁₋₆ alkylene)-X³—(C₃₋₆ cycloalkylene)-(C₀₋₆ alkylene)-O—***, -(3-7 membered monocyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₁₋₆ alkylene)-X³—(C₃₋₆ cycloalkylene)-(C₀₋₆ alkylene)-O—***, X³—(C₃₋₆ cycloalkylene)-(C₀₋₆ alkylene)-O—***, -(3-7 membered monocyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₁₋₆ alkylene)-(C₃₋₆ cycloalkylene)-(C₀₋₆ alkylene)-O—***, or -(3-7 membered monocyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₀₋₆ alkylene)-O—***, wherein X³ is —N(H)— or —N(C₁. 4 alkyl)-, and *** is the point of attachment to A².

In certain embodiments, L is -(3-7 membered monocyclic saturated heterocyclic ring containing 1 or 2 heteroatoms selected from nitrogen)-X³—(C₃₋₆ cycloalkylene)-O—***, -(3-7 membered monocyclic saturated heterocyclic ring containing 1 or 2 heteroatoms selected from nitrogen)-X³—(C₃₋₆ cycloalkylene)-N(H)—***, or -(3-7 membered monocyclic saturated heterocyclic ring containing 1 or 2 heteroatoms selected from nitrogen)-X³—(C₃₋₆ cycloalkylene)-N(C₁₋₄ alkyl)-***, wherein *** is the point of attachment to A², and X³ is C₁₋₁₀ alkylene, —O—, —N(H)—, —N(C₁₋₄ alkyl)-, or a bond.

In certain embodiments, L is -(piperidinylene)-X³—(C₃₋₆ cycloalkylene)-O—***, -(piperidinylene)-X³—(C₃₋₆ cycloalkylene)-N(H)—***, or -(piperidinylene)-X³—(C₃₋₆ cycloalkylene)-***, wherein *** is the point of attachment to A², and X³ is C₁₋₁₀ alkylene, —O—, —N(H)—, —N(C₁₋₄ alkyl)-, or a bond.

In certain embodiments, L has the formula —(C₀₋₁₂ alkylene)-(optionally substituted 3-40 membered heteroalkylene)-(C₀₋₁₂ alkylene)-.

In certain embodiments, L is

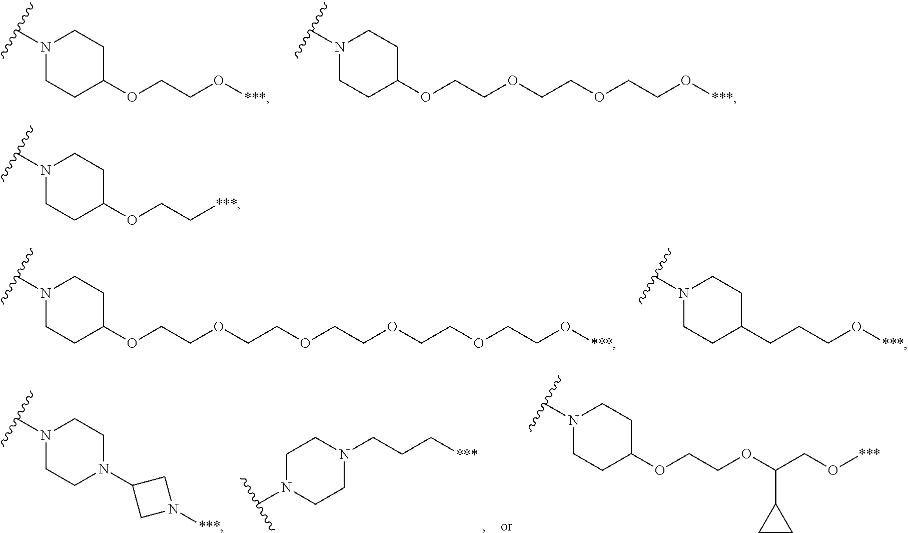

wherein *** is the point of attachment to A².

In certain embodiments, L is

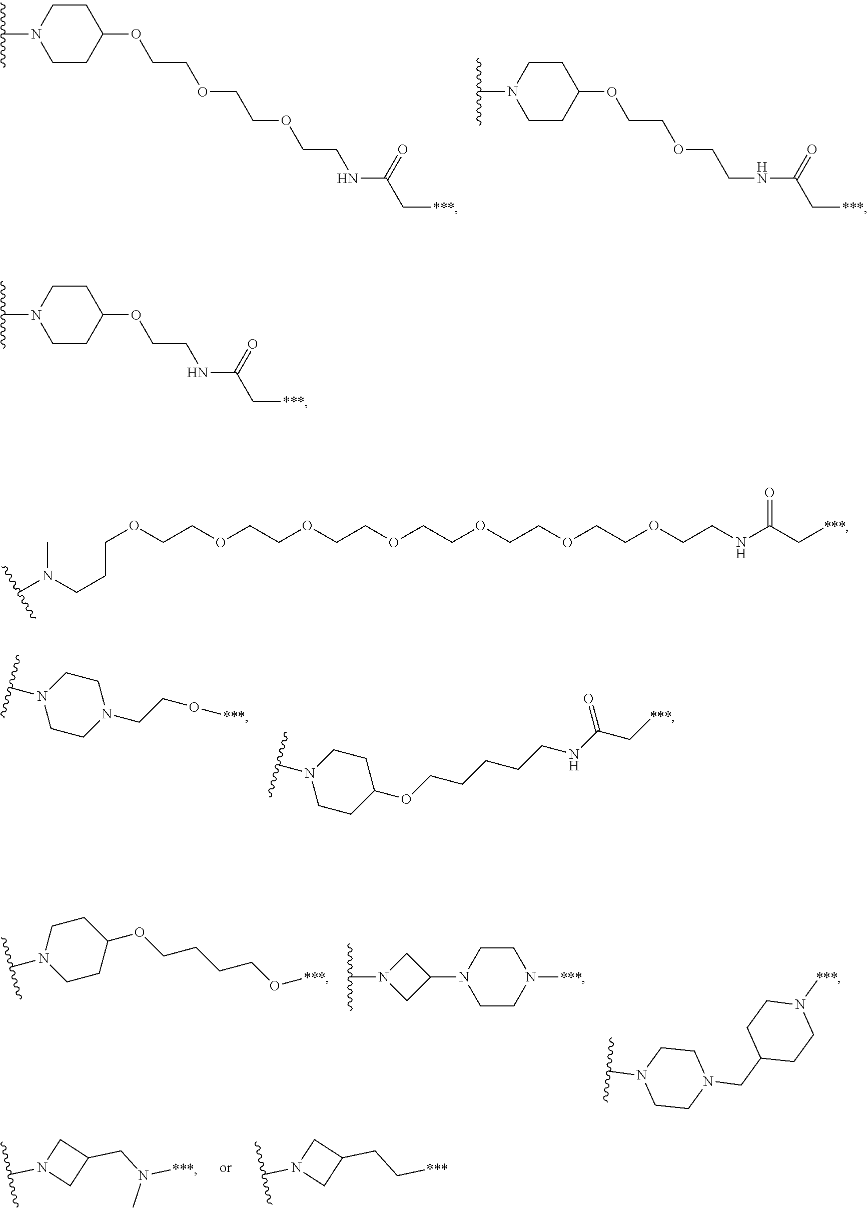

wherein *** is the point of attachment to A².

In certain embodiments, L is one of the following:

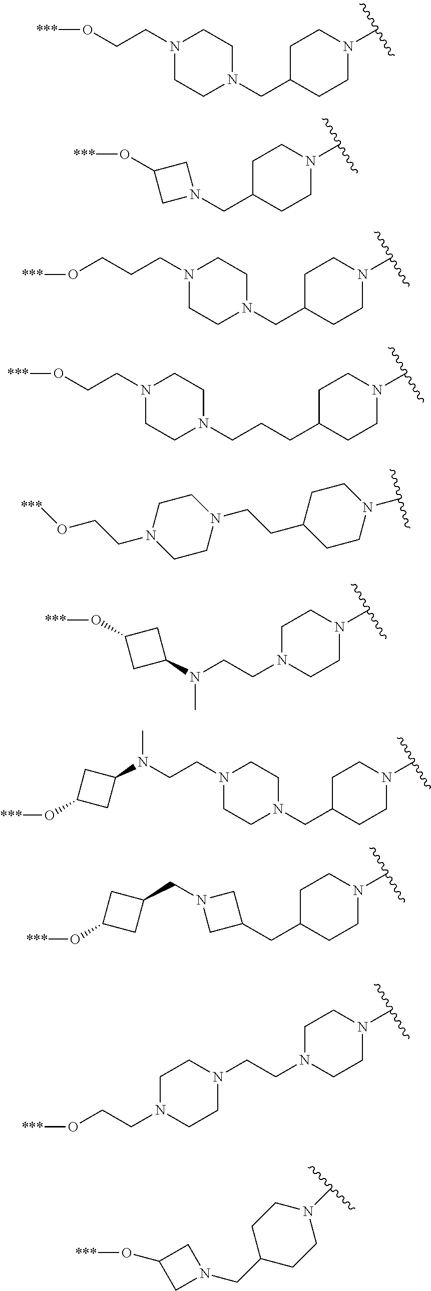

wherein *** is the point of attachment to A².

In certain embodiments, L is one of the following:

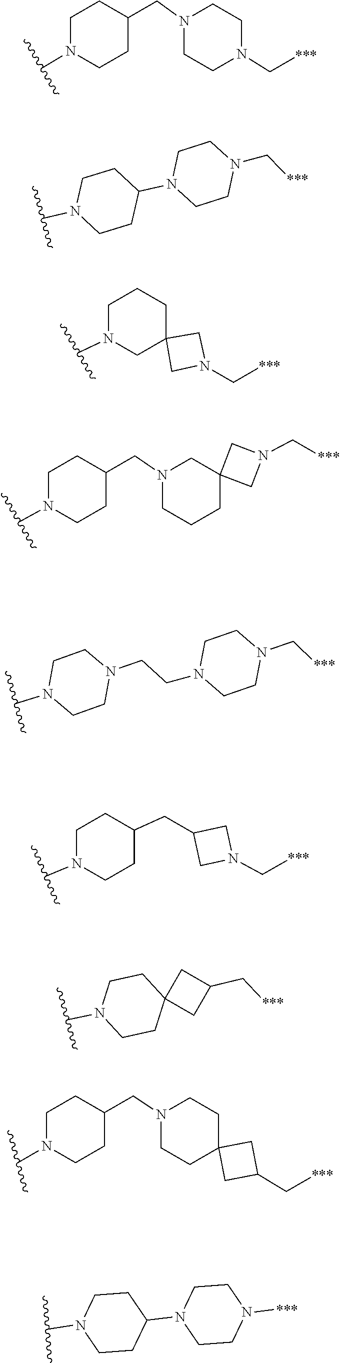

wherein *** is the point of attachment to A².

In certain embodiments, L is -(3-7 membered monocyclic saturated heterocyclic ring containing 1 or 2 heteroatoms selected from nitrogen)-(C₁₋₄ alkylene)-(3-7 membered monocyclic saturated heterocyclic ring containing 1 or 2 heteroatoms selected from nitrogen)-(C₁₋₄ alkylene)-*** or -(3-7 membered monocyclic saturated heterocyclic ring containing 1 or 2 heteroatoms selected from nitrogen)-(C₁₋₄ alkylene)-(7-11 membered spirocyclic saturated heterocyclic ring containing 1 or 2 heteroatoms selected from nitrogen)-(C₁₋₄ alkylene)-***, wherein *** is the point of attachment to A².

### Additional Exemplary Embodiments for L

In certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[(C₂₋₄ alkylene)-O-]₂₋₆-(C₁₋₆ alkylene)-C(O)—***, —N(H)—[(C₂₋₄ alkylene)-O-]₇₋₁₅-(C₁₋₆ alkylene)-C(O)—***, —N(H)—(C₁₋₆ alkylene)-C(O)—***, —N(H)—(C₇₋₁₅ alkylene)-C(O)—***, —N(H)—[(C₂₋₄ alkylene)-O-]₂₋₆-(C₁₋₆ alkylene)-***, —N(H)—[(C₂₋₄ alkylene)-O-]₇₋₁₅-(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[(C₂₋₄ alkylene)-O-]₂₋₆-(C₁₋₆ alkylene)-N(H)—(C₁₋₆ alkylene)-***, —N(H)—[(C₂₋₄ alkylene)-O-]₇₋₁₅-(C₁₋₆ alkylene)-N(H)—(C₁₋₆ alkylene)-***, —N(H)—[(C₂₋₄ alkylene)-O-]₂₋₆-(C₁₋₆ alkylene)-N(C₁₋₆ alkyl)-(C₁₋₆ alkylene)-***, or —N(H)—[(C₂₋₄ alkylene)-O-]₇₋₁₅-(C₁₋₆ alkylene)-N(C₁₋₆ alkyl)-(C₁₋₆ alkylene)-***, where *** is a point of attachment to A².

In certain embodiments, L is —N(H)—(C₂₋₉ alkylene)-O—(C₁₋₆ alkylene)-C(O)—***, —N(H)—(C₁₀₋₂₀ alkylene)-O—(C₁₋₆ alkylene)-C(O)—***, —N(H)—[CH₂CH₂—O-]₂₋₆-(C₁₋₆ alkylene)-C(O)—***, —N(H)—[CH₂CH₂—O-]₇₋₁₅-(C₁₋₆ alkylene)-C(O)—***, —N(H)—(C₁₋₆ alkylene)-C(O)—***, —N(H)—(C₇₋₁₅ alkylene)-C(O)—***, —N(H)—[CH₂CH₂—O-]₂₋₆-(C₁₋₆ alkylene)-***, —N(H)—[CH₂CH₂—O-]₇₋₁₅-(C₁₋₆ alkylene)-***, —N(H)—(C₂₋₉ alkylene)-O—(C₁₋₆ alkylene)-C(O)N(C₁₋₆ alkyl)-(C₁₋₆ alkylene)-***, —N(H)—(C₂₋₉ alkylene)-O—(C₁₋₆ alkylene)-C(O)N(H)—(C₁₋₆ alkylene)-***, —N(H)—[CH₂CH₂—O-]₂₋₆-(C₁₋₆ alkylene)-N(H)—(C₁₋₆ alkylene)-***, —N(H)—[CH₂CH₂—O-]₇₋₁₅-(C₁₋₆ alkylene)-N(H)—(C₁₋₆ alkylene)-***, —N(H)—[CH₂CH₂—O-]₂₋₆-(C₁₋₆ alkylene)-N(C₁₋₆ alkyl)-(C₁₋₆ alkylene)-***, or —N(H)—[CH₂CH₂—O-]₇₋₁₅-(C₁₋₆ alkylene)-N(C₁₋₆ alkyl)-(C₁₋₆ alkylene)-***, where *** is a point of attachment to A².

In certain embodiments, L is —N(H)—[(C₂₋₄ alkylene)-O-]₂₋₆-(C₁₋₆ alkylene)-C(O)—***, —N(H)—[(C₂₋₄ alkylene)-O-]₇₋₁₅-(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-***, —N(H)—[(C₂₋₄ alkylene)-O-]₂₋₆-(C₁₋₆ alkylene)-***, —N(H)—[(C₂₋₄alkylene)-O-]₇₋₁₅-(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to A².

In certain embodiments, L is —N(H)—[CH₂CH₂—O-]₂₋₆-(C₁₋₆ alkylene)-C(O)—***, —N(H)—[CH₂CH₂—O-]₇₋₁₅-(C₁₋₆ alkylene)-C(O)—***, —N(H)—(C₁₋₆ alkylene)-N(C₁₋₆ alkyl)C(O)—(C₁₋₆ alkylene)***, —N(H)—(C₁₋₆ alkylene)-N(H)C(O)—(C₁₋₆ alkylene)***, —N(H)—(C₂₋₆ alkylene)-***, —N(H)—(C₇₋₁₅ alkylene)-***, —N(C₁₋₆ alkyl)-(C₂₋₆ alkylene)-***, —N(C₁₋₆ alkyl)-(C₇₋₁₅ alkylene)-*** —N(H)—[CH₂CH₂—O-]₂₋₃-(C₁₋₆ alkylene)-***, —N(H)—[CH₂CH₂—O-]₇₋₁₅-(C₁₋₆ alkylene)-***, —N(H)—(C₁₋₆ alkylene)-(3-6 membered heterocycloalkylene)-(C₁₋₆ alkylene)-N(C₁₋₆ alkyl)-(C₁₋₆ alkylene)-***, —N(H)—(C₁₋₆ alkylene)-(3-6 membered heterocycloalkylene)-(C₁₋₆ alkylene)-N(H)—(C₁₋₆ alkylene)-***, —N(H)—(C₂₋₆ alkylene)-N(H)—(C₁₋₆ alkylene)-***, or —N(H)—(C₂₋₆ alkylene)-N(C₁₋₆ alkyl)-(C₁₋₆ alkylene)-***, where *** is a point of attachment to A².

In certain embodiments, L is —[(C₂₋₄alkylene)-O-]₂₋₆-(C₁₋₆ alkylene)-***, —[(C₂₋₄ alkylene)-O-]₇₋₁₅-(C₁₋₆ alkylene)-***, —[(C₂₋₄ alkylene)-O-]₂₋₆-(C₁₋₆ alkylene)-N(C₁₋₆ alkyl)(C₁₋₆ alkylene)-***, —[(C₂₋₄ alkylene)-O-]₇-(C₁₋₆ alkylene)-N(C₁₋₆ alkyl)(C₁₋₆ alkylene)-***, —[(C₂₋₄ alkylene)-O-]₂₋₆-(C₁₋₆ alkylene)-N(H)(C₁₋₆ alkylene)-***, —[(C₂₋₄ alkylene)-O-]₇₋₁₅-(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[(C₂₋₄ alkylene)-O-]₂₋₆-(C₁₋₆ alkylene)-***, —(C₁₋₉ alkylene)-N(H)C(O)—[(C₂₋₄alkylene)-O-]₂₋₆-(C₁₋₆ alkylene)-***, —(C₁₋₉ alkylene)-C(O)N(H)—[(C₂₋₄ alkylene)-O-]₇₋₁₅-(C₁₋₆ alkylene)-***, —(C₁₋₉ alkylene)-N(H)C(O)—[(C₂₋₄ alkylene)-O-]₇₋₁₅-(C₁₋₆ alkylene)-***, —(C₁₋₉ alkylene)-C(O)N(H)—[(C₂₋₄ alkylene)-O-]₂₋₆-(C₁₋₆ alkylene)-N(C₁₋₆ alkyl)-(C₁₋₆ alkylene)-***, —(C₁₋₉ alkylene)-N(H)C(O)—[(C₂₋₄ alkylene)-O-]₂₋₆-(C₁₋₆ alkylene)-N(C₁₋₆ alkyl)-(C₁₋₆ alkylene)-***, —(C₁₋₉ alkylene)-C(O)N(H)—[(C₂₋₄ alkylene)-O-]₇₋₁₅-(C₁₋₆ alkylene)-N(C₁₋₆ alkyl)-(C₁₋₆ alkylene)-***, or —(C₁₋₉ alkylene)-N(H)C(O)—[(C₂₋₄ alkylene)-O-]₇₋₁₅-(C₁₋₆ alkylene)-N(C₁₋₆ alkyl)-(C₁₋₆ alkylene)-***, where *** is a point of attachment to A².

In certain embodiments, L is —[CH₂CH₂—O-]₂₋₆-(C₁₋₆ alkylene)-***, —[CH₂CH₂—O-]₇₋₁₅-(C₁₋₆ alkylene)-***, —[CH₂CH₂—O-]₂₋₆-(C₁₋₆ alkylene)-N(C₁₋₆ alkyl)(C₁₋₆ alkylene)-***, —[CH₂CH₂-0-]₇₋₁₅-(C₁₋₆ alkylene)-N(C₁₋₆ alkyl)(C₁₋₆ alkylene)-***, —[CH₂CH₂—O-]₂₋₆-(C₁₋₆ alkylene)-N(H)(C₁₋₆ alkylene)-***, —[CH₂CH₂—O-]₇₋₁₅-(C₁₋₆ alkylene)-N(H)(C₁₋₆ alkylene)-***, —(C₁₋₉ alkylene)-C(O)N(H)—(C₁₋₆ alkylene)-***, —(C₁₋₉ alkylene)-N(H)C(O)—(C₁₋₆ alkylene)-***, —(C₁₋₉ alkylene)-C(O)N(H)—[CH₂CH₂—O-]₂₋₆-(C₁₋₆ alkylene)-***, —(C₁₋₉ alkylene)-N(H)C(O)—[CH₂CH₂—O-]₂₋₆-(C₁₋₆ alkylene)-***, —(C₁₋₉ alkylene)-C(O)N(H)—[CH₂CH₂—O-]₇₋₁₅-(C₁₋₆ alkylene)-***, —(C₁₋₉ alkylene)-N(H)C(O)—[CH₂CH₂—O-]₇₋₁₅-(C₁₋₆ alkylene)-***, —(C₁₋₉ alkylene)-C(O)N(H)—[CH₂CH₂—O-]₂₋₆-(C₁₋₆ alkylene)-N(C₁₋₆ alkyl)-(C₁₋₆ alkylene)-***, —(C₁₋₉ alkylene)-N(H)C(O)—[CH₂CH₂—O-]₂₋₆-(C₁₋₆ alkylene)-N(C₁₋₆ alkyl)-(C₁₋₆ alkylene)-***, —(C₁₋₉ alkylene)-C(O)N(H)—[CH₂CH₂—O-]₇₋₁₅-(C₁₋₆ alkylene)-N(C₁₋₆ alkyl)-(C₁₋₆ alkylene)-***, or —(C₁₋₉ alkylene)-N(H)C(O)—[(CH₂CH₂—O-]₇₋₁₅-(C₁₋₆ alkylene)-N(C₁₋₆ alkyl)-(C₁₋₆ alkylene)-***, where *** is a point of attachment to A².

In certain embodiments, L is —N(H)—[(C₂₋₄ alkylene)-O-]₂₋₆-(C₁₋₆ alkylene)-N(H)—***, —N(H)—[(C₂₋₄ alkylene)-O-]₇₋₁₅-(C₁₋₆ alkylene)-N(H)—***, —N(C₁₋₆ alkyl)-[(C₂₋₄ alkylene)-O-]₂₋₆-(C₁₋₆ alkylene)-N(H)—***, —N(C₁₋₆ alkyl)-[(C₂₋₄ alkylene)-O-]₇₋₁₅-(C₁₋₆ alkylene)-N(H)—***, —N(C₁₋₆ alkyl)-[(C₂₋₄ alkylene)-O-]₂₋₆-(C₁₋₆ alkylene)-N(C₁₋₆ alkyl)-***, or —N(C₁₋₆ alkyl)-[(C₂₋₄ alkylene)-O-]₇₋₁₅-(C₁₋₆ alkylene)-N(C₁₋₆ alkyl)-***, where *** is a point of attachment to A².

In certain embodiments, L is —N(H)—[CH₂CH₂—O-]₂₋₆-(C₁₋₆ alkylene)-N(H)—***, —N(H)—[CH₂CH₂—O-]₇₋₁₅-(C₁₋₆ alkylene)-N(H)—***, —N(C₁₋₆ alkyl)-[CH₂CH₂—O-]₂₋₆-(C₁₋₆ alkylene)-N(H)—***, —N(C₁₋₆ alkyl)-[CH₂CH₂—O-]₇₋₁₅-(C₁₋₆ alkylene)-N(H)—***, —N(C₁₋₆ alkyl)-[CH₂CH₂—O-]₂₋₆-(C₁₋₆ alkylene)-N(C₁₋₆ alkyl)-***, or —N(C₁₋₆ alkyl)-[CH₂CH₂—O-]₇₋₁₅-(C₁₋₆ alkylene)-N(C₁₋₆ alkyl)-***, where *** is a point of attachment to A².

In some embodiments, L is one of the following:

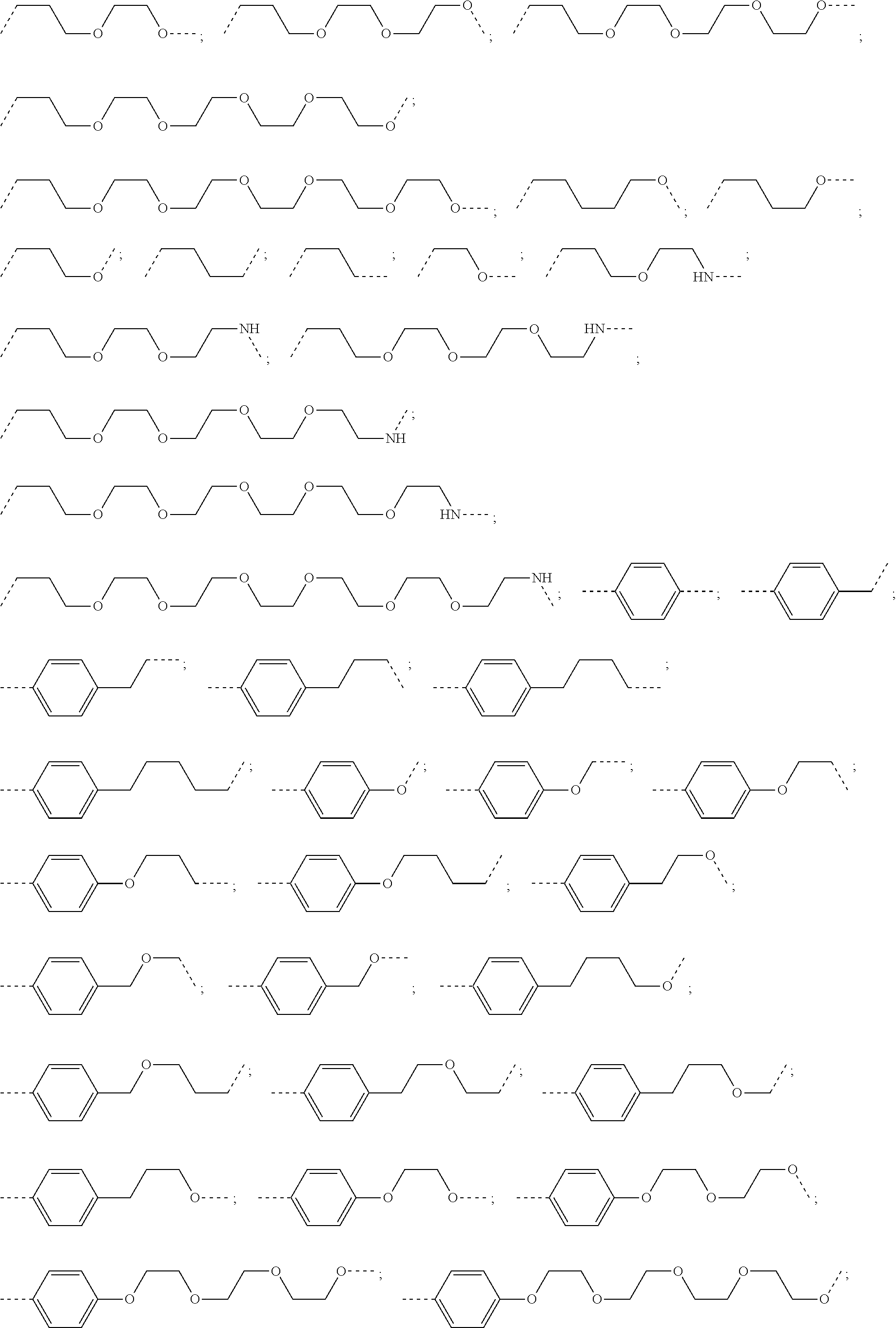

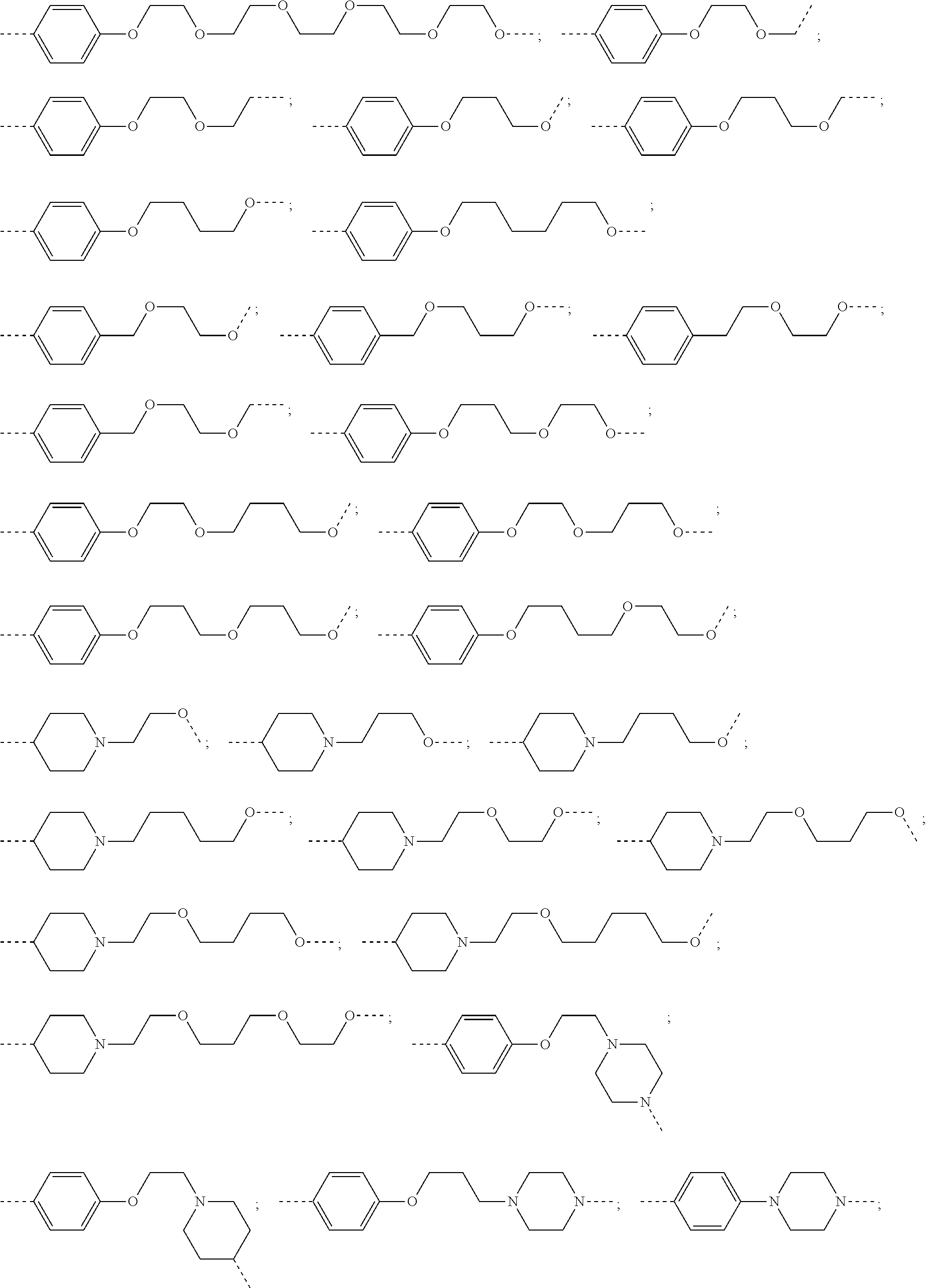

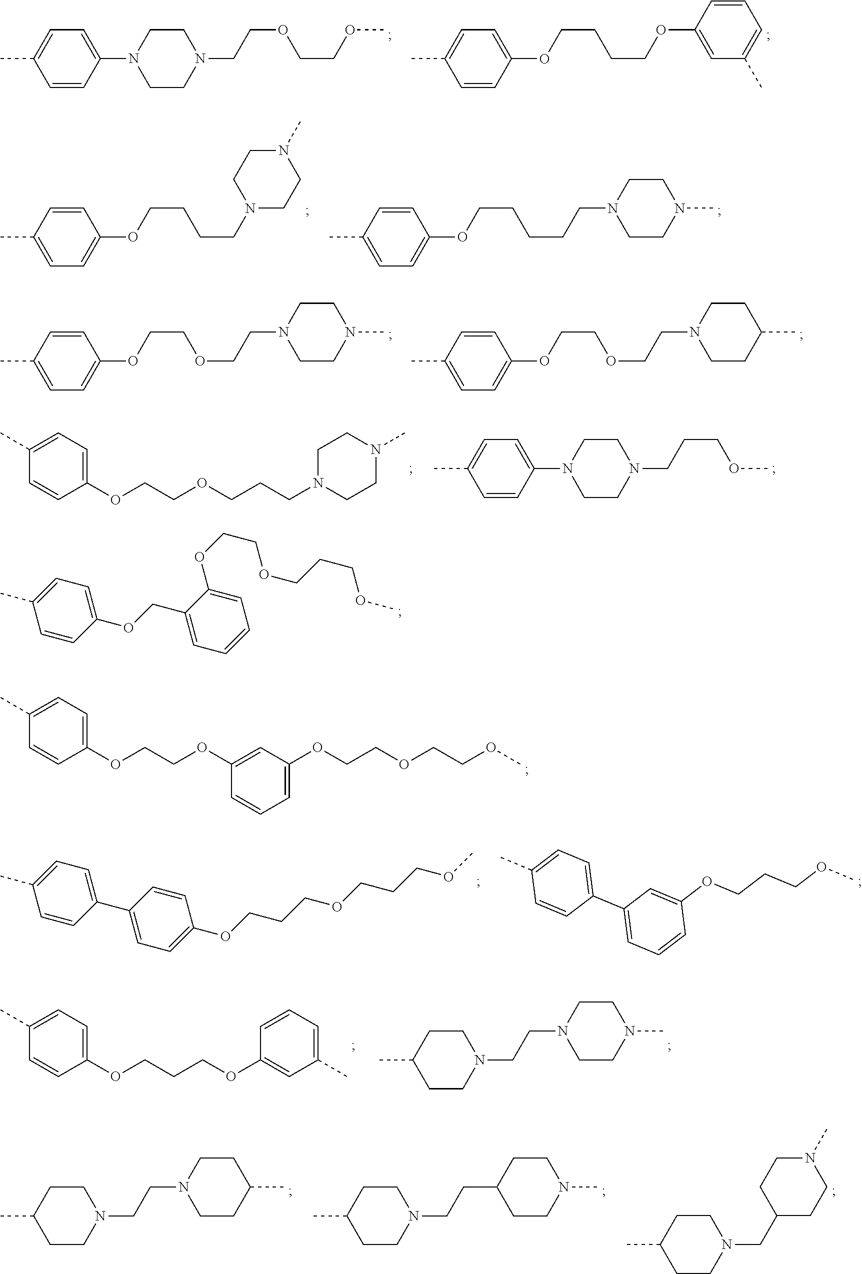

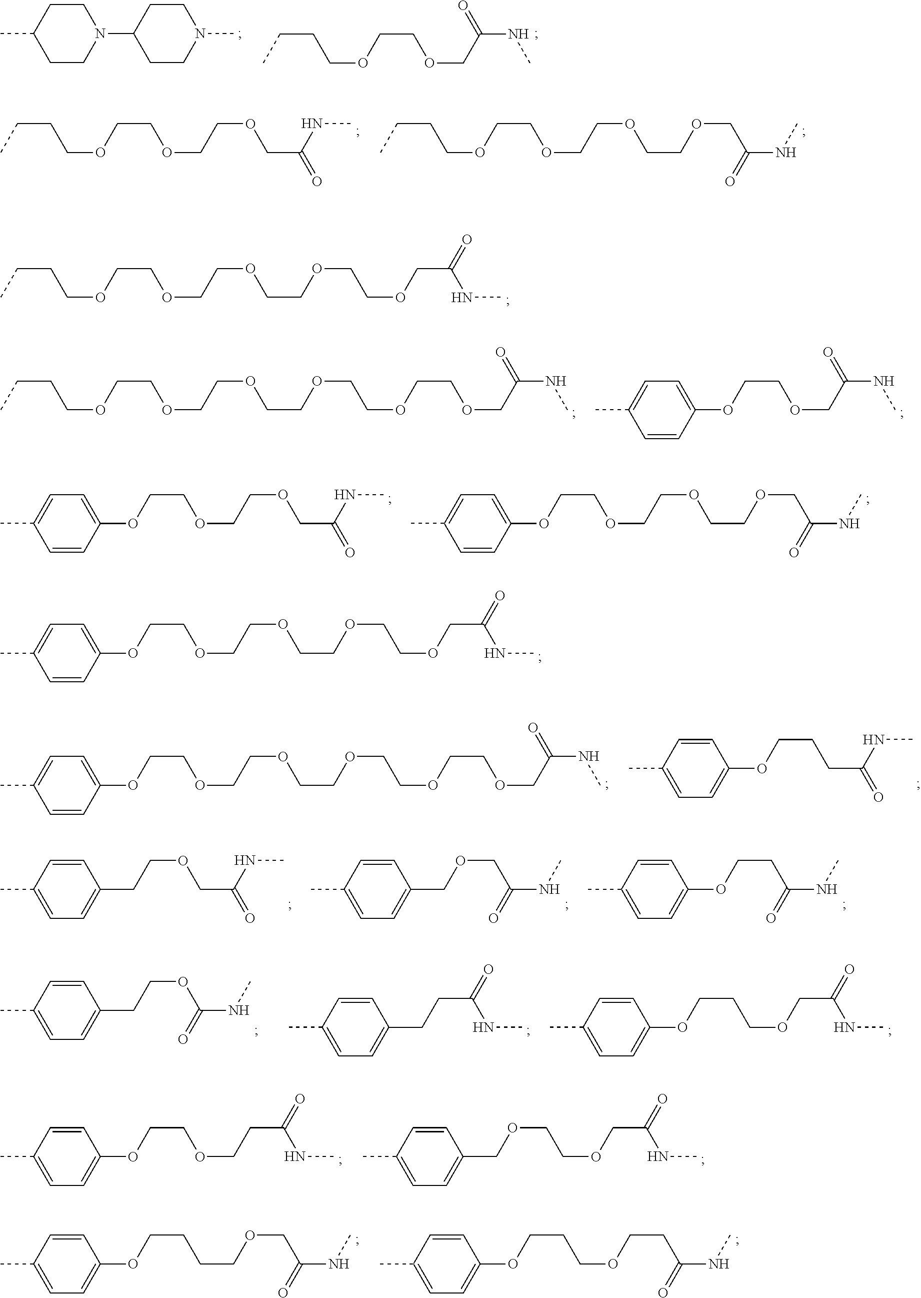

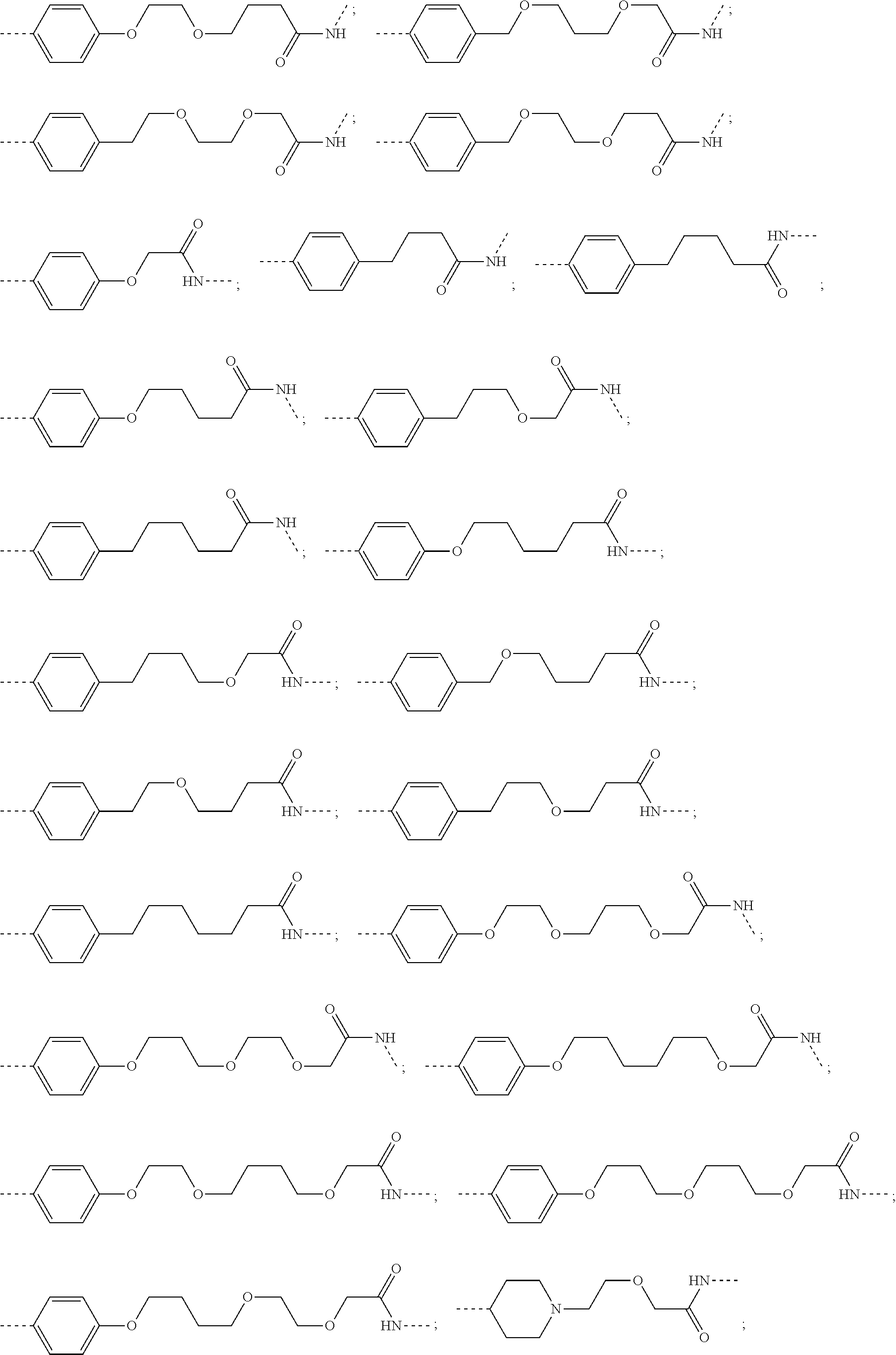

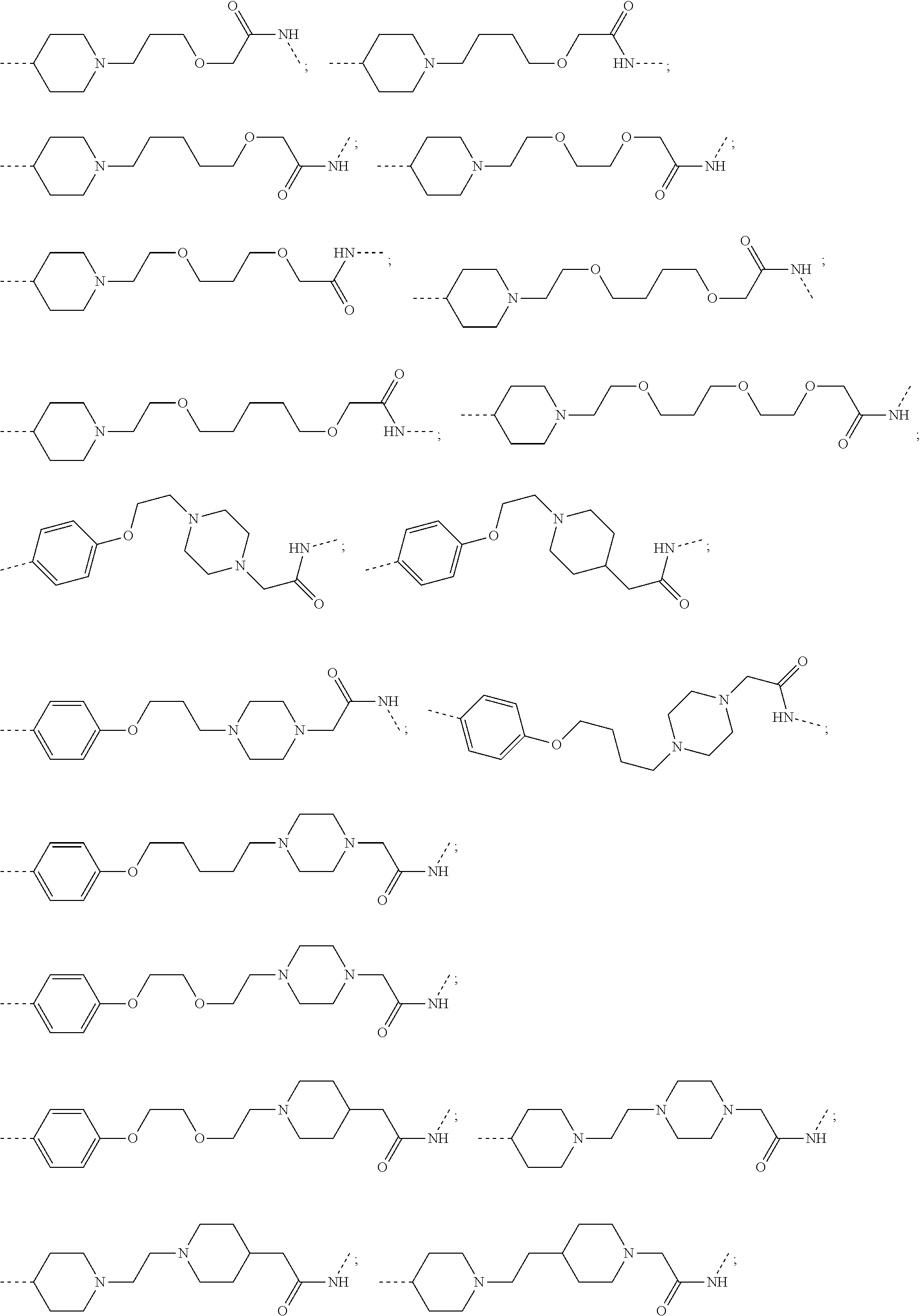

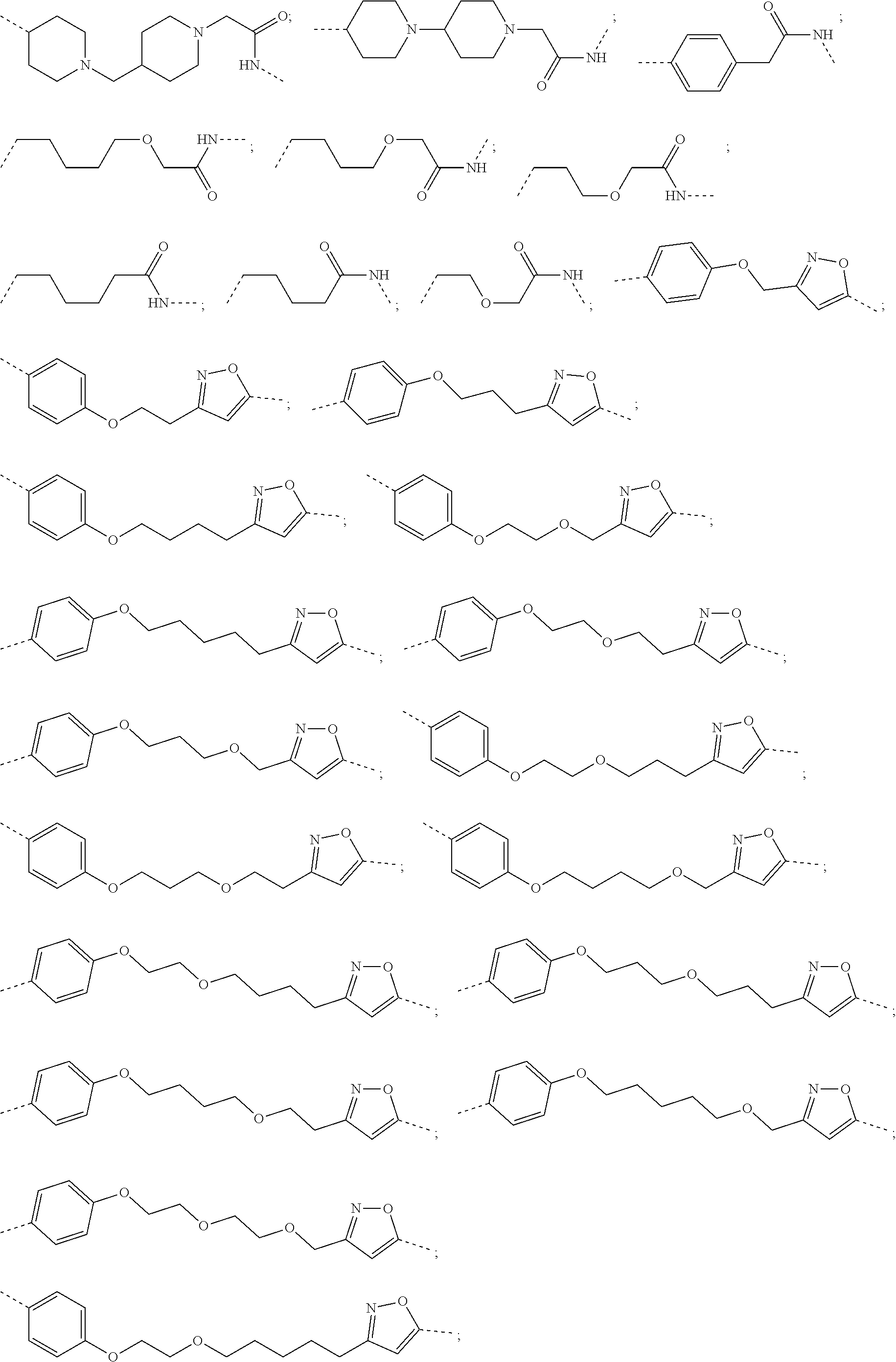

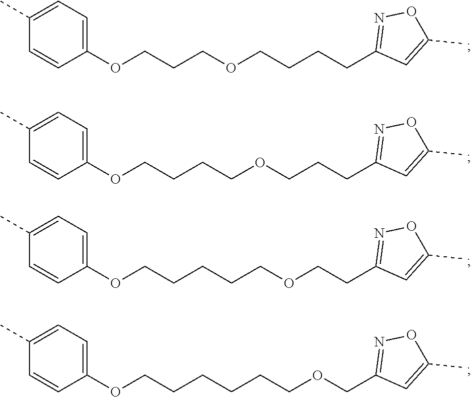

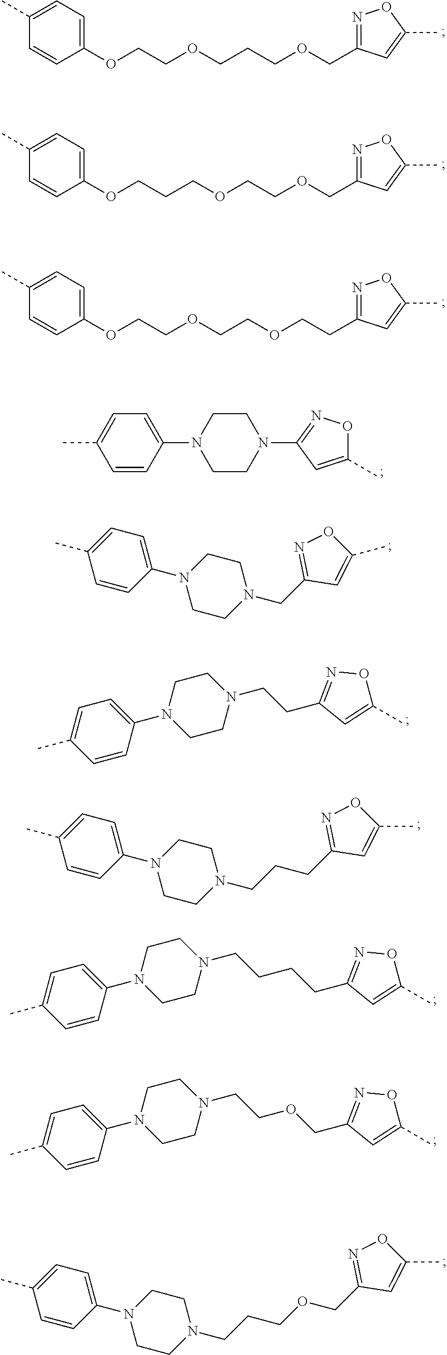

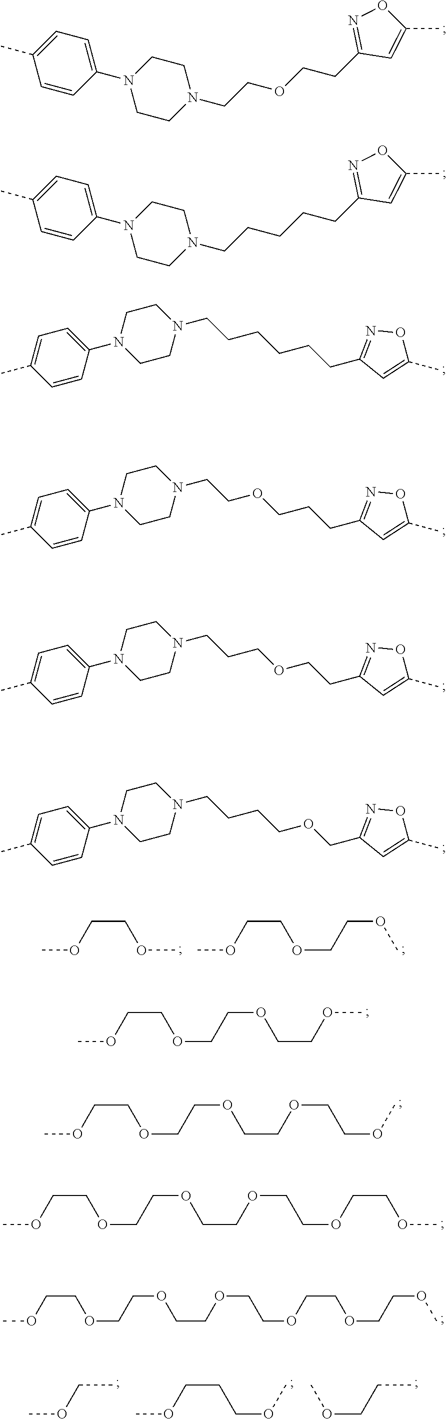

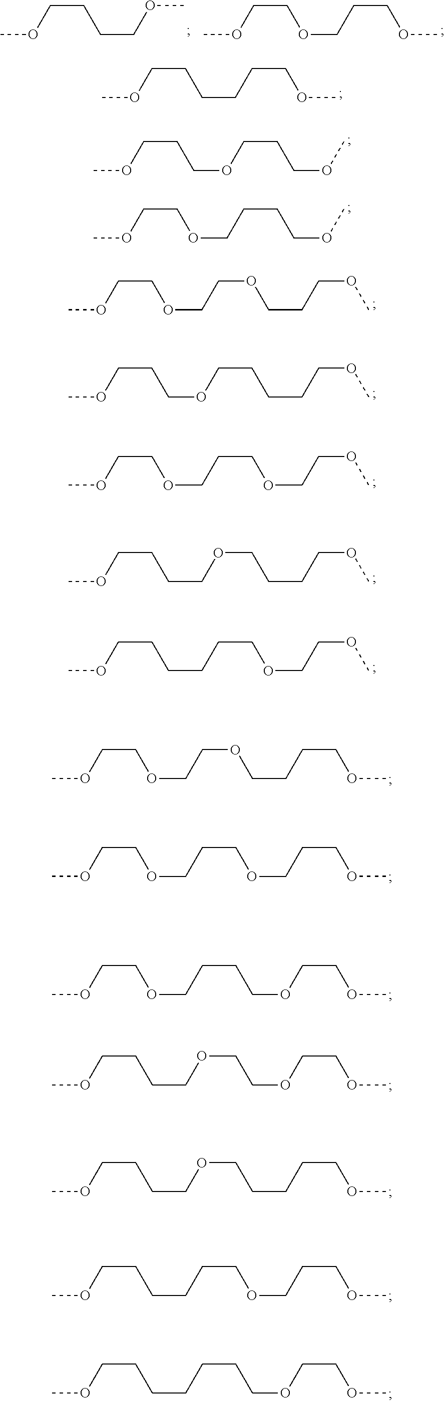

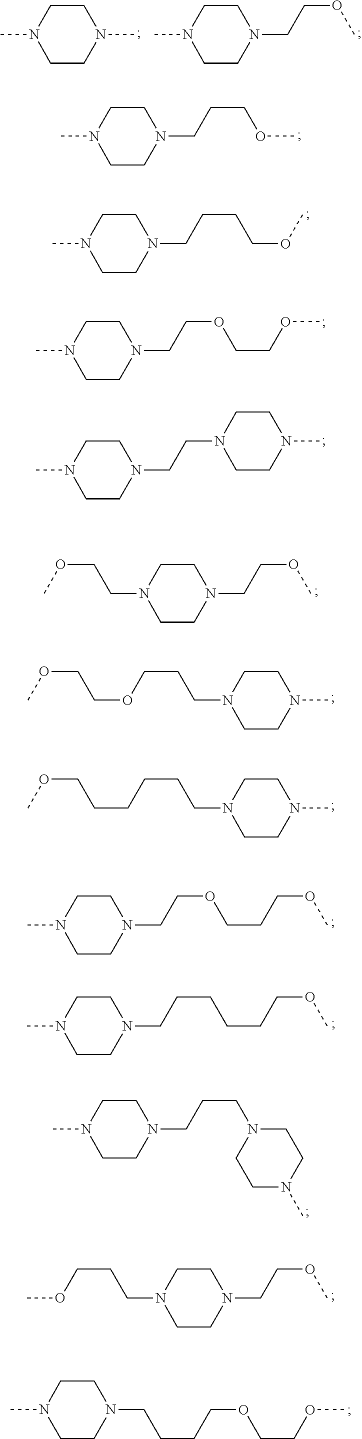

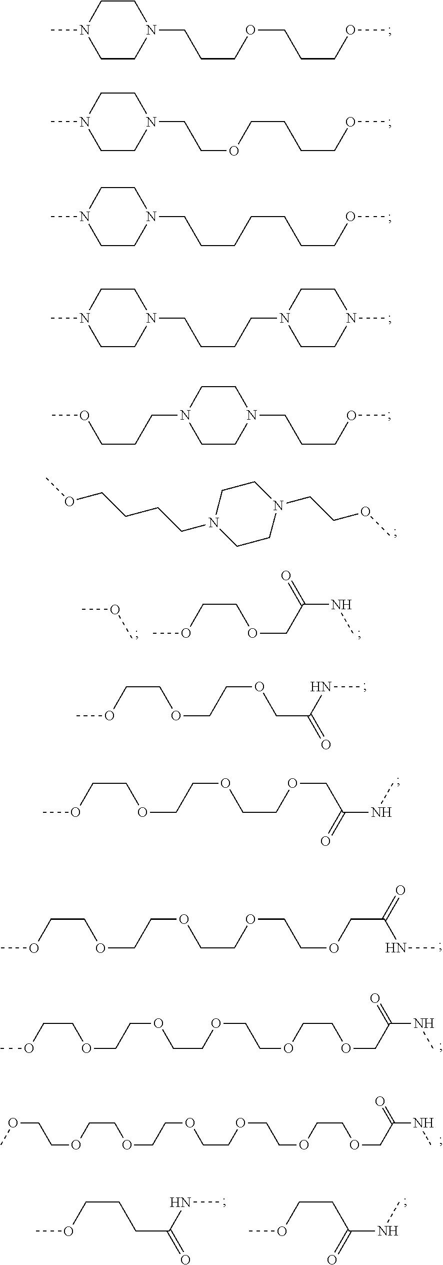

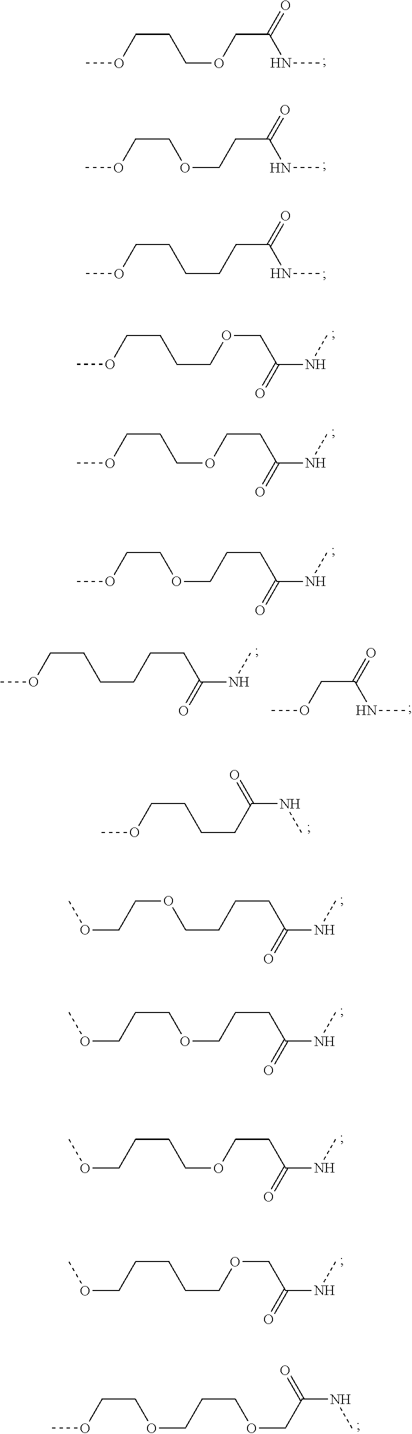

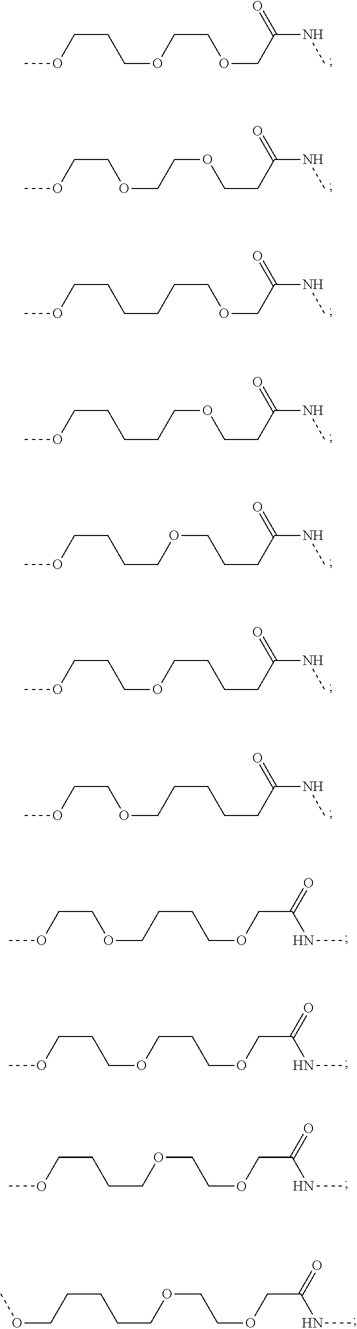

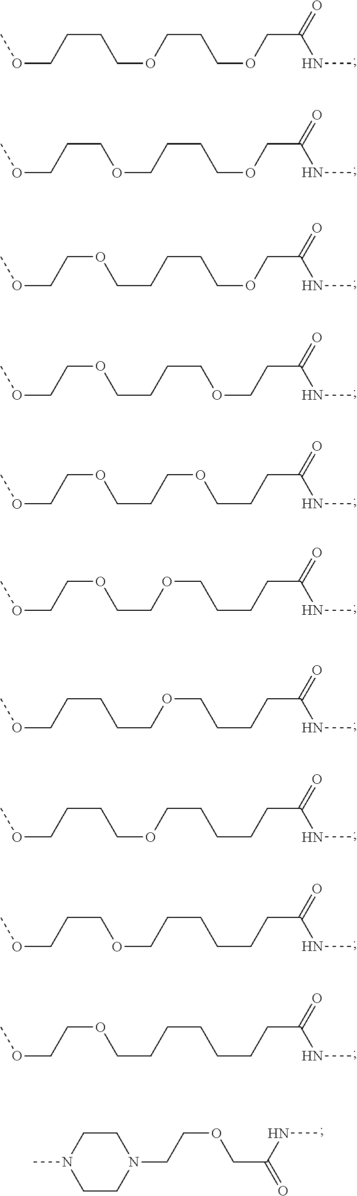

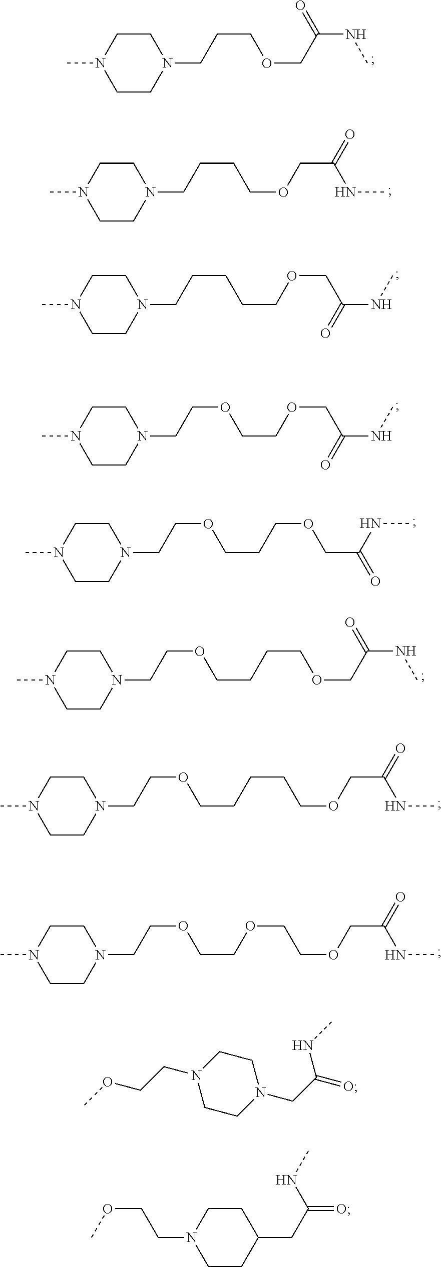

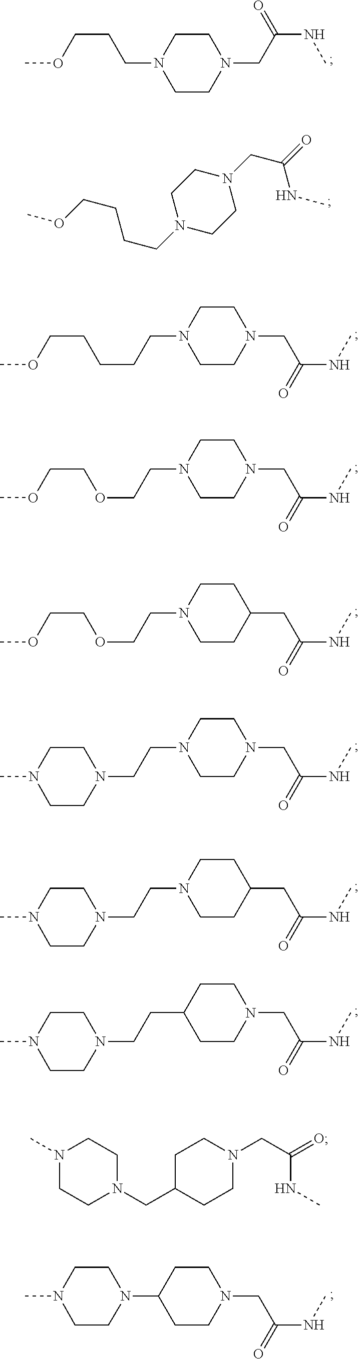

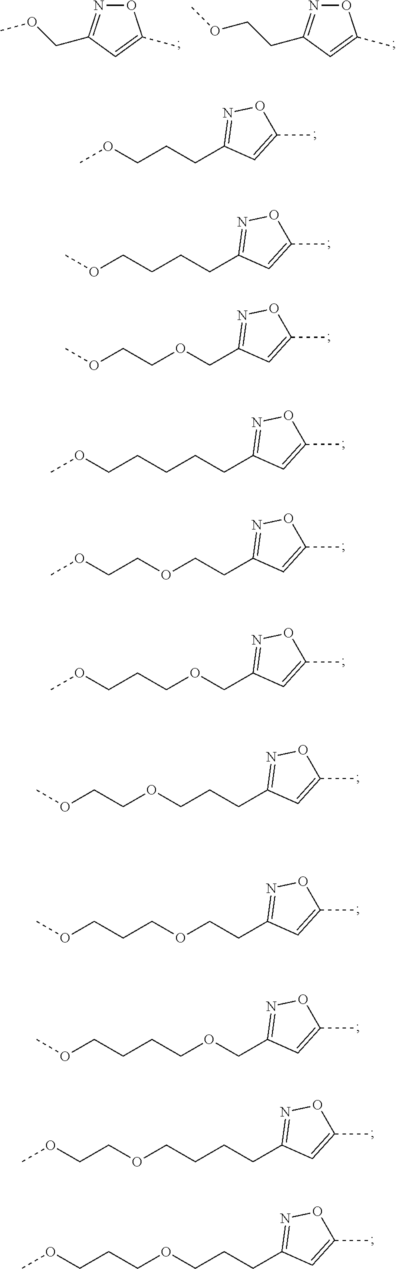

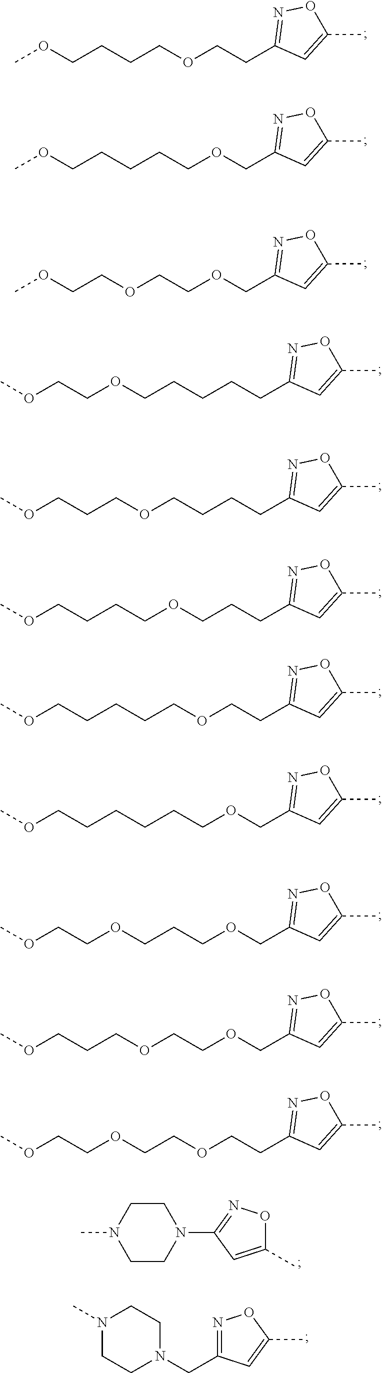

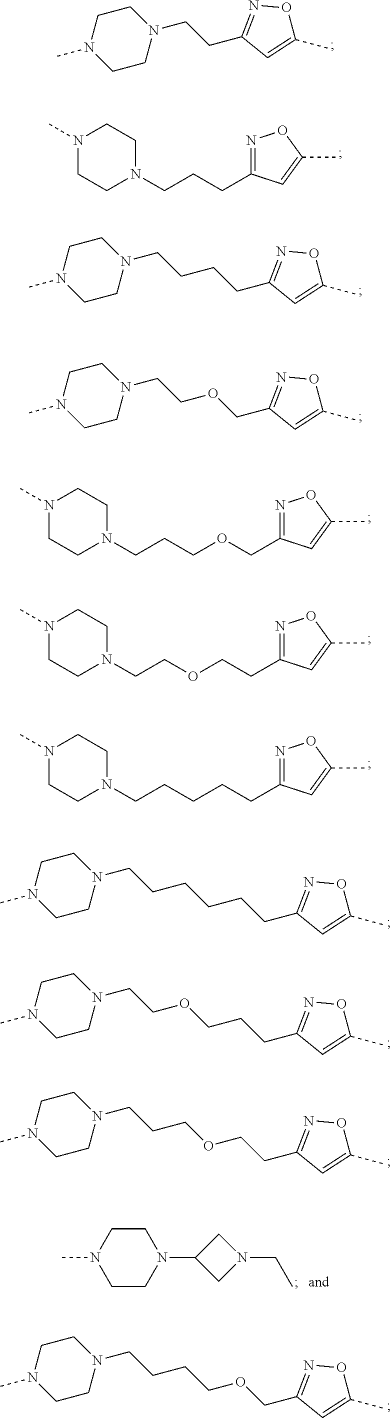

wherein a dashed bond indicates a point of attachment.

In certain embodiments, L is —C(H)(R¹⁰⁰)—, —C(R¹⁰⁰)₂—, O, —N(R¹⁰⁰)—, —S(O)₂—, an optionally substituted C₃₋₇ cycloalkylene, an optionally substituted C₄₋₇ cycloalkenylene, or an optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is —CH₂—Y²⁰—, —C(H)(R¹⁰⁰)—Y²⁰—, —C(R¹⁰⁰)₂—Y²⁰—, —O—Y²⁰—, —N(R¹⁰¹)—Y²⁰—, —S(O)₂—Y²⁰—, —C(O)—Y²⁰—, -(optionally substituted C₃₋₇ cycloalkylene)-Y²⁰—, -(optionally substituted C₄₋₇ cycloalkenylene)-Y²⁰—, -(optionally substituted 3-7 membered heterocyclylene containing 1, 2 or 3 heteroatoms selected from oxygen, nitrogen, and sulfur)-Y²⁰-, —Y²⁰—CH₂—, —Y²⁰—C(H)(R¹⁰¹)—, —Y²⁰—C(R¹⁰⁰)₂—, —Y²⁰—O—, —Y²⁰—N(R¹⁰¹)—, —Y²⁰—S(O)₂—, —Y²⁰—C(O)—, —Y²⁰-(optionally substituted C₃₋₇ cycloalkylene)-, —Y²⁰-(optionally substituted C₄₋₇ cycloalkenylene)-, or —Y²⁰-(optionally substituted 3-7 membered heterocyclylene containing 1, 2 or 3 heteroatoms selected from oxygen, nitrogen, and sulfur)-; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

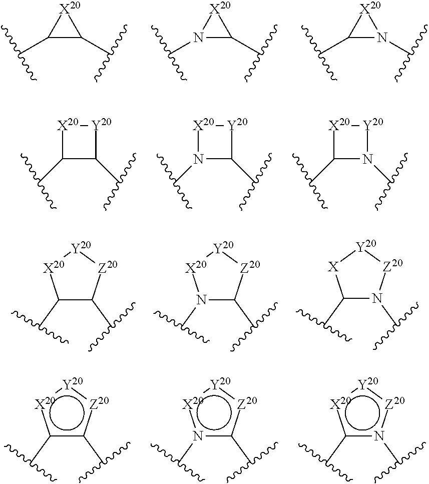

wherein X²⁰, Y²⁰, and Z²⁰ are independently —CH₂—, —C(H)(R¹⁰⁰), —C(R¹⁰⁰)₂, O, —N(R¹⁰¹)—, —S(O)₂—, —C(O)—, an optionally substituted C₃₋₇ cycloalkylene, or an optionally substituted C₄₋₇ cycloalkenylene; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following.

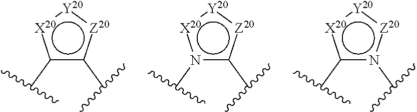

wherein X²⁰, Y²⁰, and Z²⁰ are independently —C(R¹⁰⁰)— or —N—; wherein R¹⁰⁰ represents independently for each occurrence hydrogen, halogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl.

In certain embodiments, L is —X²⁰—Y²⁰—Z²⁰—, wherein X²⁰, Y²⁰, and Z²⁰ are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is —X²⁰=Y²⁰−Z²¹—, wherein X²⁰ and Y²⁰ are independently —C(R¹⁰⁰)— or —N—, and Z²¹ is —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, or an optionally substituted C₄₋₇ cycloalkenylene; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is —C≡C—Z²⁰—, wherein Z²⁰ is —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, or an optionally substituted C₄₋₇ cycloalkenylene; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

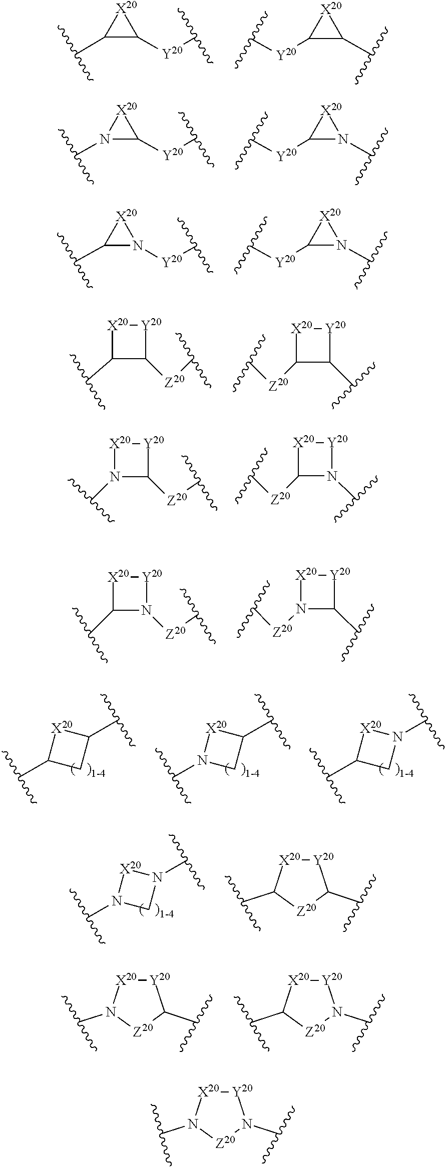

wherein X²⁰, Y²⁰, and Z²⁰ are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

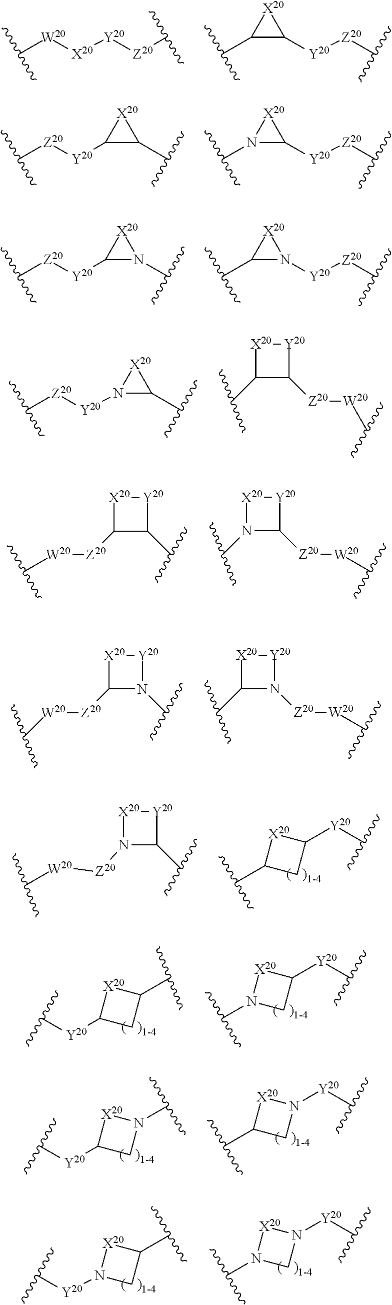

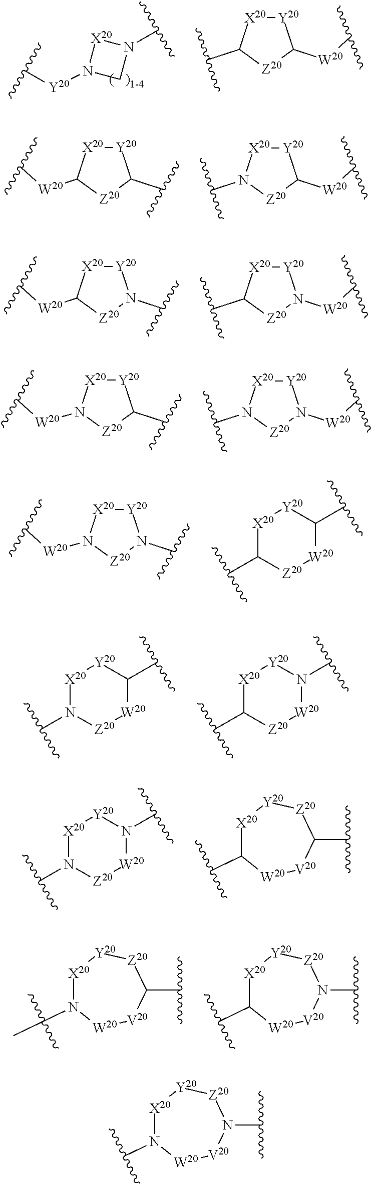

wherein V²⁰, W²⁰, X²⁰, Y²⁰, and Z²⁰ are independently —CH₂—, —C(H)(R¹⁰⁰)—, —C(R¹⁰⁰)₂—O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl, and R¹⁰¹ is hydrogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

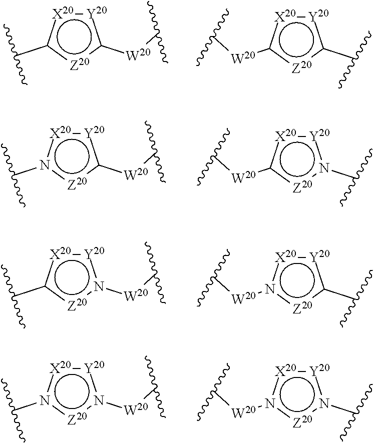

wherein W²⁰, X²⁰, Y²⁰, and Z²⁰ are independently —C(R¹⁰⁰)— or —N—; wherein R¹⁰⁰ represents independently for each occurrence hydrogen, halogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl.

In certain embodiments, L is one of the following:

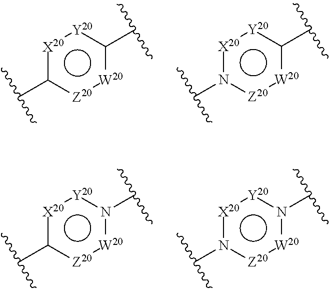

wherein W²⁰, X²⁰, Y²⁰, and Z²⁰ are independently —C(R¹⁰⁰)— or —N—; wherein R¹⁰⁰ represents independently for each occurrence hydrogen, halogen, C₁₋₆ alkyl, or C₃₋₆ cycloalkyl. In certain embodiments, R¹⁰⁰ represents independently for each occurrence hydrogen, methyl, ethyl, isopropyl, cyclopropyl, cyclobutyl, or cyclopentyl.

In certain embodiments, L is one of the following:

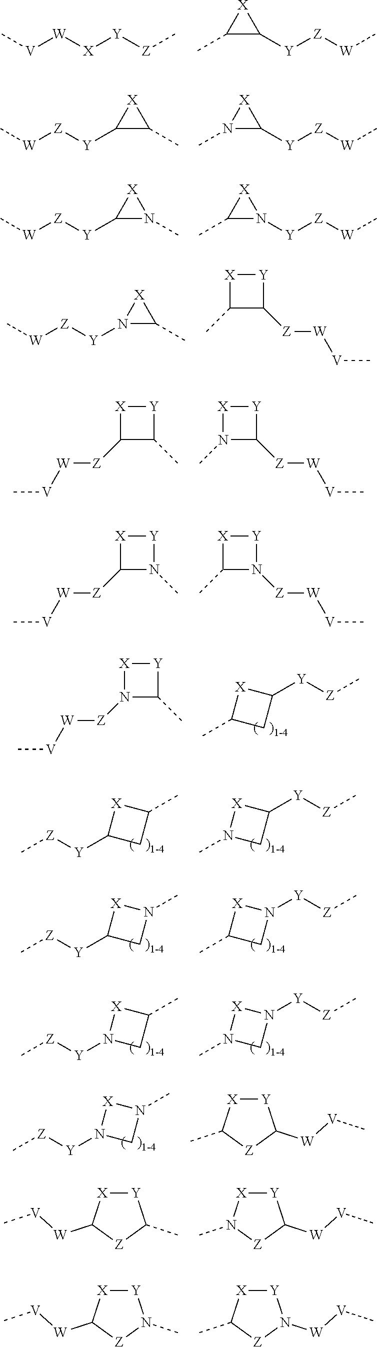

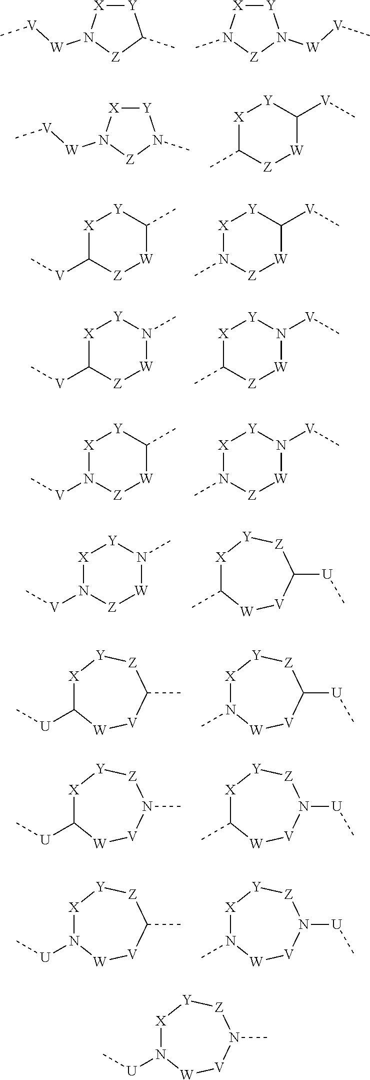

wherein U, V, W, X, Y, and Z are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

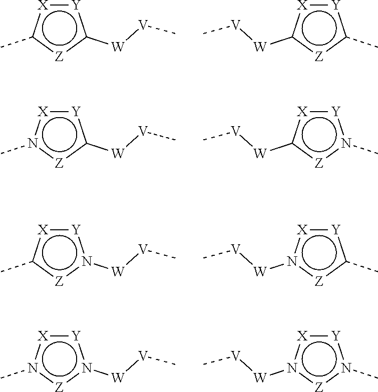

wherein X, Y, and Z are independently —C(R¹⁰⁰)— or —N—; V and W are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

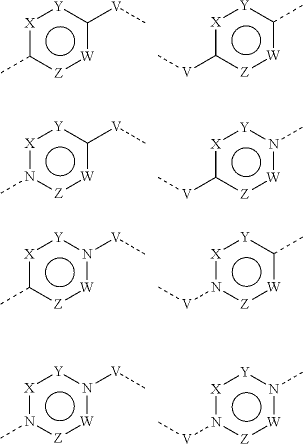

wherein W, X, Y, and Z are independently —C(R¹⁰⁰)— or —N—; V is —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

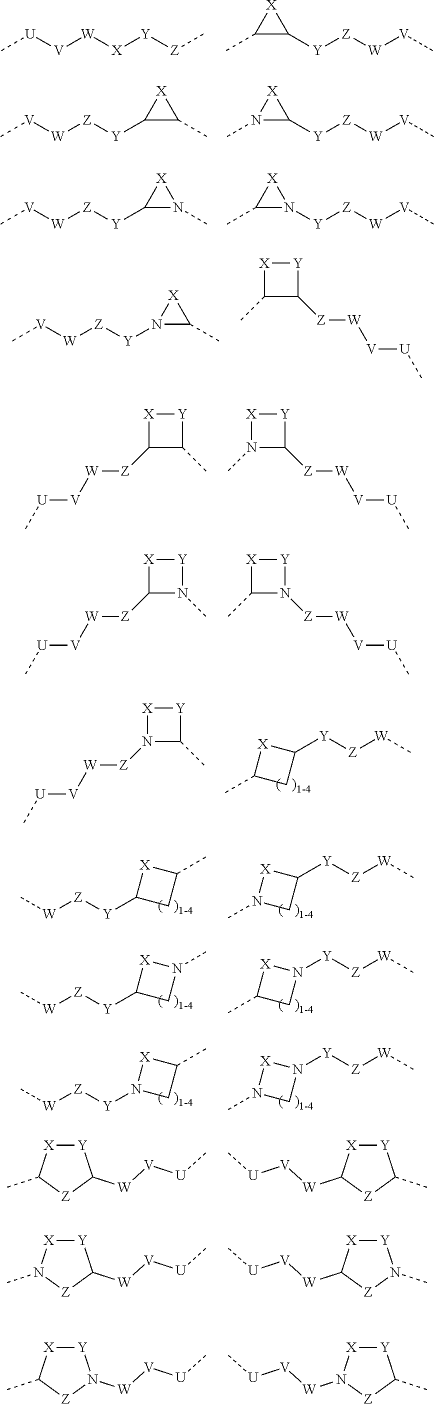

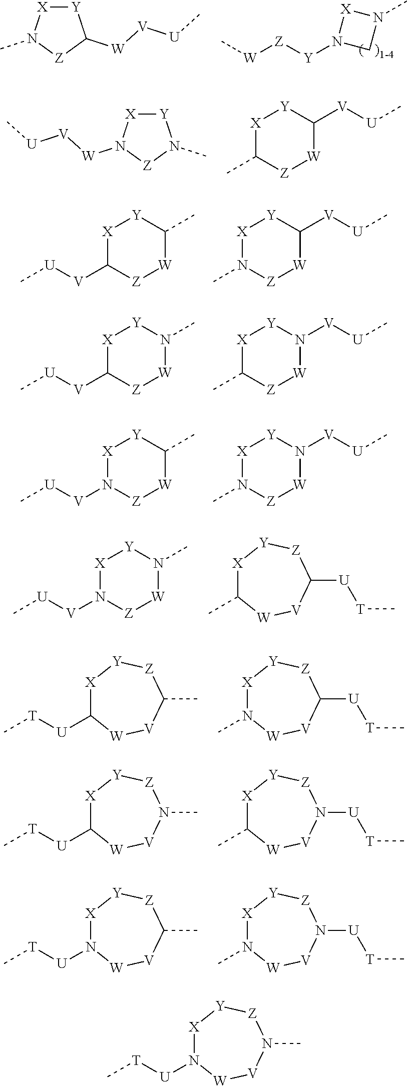

wherein T, U, V, W, X, Y, and Z are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

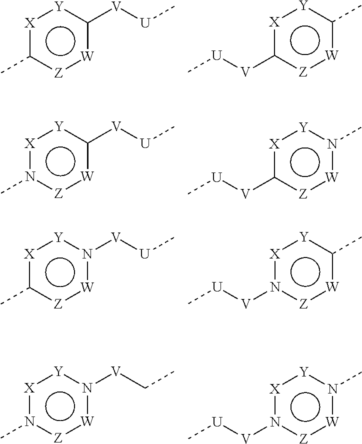

wherein W, X, Y, and Z are independently —C(R¹⁰⁰)— or —N—; U and V are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

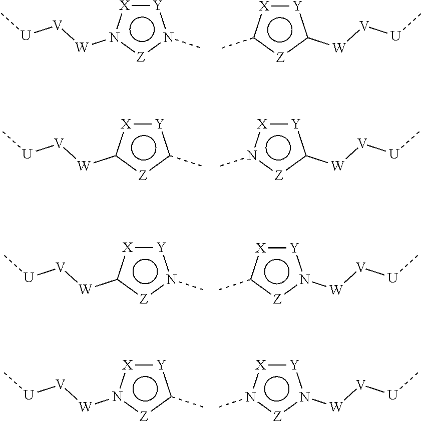

wherein X, Y, and Z are independently —C(R¹⁰⁰)— or —N—; U, V, and W are independently —CH₂—, —C(H)(R¹⁰⁰)—, —C(R¹⁰⁰)₂—, O, —N(R¹⁰¹)—, —S(O)₂—, —C(O)—, an optionally substituted C₃₋₇ cycloalkylene, an optionally substituted C₄₋₇ cycloalkenylene, or optionally substituted 3-7 membered heterocyclylene containing 1, 2 or 3 heteroatoms selected from oxygen, nitrogen, and sulfur; wherein R¹⁰⁰ represents independently for each occurrence hydrogen, halogen, C₁₋₆ alkyl, or C₃₋₆ cycloalkyl; R¹⁰¹ is hydrogen, C₁₋₆ alkyl, or C₃₋₆ cycloalkyl; and a dashed bond indicates a point of attachment. In certain embodiments, R¹⁰⁰ represents independently for each occurrence hydrogen, methyl, ethyl, isopropyl, cyclopropyl, cyclobutyl, or cyclopentyl. In certain embodiments, R¹⁰¹ is hydrogen, methyl, ethyl, isopropyl, cyclopropyl, cyclobutyl, or cyclopentyl.

In certain embodiments, L is one of the following:

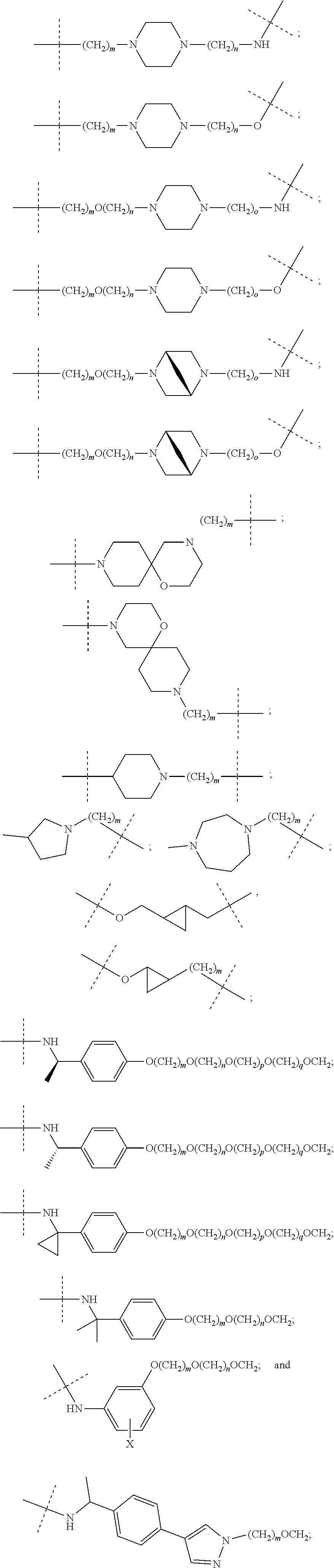

wherein
variables m, n, o, p, and q are independently 0, 1, 2, 3, 4, 5, 6, 7, 8, 9, 10, 11, 12, 13, 14, 15, 16, 17, 18, 19, or 20.

In certain embodiments, L is one of the following:

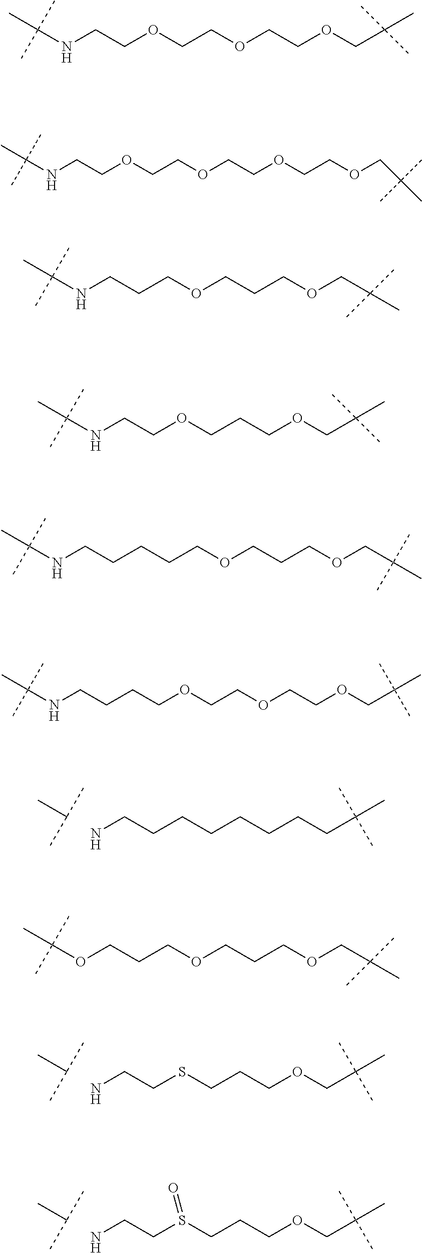

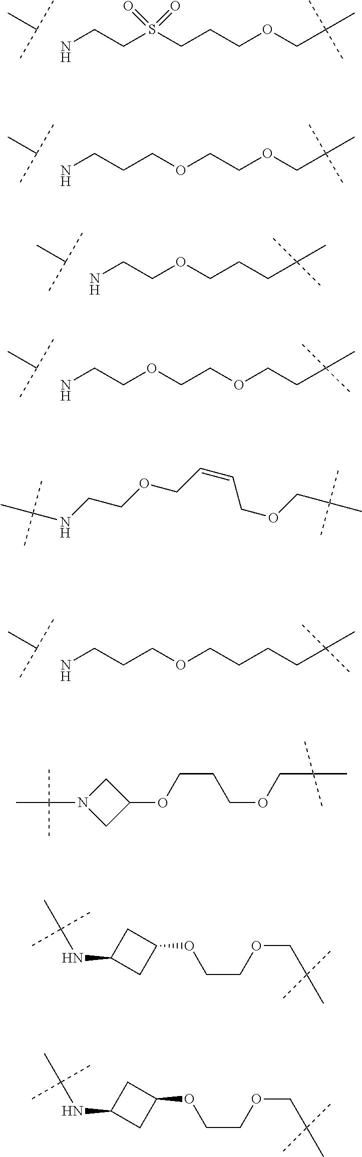

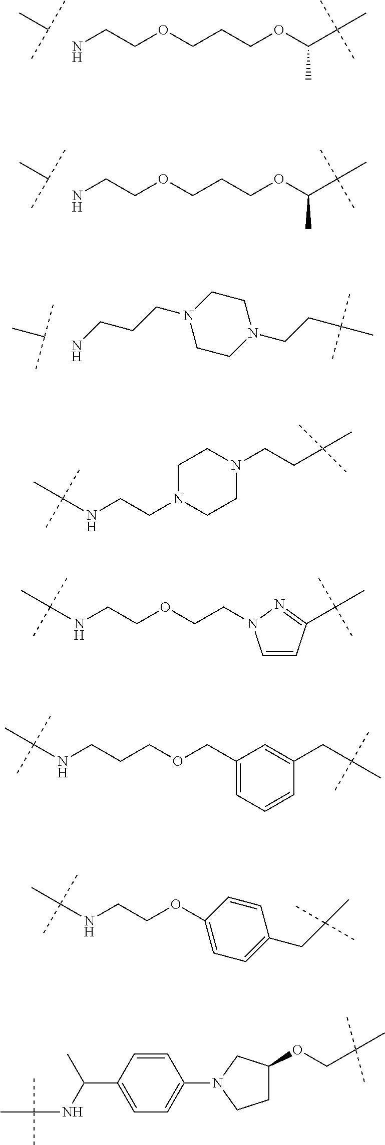

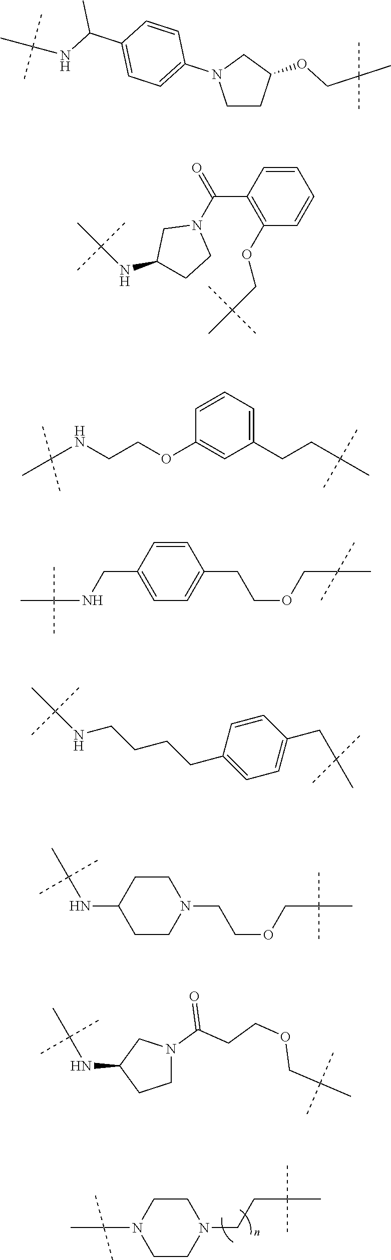

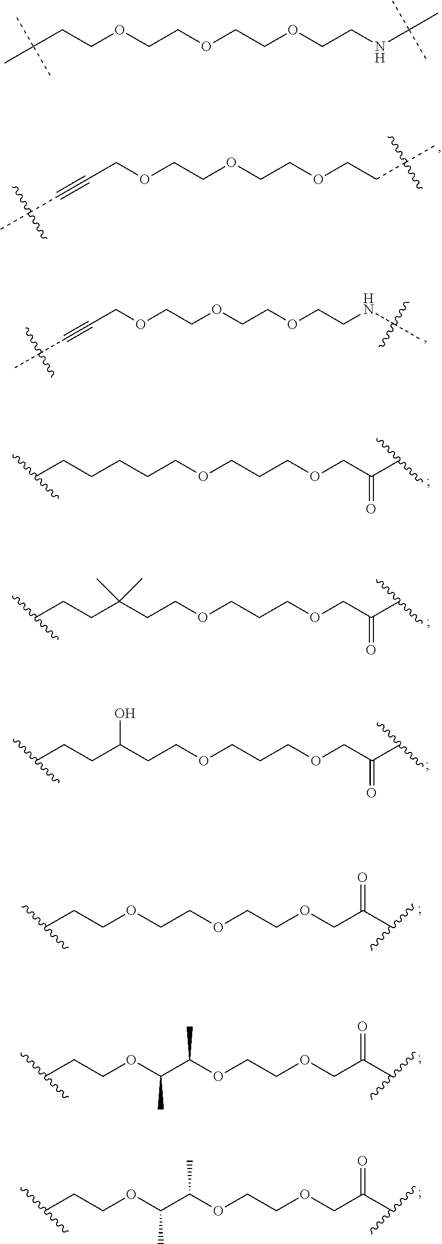

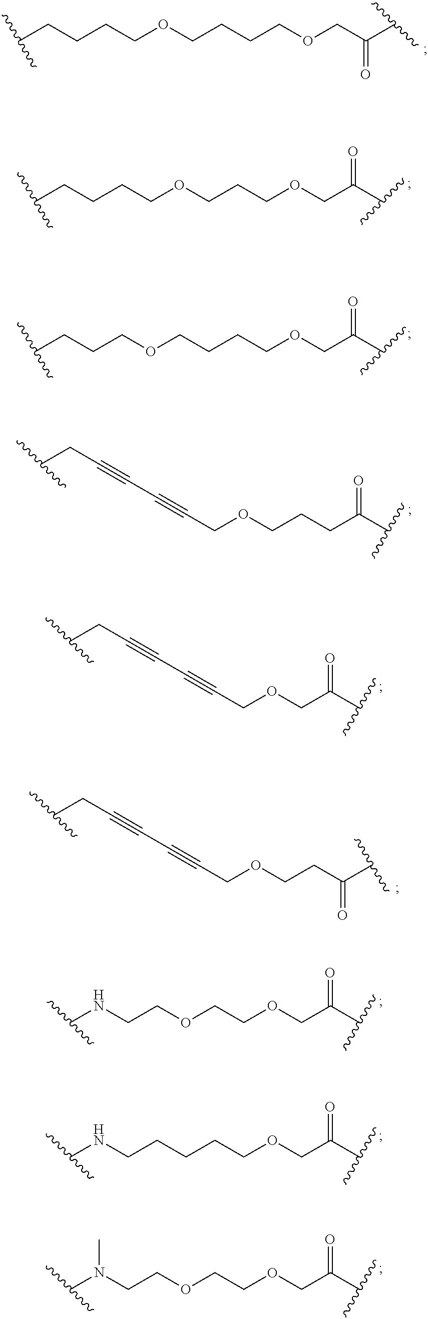

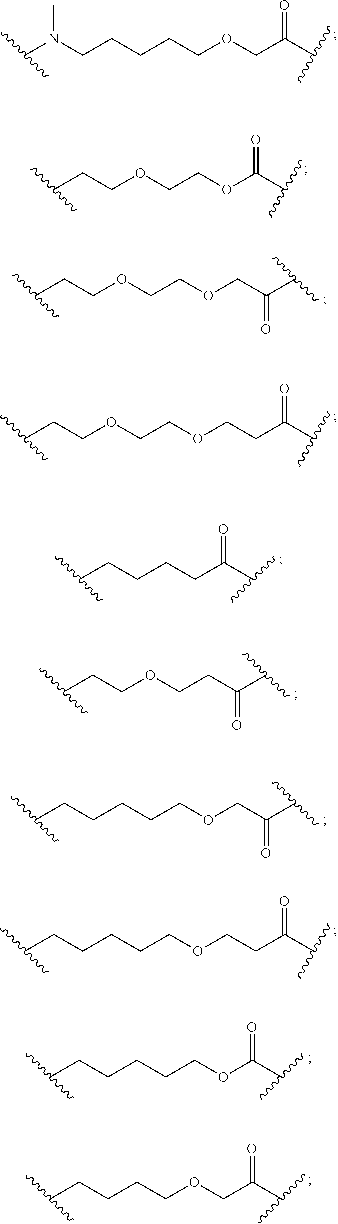

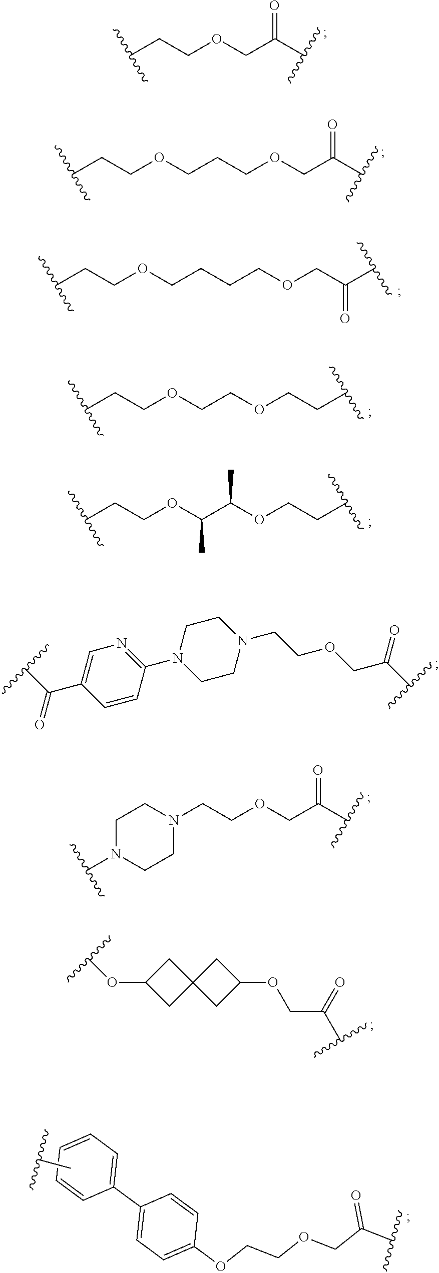

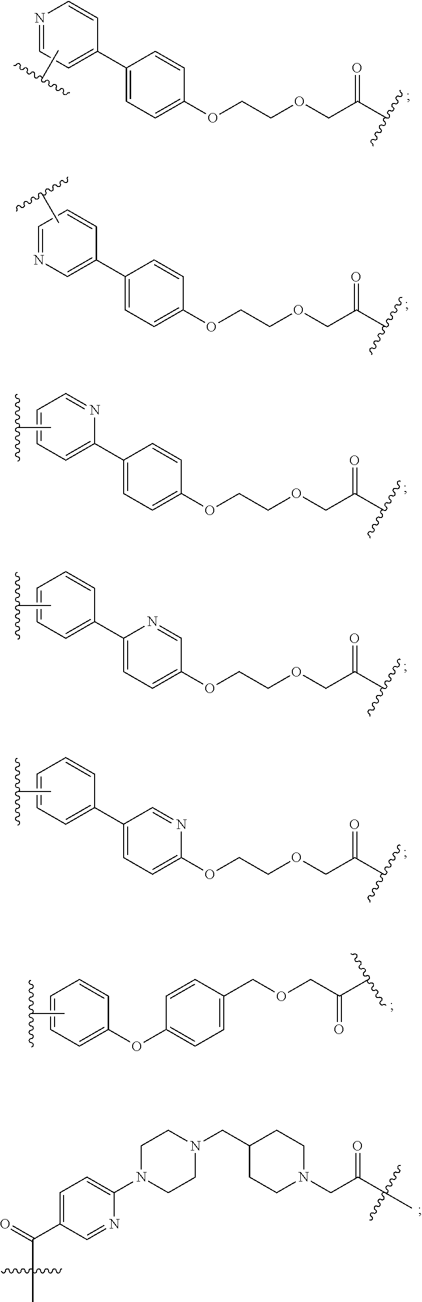

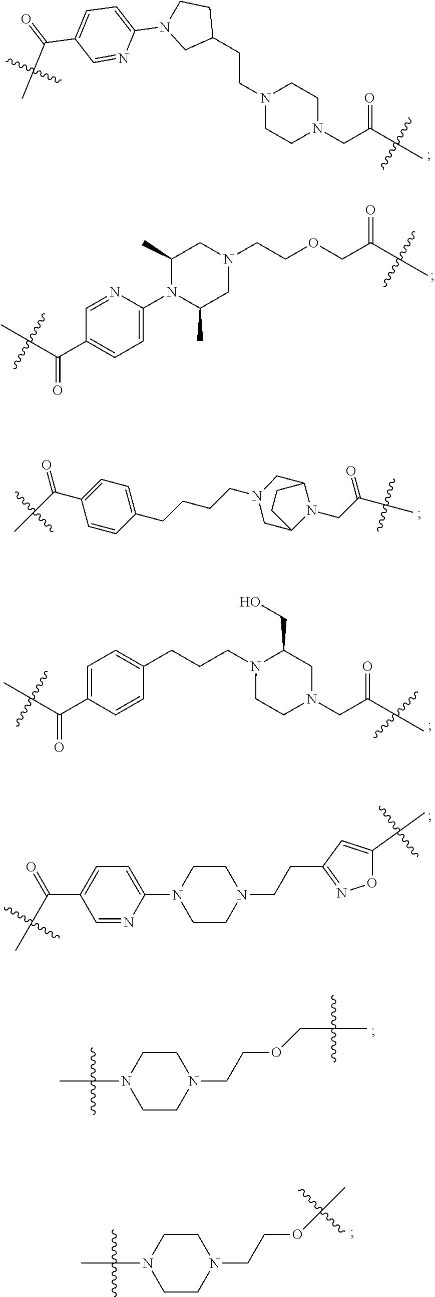

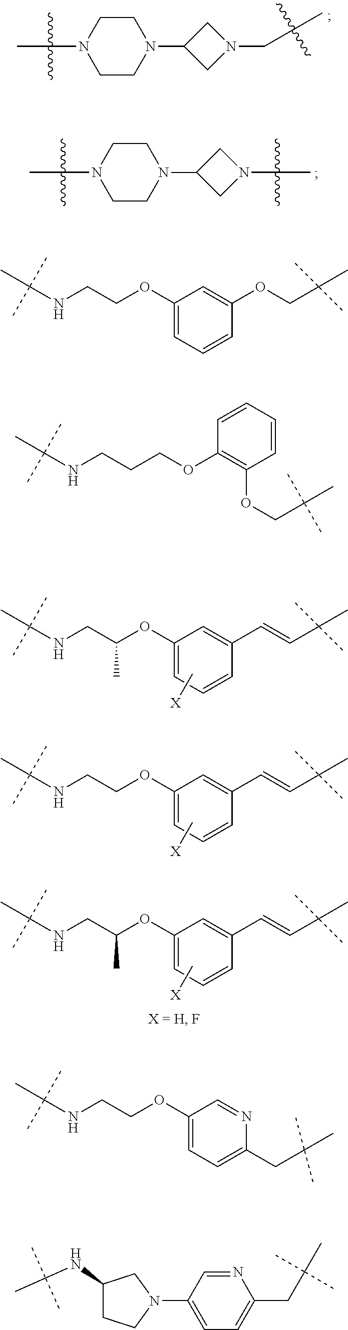

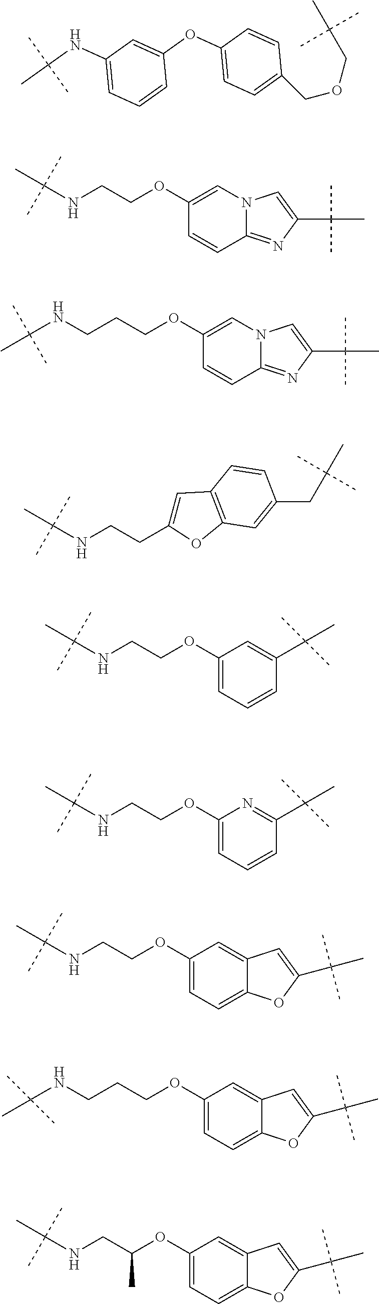

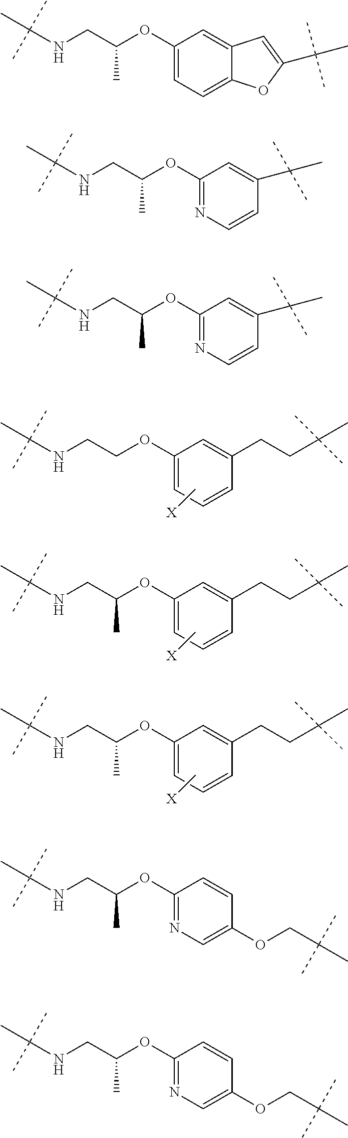

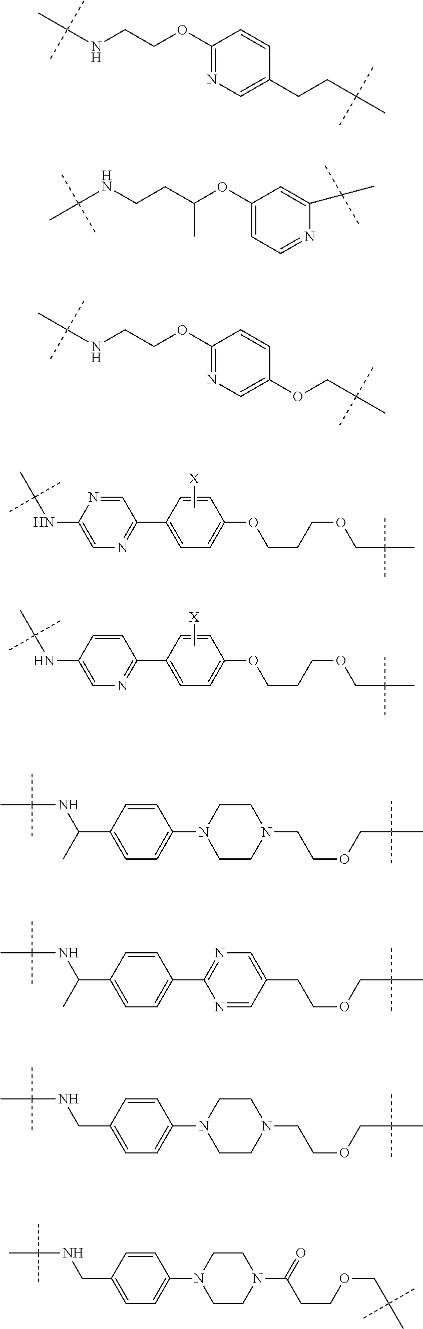

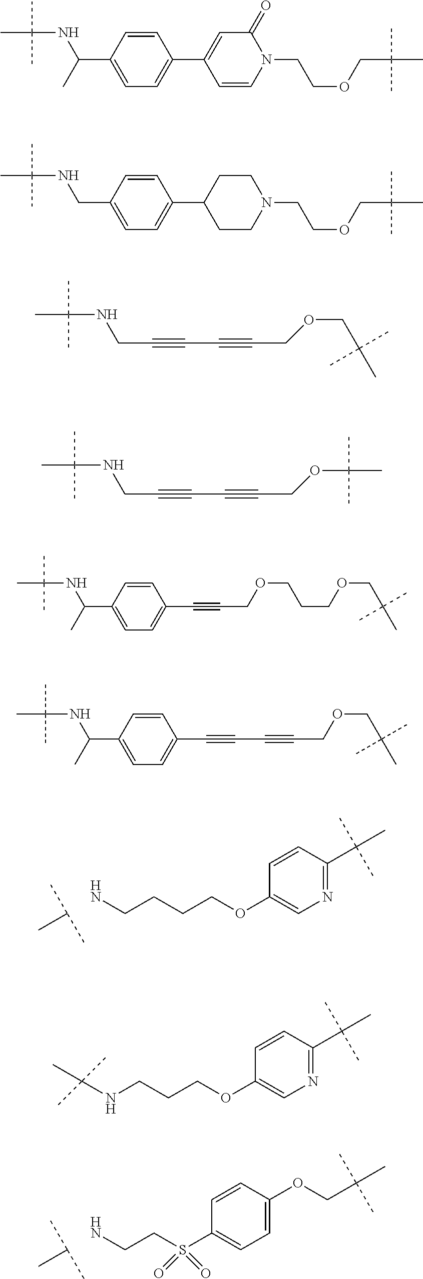

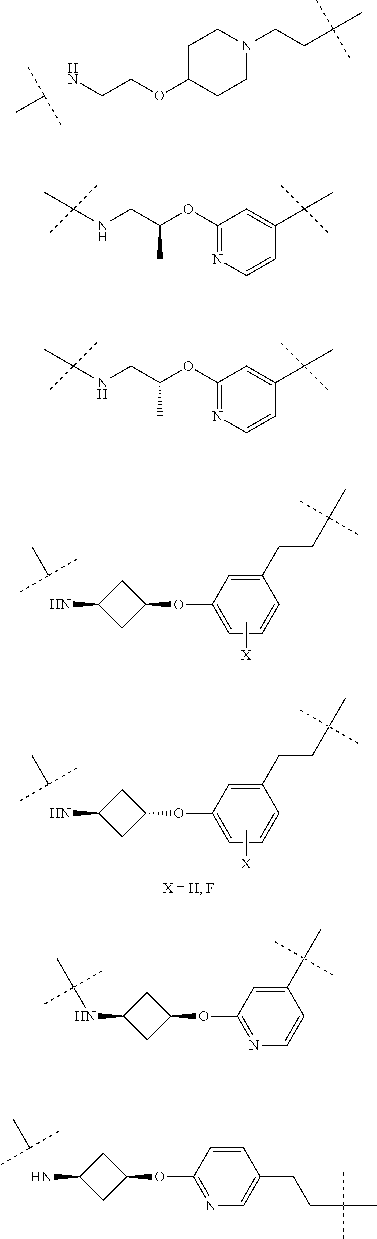

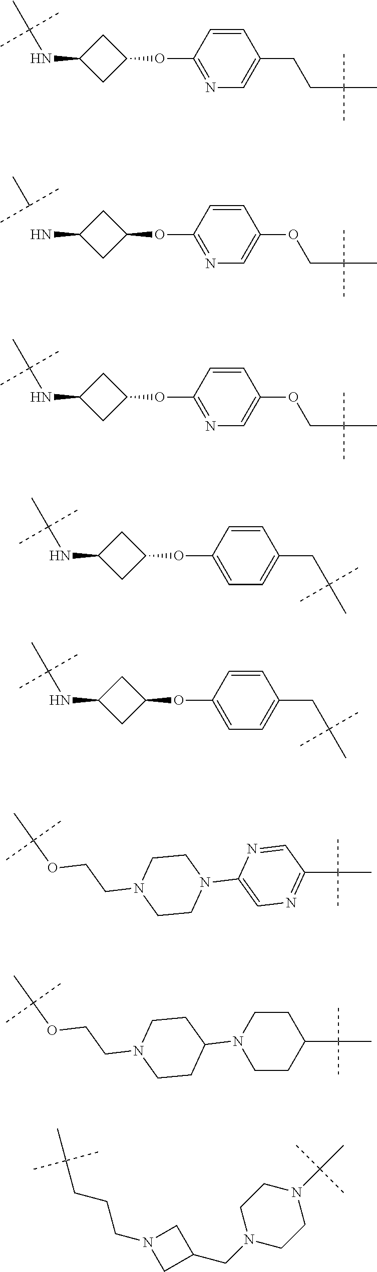

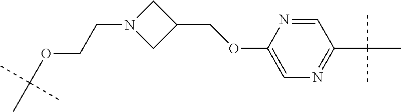

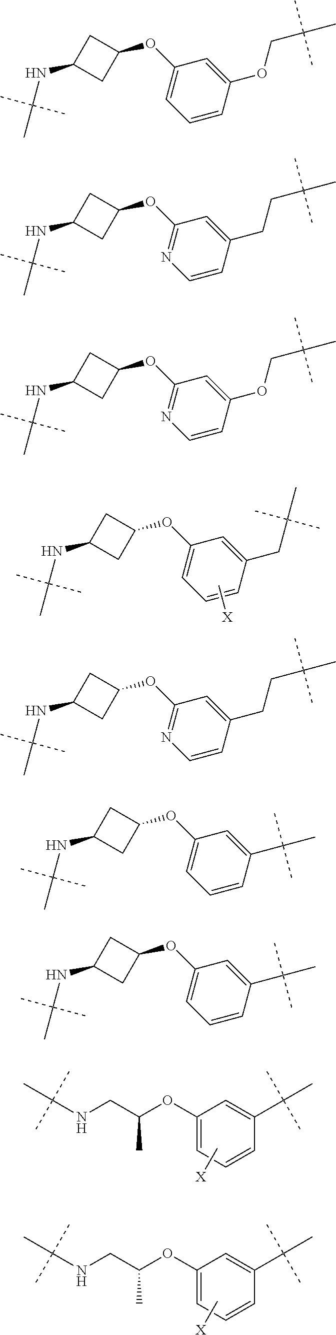

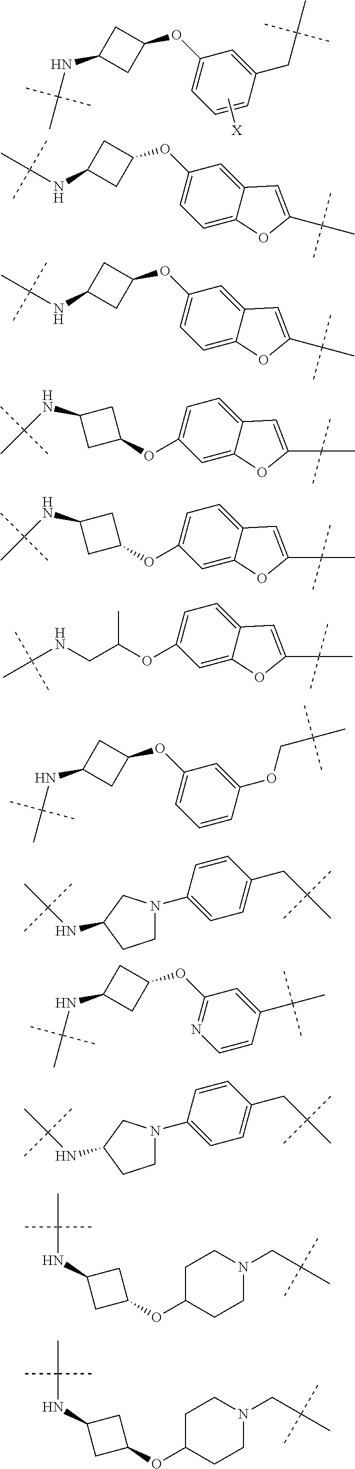

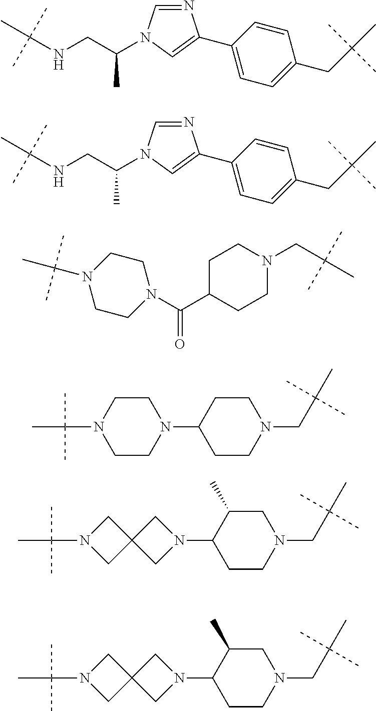

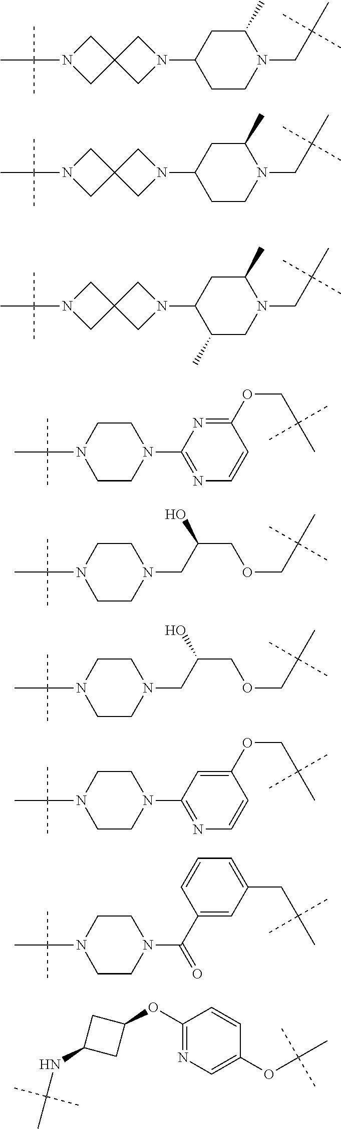

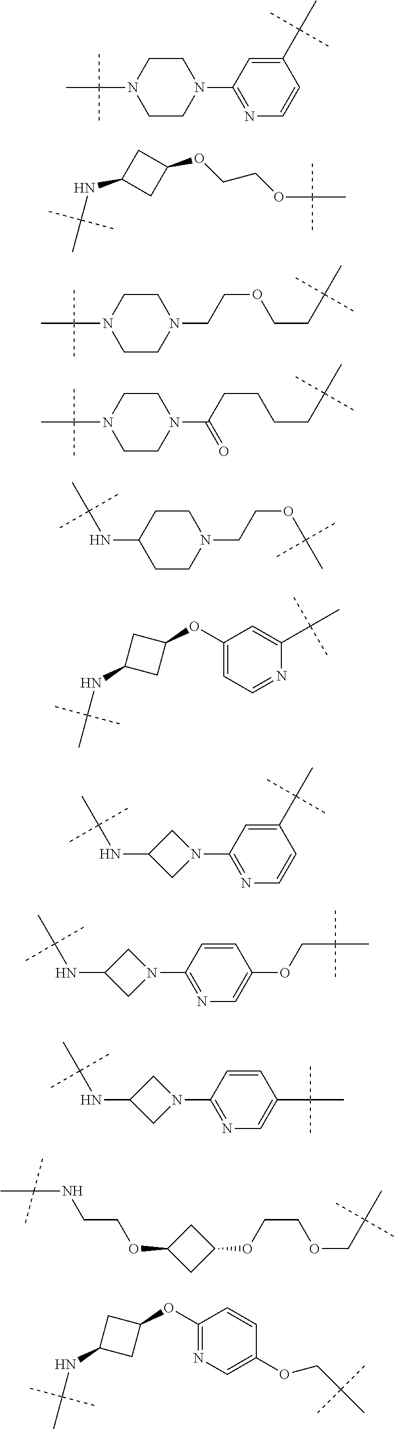

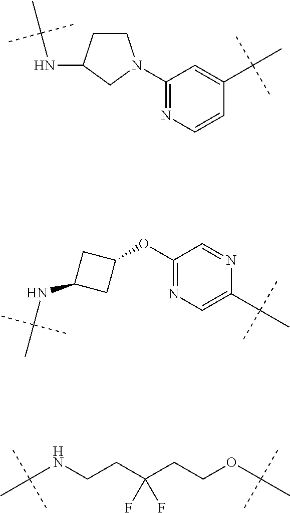

wherein any m or n are independently 0, 1, 2, 3, 4, 5, or 6; and any X is H or F.

In certain embodiments, L is one of the following:

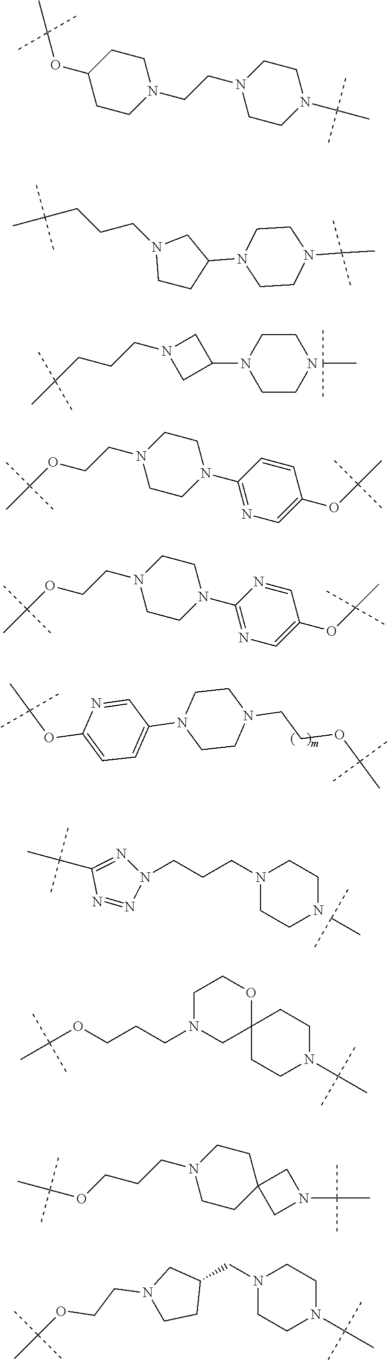

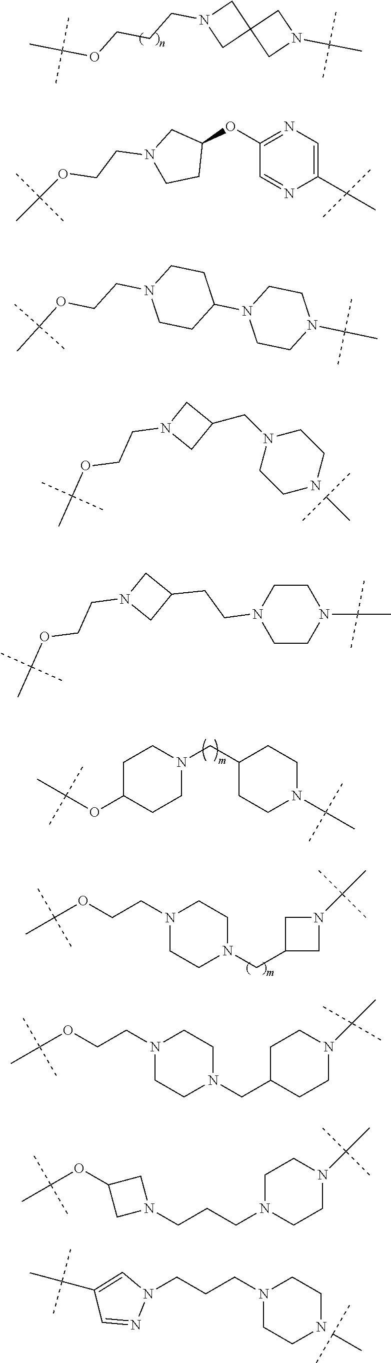

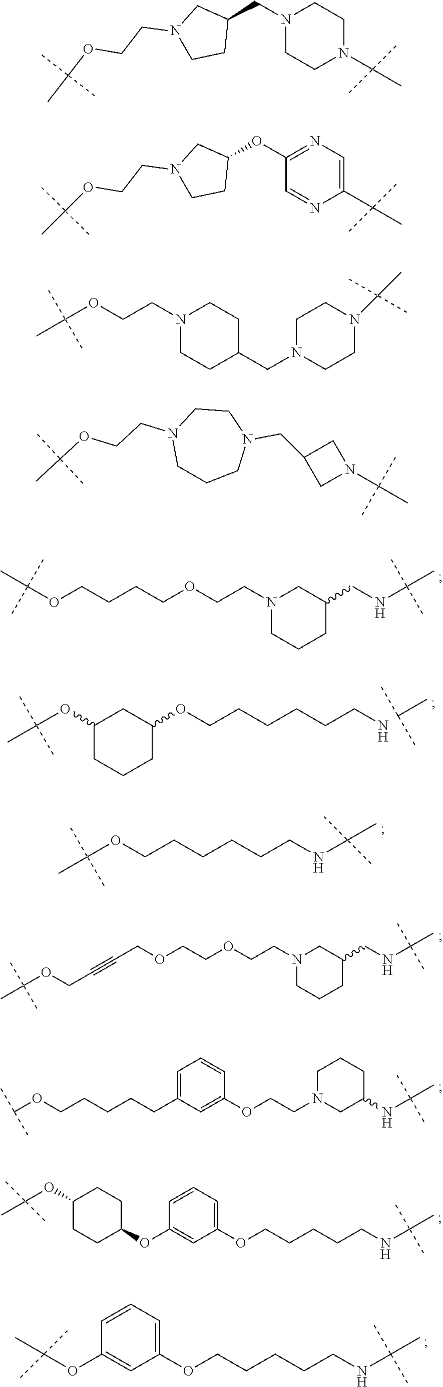

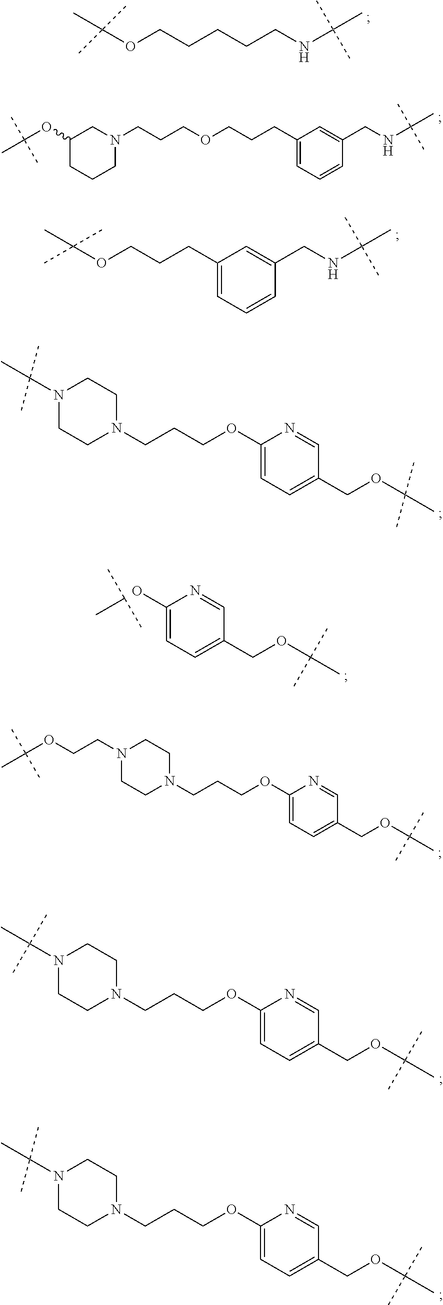

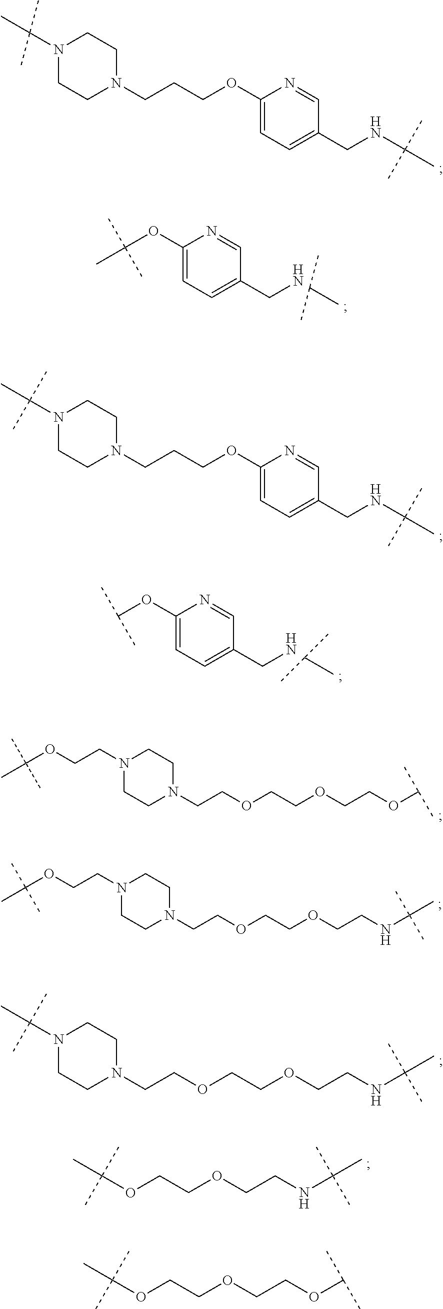

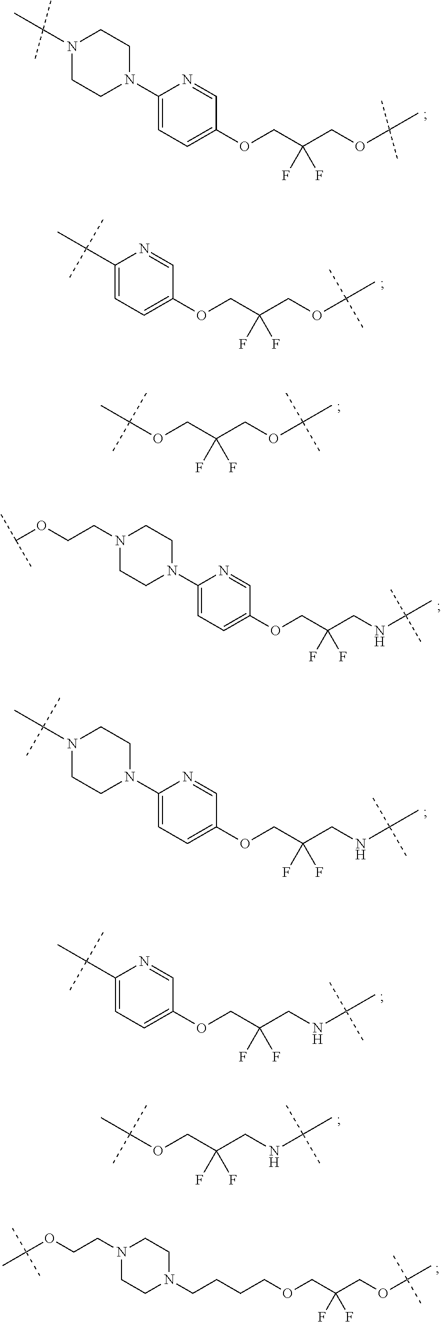

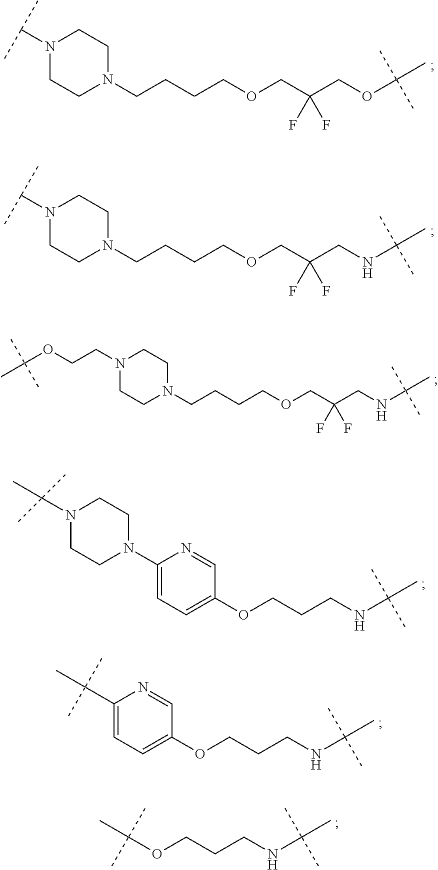

wherein any m or n are independently 0, 1, 2, 3, 4, 5, or 6.

In certain embodiments, L is one of the following:

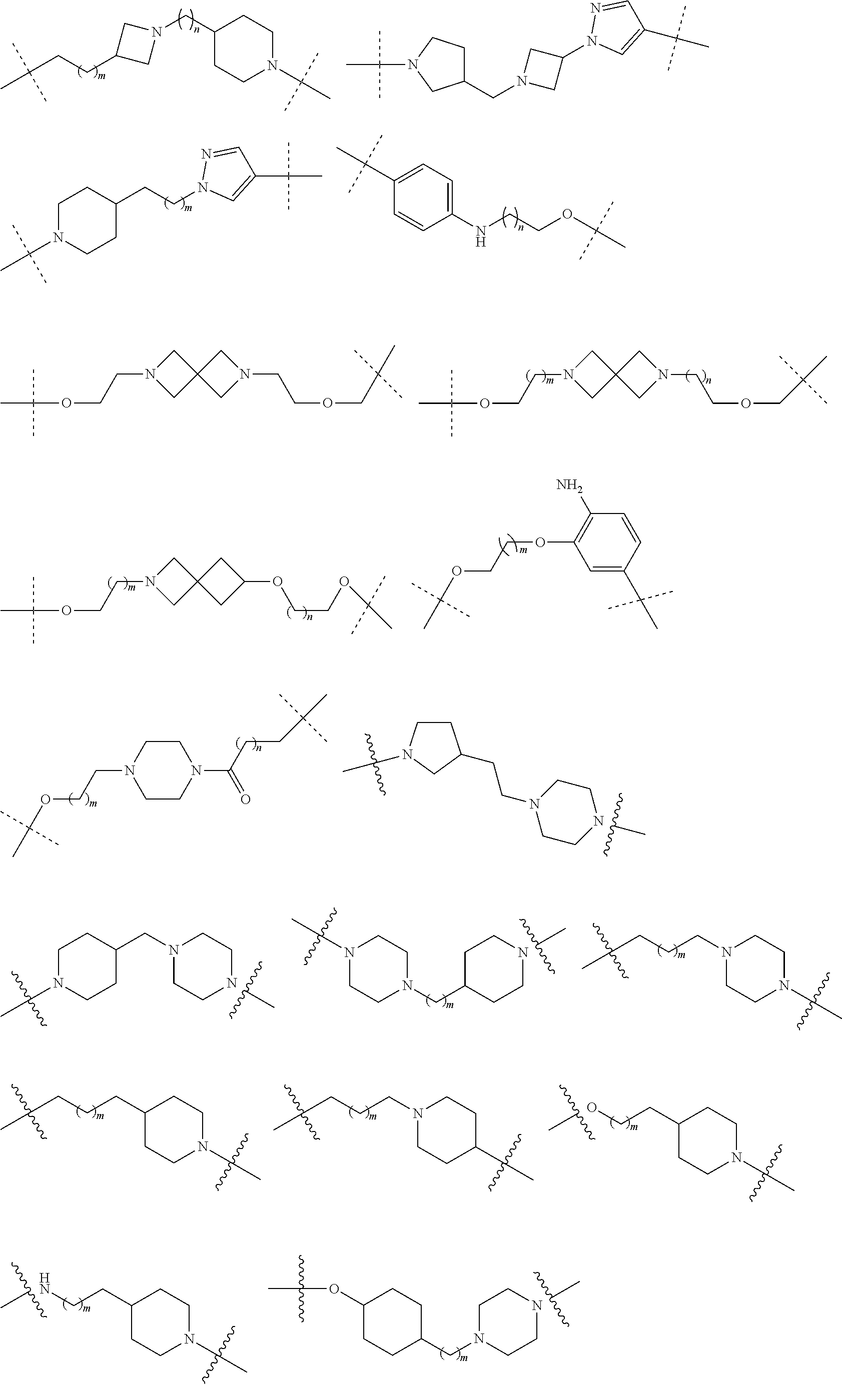

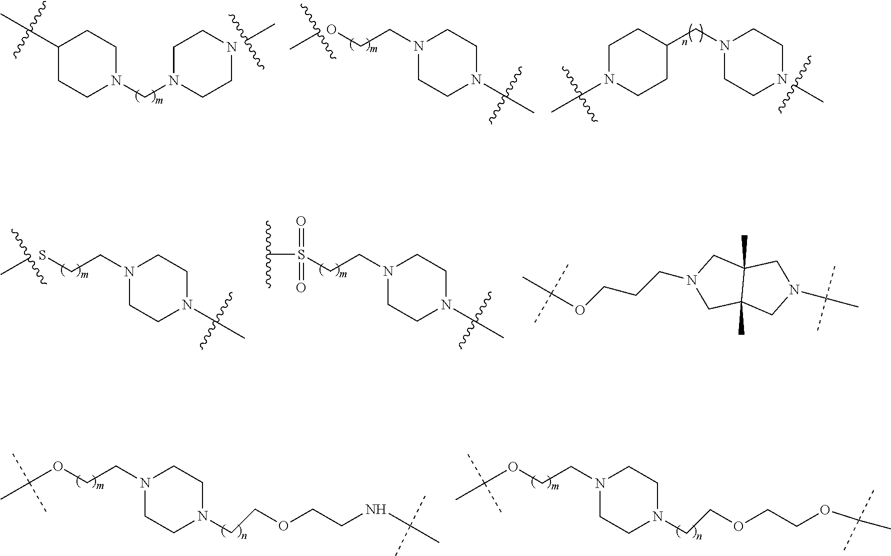

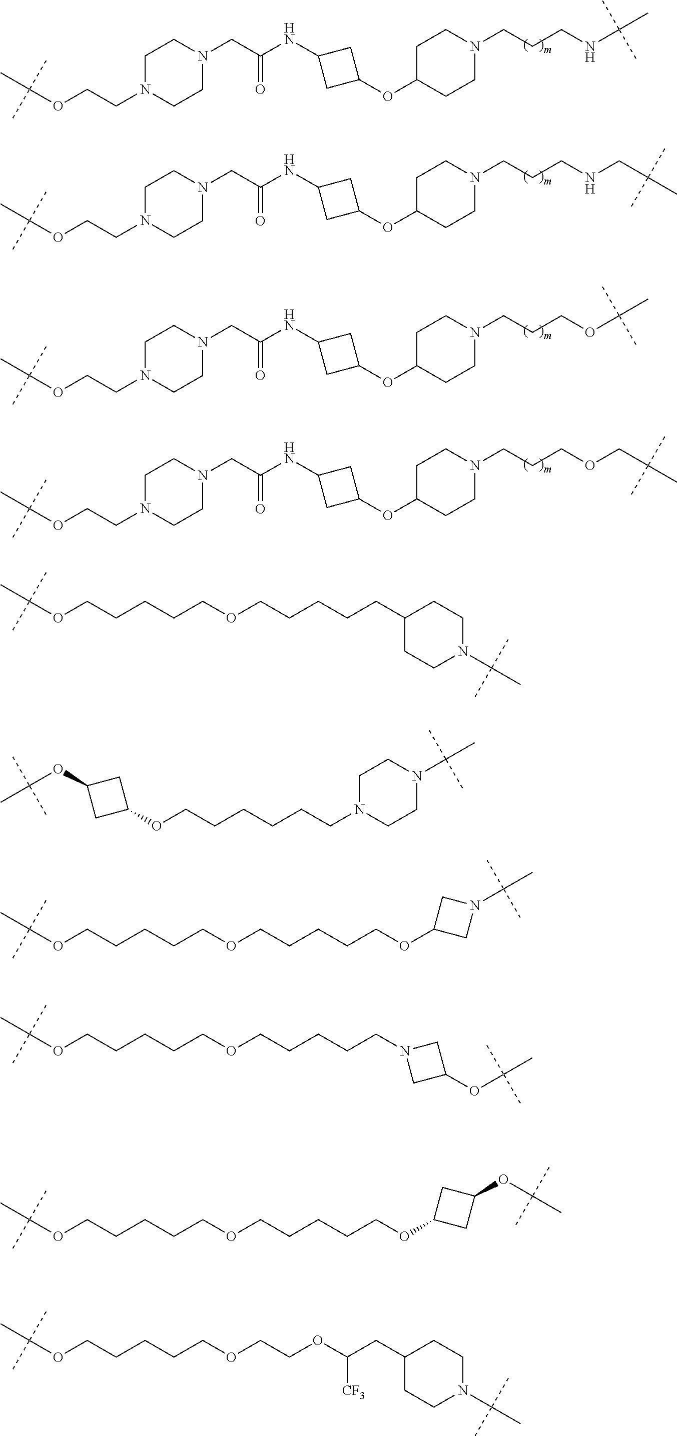

wherein any m or n are independently 0, 1, 2, 3, 4, 5, or 6.

In certain embodiments, L is one of the following:

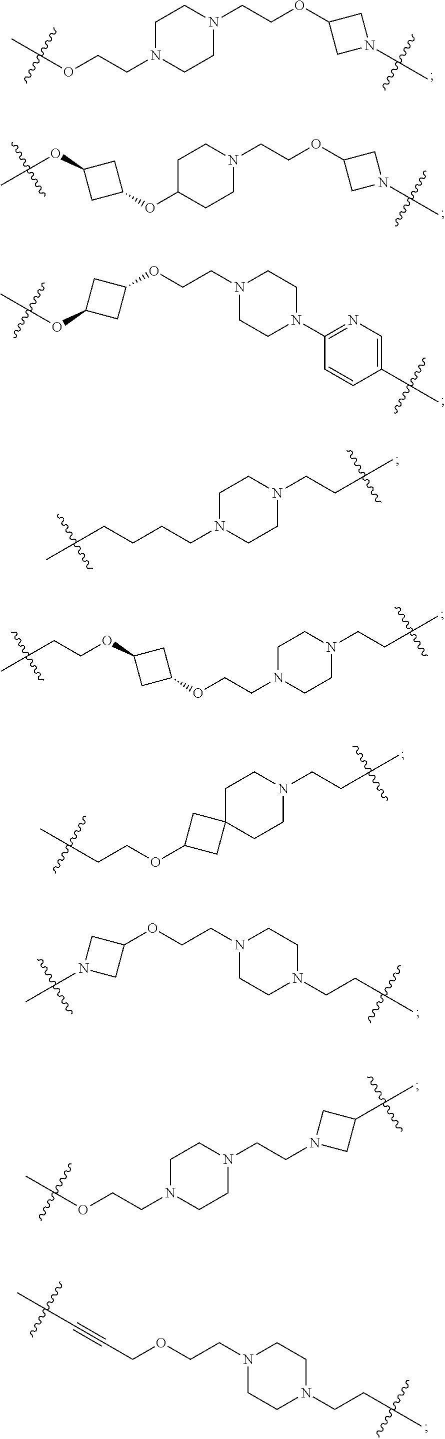

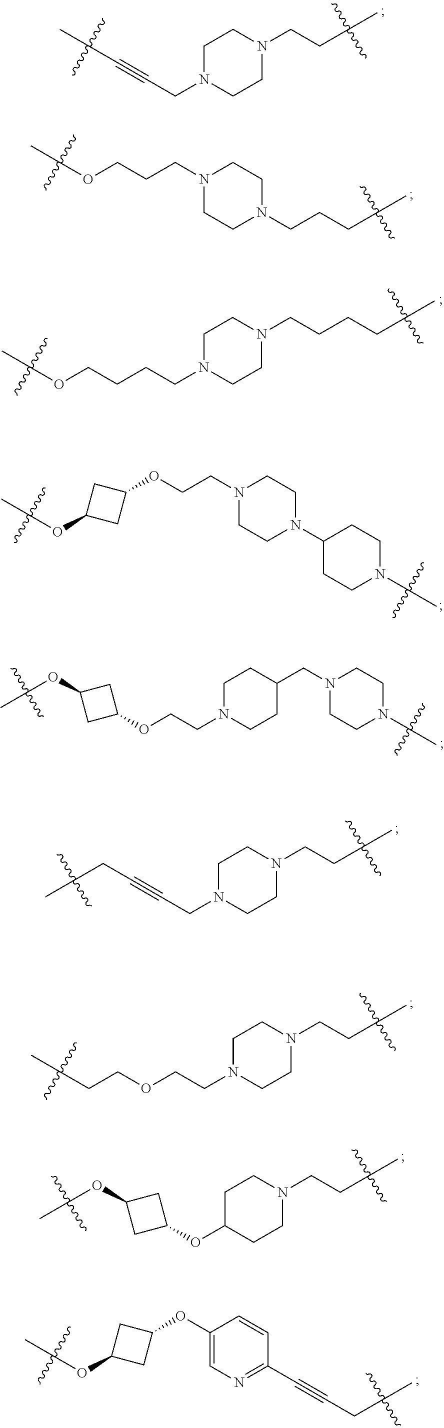

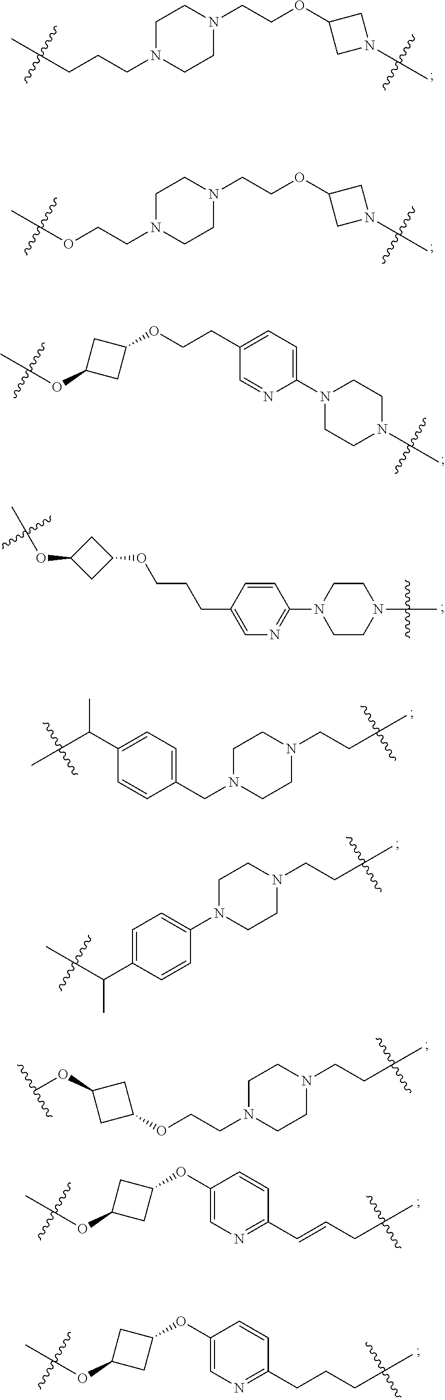

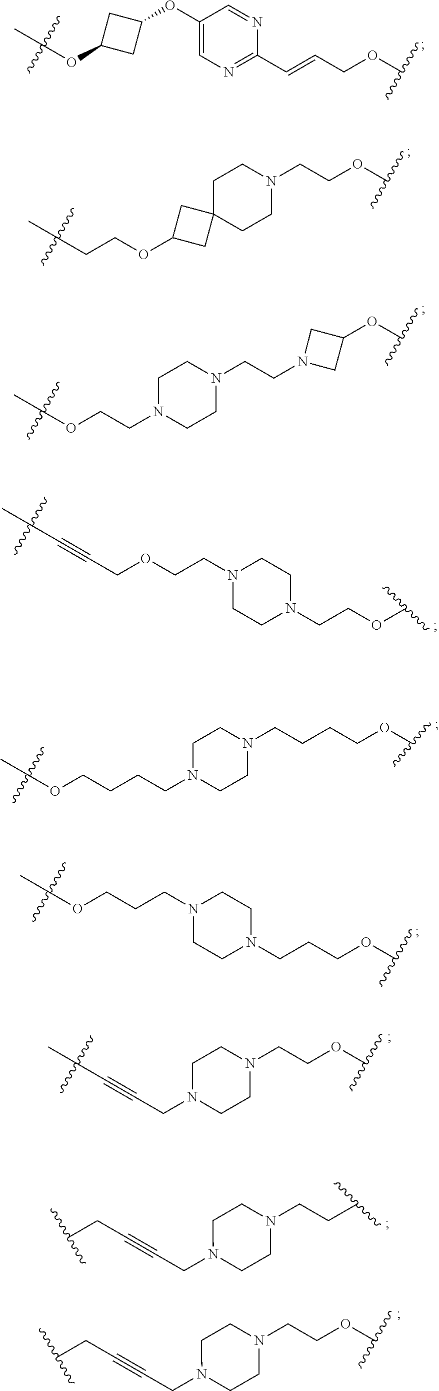

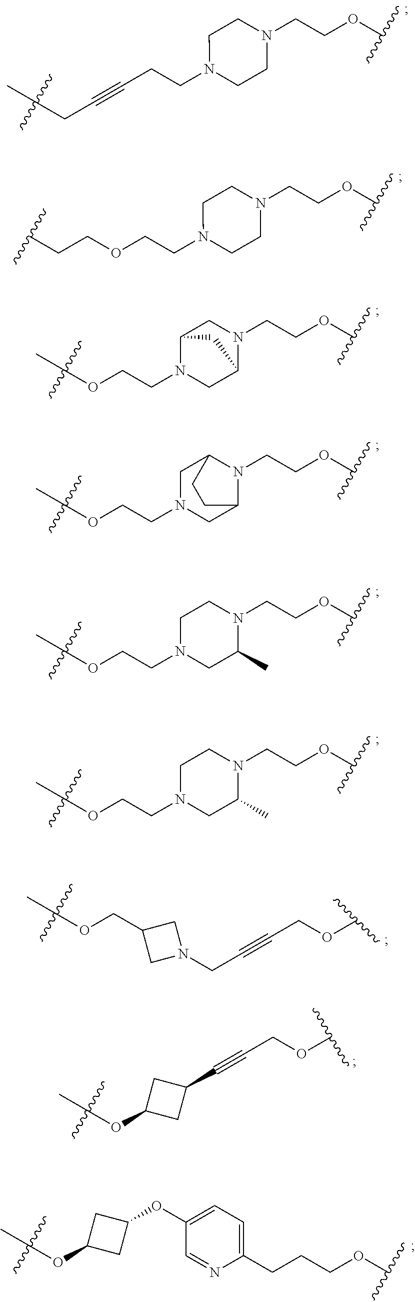

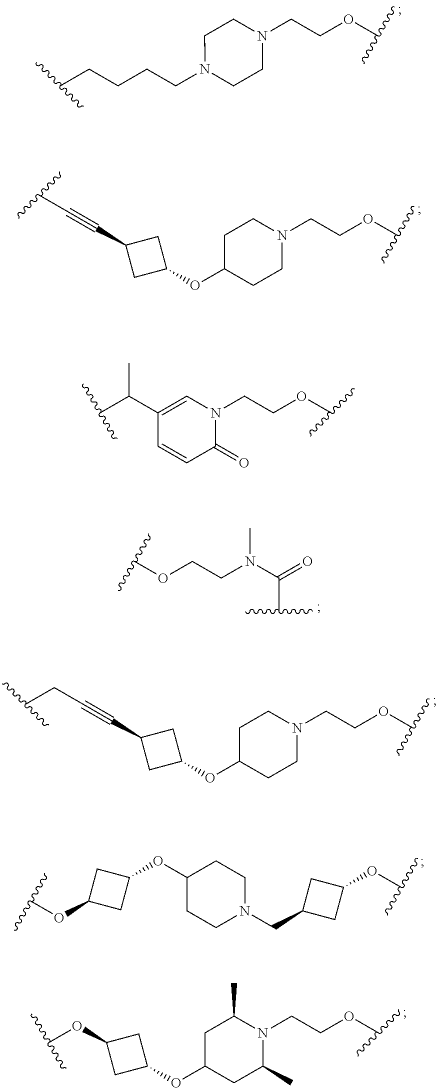

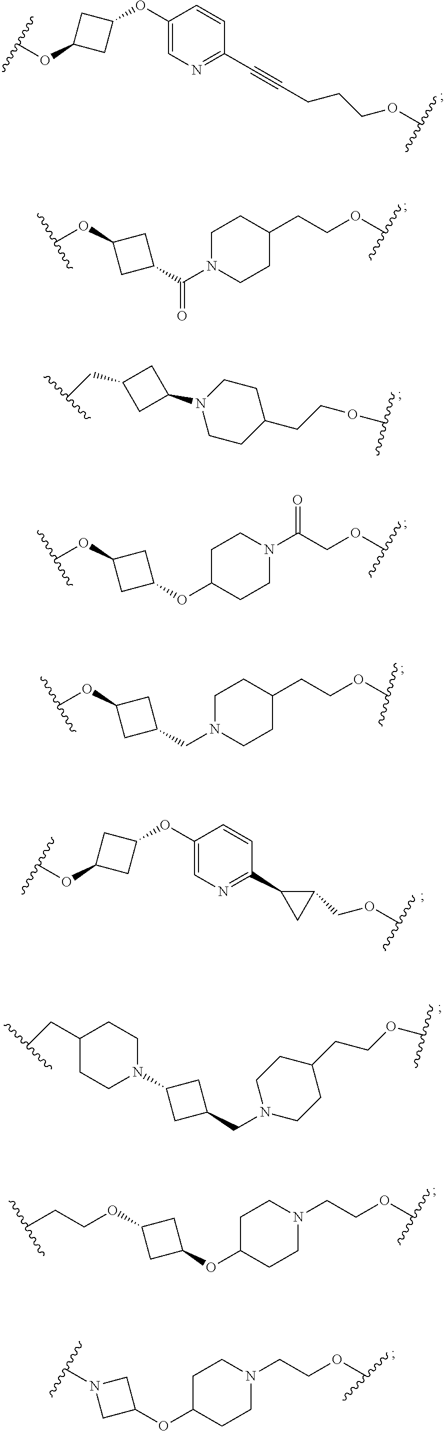

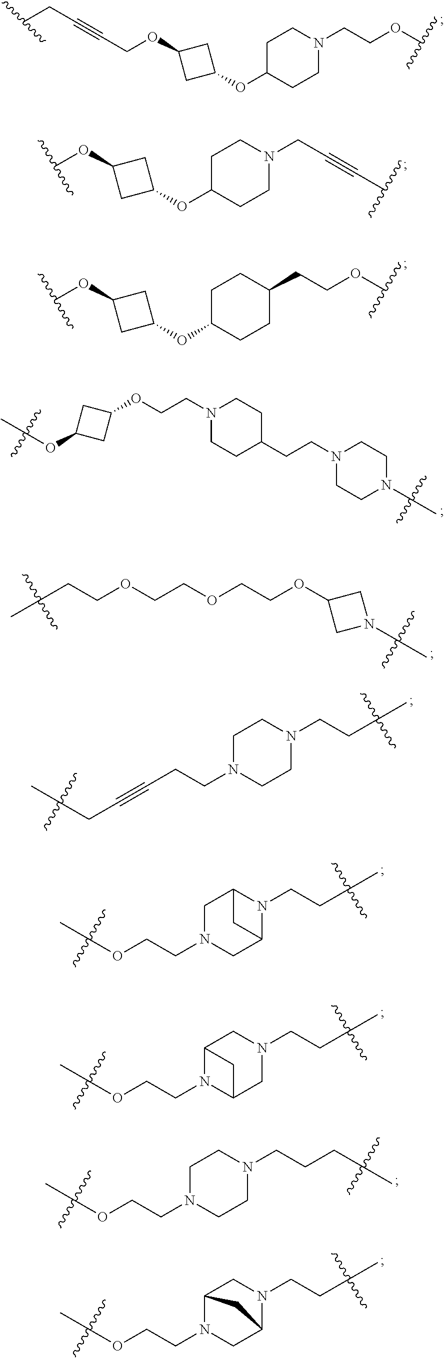

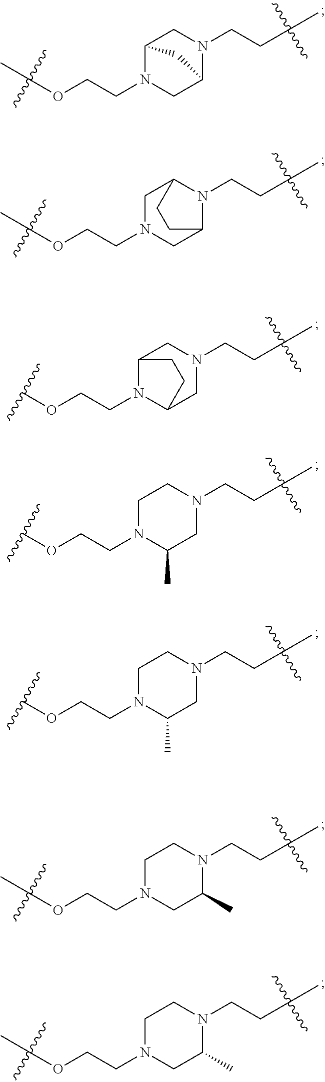

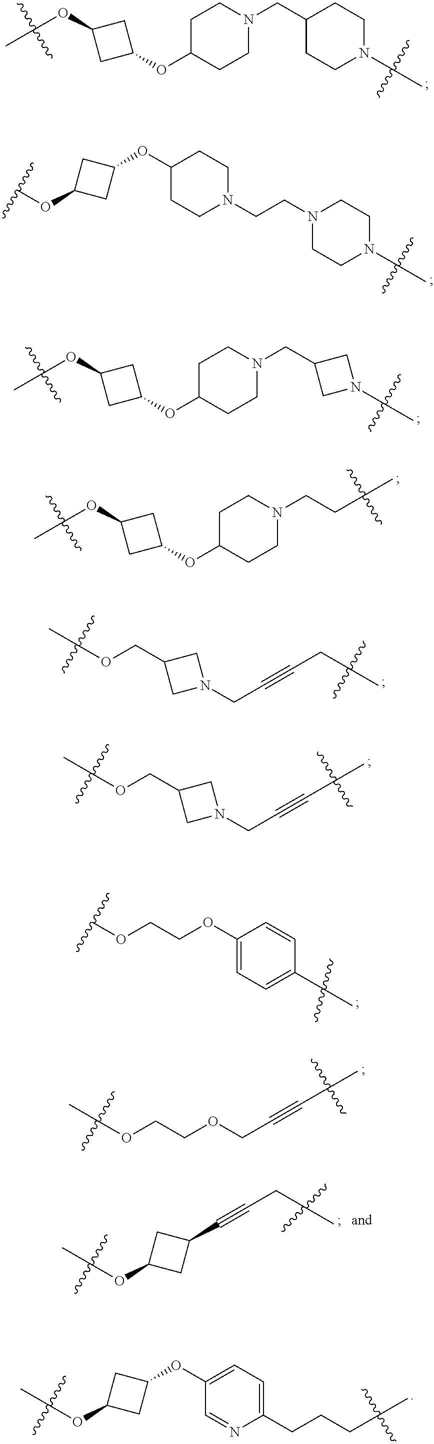

In certain embodiments, L is one of the following:

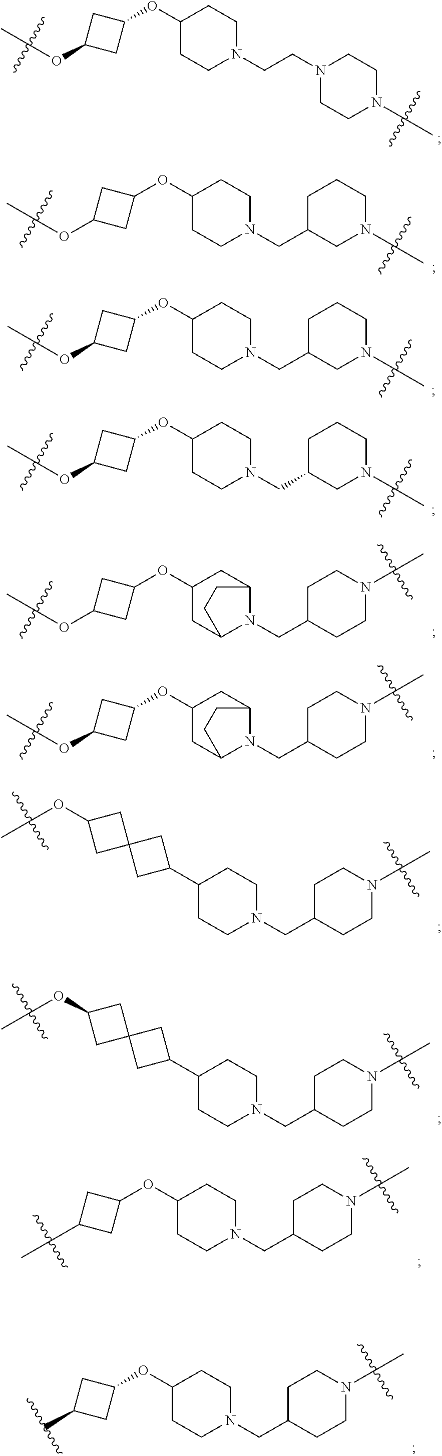

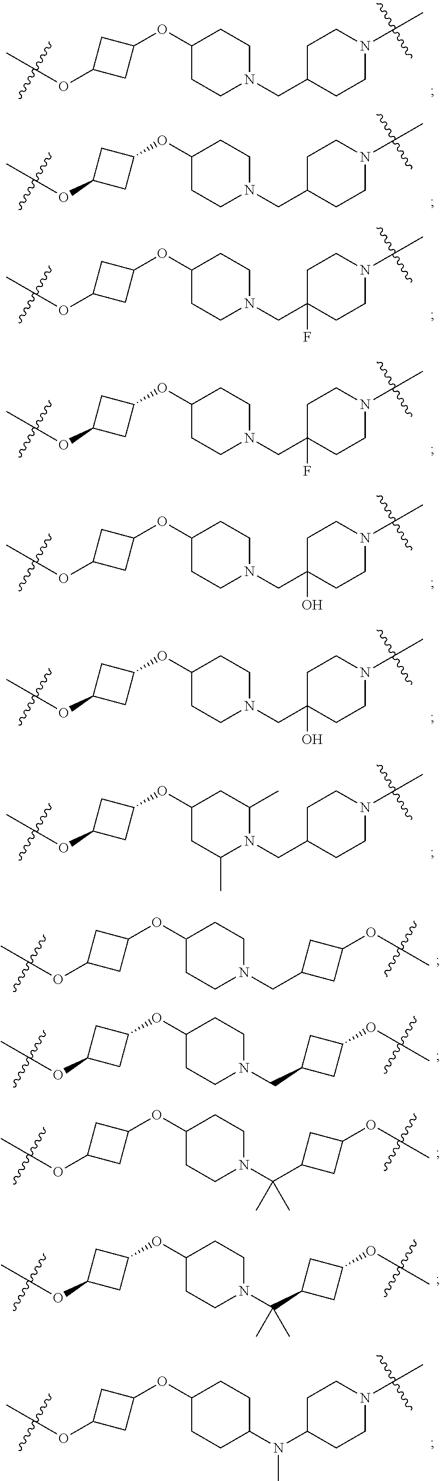

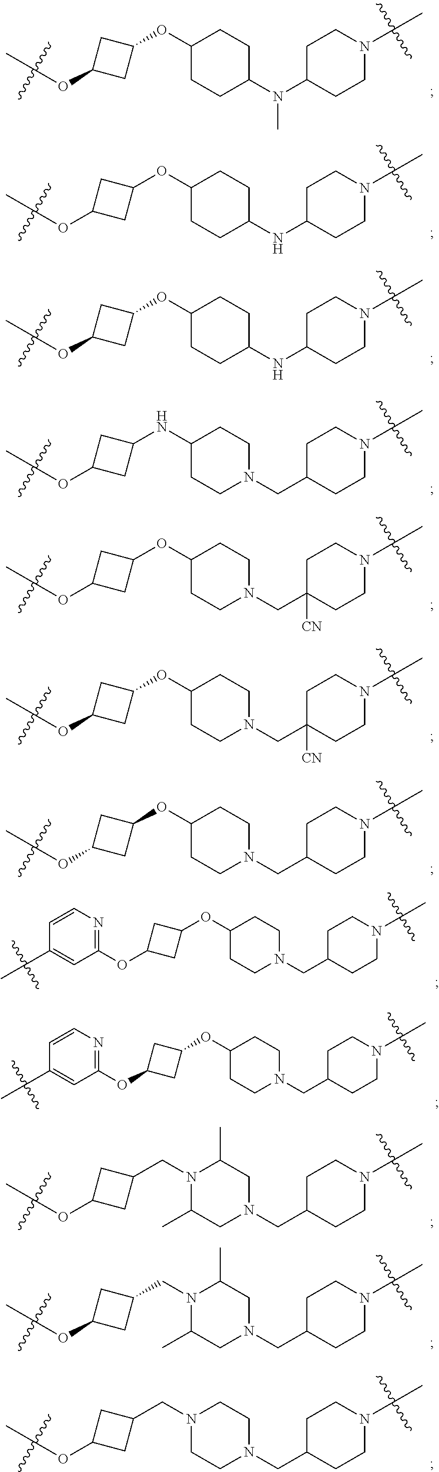

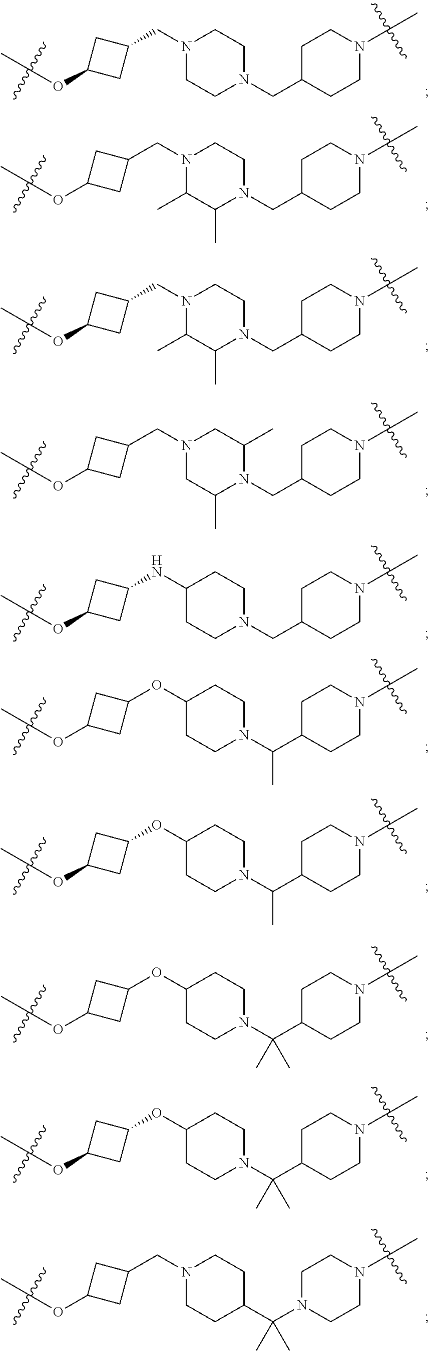

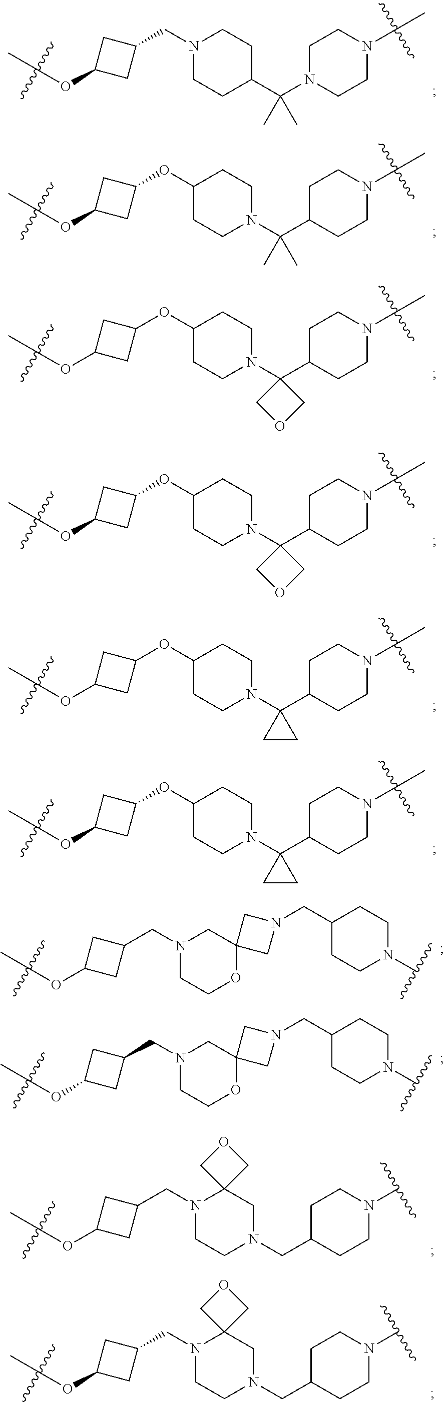

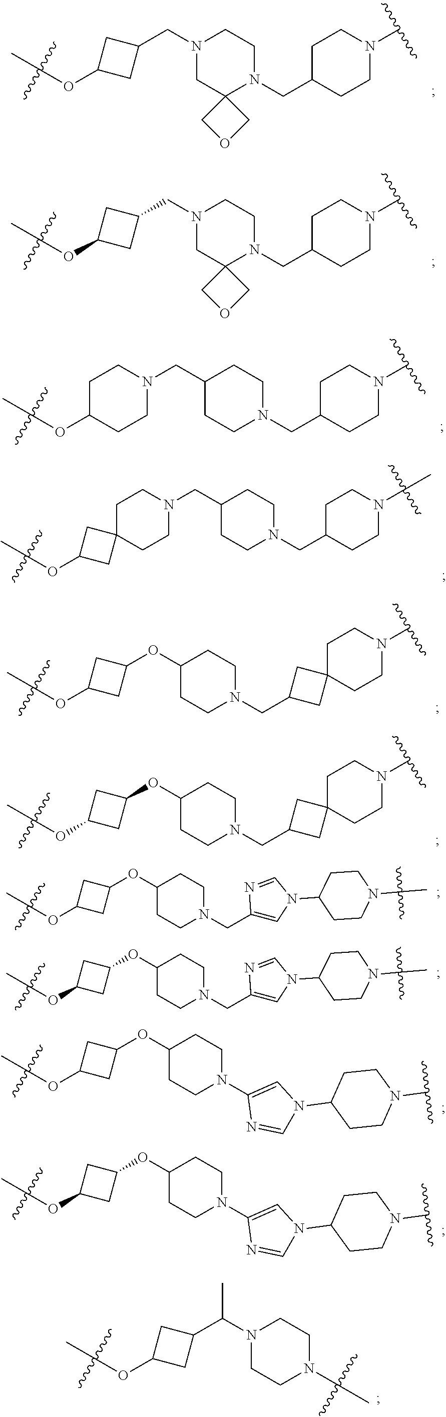

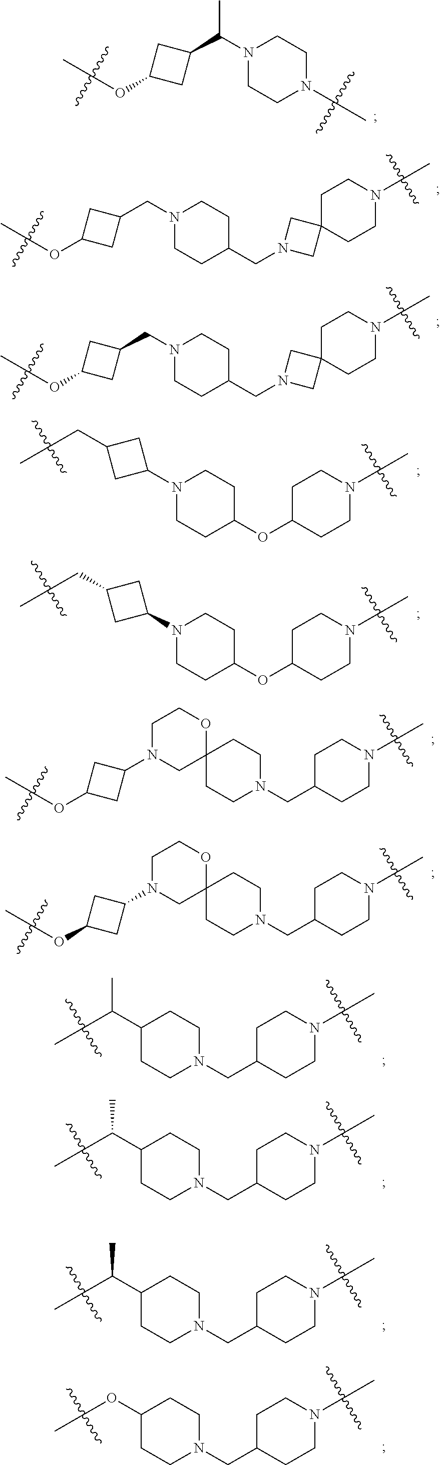

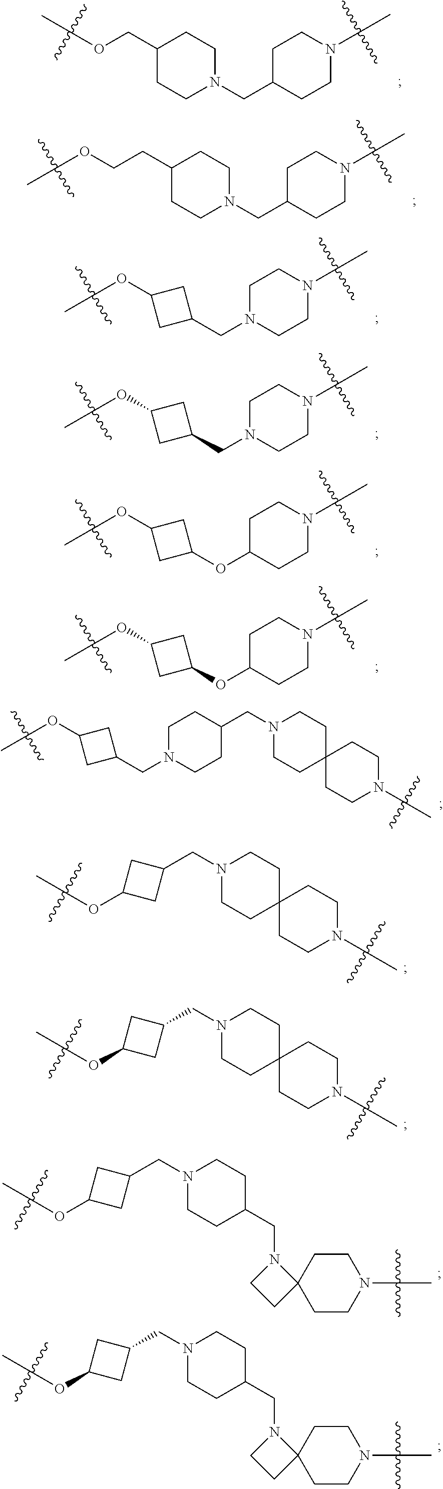

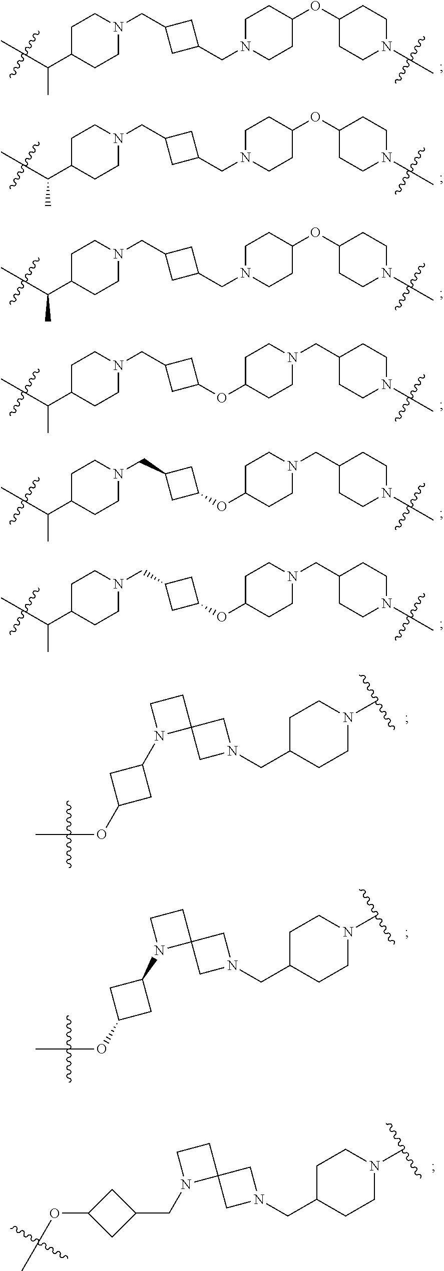

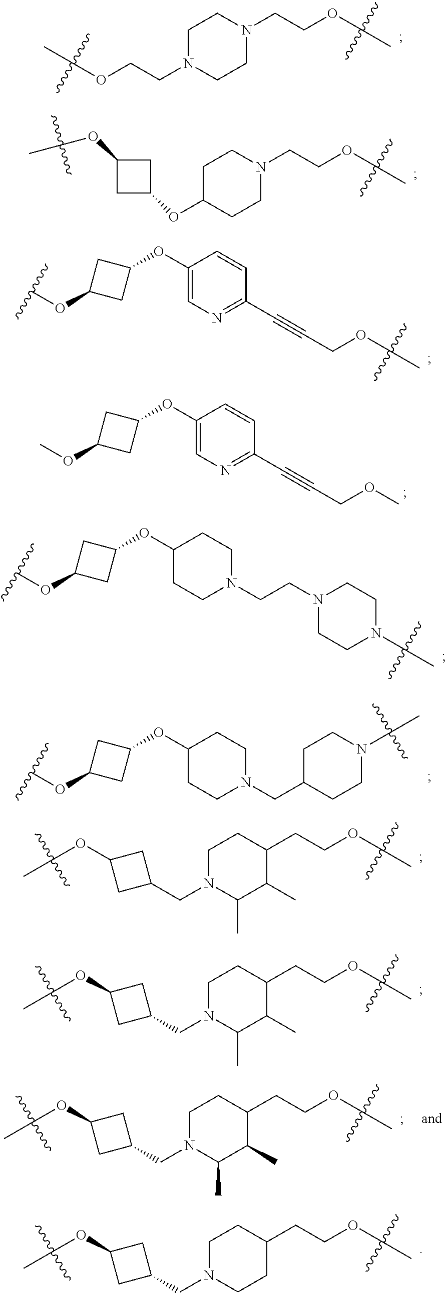

In certain embodiments, L has the formula —(C₀₋₁₂ alkylene)-(optionally substituted 3-40 membered heteroalkylene)-(C₀₋₁₂ alkylene)-. In certain embodiments, L is C₄₋₁₄ alkylene. In certain embodiments, L is —(CH₂)₆₋₁₀—.

In certain embodiments, L is —CH₂CH₂(OCH₂CH₂)—***, —CH₂CH₂(OCH₂CH₂)₂—***, —CH₂CH₂(OCH₂CH₂)₃—***, —CH₂CH₂(OCH₂CH₂)₄—***, —CH₂CH₂(OCH₂CH₂)₅—***, —CH₂CH₂(OCH₂CH₂)₆—***, —CH₂CH₂(OCH₂CH₂)₇—***, —CH₂CH₂(OCH₂CH₂)₈—***, —CH₂CH₂(OCH₂CH₂)₉—***, —CH₂CH₂(OCH₂CH₂)₁₀—***, —CH₂CH₂(OCH₂CH₂)₁₁—***, —CH₂CH₂(OCH₂CH₂)₁₂—***, —CH₂CH₂(OCH₂CH₂)₁₃—***, —CH₂CH₂(OCH₂CH₂)₁₄—***, —CH₂CH₂(OCH₂CH₂)₁₅—***, or —CH₂CH₂(OCH₂CH₂)₁₆₋₂₀—***, where *** is a point of attachment to A².

In certain embodiments, L is —(C₂₋₂₀ alkylene)-(OCH₂CH₂)₂₋₄—(C₀₋₄ alkylene)-***, —(C₂₋₂₀ alkylene)-(OCH₂CH₂)₅₋₇—(C₀₋₄ alkylene)-***, —(C₂₋₂₀ alkylene)-(OCH₂CH₂)₈₋₁₀—(C₀₋₄ alkylene)-***, —(C₂₋₂₀ alkylene)-(OCH₂CH₂)₁₁₋₁₃—(C₀₋₄ alkylene)-***, —(C₂₋₂₀ alkylene)-(OCH₂CH₂)₁₄₋₁₆—(C₀₋₄ alkylene)-***, —(C₂₋₂₀ alkylene)-(OCH₂CH₂)₁₇₋₂₀—(C₀₋₄ alkylene)-***, —(C₁₋₂₀ alkylene)-(OCH₂CH₂)₁₋₁₀—(C₀₋₄ alkylene)-C(O)—***, or —(C₁₋₂₀ alkylene)-(OCH₂CH₂)₁₁₋₂₀—(C₀₋₄ alkylene)-C(O)—***, where *** is a point of attachment to A².

In certain embodiments, L is —O(CH₂CH₂O)₂₋₄—(C₀₋₄ alkylene)-***, —O(CH₂CH₂O)₅₋₇—(C₀₋₄ alkylene)-***, —O(CH₂CH₂O)₈₋₁₀—(C₀₋₄ alkylene)-***, —O(CH₂CH₂O)₁₁₋₁₃—(C₀₋₄ alkylene)-***, —O(CH₂CH₂O)₁₄₋₁₆—(C₀₋₄alkylene)-***, —O(CH₂CH₂O)₁₆₋₂₀—(C₀₋₄ alkylene)-***, —O(CH₂CH₂O)₂₋₁₀—(C₀₋₄ alkylene)C(O)—***, or —O(CH₂CH₂O)₁₁₋₂₀—(C₀₋₄ alkylene)C(O)—***, where *** is a point of attachment to A².

In certain embodiments, L is —(C₀₋₂₀ alkylene)-(OCH₂CH₂)₁₋₁₀—(N(C₁₋₄ alkyl))-***, —(C₀₋₂₀ alkylene)-(OCH₂CH₂)₁₁₋₂₀—(N(C₁₋₄ alkyl))-***, —(C₀₋₂₀ alkylene)-(CH₂CH₂O)₁₋₁₀—(C₂₋₁₀ alkylene)-(N(C₁₋₄ alkyl))-(C₀₋₁₀ alkylene)-***, or —(C₀₋₂₀ alkylene)-(CH₂CH₂O)₁₁₋₂₀—(C₂₋₁₀ alkylene)-(N(C₁₋₄ alkyl))-(C₀₋₁₀ alkylene)-***, where *** is a point of attachment to A².

In certain embodiments, L is selected from those depicted in the compounds in Table 1, below.

### Exemplary Specific Compounds

In certain embodiments, the compound is a compound in Table 1, or a pharmaceutically acceptable salt thereof. In certain embodiments, the compound is a compound in Table 1. In certain embodiments, the compound is any one of compounds I-1 to I-37 in Table 1, or a pharmaceutically acceptable salt thereof. In certain embodiments, the compound is any one of compounds I-1 to I-37 in Table 1.

TABLE 1

| Com- | |
|---|---|
| pound | |
| No. | Chemical Structure |
| I-1 | 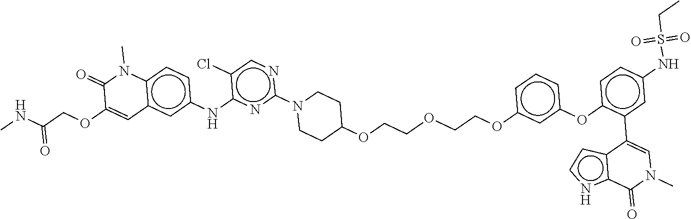 |
| I-2 | 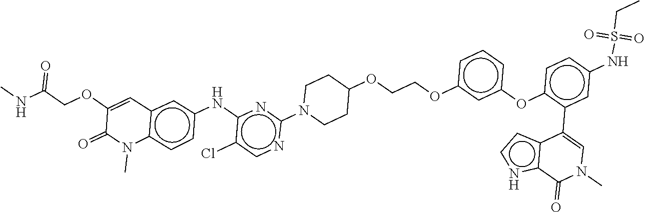 |
| I-3 | 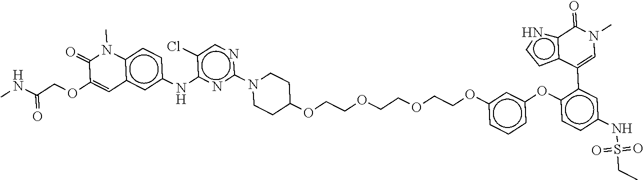 |
| I-4 | 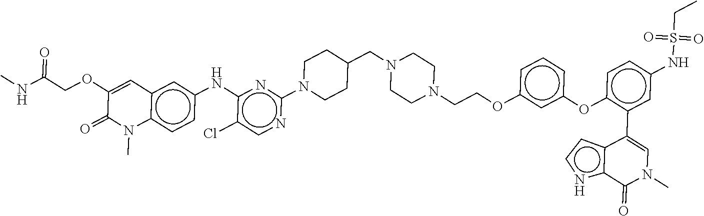 |
| I-5 | 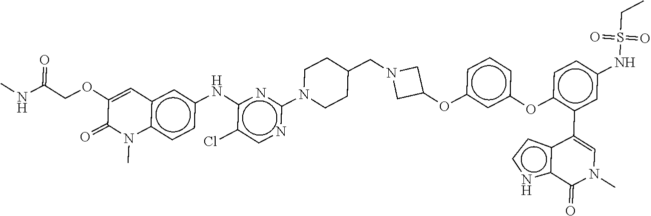 |
| I-6 | 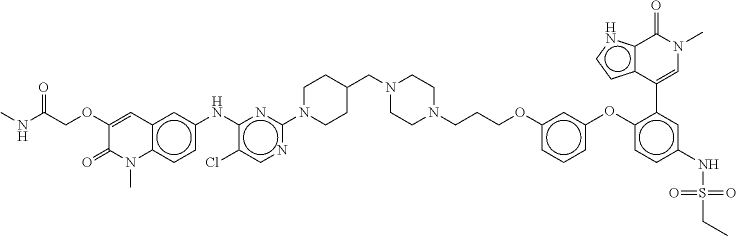 |
| I-7 | 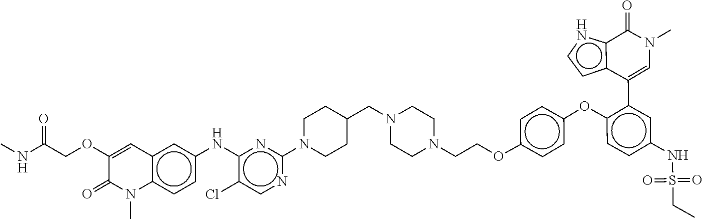 |
| I-8 | 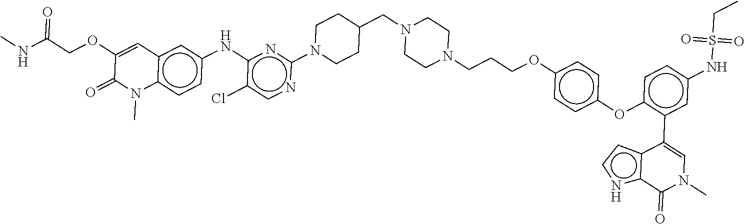 |
| I-9 | 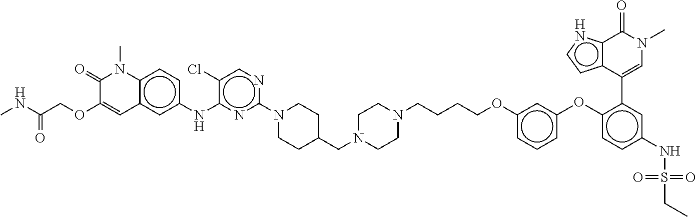 |
| I-10 | 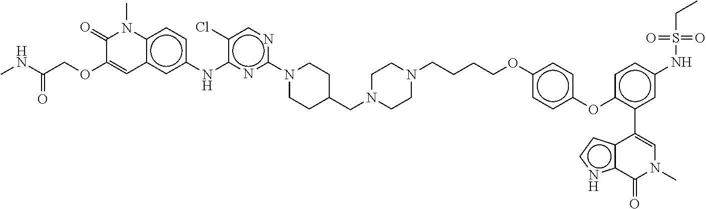 |
| I-11 | 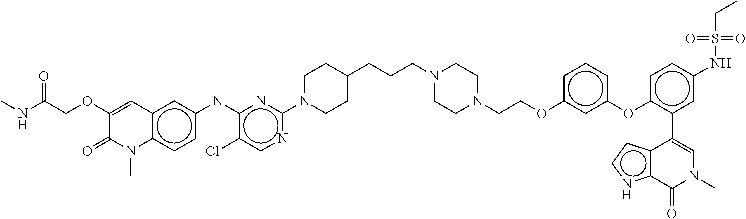 |
| I-12 | 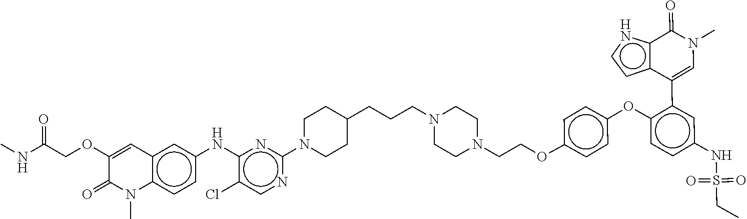 |
| I-13 | 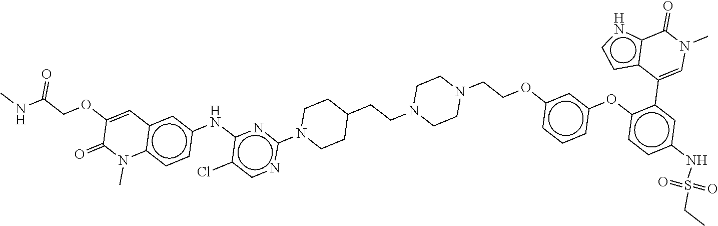 |
| I-14 | 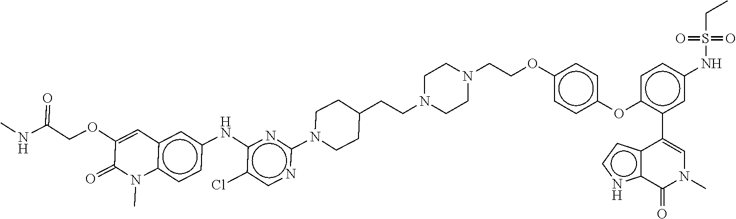 |
| I-15 | 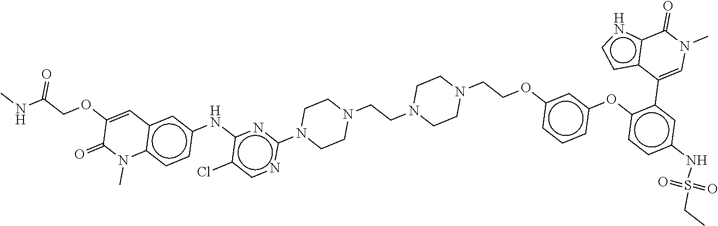 |
| I-16 | 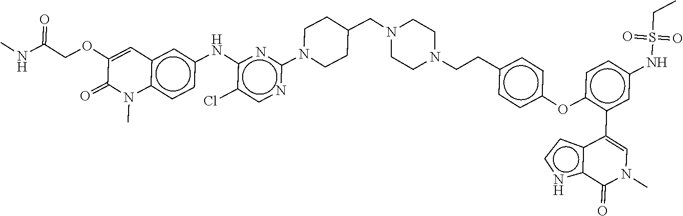 |
| I-17 | 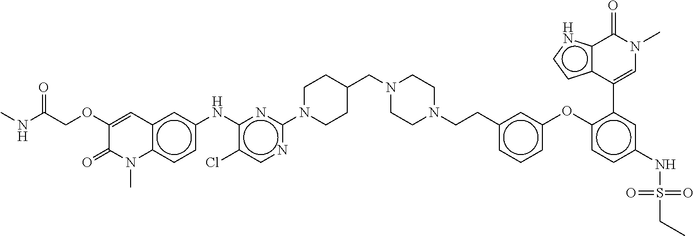 |
| I-18 | 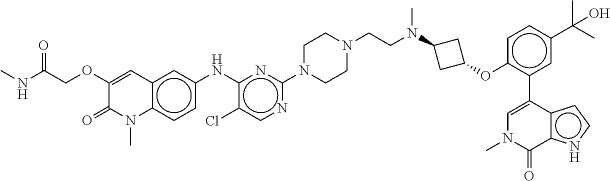 |
| I-19 | 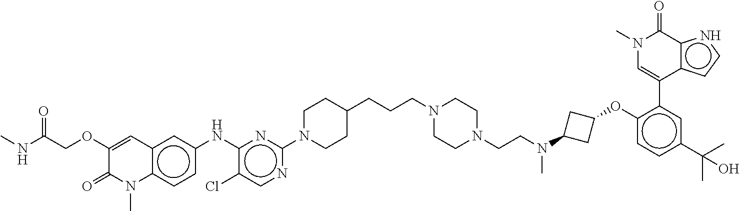 |
| I-20 | 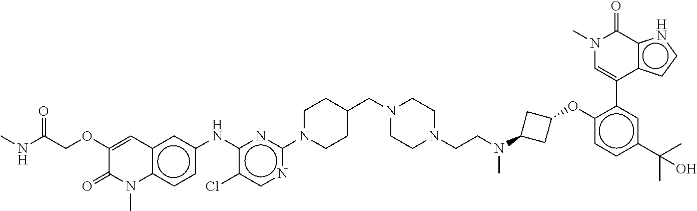 |
| I-21 | 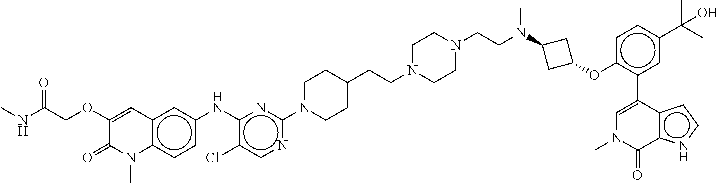 |
| I-22 | 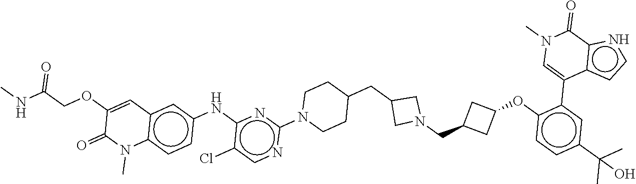 |
| I-23 | 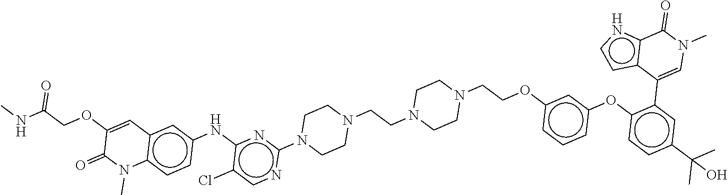 |
| I-24 | 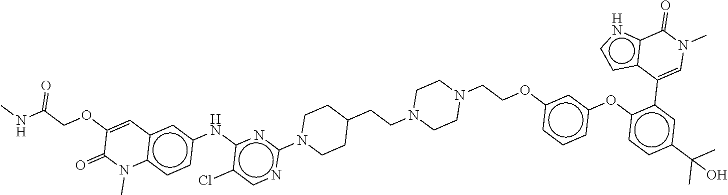 |
| I-25 | 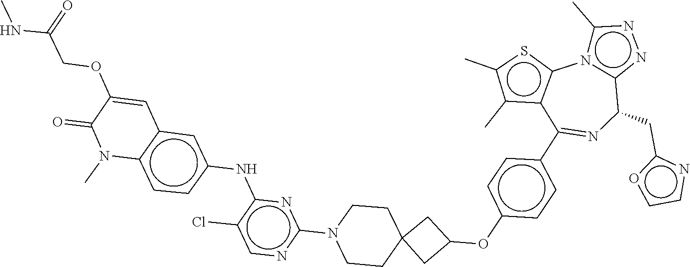 |
| I-26 | 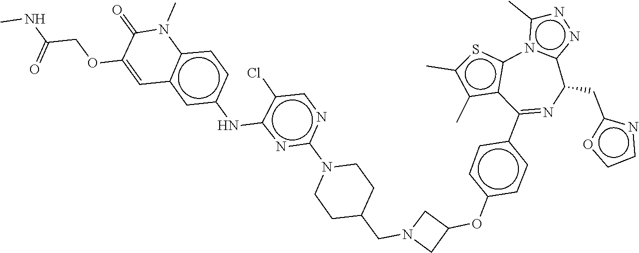 |
| I-27 | 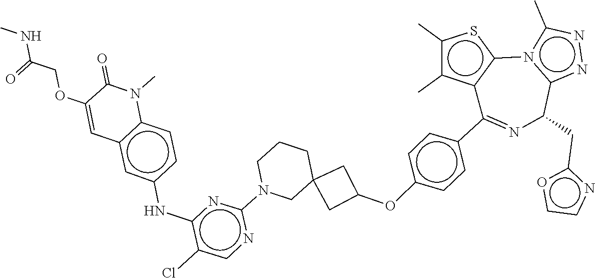 |
| I-28 | 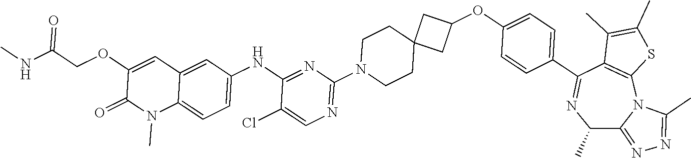 |
| I-29 | 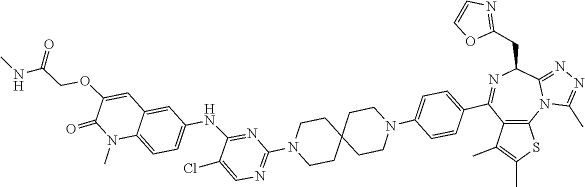 |
| I-30 | 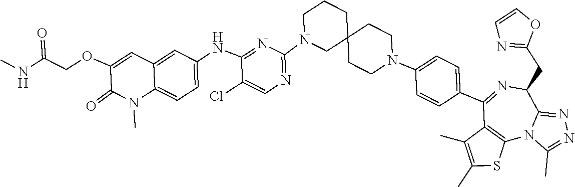 [SMILES: CNC(=O)COc1cc2cc(Nc3nc(N4CCCC5(CCN(c6ccc(C7=N[C@@H](Cc8ncco8)c8nnc(C)n8-c8sc(C)c(C)c87)cc6)CC5)C4)ncc3Cl)ccc2n(C)c1=O] |
| I-31 | 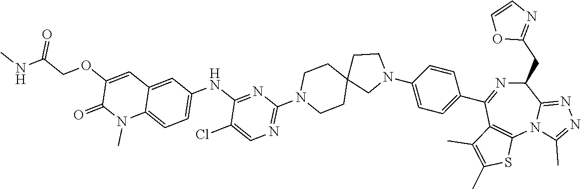 [SMILES: CNC(=O)COc1cc2cc(Nc3nc(N4CCC5(CCN(c6ccc(C7=N[C@@H](Cc8ncco8)c8nnc(C)n8-c8sc(C)c(C)c87)cc6)C5)CC4)ncc3Cl)ccc2n(C)c1=O] |
| I-32 | 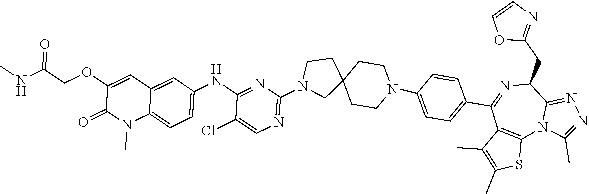 [SMILES: CNC(=O)COc1cc2cc(Nc3nc(N4CCC5(CCN(c6ccc(C7=N[C@@H](Cc8ncco8)c8nnc(C)n8-c8sc(C)c(C)c87)cc6)CC5)C4)ncc3Cl)ccc2n(C)c1=O] |
| I-33 | 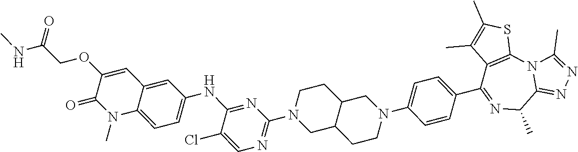 [SMILES: CNC(=O)COc1cc2cc(Nc3nc(N4CCC5CN(c6ccc(C7=N[C@@H](C)c8nnc(C)n8-c8sc(C)c(C)c87)cc6)CCC5C4)ncc3Cl)ccc2n(C)c1=O] |
| I-34 | 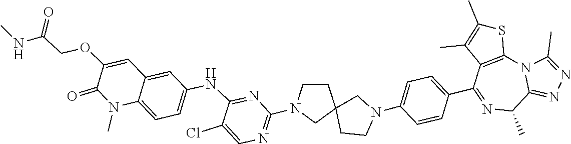 [SMILES: CNC(=O)COc1cc2cc(Nc3nc(N4CCC5(CCN(c6ccc(C7=N[C@@H](C)c8nnc(C)n8-c8sc(C)c(C)c87)cc6)C5)C4)ncc3Cl)ccc2n(C)c1=O] |
| I-35 | 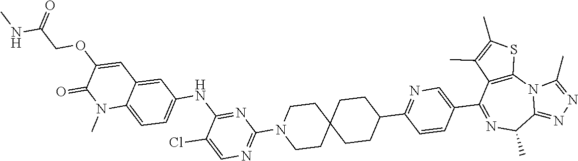 [SMILES: CNC(=O)COc1cc2cc(Nc3nc(N4CCC5(CCC(c6ccc(C7=N[C@@H](C)c8nnc(C)n8-c8sc(C)c(C)c87)cn6)CC5)CC4)ncc3Cl)ccc2n(C)c1=O] |
| I-36 | 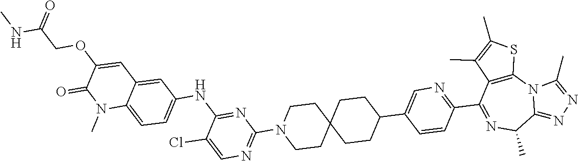 [SMILES: CNC(=O)COc1cc2cc(Nc3nc(N4CCC5(CCC(c6ccc(C7=N[C@@H](C)c8nnc(C)n8-c8sc(C)c(C)c87)nc6)CC5)CC4)ncc3Cl)ccc2n(C)c1=O] |
| I-37 | 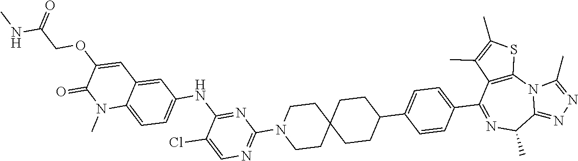 [SMILES: CNC(=O)COc1cc2cc(Nc3nc(N4CCC5(CCC(c6ccc(C7=N[C@@H](C)c8nnc(C)n8-c8sc(C)c(C)c87)cc6)CC5)CC4)ncc3Cl)ccc2n(C)c1=O] |
| I-38 | 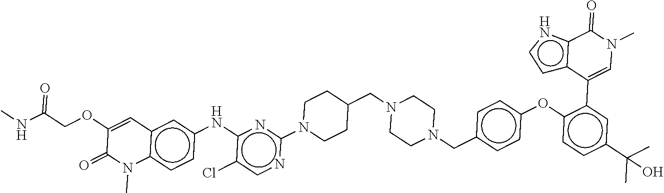 |
| I-39 | 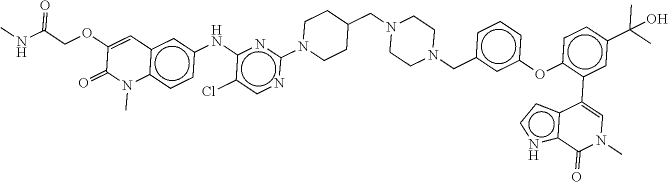 |
| I-40 | 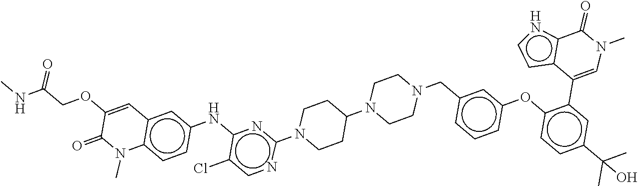 |
| I-41 | 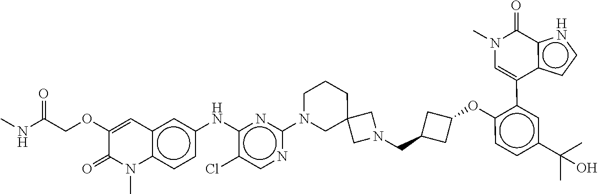 |
| I-42 | 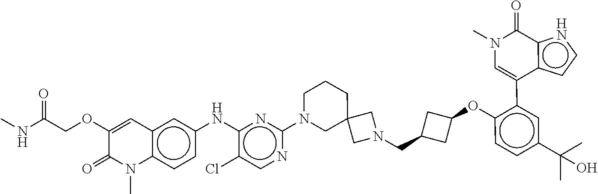 |
| I-43 | 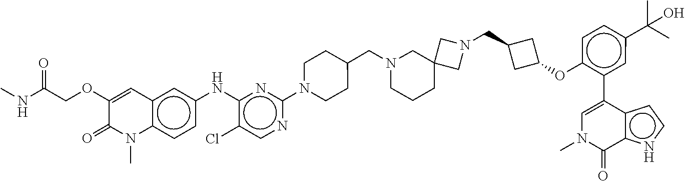 |
| I-44 | 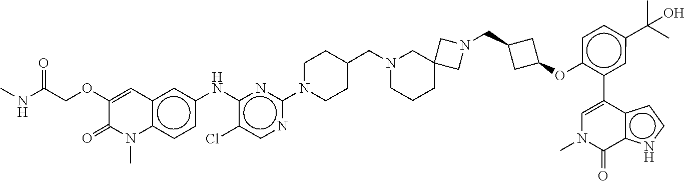 |
| I-45 | 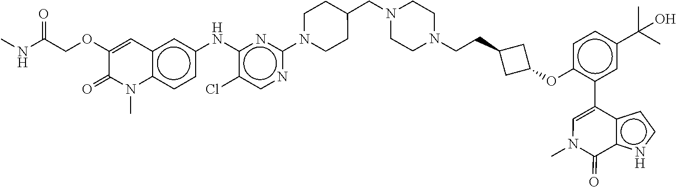 |
| I-46 | 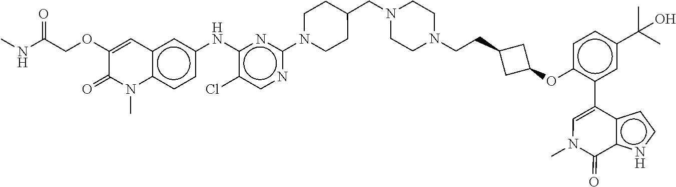 |
| I-47 | 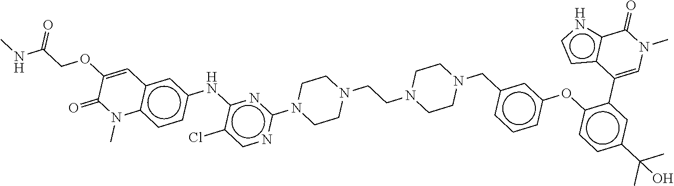 |
| I-48 | 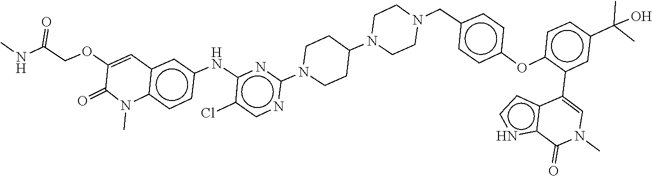 |
| I-49 | 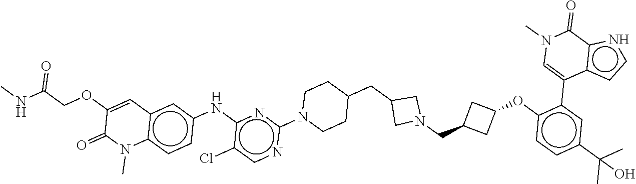 |
| I-50 | 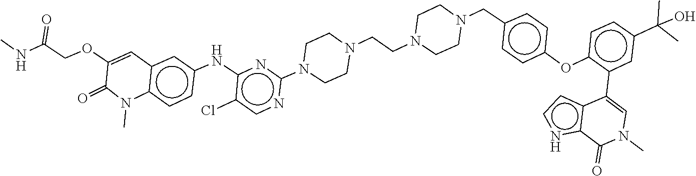 |
| I-51 | 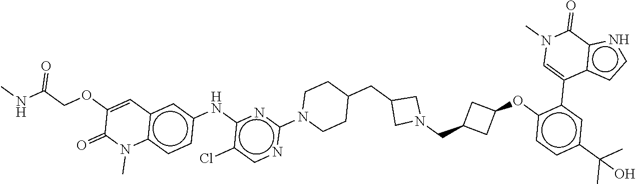 |
| I-52 | 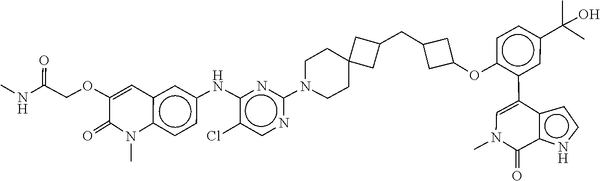 |
| I-53 | 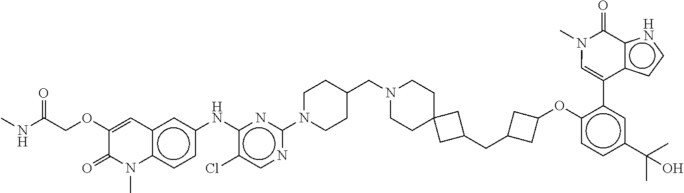 |
| I-54 | 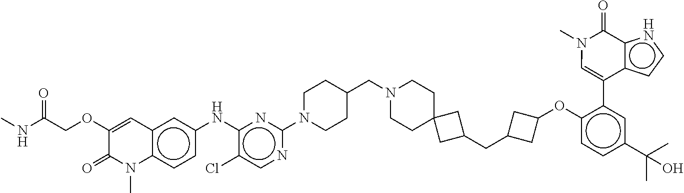 |
| I-55 | 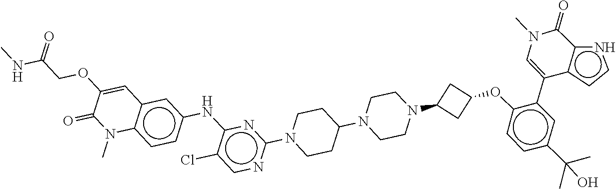 |

### Synthetic Methods

Methods for preparing compounds described herein are illustrated in the following synthetic Scheme. The Scheme is given for the purpose of illustrating the invention, and are not intended to limit the scope or spirit of the invention. Starting materials shown in the Scheme can be obtained from commercial sources or can be prepared based on procedures described in the literature.

In the Scheme, it is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated (for example, use of protecting groups or alternative reactions). Protecting group chemistry and strategy is well known, such as described in, for example, “Protecting Groups in Organic Synthesis”, T. W. Greene and P. G. M. Wuts, 3^{rd} edition, John Wiley & Sons, 1999, the entire contents of which are hereby incorporated by reference.

The synthetic route illustrated in Scheme 1 is a general method for preparing compounds of Formula C. Reaction of amine A and chloropyrimidine B under coupling conditions provides amide C.

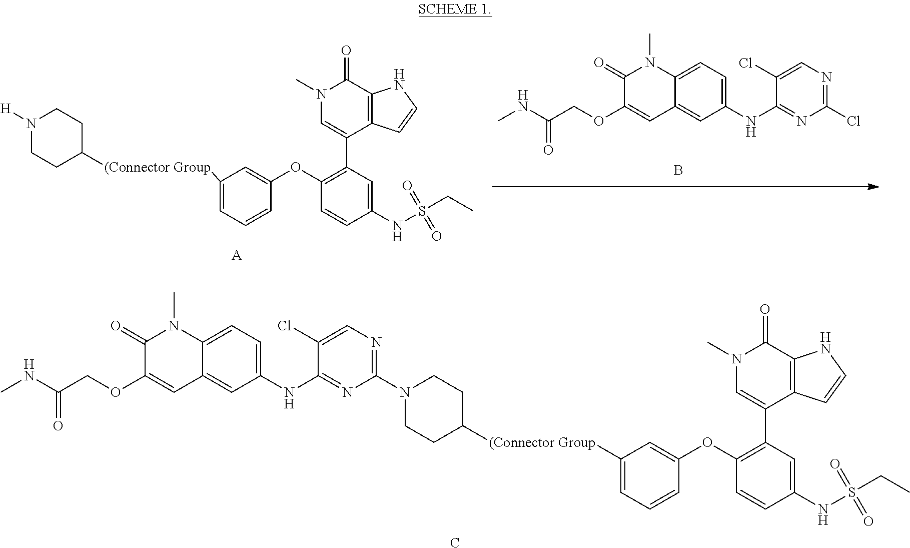

The modular synthetic route illustrated in Scheme 1 can be readily modified to provide additional compounds by conducting functional group transformations on the intermediate and/or final compounds. Such functional group transformations are well known in the art, as described in, for example, Comprehensive Organic Synthesis (B. M. Trost & I. Fleming, eds., 1991-1992); Organic Synthesis, 3^{rd} Ed. (Michael B. Smith, Wavefunction, Inc., Irvine: 2010); Modern Methods of Organic Synthesis, 4^{th} Ed. (William Carruthers and lain Coldham, Cambridge University Press, Cambridge: 2004); March's Advanced Organic Chemistry: Reactions, Mechanisms, and Structure, 8^{th} Ed., (Michael B. Smith, John Wiley & Sons, New York: 2020); and Comprehensive Organic Transformations: A Guide to Functional Group Preparations, 3rd Ed (Richard C. Larock, ed., John Wiley & Sons, New York: 2018). Protecting group strategies may be deployed as appropriate to accommodate differing functional groups in the molecules used in the synthetic route. Protecting group chemistry and strategy is described in, for example, Protecting Groups in Organic Synthesis, 3^{rd} Edition, T. W. Greene and P. G. M. Wuts, John Wiley & Sons, 1999 and Greene's Protective Groups in Organic Synthesis, 5th Ed., (Peter G. M. Wuts, John Wiley & Sons: 2014).

### II. Therapeutic Applications

The heterobifunctional compounds described herein, such as a compound of Formula I, II, or other compounds in Section I, provide therapeutic benefits to patients suffering from cancer. Accordingly, one aspect of the invention provides a method of treating cancer. The method comprises administering to a patient in need thereof a therapeutically effective amount of a compound described herein, such as a compound of Formula I, II, or other compound in Section I, to treat the cancer. In certain embodiments, the compound is a compound of Formula I. In certain embodiments, the particular compound of Formula I is a compound defined by one of the embodiments described above.

### Cancer

In certain embodiments, the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia.

In certain embodiments, the cancer is squamous cell cancer, lung cancer including small cell lung cancer, non-small cell lung cancer, vulval cancer, thyroid cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, hepatic carcinoma, anal carcinoma, penile carcinoma, and head and neck cancer. In certain embodiments, the cancer is at least one selected from the group consisting of ALL, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL, Philadelphia chromosome positive CML, lymphoma, leukemia, multiple myeloma myeloproliferative diseases, large B cell lymphoma, or B cell Lymphoma.

In certain embodiments, the cancer is a solid tumor or leukemia. In certain other embodiments, the cancer is colon cancer, pancreatic cancer, breast cancer, ovarian cancer, prostate cancer, squamous cell carcinoma, basal cell carcinoma, adenocarcinoma, sweat gland carcinoma, sebaceous gland carcinoma, lung cancer, leukemia, bladder cancer, stomach cancer, cervical cancer, testicular cancer, skin cancer, rectal cancer, thyroid cancer, kidney cancer, uterus cancer, esophagus cancer, liver cancer, an acoustic neuroma, oligodendroglioma, meningioma, neuroblastoma, or retinoblastoma. In certain other embodiments, the cancer is small cell lung cancer, non-small cell lung cancer, melanoma, cancer of the central nervous system tissue, brain cancer, Hodgkin's lymphoma, non-Hodgkin's lymphoma, cutaneous T-Cell lymphoma, cutaneous B-Cell lymphoma, or diffuse large B-Cell lymphoma. In certain other embodiments, the cancer is breast cancer, colon cancer, small-cell lung cancer, non-small cell lung cancer, prostate cancer, renal cancer, ovarian cancer, leukemia, melanoma, or cancer of the central nervous system tissue. In certain other embodiments, the cancer is colon cancer, small-cell lung cancer, non-small cell lung cancer, renal cancer, ovarian cancer, renal cancer, or melanoma.

In certain embodiments, the cancer is a fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, epithelial carcinoma, glioma, astrocytoma, medulloblastoma, or hemangioblastoma.

In certain embodiments, the cancer is a neuroblastoma, meningioma, hemangiopericytoma, multiple brain metastase, glioblastoma multiforms, glioblastoma, brain stem glioma, poor prognosis malignant brain tumor, malignant glioma, anaplastic astrocytoma, anaplastic oligodendroglioma, neuroendocrine tumor, rectal adeno carcinoma, Dukes C & D colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, metastatic melanoma, localized melanoma, malignant mesothelioma, malignant pleural effusion mesothelioma syndrome, peritoneal carcinoma, papillary serous carcinoma, gynecologic sarcoma, soft tissue sarcoma, scelroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waidenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, castrate resistant prostate cancer, castrate resistant metastatic prostate cancer, papillary thyroid carcinoma, follicular thyroid carcinoma, medullary thyroid carcinoma, or leiomyoma.

In certain embodiments, the cancer is bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In certain embodiments, the cancer is hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is a solid tumor, such as a sarcoma, carcinoma, or lymphoma. In certain embodiments, the cancer is kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In certain embodiments, the cancer is renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In certain embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

### Causing Death of Cancer Cell

Another aspect of the invention provides a method of causing death of a cancer cell. The method comprises contacting a cancer cell with an effective amount of a compound described herein, such as a compound of Formula I or II, or other compounds in Section I, to cause death of the cancer cell. In certain embodiments, the particular compound of Formula I or II is a compound defined by one of the embodiments described above.

In certain embodiments, the cancer cell is selected from ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct and gallbladder cancers, liver cancer, pancreatic cancer, bladder cancer, urinary tract cancer, kidney cancer, eye cancer, thyroid cancer, lymphoma, or leukemia. In certain embodiments, the cancer cell is one or more of the cancers recited in the section above entitled “Cancer.”

### Combination Therapies

The compounds useful within the methods of the invention may be used in combination with one or more additional therapeutic agents useful for treating any disease contemplated herein. These additional therapeutic agents may comprise compounds that are commercially available or synthetically accessible to those skilled in the art. These additional therapeutic agents are known to treat, prevent, or reduce the symptoms, of a disease or disorder contemplated herein.

Accordingly, in certain embodiments, the method further comprises administering to the subject an additional therapeutic agent that treats the disease contemplated herein.

In certain embodiments, administering the compound of the invention to the subject allows for administering a lower dose of the additional therapeutic agent as compared to the dose of the additional therapeutic agent alone that is required to achieve similar results in treating the disease contemplated herein. For example, in certain embodiments, the compound of the invention enhances the therapeutic activity of the additional therapeutic compound, thereby allowing for a lower dose of the additional therapeutic compound to provide the same effect.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In certain embodiments, the compound of the invention and the therapeutic agent are co-administered to the subject. In other embodiments, the compound of the invention and the therapeutic agent are coformulated and co-administered to the subject.

In certain embodiments, the compound is administered in combination with a second therapeutic agent having activity against cancer. In certain embodiments, the second therapeutic agent is mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

In certain embodiments, the second therapeutic agent is an mTOR inhibitor, which inhibits cell proliferation, angiogenesis and glucose uptake. Approved mTOR inhibitors useful in the present invention include everolimus (Afinitor®, Novartis); temsirolimus (Torisel®, Pfizer); and sirolimus (Rapamune®, Pfizer).

In certain embodiments, the second therapeutic agent is a Poly ADP ribose polymerase (PARP) inhibitor. Approved PARP inhibitors useful in the present invention include olaparib (Lynparza®, AstraZeneca); rucaparib (Rubraca®, Clovis Oncology); and niraparib (Zejula®, Tesaro). Other PARP inhibitors being studied which may be used in the present invention include talazoparib (MDV3800/BMN 673/LT00673, Medivation/Pfizer/Biomarin); veliparib (ABT-888, AbbVie); and BGB-290 (BeiGene, Inc.).

In certain embodiments, the second therapeutic agent is a phosphatidylinositol 3 kinase (PI3K) inhibitor. Approved PI3K inhibitors useful in the present invention include idelalisib (Zydelig®, Gilead). Other PI3K inhibitors being studied which may be used in the present invention include alpelisib (BYL719, Novartis); taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In certain embodiments, the second therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In certain embodiments, the second therapeutic agent is a histone deacetylase (HDAC) inhibitor. Approved HDAC inhibitors useful in the present invention include vorinostat (Zolinza®, Merck); romidepsin (Istodax®, Celgene); panobinostat (Farydak®, Novartis); and belinostat (Beleodaq®, Spectrum Pharmaceuticals). Other HDAC inhibitors being studied which may be used in the present invention include entinostat (SNDX-275, Syndax Pharmaceuticals) (NCT00866333); and chidamide (Epidaza®, HBI-8000, Chipscreen Biosciences, China).

In certain embodiments, the second therapeutic agent is a CDK inhibitor, such as a CDK 4/6 inhibitor. Approved CDK 4/6 inhibitors useful in the present invention include palbociclib (Ibrance®, Pfizer); and ribociclib (Kisqali®, Novartis). Other CDK 4/6 inhibitors being studied which may be used in the present invention include abemaciclib (Ly2835219, Eli Lilly); and trilaciclib (G1T28, G1 Therapeutics).

In certain embodiments, the second therapeutic agent is an indoleamine (2,3)-dioxygenase (IDO) inhibitor. IDO inhibitors being studied which may be used in the present invention include epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); and an enzyme that breaks down kynurenine (Kynase, Kyn Therapeutics).

In certain embodiments, the second therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In certain embodiments, the second therapeutic agent is an aromatase inhibitor. Approved aromatase inhibitors which may be used in the present invention include exemestane (Aromasin®, Pfizer); anastazole (Arimidex®, AstraZeneca) and letrozole (Femara®, Novartis).

In certain embodiments, the second therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In certain embodiments, the second therapeutic agent is a folic acid inhibitor. Approved folic acid inhibitors useful in the present invention include pemetrexed (Alimta®, Eli Lilly).

In certain embodiments, the second therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In certain embodiments, the second therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In certain embodiments, the second therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In certain embodiments, the second therapeutic agent is a glutaminase inhibitor. Glutaminase inhibitors being studied which may be used in the present invention include CB-839 (Calithera Biosciences).

In certain embodiments, the second therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In certain embodiments, the second therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In certain embodiments, the second therapeutic agent is a nucleoside inhibitor, or other therapeutic that interfere with normal DNA synthesis, protein synthesis, cell replication, or will otherwise inhibit rapidly proliferating cells. Such nucleoside inhibitors or other therapeutics include trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In certain embodiments, the second therapeutic agent is a platinum-based therapeutic, also referred to as platins. Platins cause cross-linking of DNA, such that they inhibit DNA repair and/or DNA synthesis, mostly in rapidly reproducing cells, such as cancer cells. Approved platinum-based therapeutics which may be used in the present invention include cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); and nedaplatin (Aqupla®, Shionogi). Other platinum-based therapeutics which have undergone clinical testing and may be used in the present invention include picoplatin (Poniard Pharmaceuticals); and satraplatin (JM-216, Agennix).

In certain embodiments, the second therapeutic agent is a taxane compound, which causes disruption of microtubules, which are essential for cell division. Approved taxane compounds which may be used in the present invention include paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), and cabazitaxel (Jevtana®, Sanofi-Aventis). Other taxane compounds which have undergone clinical testing and may be used in the present invention include SID530 (SK Chemicals, Co.) (NCT00931008).

In certain embodiments, the second therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In certain embodiments, the second therapeutic agent is a selective estrogen receptor modulator (SERM), which interferes with the synthesis or activity of estrogens. Approved SERMs useful in the present invention include raloxifene (Evista®, Eli Lilly).

In certain embodiments, the second therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In certain embodiments, the second therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In certain embodiments, the second therapeutic agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the additional therapeutic agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In certain embodiments, the second therapeutic agent is an immune checkpoint inhibitor selected from a PD-1 antagonist, a PD-L1 antagonist, or a CTLA-4 antagonist. In some embodiments, a compound disclosed herein or a pharmaceutically acceptable salt thereof is administered in combination with nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); or atezolizumab (anti-PD-L1 antibody, Tecentriq®, Genentech). Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Another aspect of the invention provides for the use of a compound described herein (such as a compound of Formula I, Formula II, or other compounds in Section I) in the manufacture of a medicament. In certain embodiments, the medicament is for treating a disease described herein, such as cancer.

Another aspect of the invention provides for the use of a compound described herein (such as a compound of Formula I, Formula II, or other compounds in Section I) for treating a medical disease, such a disease described herein (e.g., cancer).

### Evaluation of Cellular Growth Inhibition of HEK293 Cells and HeLa Cells

Compounds can be evaluated for ability to inhibit the proliferation of HEK293 cells or HeLa cells according to the following procedure. HEK293 and HeLa cells are cultured in DMEM medium supplemented with 10% fetal bovine serum and 1% Penn/Strep. Cells are seeded in white 384-well plates at 500 cells/well in 25 μL complete medium. Following seeding, plates are spun at 300×g for three minutes and cultured at 37° C. with 5% CO₂ in a humidified tissue culture incubator. After 24 hours, compounds are titrated in 100% DMSO and diluted in complete cell culture medium. A 25 μL aliquot of compound/media mixture is added to cells to bring total volume in well to 50 μL. DMSO alone is used as a negative control. Plates are then spun at 300×g for three minutes and stored at 37° C. with 5% CO₂ for three days. On Day 0 and Day 3 of compound treatment, cell viability is quantified with CellTiter-Glo 2.0 reagent (Promega). After equilibrating microplates at room temperature for 30 minutes, 25 μL CellTiter-Glo 2.0 reagent is dispensed into each well to bring total volume to 75 μL. Plates are mixed on shaker for 2 minutes at 500 rpm, followed by a 10-minute incubation at room temperature. Following a quick spin, luminescence readings are measured with an EnVision Plate Reader. Data is normalized to DMSO treated Day 0 and Day 3 readings. A four-parameter non-linear regression curve fit is applied to dose-response data in GraphPad Prism data analysis software to determine the half maximal growth inhibitory concentration (GI₅₀) for each compound.

### III. Pharmaceutical Compositions and Dosing Considerations

As indicated above, the invention provides pharmaceutical compositions, which comprise a therapeutically-effective amount of one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally. In certain embodiments, the invention provides a pharmaceutical composition comprising a compound described herein (e.g., a compound of Formula I) and a pharmaceutically acceptable carrier.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a heterobifunctional substituted phenylpyrimidinone or related compound described herein in a therapeutically effective amount for the treatment of a medical disorder described herein.

### IV. Medical Kits

Another aspect of this invention is a kit comprising (i) a compound described herein, such as a compound of Formula I, and (ii) instructions for use, such as treating cancer.

### EXAMPLES

The invention now being generally described, will be more readily understood by reference to the following examples, which are included merely for purposes of illustration of certain aspects and embodiments of the present invention, and is not intended to limit the invention.

### General Methods

All reactions were carried out under an atmosphere of dry nitrogen or argon. Glassware was oven-dried prior to use. Unless otherwise indicated, common reagents or materials were obtained from commercial sources and used without further purification. N,N-Diisopropylethylamine (DIPEA) was obtained anhydrous by distillation over potassium hydroxide. Tetrahydrofuran (THF), dichloromethane (CH₂Cl₂), and dimethylformamide (DMF) was dried by a PureSolv™ solvent drying system. PTLC refers to preparatory thin layer chromatographic separation. Abbreviations: HFIP (hexafluoroisopropanol), HEPES (4-(2-hydroxyethyl)-1-piperazineethanesulfonic acid. Flash column chromatography was performed using silica gel 60 (230-400 mesh). Analytical thin layer chromatography (TLC) was carried out on Merck silica gel plates with QF-254 indicator and visualized by UV or KMnO₄.

¹H and ¹³C NMR spectra were recorded on an Agilent DD₂ 500 (500 MHz ¹H; 125 MHz ¹³C) or Agilent DD₂ 600 (600 MHz ¹H; 150 MHz ¹³C) or Agilent DD₂ 400 (400 MHz ¹H; 100 MHz ¹³C) spectrometer at room temperature. Chemical shifts were reported in ppm relative to the residual CDCl₃ (δ 7.26 ppm ¹H; δ 77.0 ppm ¹³C), CD₃OD (δ 3.31 ppm ¹H; δ 49.00 ppm ¹³C), or d₆-DMSO (δ 2.50 ppm ¹H; δ 39.52 ppm ¹³C). NMR chemical shifts were expressed in ppm relative to internal solvent peaks, and coupling constants were measured in Hz (bs=broad signal). In most cases, only peaks of the major rotamer are reported.

Mass spectra were obtained using Agilent 1100 series LC/MSD spectrometers. Analytical HPLC analyses were carried out on 250×4.6 mm C-18 column using gradient conditions (10-100% B, flow rate=1.0 mL/min, 20 min), or as as described in the LC-MS Method tables.

Unless indicated otherwise, preparative HPLC was carried out on 250×21.2 mm C-18 column using gradient conditions (10-100% B, flow rate=10.0 mL/min, 20 min). The eluents used were: solvent A (H₂O with 0.1% TFA) and solvent B (CH₃CN with 0.1% TFA). Final products were typically purified via reversed-phase HPLC, PTLC, or flash column chromatography.

Method 01

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10(L/min) |
| | Nebulizer | 40(psi) |
| | Pressure | |
| | Drying Gas | 350° C. |
| | Temperature | |
| | Capillary Voltage | 2500(V) |
| | MS Polarity | Positive |
| | MS Mode | Scan |
| | Mass Range | 100-1500 |

Method 10

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N₂ |
| | Drying Gas Flow | 10(L/min) |
| | Nebulizer | 40(psi) |
| | Pressure | |
| | Drying Gas | 350° C. |
| | Temperature | |
| | Capillary Voltage | 2500(V) |
| | MS Polarity | Positive |
| | MS Mode | Scan |
| | Mass Range | 100-1500 |

Method 25

| Instrument | | Agilent 1100 LC & Agilent G1956A | | |
|---|---|---|---|---|
| Software | | Agilent Chemstation Rev. B. 04.03[54] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 25 | 0.8 |
| | | 0.40 | 25 | 0.8 |
| | | 3.40 | 100 | 0.8 |
| | | 3.90 | 100 | 0.8 |
| | | 3.91 | 25 | 0.8 |
| | | 4.00 | 25 | 1.0 |
| | | 4.50 | 25 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10(L/min) |
| | Nebulizer | 40(psi) |
| | Pressure | |
| | Drying Gas | 350° C. |
| | Temperature | |
| | Capillary Voltage | 2500(V) Positive |
| | MS Polarity | Positive |
| | MS Mode | Scan |
| | Mass Range | 100-1500 |

Method 55

| Instrument | | SHIMADZU LCMS-2020 | | |
|---|---|---|---|---|
| Software | | LabSolutions Version 5.93 | | |
| HPLC | Column | Kinetex EVO C18 2.1 × 30 mm, 5 um | | |
| | Mobile Phase | A: 0.025% NH₃•H₂O in water (v/v) | | |
| | | B: Acetonitrile | | |
| | Gradient | Time(min) | B (%) | Flow(mL/min) |
| | | 0.00 | 5 | 1.5 |
| | | 0.80 | 95 | 1.5 |
| | | 1.2 | 95 | 1.5 |
| | | 1.21 | 5 | 1.5 |
| | | 1.55 | 5 | 1.5 |

| | | |
|---|---|---|
| | Column Temp | 40° C. |
| | Detector | PDA (220&254 nm) |
| MS | Ionization source | ESI |
| | Drying Gas | N2 |
| | Drying Gas Flow | 15(L/min) |
| | DL Voltage | 120(V) |
| | Qarray DC Voltage | 20(V) |
| | MS Polarity | Positive |
| | MS Mode | Scan |
| | Mass range | 100-1000 |

The following abbreviations are used herein: ACN: acetonitrile; Bn: benzyl; Boc: tert-butoxycarbonyl; DCM: dichloromethane; DIEA: diisopropylethylamine; DMF: dimethylformamide; DMSO: dimethylsulfoxide; EtOH: ethanol; EA or EtOAc: ethyl acetate; equiv. or eq.: molar equivalents; FA: formic acid; h: hour or hours; HATU: 1-[bis(dimethylamino)methylene]-1H-1,2,3-triazolo[4,5-b]pyridinium 3-oxide hexafluorophosphate; HPLC: high-pressure liquid chromatography; LCMS or LC-MS: liquid chromatography-mass spectrometry; MeCN: acetonitrile; MeOH: methanol; MS: mass spectrometry; NMP: N-methylpyrrolidone; NMR: nuclear magnetic resonance; PE: petroleum ether; rt: room temperature; TEA: triethylamine; TFA: trifluoroacetic acid; THF: tetrahydrofuran; TLC: thin-layer chromatography; psi: pounds-per-square inch; and Tos or Ts: p-toluenesulfonyl.

### Example 1—Synthesis of 2-[[6-[[5-chloro-2-[4-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]-1-piperidyl]pyrimidin-4-yl]amino]-1-methyl-2-oxo-3-quinolyl]oxy]-N-methyl-acetamide (I-1)

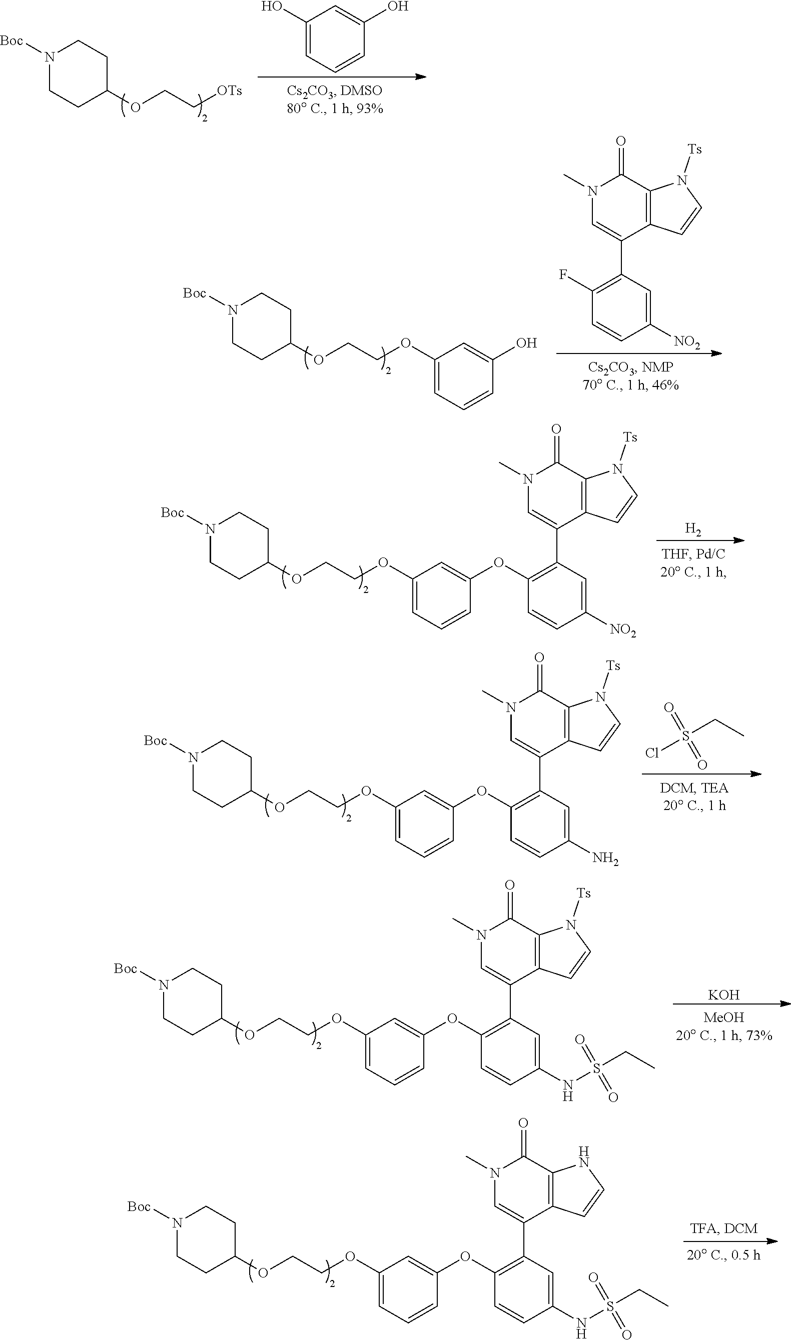

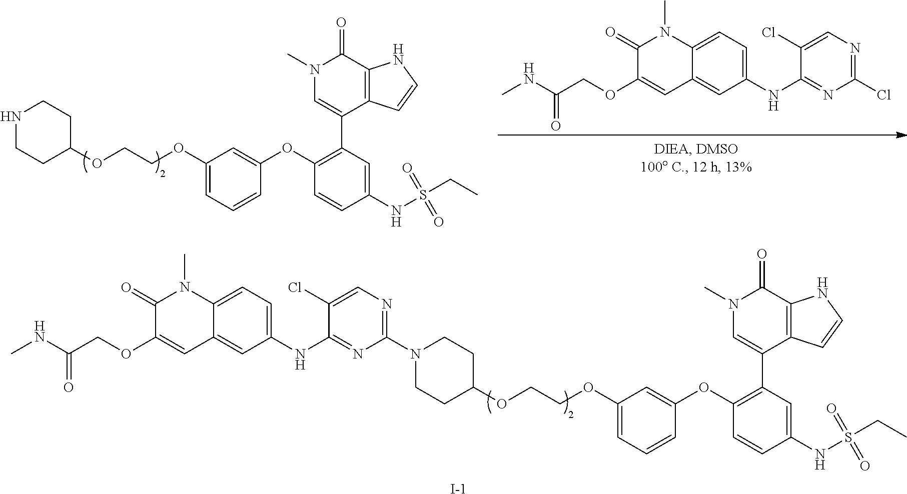

Step 1: Preparation of tert-butyl 4-[2-[2-(3-hydroxyphenoxy)ethoxy]ethoxy]piperidine-1-carboxylate. To a solution of tert-butyl 4-[2-[2-(p-tolylsulfonyloxy)ethoxy]ethoxy]piperidine-1-carboxylate (500 mg, 1.13 mmol, 1 equiv) (known compound from WO 2019/195609) and benzene-1,3-diol (372 mg, 3.38 mmol, 564 μL, 3 equiv) in DMSO (5 mL) was added Cs₂CO₃ (918 mg, 2.82 mmol, 2.5 equiv). The mixture was stirred at 80° C. for 1 h. The reaction mixture was filtered and concentrated under reduced pressure to give a residue. The residue was purified by prep-HPLC (column: Waters Xbridge C18 150*50 mm*10 um; mobile phase: [water (NH₄HCO₃)-ACN]; B %: 35%-65%, 10 min) to give the compound tert-butyl 4-[2-[2-(3-hydroxyphenoxy)ethoxy]ethoxy]piperidine-1-carboxylate (400 mg, 93% yield) as a yellow oil.

Step 2: Preparation of tert-butyl 4-[2-[2-[3-[2-[6-methyl-7-oxo-1-(p-tolylsulfonyl)pyrrolo[2,3-c]pyridin-4-yl]-4-nitro-phenoxy]phenoxy]ethoxy]ethoxy]piperidine-1-carboxylate. To a solution of tert-butyl 4-[2-[2-(3-hydroxyphenoxy)ethoxy]ethoxy]piperidine-1-carboxylate (185 mg, 484 umol, 1 equiv) and 4-(2-fluoro-5-nitro-phenyl)-6-methyl-1-(p-tolylsulfonyl)pyrrolo[2,3-c]pyridin-7-one (214 mg, 484 umol, 1 equiv) (known compound from Journal of Medicinal Chemistry, 2017, vol. 60, #20, p. 8369-8384) in NMP (5 mL) was added Cs₂CO₃ (395 mg, 1.21 mmol, 2.5 equiv). The mixture was stirred at 70° C. for 1 h. The reaction mixture was filtered and concentrated under reduced pressure to give a residue. The residue was purified by prep-HPLC (column: Waters Xbridge C18 150*50 mm*10 um; mobile phase: [water (NH₄HCO₃)-ACN]; B %: 54%-84%, 10 min) to give the compound tert-butyl 4-[2-[2-[3-[2-[6-methyl-7-oxo-1-(p-tolylsulfonyl)pyrrolo[2,3-c]pyridin-4-yl]-4-nitro-phenoxy]phenoxy]ethoxy]ethoxy]piperidine-1-carboxylate (180 mg, 46% yield) as a yellow oil.

Step 3: Preparation of tert-butyl 4-[2-[2-[3-[4-amino-2-[6-methyl-7-oxo-1-(p-tolylsulfonyl)pyrrolo[2,3-c]pyridin-4-yl]phenoxy]phenoxy]ethoxy]ethoxy]piperidine-1-carboxylate. To a solution of tert-butyl 4-[2-[2-[3-[2-[6-methyl-7-oxo-1-(p-tolylsulfonyl) pyrrolo[2,3-c]pyridin-4-yl]-4-nitro-phenoxy]phenoxy]ethoxy]ethoxy]piperidine-1-carboxylate (180 mg, 224 umol, 1 equiv) in THE (5 mL) was added Pd/C (100 mg, 224 umol, 10% purity) under N₂. The suspension was degassed under vacuum and purged with H₂ several times. The mixture was stirred under H₂ (15 psi) at 20° C. for 1 h. The reaction mixture was filtered and concentrated under reduced pressure to give the compound tert-butyl 4-[2-[2-[3-[4-amino-2-[6-methyl-7-oxo-1-(p-tolylsulfonyl)pyrrolo[2,3-c]pyridin-4-yl]phenoxy]phenoxy]ethoxy]ethoxy]piperidine-1-carboxylate (150 mg, crude) as a yellow oil.

Step 4: Preparation of tert-butyl 4-[2-[2-[3-[4-(ethylsulfonylamino)-2-[6-methyl-7-oxo-1-(p-tolylsulfonyl)pyrrolo[2,3-c]pyridin-4-yl]phenoxy]phenoxy]ethoxy]ethoxy]piperidine-1-carboxylate. To a solution of tert-butyl 4-[2-[2-[3-[4-amino-2-[6-methyl-7-oxo-1-(p-tolylsulfonyl)pyrrolo[2,3-c]pyridin-4-yl]phenoxy]phenoxy]ethoxy]ethoxy]piperidine-1-carboxylate (140 mg, 181 umol, 1 equiv) in DCM (3 mL) was added ethanesulfonyl chloride (93 mg, 724 umol, 68 μL, 4 equiv) and TEA (183 mg, 1.81 mmol, 252 μL, 10 equiv). The mixture was stirred at 20° C. for 2 h. The reaction mixture was filtered and concentrated under reduced pressure to give the compound tert-butyl 4-[2-[2-[3-[4-(ethylsulfonylamino)-2-[6-methyl-7-oxo-1-(p-tolylsulfonyl)pyrrolo[2,3-c]pyridin-4-yl]phenoxy]phenoxy]ethoxy]ethoxy]piperidine-1-carboxylate (150 mg, crude) as a yellow oil.

Step 5: Preparation of tert-butyl 4-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]piperidine-1-carboxylate. To a solution of tert-butyl 4-[2-[2-[3-[4-(ethylsulfonylamino)-2-[6-methyl-7-oxo-1-(p-tolylsulfonyl)pyrrolo[2,3-c]pyridin-4-yl]phenoxy]phenoxy]ethoxy]ethoxy]piperidine-1-carboxylate (150 mg, 173 umol, 1 equiv) in MeOH (3 mL) was added KOH (29 mg, 520 umol, 3 equiv). The mixture was stirred at 20° C. for 1 h. The reaction mixture was filtered and concentrated under reduced pressure to give a residue. The residue was purified by prep-TLC (SiO2, EA:MeOH=10:1) to give the compound tert-butyl 4-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]piperidine-1-carboxylate (90 mg, 126 umol, 73% yield) as a yellow oil.

Step 6: Preparation of N-[3-(6-methyl-7-oxo-1H-pyrrolo[2,3-c]pyridin-4-yl)-4-[3-[2-[2-(4-piperidyloxy)ethoxy]ethoxy]phenoxy]phenyl]ethanesulfonamide. To a solution of tert-butyl 4-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]piperidine-1-carboxylate (70 mg, 98 umol, 1 equiv) in DCM (4 mL) was added TFA (3.08 g, 27.01 mmol, 2 mL, 274 equiv). The mixture was stirred at 20° C. for 0.5 h. The reaction mixture was filtered and concentrated under reduced pressure to give the compound N-[3-(6-methyl-7-oxo-1H-pyrrolo[2,3-c]pyridin-4-yl)-4-[3-[2-[2-(4-piperidyloxy)ethoxy]ethoxy]phenoxy]phenyl]ethanesulfonamide (70 mg, crude, TFA) as a yellow oil.

Step 7: Preparation of 2-[[6-[[5-chloro-2-[4-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]-1-piperidyl]pyrimidin-4-yl]amino]-1-methyl-2-oxo-3-quinolyl]oxy]-N-methyl-acetamide. To a solution of N-[3-(6-methyl-7-oxo-1H-pyrrolo[2,3-c]pyridin-4-yl)-4-[3-[2-[2-(4-piperidyloxy) ethoxy]ethoxy]phenoxy]phenyl]ethanesulfonamide (70 mg, 96 umol, 1 equiv, TFA) and 2-[[6-[(2,5-dichloropyrimidin-4-yl)amino]-1-methyl-2-oxo-3-quinolyl]oxy]-N-methyl-acetamide (39 mg, 96 umol, 1 equiv) (known compound from WO2018/108704, 2018, A1) in DMSO (1 mL) was added DIEA (62 mg, 482 umol, 84 μL, 5 equiv). The mixture was stirred at 100° C. for 12 h. The reaction mixture was filtered and concentrated under reduced pressure to give a residue. The residue was purified by prep-HPLC (column: Waters Xbridge 150*25 mm*5 um; mobile phase: [water (NH₄HCO₃)-ACN]; B %: 30%-63%, 9 min) to give the compound 2-[[6-[[5-chloro-2-[4-[2-[2-[3-[4-(ethylsulfonylamino)-2-(6-methyl-7-oxo-1H-pyrrolo[2,3-c]pyridin-4-yl)phenoxy]phenoxy]ethoxy]ethoxy]-1-piperidyl]pyrimidin-4-yl]amino]-1-methyl-2-oxo-3-quinolyl]oxy]-N-methyl-acetamide (13 mg, 13% yield) as a yellow solid. ¹H NMR (400 MHz, MeOD): δ 7.9-8.0 (m, 4H), 7.59 (d, 1H, J=9.2 Hz), 7.46 (d, 1H, J=2.4 Hz), 7.31 (d, 1H, J=2.8 Hz), 7.25 (dd, 1H, J=2.8, 8.8 Hz), 7.17 (s, 1H), 7.0-7.1 (m, 2H), 6.54-6.17 (m, 1H), 6.3-6.4 (m, 3H), 3.9-4.2 (m, 6H), 3.79 (s, 6H), 3.6-3.7 (m, 6H), 3.56 (s, 3H), 3.21 (s, 3H), 3.1-3.2 (m, 2H), 1.8-1.9 (m, 2H), 1.5-1.5 (m, 2H), 1.36 (t, 3H, J=7.2 Hz). LC-MS: MS (ES⁺): RT=2.307 min, m/z=982.2 [M+H⁺]; LCMS method: Method 10.

### Example 2—Synthesis of 2-[[6-[[5-chloro-2-[4-[[4-[2-[4-[4-(ethylsulfonylamino)-2-(6-methyl-7-oxo-1H-pyrrolo[2,3-c]pyridin-4-yl)phenoxy]phenyl]ethyl]piperazin-1-yl]methyl]-1-piperidyl]pyrimidin-4-yl]amino]-1-methyl-2-oxo-3-quinolyl]oxy]-N-methyl-acetamide (I-16)

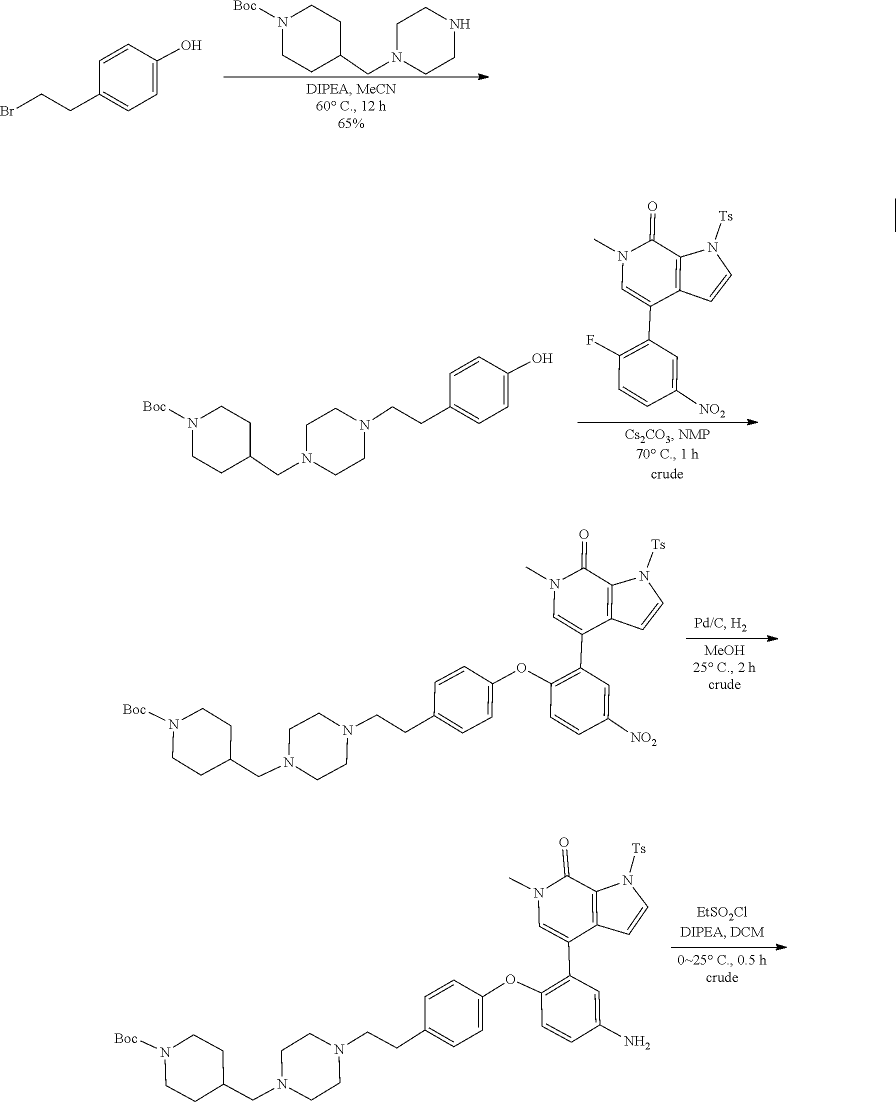

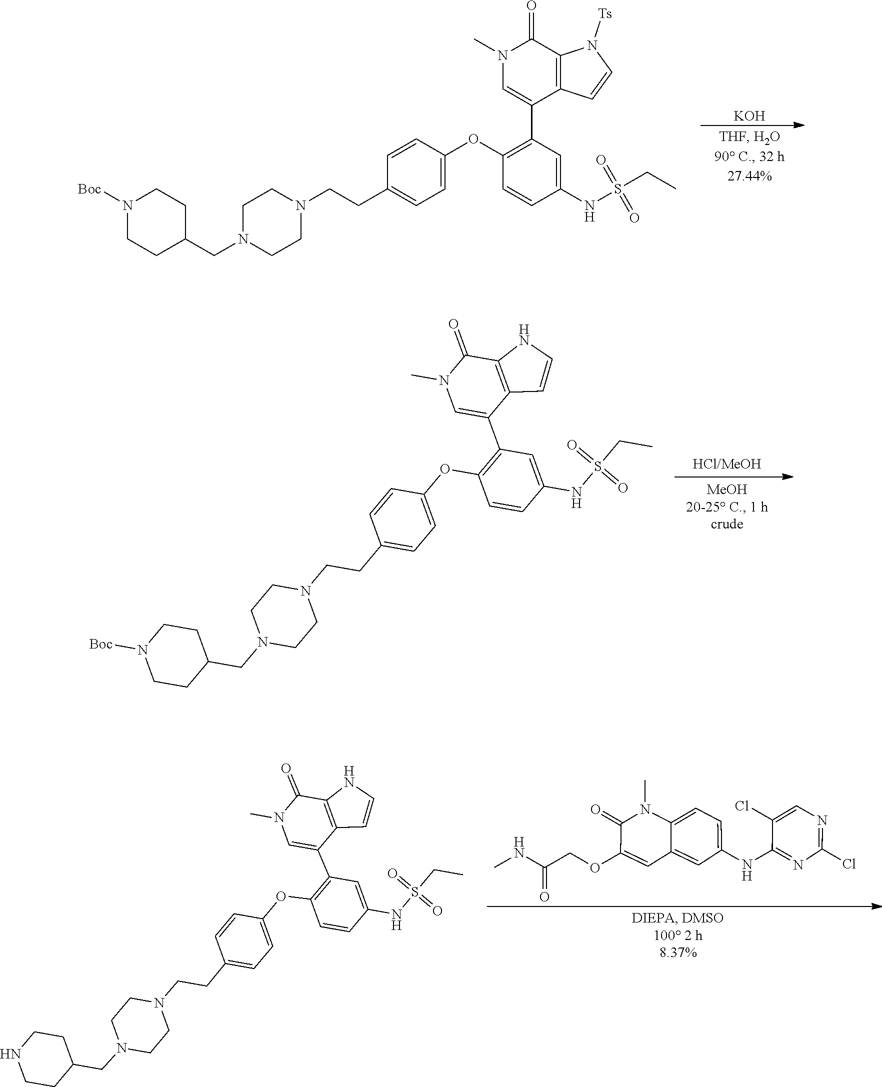

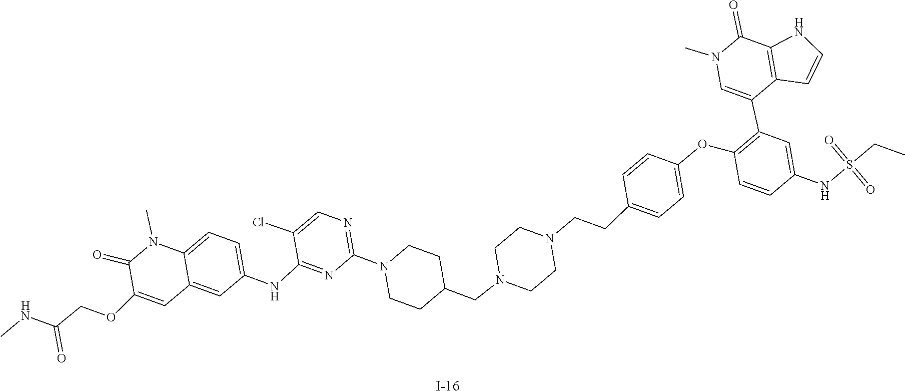

Step 1: Preparation of tert-butyl 4-[[4-[2-(4-hydroxyphenyl)ethyl]piperazin-1-yl]methyl]piperidine-1-carboxylate. To a solution of 4-(2-bromoethyl)phenol (500 mg, 2.49 mmol, 1 eq) and tert-butyl 4-(piperazin-1-ylmethyl)piperidine-1-carboxylate (845.74 mg, 2.98 mmol, 1.2 eq) in MeCN (10 mL) was added DIEA (964.21 mg, 7.46 mmol, 1.30 mL, 3 eq). The mixture was stirred at 60° C. for 12 hours. The mixture was evaporated. The residue was purified by prep-HPLC (column: Welch Ultimate XB-CN 250*70*10 um;mobile phase: [Hexane-EtOH 0.1% NH₃·H₂O];B %: 5%-45%,15 min) to give the tert-butyl 4-[[4-[2-(4-hydroxyphenyl) ethyl]piperazin-1-yl]methyl]piperidine-1-carboxylate (650 mg, 1.61 mmol, 64.77% yield) as a white solid.

Step 2: Preparation of tert-butyl 4-((4-(4-(2-(6-methyl-7-oxo-1-tosyl-6,7-dihydro-1H-pyrrolo[2,3-c]pyridin-4-yl)-4-nitrophenoxy)phenethyl)piperazin-1-yl)methyl) piperidine-1-carboxylate. To a solution of tert-butyl 4-[[4-[2-(4-hydroxyphenyl)ethyl]piperazin-1-yl]methyl]piperidine-1-carboxylate (220 mg, 545.15 umol, 1 eq) in NMP (1.2 mL) was added Cs₂CO₃ (444.05 mg, 1.36 mmol, 2.5 eq) and 4-(2-fluoro-5-nitro-phenyl)-6-methyl-1-(p-tolylsulfonyl)pyrrolo[2,3-c]pyridin-7-one (240.65 mg, 545.15 umol, 1 eq). The mixture was stirred at 70° C. for 1 hour. The reaction mixture was quenched by addition H₂O 10 mL, and then extracted with EA 20 mL (10 mL*2). The combined organic layers were washed with brine 20 mL (10 mL*2), dried over Na₂SO₄, filtered and concentrated under reduced pressure to give tert-butyl 4-((4-(4-(2-(6-methyl-7-oxo-1-tosyl-6,7-dihydro-1H-pyrrolo[2,3-c]pyridin-4-yl)-4-nitrophenoxy)phenethyl)piperazin-1-yl)methyl)piperidine-1-carboxylate (480 mg, crude) as a yellow oil.

Step 3: Preparation of tert-butyl 4-[[4-[2-[4-[4-amino-2-[6-methyl-7-oxo-1-(p-tolylsulfonyl)pyrrolo[2,3-c]pyridin-4-yl]phenoxy]phenyl]ethyl]piperazin-1-yl]methyl]piperidine-1-carboxylate. To a solution of tert-butyl 4-[[4-[2-[4-[2-[6-methyl-7-oxo-1-(p-tolylsulfonyl)pyrrolo[2,3-c]pyridin-4-yl]-4-nitrophenoxy]phenyl]ethyl]piperazin-1-yl]methyl]piperidine-1-carboxylate (600 mg, 727.29 umol, 1 eq) in MeOH (20 mL) was added Pd/C (100 mg, 10% purity) (10%) under N₂. The suspension was degassed under vacuum and purged with H₂ several times. The mixture was stirred under H₂ (50 psi) at 20-25° C. for 2 hours. The reaction mixture was concentrated under reduced pressure to give a residue. Compound tert-butyl 4-[[4-[2-[4-[4-amino-2-[6-methyl-7-oxo-1-(p-tolylsulfonyl)pyrrolo[2,3-c]pyridin-4-yl]phenoxy]phenyl]ethyl]piperazin-1-yl]methyl]piperidine-1-carboxylate (580 mg, crude) was obtained as black oil. The crude product was used into the next step without further purification.

Step 4: Preparation of tert-butyl4-[[4-[2-[4-[4-(ethylsulfonylamino)-2-[6-methyl-7-oxo-1-(p-tolylsulfonyl)pyrrolo[2,3-c]pyridin-4-yl]phenoxy]phenyl]ethyl]piperazin-1-yl]methyl]piperidine-1-carboxylate. To a solution of tert-butyl 4-[[4-[2-[4-[4-amino-2-[6-methyl-7-oxo-1-(p-tolylsulfonyl)pyrrolo[2,3-c]pyridin-4-yl]phenoxy]phenyl]ethyl]piperazin-1-yl]methyl]piperidine-1-carboxylate (330 mg, 415.09 umol, 1 eq) in DCM (6 mL) was added DIPEA (268.23 mg, 2.08 mmol, 361.50 uL, 5 eq) and ethanesulfonyl chloride (266.86 mg, 2.08 mmol, 196.22 uL, 5 eq) at 0-5° C. The mixture was stirred at 0-5° C. for 10 min, then warmed to 20-25° C. and stirred for another 20 min. The reaction mixture was quenched by addition H₂O 5 mL, and then extracted with DCM 5 mL. The combined organic layers were concentrated under reduced pressure to give tert-buty14-[[4-[2-[4-[4-(ethylsulfonylamino)-2-[6-methyl-7-oxo-1-(p-tolylsulfonyl)pyrrolo[2,3-c]pyridin-4-yl]phenoxy]phenyl]ethyl]piperazin-1-yl]methyl]piperidine-1-carboxylate (370 mg, crude) obtained as a yellow oil.

Step 5: Preparation of tert-butyl 4-[[4-[2-[4-[4-(ethylsulfonylamino)-2-(6-methyl-7-oxo-1H-pyrrolo[2,3-c]pyridin-4-yl)phenoxy]phenyl]ethyl]piperazin-1-yl]methyl]piperidine-1-carboxylate. To a solution of tert-butyl 4-[[4-[2-[4-[4-(ethylsulfonylamino)-2-[6-methyl-7-oxo-1-(p-tolylsulfonyl)pyrrolo[2,3-c]pyridin-4-yl]phenoxy]phenyl]ethyl]piperazin-1-yl]methyl]piperidine-1-carboxylate (370 mg, 417.08 umol, 1 eq) in THF (4 mL) and H₂O (2 mL) was added KOH (468.05 mg, 8.34 mmol, 20 eq) and hexadecyl(trimethyl)ammonium;bromide (3.04 mg, 8.34 umol, 0.02 eq). The mixture was stirred at 90° C. for 16 hours. Then KOH (234.01 mg, 4.17 mmol, 10 eq) and hexadecyl(trimethyl)ammonium;bromide (3.04 mg, 8.34 umol, 0.02 eq) was added and the mixture was stirred at 90° C. for another 16 hours. The mixture was purified by prep-HPLC (TFA.column: Phenomenex Synergi Polar-RP 100*25 mm*4 um; mobile phase: [water(FA)-MeOH];B %: 26%-56%,9 min) to give tert-butyl 4-[[4-[2-[4-[4-(ethylsulfonylamino)-2-(6-methyl-7-oxo-1H-pyrrolo[2,3-c]pyridin-4-yl)phenoxy]phenyl]ethyl]piperazin-1-yl]methyl]piperidine-1-carboxylate (110 mg, 114.47 umol, 27.44% yield, 2TFA) as an off-white solid.

Step 6: Preparation of N-[3-(6-methyl-7-oxo-1H-pyrrolo[2,3-c]pyridin-4-yl)-4-[4-[2-[4-(4-piperidylmethyl)piperazin-1-yl]ethyl]phenoxy]phenyl]ethanesulfonamide. To a solution of tert-butyl 4-[[4-[2-[4-[4-(ethylsulfonylamino)-2-(6-methyl-7-oxo-1H-pyrrolo[2,3-c]pyridin-4-yl)phenoxy]phenyl]ethyl]piperazin-1-yl]methyl]piperidine-1-carboxylate (110 mg, 114.47 umol, 1 eq, 2TFA) in MeOH (1 mL) was added HCl/MeOH (4 M, 2 mL, 69.89 eq). The mixture was stirred at 20-25° C. for another 1 hour. The reaction mixture was concentrated under reduced pressure to give N-[3-(6-methyl-7-oxo-1H-pyrrolo[2,3-c]pyridin-4-yl)-4-[4-[2-[4-(4-piperidylmethyl)piperazin-1-yl]ethyl]phenoxy]phenyl]ethanesulfonamide (80 mg, crude, 3HCl) (80 mg, crude, 3HCl) was obtained as a yellow solid.

Step 7: Preparation of 2-[[6-[[5-chloro-2-[4-[[4-[2-[4-[4-(ethylsulfonylamino)-2-(6-methyl-7-oxo-1H-pyrrolo[2,3-c]pyridin-4-yl)phenoxy]phenyl]ethyl]piperazin-1-yl]methyl]-1-piperidyl]pyrimidin-4-yl]amino]-1-methyl-2-oxo-3-quinolyl]oxy]-N-methyl-acetamide. To a solution of N-[3-(6-methyl-7-oxo-1H-pyrrolo[2,3-c]pyridin-4-yl)-4-[4-[2-[4-(4-piperidyl-methyl)piperazin-1-yl]ethyl]phenoxy]phenyl]ethanesulfonamide (80 mg, 107.79 umol, 1 eq, 3HCl) in DMSO (1 mL) was added DIPEA (139.31 mg, 1.08 mmol, 187.74 uL, 10 eq) and 2-[[6-[(2,5-dichloropyrimidin-4-yl)amino]-1-methyl-2-oxo-3-quinolyl]oxy]-N-methyl-acetamide (26.40 mg, 64.67 umol, 0.6 eq). The mixture was stirred at 100° C. for 2 hours. The residue was purified by prep-HPLC (NEU. column:Waters Xbridge 150*25 mm*5 um;mobile phase: [water(NH₄HCO₃)-ACN];B %: 40%-70%,8 min) to give 2-[[6-[[5-chloro-2-[4-[[4-[2-[4-[4-(ethylsulfonylamino)-2-(6-methyl-7-oxo-1H-pyrrolo[2,3-c]pyridin-4-yl)phenoxy]phenyl]ethyl]piperazin-1-yl]methyl]-1-piperidyl]pyrimidin-4-yl]amino]-1-methyl-2-oxo-3-quinolyl]oxy]-N-methyl-acetamide (9.48 mg, 9.03 umol, 8.37% yield, 95.66% purity) obtained as an off-white solid. ¹H NMR (400 MHz, DMSO) 12.04 (br s, 1H), 9.70-9.84 (m, 1H), 8.93 (s, 1H), 7.99-8.06 (m, 3H), 7.95 (dd, J=9.0, 2.0 Hz, 1H), 7.57 (d, J=9.1 Hz, 1H), 7.38 (d, J=2.6 Hz, 1H), 7.26-7.32 (m, 2H), 7.21 (dd, J=8.9, 2.3 Hz, 1H), 7.11 (br d, J=8.4 Hz, 2H), 6.98 (d, J=8.6 Hz, 1H), 6.78 (d, J=8.5 Hz, 2H), 6.26 (s, 1H), 4.64-4.76 (m, 1H), 4.47 (br d, J=13.1 Hz, 2H), 3.80-3.92 (m, 1H), 3.69 (s, 3H), 3.50 (s, 3H), 3.08-3.14 (m, 5H), 2.74-2.89 (m, 3H), 2.59-2.70 (m, 4H), 2.27-2.46 (m, 11H), 2.06-2.15 (m, 2H), 1.66-1.83 (m, 3H), 1.24 (t, J=7.3 Hz, 4H), 1.10-1.18 (m, 1H), 0.92-1.07 (m, 3H). LC-MS: MS(ES⁺): RT=0.612 min, m/z=1004.5 [M+H⁺]; LCMS Method: Method 55.

### Example 3—Synthesis of 2-[[6-[[5-chloro-2-[4-[2-[[3-[4-(1-hydroxy-1-methyl-ethyl)-2-(6-methyl-7-oxo-1H-pyrrolo[2,3-c]pyridin-4-yl)phenoxy]cyclobutyl]-methyl-amino]ethyl]piperazin-1-yl]pyrimidin-4-yl]amino]-1-methyl-2-oxo-3-quinolyl]oxy]-N-methyl-acetamide(I-18)

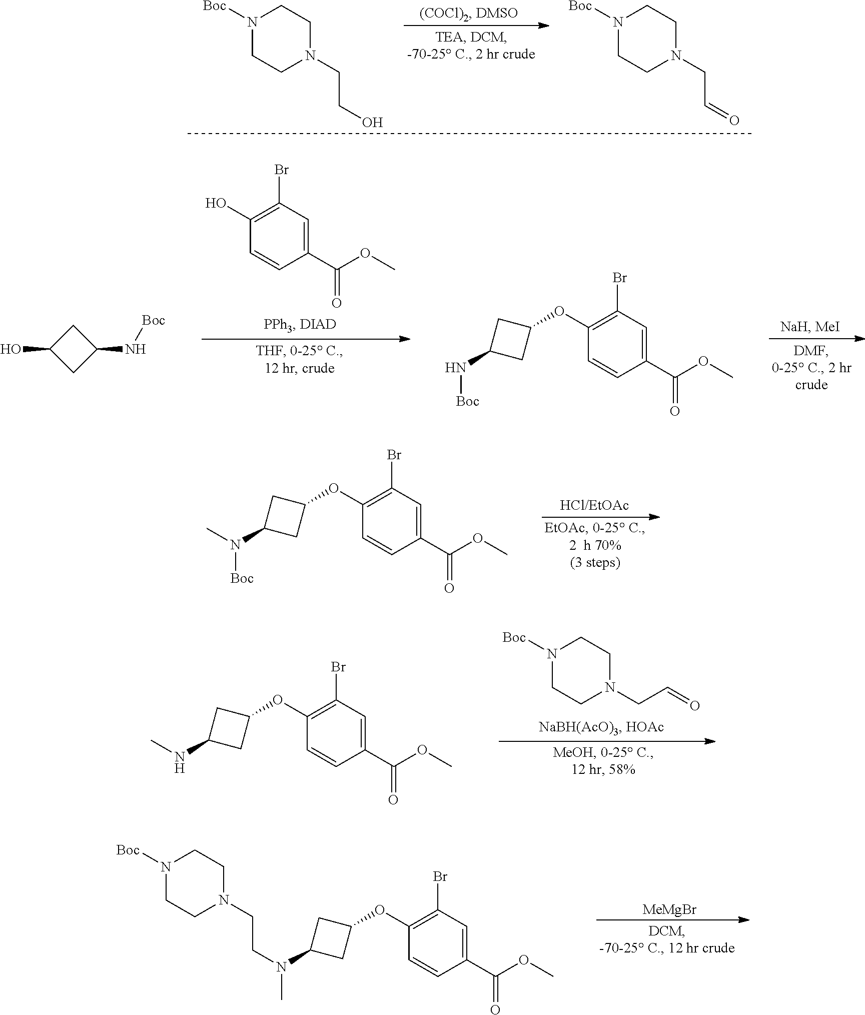

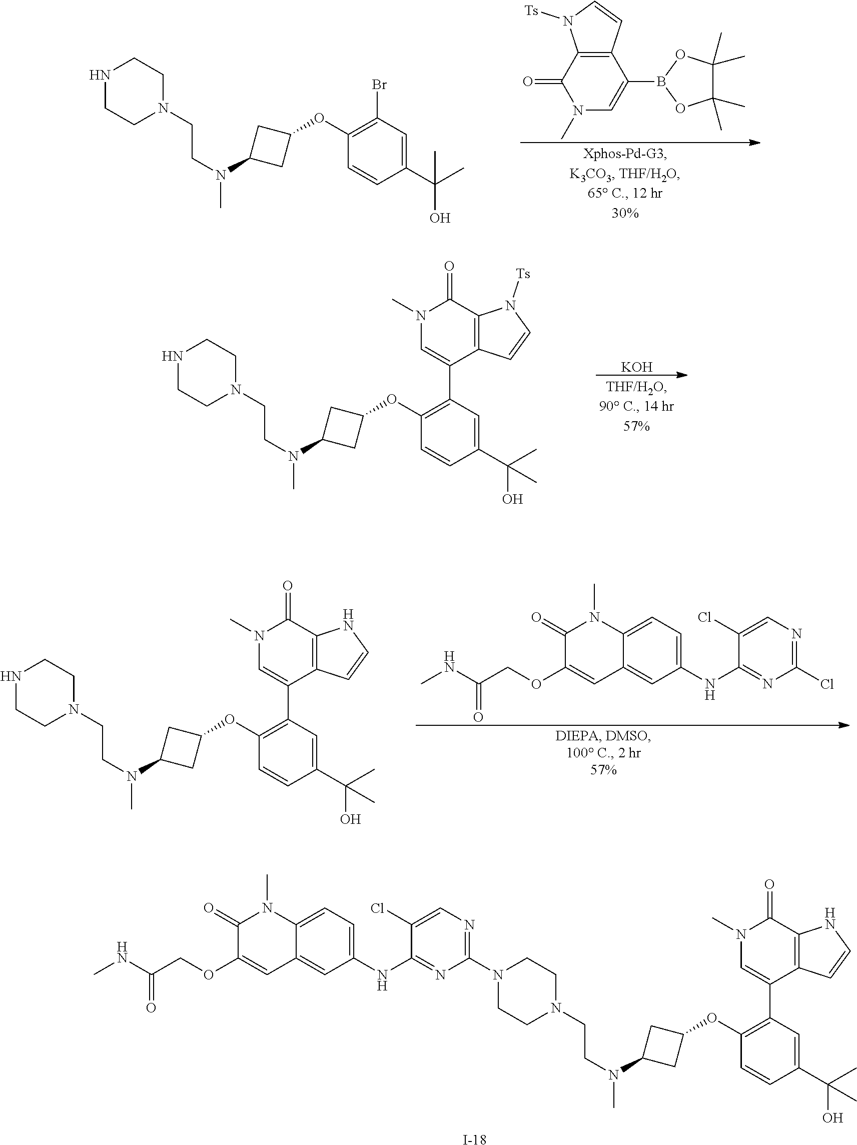

Step 1: Preparation of tert-butyl 4-(2-oxoethyl) piperazine-1-carboxylate. To a solution of (COCl)₂ (16.53 g, 130.26 mmol, 11.40 mL, 2 eq) in DCM (150 mL) was cooled to −70-−60° C., then DMSO (17.81 g, 227.96 mmol, 17.81 mL, 3.5 eq) was added slowly at −70-−60° C. The mixture was stirred at −70-−60° C. for 10 min, then tert-butyl 4-(2-hydroxyethyl) piperazine-1-carboxylate (15 g, 65.13 mmol, 1 eq) in DCM (50 mL) was added and the mixture was stirred at −70-−60° C. for 1 hour, then TEA (52.72 g, 521.05 mmol, 72.52 mL, 8 eq) was added and then the mixture was warmed to 20-25° C. slowly over 30 min, and the mixture was stirred at 20-25° C. for another 20 min. The reaction mixture was quenched by addition H₂O 200 mL and then extracted with DCM 100 mL. The organic layer was washed with H₂O 100 mL, brine 100 mL, dried over Na₂SO₄, filtered and concentrated under reduced pressure to give a residue. Compound tert-butyl 4-(2-oxoethyl) piperazine-1-carboxylate (14.5 g, crude) was obtained as yellow oil. The crude product was used into the next step without further purification.

Step 2: Preparation of methyl 3-bromo-4-[3-(tert-butoxycarbonylamino) cyclobutoxy]benzoate. To a solution of tert-butyl N-(3-hydroxycyclobutyl)carbamate (4.9 g, 26.17 mmol, 1 eq), methyl 3-bromo-4-hydroxybenzoate (5.93 g, 25.65 mmol, 0.98 eq) and PPh₃ (8.92 g, 34.02 mmol, 1.3 eq) in THF (100 mL) was added DIAD (6.88 g, 34.02 mmol, 6.61 mL, 1.3 eq) at 0-5° C. The mixture was stirred at 20-25° C. for 12 hours. The reaction mixture was quenched by addition H₂O 100 mL, and then extracted with EA 200 mL. The organic layer was washed with brine 50 mL, dried over Na₂SO₄, filtered and concentrated under reduced pressure to give a residue. The residue was triturated with EA(60 mL), filtered to give the solid. Compound methyl 3-bromo-4-[3-(tert-butoxycarbonylamino)cyclobutoxy]benzoate (12 g, crude) was obtained as an off-white solid.

Step 3: Preparation of 3-bromo-4-[3-[tert-butoxycarbonyl (methyl)amino]cyclobutoxy]benzoate. To a solution of methyl 3-bromo-4-[3-(tert-butoxycarbonylamino) cyclobutoxy]benzoate (13 g, 32.48 mmol, 1 eq) in DMF (120 mL) and THE (30 mL) was added NaH (1.95 g, 48.72 mmol, 60% purity, 1.5 eq) at 0-5° C. The mixture was stirred at 0-5° C. for 0.5 h, then Mel (6.91 g, 48.72 mmol, 3.03 mL, 1.5 eq) was added and the mixture was stirred at 20-25° C. for another 1.5 hours. The reaction mixture was quenched by addition NH₄Cl.aq 100 mL, and then diluted with brine 100 mL and extracted with EA 200 mL. The organic layer was washed with brine 100 mL (50 mL*2), dried over Na₂SO₄, filtered and concentrated under reduced pressure to give a residue. Compound methyl 3-bromo-4-[3-[tert-butoxycarbonyl (methyl)amino]cyclobutoxy]benzoate (13.5 g, crude) was obtained as yellow oil. The crude product was used into the next step without further purification.

Step 4: Preparation of 3-bromo-4-[3-(methylamino)cyclobutoxy]benzoate. To a solution of methyl 3-bromo-4-[3-[tert-butoxycarbonyl (methyl)amino]cyclobutoxy]benzoate (14.5 g, 35.00 mmol, 1 eq) in EtOAc (100 mL) was added HCl/EtOAc (4 M, 100 mL, 11.43 eq). The mixture was stirred at 20-25° C. for 1 hour. The reaction mixture was concentrated under reduced pressure to remove solvent. The residue was diluted with H₂O 100 mL and washed with EA 100 mL, then the water layer was adjusted pH>8 with NaHCO₃.aq, then extracted with EA 200 mL. The organic layer was washed with brine 50 mL, dried over Na₂SO₄, filtered and concentrated under reduced pressure to give a residue. Compound methyl 3-bromo-4-[3-(methylamino)cyclobutoxy]benzoate (7.7 g, 24.35 mmol, 69.57% yield, 99.347% purity) was obtained as colorless oil.

Step 5: Preparation of tert-butyl 4-[2-[[3-(2-bromo-4-methoxycarbonyl-phenoxy) cyclobutyl]-methyl-amino]ethyl]piperazine-1-carboxylate. To a solution of methyl 3-bromo-4-[3-(methylamino)cyclobutoxy]benzoate (7.2 g, 22.92 mmol, 1 eq) in MeOH (100 mL) was added HOAc (4.13 g, 68.75 mmol, 3.93 mL, 3 eq) and tert-butyl 4-(2-oxoethyl) piperazine-1-carboxylate (13.08 g, 57.29 mmol, 2.5 eq) at 0-5° C., the mixture was stirred at 0-5° C. for 1 hour, then NaBH(OAc)3 (9.71 g, 45.83 mmol, 2 eq) was added and then the mixture was stirred at 20-25° C. for another 11 hours. The reaction mixture was quenched by addition H₂O 10 mL, and then extracted with EA 20 mL. The organic layer was washed with brine 10 mL, dried over Na₂SO₄, filtered and concentrated under reduced pressure to give a residue. The residue was purified by prep-TLC (SiO₂, DCM:MeOH=10:1). Then purified by prep-HPLC (TFA. column: Phenomenex luna c18250 mm*100 mm*10 um; mobile phase: [water(TFA)-ACN];B %: 30%-50%,15 min). Compound tert-butyl 4-[2-[[3-(2-bromo-4-methoxycarbonyl-phenoxy) cyclobutyl]-methyl-amino]ethyl]piperazine-1-carboxylate (7.0 g, 13.30 mmol, 58.02% yield) was obtained as a brown solid.

Step 6: Preparation of 2-[3-bromo-4-[3-[methyl (2-piperazin-1-ylethyl) amino]cyclobutoxy]phenyl]propan-2-ol. To a solution of tert-butyl 4-[2-[[3-(2-bromo-4-methoxycarbonyl-phenoxy)cyclobutyl]-methylamino]ethyl]piperazine-1-carboxylate (400 mg, 759.79 umol, 1 eq) in DCM (10 mL) was added MeMgBr (3 M, 5.07 mL, 20 eq) at −70-−60° C. The mixture was stirred at 20-25° C. for 12 hour. The reaction mixture was quenched by addition NH₄Cl.aq 20 mL at 0-5° C., and then extracted with DCM/MeOH 50 mL (25 mL*2, 10:1). The combined organic layers were washed with brine 10 mL, dried over Na₂SO₄, filtered and concentrated under reduced pressure to give a residue. Compound 2-[3-bromo-4-[3-[methyl (2-piperazin-1-ylethyl) amino]cyclobutoxy]phenyl]propan-2-ol (310 mg, crude) was obtained as a yellow solid. The crude product was used into the next step without further purification.

Step 7: Preparation of 4-[5-(1-hydroxy-1-methyl-ethyl)-2-[3-[methyl (2-piperazin-1-ylethyl)amino]cyclobutoxy]phenyl]-6-methyl-1-(p-tolyl sulfonyl) pyrrolo[2,3-c]pyridin-7-one. A mixture of 2-[3-bromo-4-[3-[methyl(2-piperazin-1-ylethyl)amino]cyclobutoxy]phenyl]propan-2-ol (100 mg, 234.53 umol, 1 eq), 6-methyl-1-(p-tolylsulfonyl)-4-(4,4,5,5-tetramethyl-1,3,2-dioxaborolan-2-yl)pyrrolo[2,3-c]pyridin-7-one(100.45 mg, 234.53 umol, 1 eq), K₂CO₃ (97.24 mg, 703.58 umol, 3 eq), [2-(2-aminophenyl) phenyl]palladium(1+);dicyclohexyl-[2-(2,4,6-triisopropylphenyl) phenyl]phosphane; methanesulfonate (19.85 mg, 23.45 umol, 0.1 eq) in THE (2 mL) and H₂O (0.2 mL) was degassed and purged with N₂ for 3 times, and then the mixture was stirred at 65° C. for 12 hours under N₂ atmosphere. The reaction mixture was quenched by addition NH₃ aq. 5 mL, and then extracted with EA 20 mL (10 mL*2). The combined organic layers were dried over Na₂SO₄, filtered and concentrated under reduced pressure to give a residue. The residue was purified by prep-HPLC (FA. column: UniSil 3-100 C18 Ultra (150*25 mm*3 um); mobile phase: [water(FA)-ACN];B %: 14%-34%,7 min). Compound 4-[5-(1-hydroxy-1-methyl-ethyl)-2-[3-[methyl (2-piperazin-1-ylethyl)amino]cyclobutoxy]phenyl]-6-methyl-1-(p-tolyl sulfonyl) pyrrolo[2,3-c]pyridin-7-one (90 mg, 138.93 umol, 29.62% yield) was obtained as an off-white solid.

Step 8: Preparation of 4-[5-(1-hydroxy-1-methyl-ethyl)-2-[3-[methyl (2-piperazin-1-ylethyl) amino]cyclobutoxy]phenyl]-6-methyl-11H-pyrrolo[2, 3-c]pyridin-7-one. A mixture of 4-[5-(1-hydroxy-1-methyl-ethyl)-2-[3-[methyl(2-piperazin-1-ylethyl)amino]cyclobutoxy]phenyl]-6-methyl-1-(p-tolylsulfonyl)pyrrolo[2,3-c]pyridin-7-one (90 mg, 138.93 umol, 1 eq), KOH (389.76 mg, 6.95 mmol, 50 eq), hexadecyl(trimethyl)ammonium bromide (15.19 mg, 41.68 umol, 0.3 eq) in THF (1 mL) and H₂O (0.5 mL) as degassed and purged with N₂ for 3 times, and then the mixture was stirred at 90° C. for 14 hours under N₂ atmosphere. The reaction mixture was extracted with DCM/MeOH 20 mL (10 mL*2, 10:1). The combined organic layers were dried over Na₂SO₄, filtered and concentrated under reduced pressure to give a residue. The residue was purified by prep-HPLC (FA. column: Phenomenex luna C_{18 150*25} mm*10 um; mobile phase: [water(FA)-ACN];B %: 1%-25%,10 min). Compound 4-[5-(1-hydroxy-1-methyl-ethyl)-2-[3-[methyl (2-piperazin-1-ylethyl) amino]cyclobutoxy]phenyl]-6-methyl-1H-pyrrolo[2, 3-c]pyridin-7-one (50 mg, 79.15 umol, 56.97% yield, 3HCOOH) was obtained as a yellow solid.

Step 9: Preparation of 2-[[6-[[5-chloro-2-[4-[2-[[3-[4-(1-hydroxy-1-methyl-ethyl)-2-(6-methyl-7-oxo-1H-pyrrolo[2,3-c]pyridin-4-yl)phenoxy]cyclobutyl]-methyl-amino]ethyl]piperazin-1-yl]pyrimidin-4-yl]amino]-1-methyl-2-oxo-3-quinolyl]oxy]-N-methyl-acetamide. A mixture of 4-[5-(1-hydroxy-1-methyl-ethyl)-2-[3-[methyl(2-piperazin-1-ylethyl)amino]cyclobutoxy]phenyl]-6-methyl-1H-pyrrolo[2,3-c]pyridin-7-one (50 mg, 79.15 umol, 1 eq, 3HCOOH), 2-[[6-[(2,5-dichloropyrimidin-4-yl)amino]-1-methyl-2-oxo-3-quinolyl]oxy]-N-methyl-acetamide (38.77 mg, 94.98 umol, 1.2 eq), DIPEA (102.30 mg, 791.50 umol, 137.86 uL, 10 eq) in DMSO (1 mL) was degassed and purged with N₂ for 3 times, and then the mixture was stirred at 100° C. for 2 hours under N₂ atmosphere. The reaction mixture was filtered to give a residue. The residue was purified by prep-HPLC (NEU. column: Waters xbridge 150*25 mm 10 um; mobile phase: [water (NH₄HCO₃)-ACN];B %: 35%-65%,10 min). The residue was purified by prep-HPLC (NEU. column: Waters Xbridge 150*25 mm*5 um; mobile phase: [water (NH₄HCO₃)-ACN];B %: 36%-66%,9 min). Compound 2-[[6-[[5-chloro-2-[4-[2-[[3-[4-(1-hydroxy-1-methyl-ethyl)-2-(6-methyl-7-oxo-1H-pyrrolo[2,3-c]pyridin-4-yl)phenoxy]cyclobutyl]-methyl-amino]ethyl]piperazin-1-yl]pyrimidin-4-yl]amino]-1-methyl-2-oxo-3-quinolyl]oxy]-N-methyl-acetamide (40.75 mg, 45.43 umol, 57.40% yield, 96.48% purity) was obtained as an off-white solid. ¹H NMR (400 MHz, CDCl₃) δ (ppm) 10.30 (br s, 1H), 8.03 (s, 1H), 7.82-7.71 (m, 2H), 7.65 (br d, J=4.4 Hz, 1H), 7.56 (d, J=2.0 Hz, 1H), 7.42 (dd, J=8.4, 2.0 Hz, 1H), 7.35 (d, J=9.2 Hz, 1H), 7.26-7.22 (m, 1H), 7.07 (d, J=7.2 Hz, 2H), 6.93 (s, 1H), 6.73 (d, J=8.8 Hz, 1H), 6.30 (br s, 1H), 4.74 (br s, 1H), 4.56 (s, 2H), 3.74-3.82 (m, 7H), 3.71 (s, 3H), 2.98-3.10 (m, 1H), 2.92 (d, J=4.75 Hz, 3H), 2.39-2.54 (m, 8H), 2.26-2.38 (m, 2H), 2.10-2.25 (m, 5H), 1.64 (s, 6H). LC-MS: MS (ES⁺): RT=1.536 min, m/z=865.4[M+H⁺]; LCMS Method: Method 25.

### Example 4—Synthesis of 2-[[6-[[5-chloro-2-[4-[[1-[[3-[4-(1-hydroxy-1-methyl-ethyl)-2-(6-methyl-7-oxo-1H-pyrrolo[2,3-c]pyridin-4-yl)phenoxy]cyclobutyl]methyl]azetidin-3-yl]methyl]-1-piperidyl]pyrimidin-4-yl]amino]-1-methyl-2-oxo-3-quinolyl]oxy]-N-methyl-acetamide (I-22)

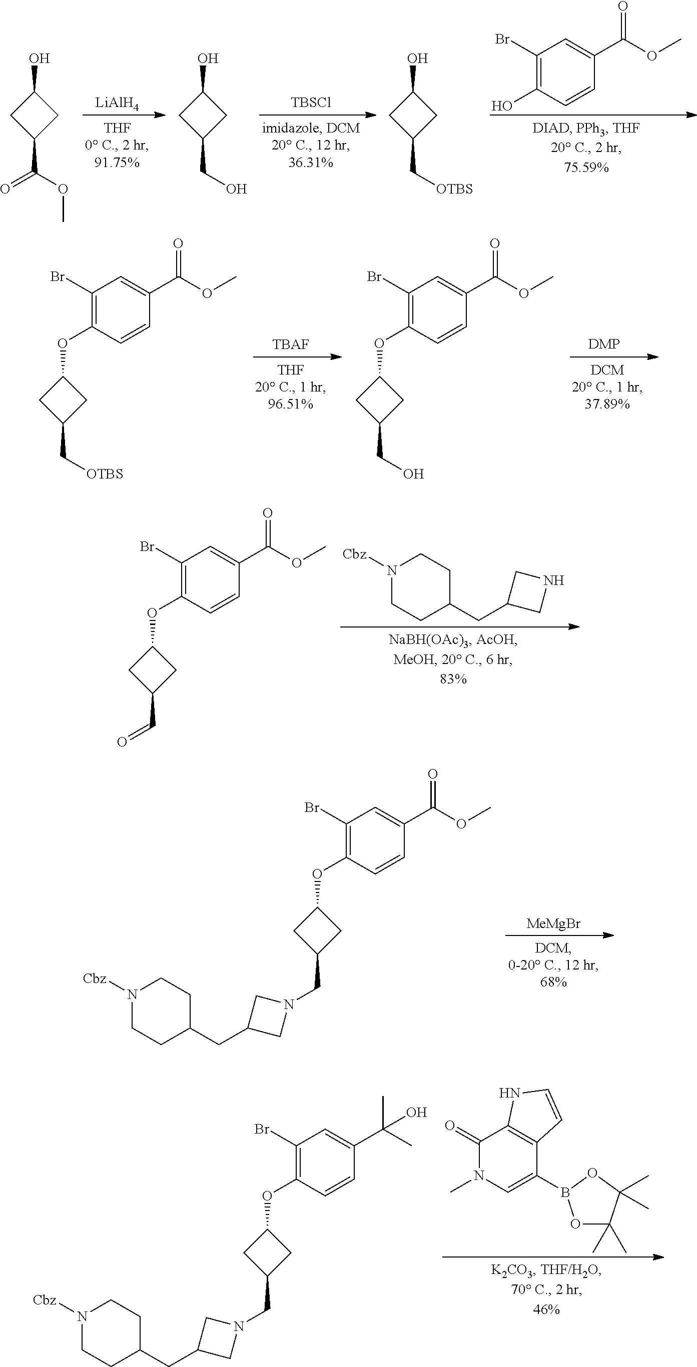

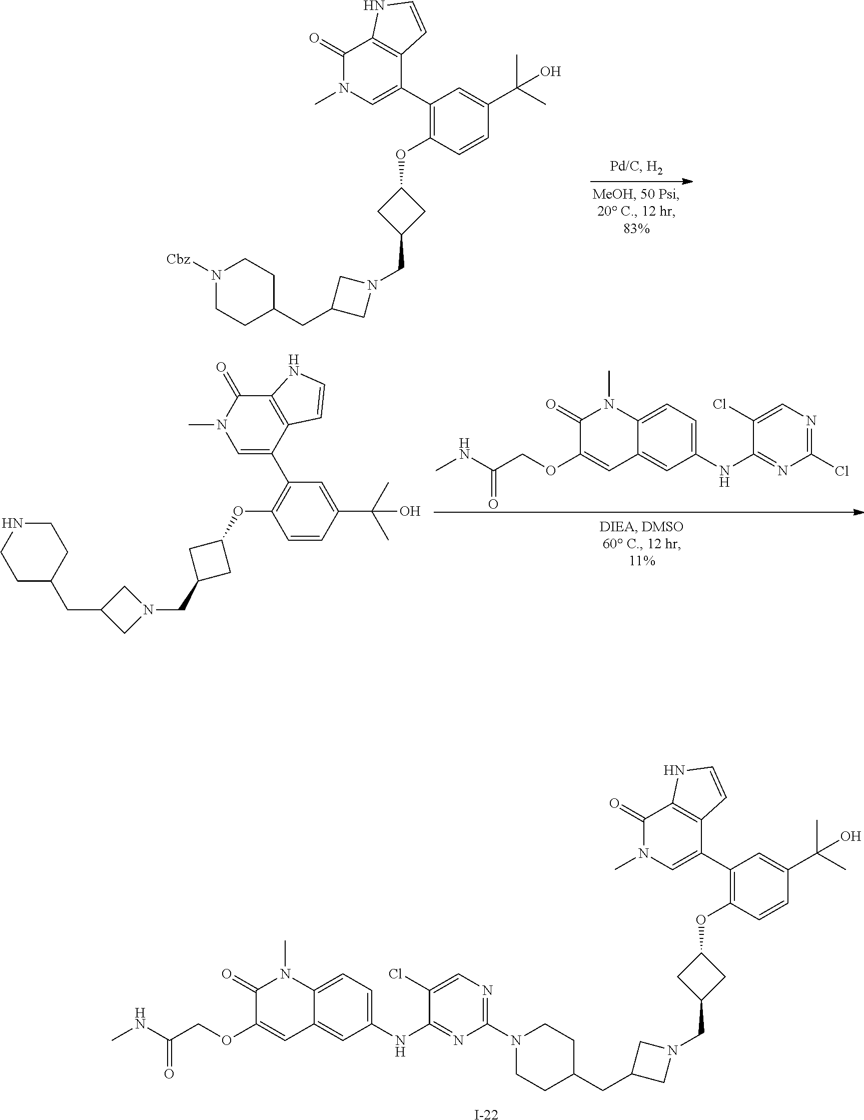

Step 1: Preparation of 3-(hydroxymethyl)cyclobutanol. To a solution of ethyl 3-hydroxycyclobutanecarboxylate (2 g, 13.87 mmol, 1 eq) in THF (1 mL) was added LAH (2.5 M, 5.80 mL, 1.05 eq) at 0° C. The mixture was stirred at 0° C. for 2 hours. The reaction mixture was added with water (0.2 ml) dropwise, and then added with 15% aqueous sodium hydroxide solution (0.5 ml), water (0.5 ml) was added dropwise, stirred and warmed to room temperature, then tetrahydrofuran(10 mL) and Na₂SO₄ were added, stirred for 1 hour, filtered, and concentrated under reduced pressure to remove the solvent. Compound 3-(hydroxymethyl) cyclobutanol (1.3 g, 12.73 mmol, 91.75% yield) was obtained as a colourless liquid without purification.

Step 2: Preparation of 3-1[[tert-butyl(dimethyl)silyl]oxymethyl]cyclobutanol. To a solution of 3-(hydroxymethyl)cyclobutanol (1.3 g, 12.73 mmol, 1 eq) in DCM (30 mL) was added imidazole (1.30 g, 19.10 mmol, 1.50 eq) and tert-butyl-chloro-dimethyl-silane (2.10 g, 13.96 mmol, 1.72 mL, 1.10 eq) at 0° C. The mixture was stirred at 20° C. for 12 hours. The reaction mixture was diluted with H₂O 20 mL and extracted with DCM 20 mL (10 mL*2). The combined organic layers were washed with brine 20 mL, dried over Na₂SO₄, filtered and concentrated under reduced pressure to give a residue. The residue was purified by flash silica gel chromatography (ISCO®; 40 g SepaFlash® Silica Flash Column, Eluent of 0˜25% Ethylacetate/Petroleum ethergradient @60 mL/min). Compound 3-[[tert-butyl(dimethyl)silyl]oxymethyl]cyclobutanol (1 g, 4.62 mmol, 36.31% yield) was obtained as a colourless liquid.

Step 3: Preparation of methyl 3-bromo-4-[3-[[tert-butyl (dimethyl)silyl]oxymethyl]cyclobutoxy]benzoate. A mixture of 3-[[tert-butyl(dimethyl)silyl]oxymethyl]cyclobutanol (800 mg, 3.70 mmol, 1 eq), methyl 3-bromo-4-hydroxy-benzoate (800.00 mg, 3.46 mmol, 9.37e-1 eq), DIAD (2.09 g, 10.32 mmol, 2.00 mL, 2.79 eq), PPh3 (2.80 g, 10.68 mmol, 2.89 eq) in THE (30 mL) was degassed and purged with N₂ for 3 times, and then the mixture was stirred at 20° C. for 2 hours under N₂ atmosphere. The reaction mixture was diluted with H₂O 10 mL and extracted with EA 20 mL (10 mL*2). The combined organic layers were washed with brine 30 mL, dried over Na₂SO₄, filtered and concentrated under reduced pressure to give a residue. The residue was purified by flash silica gel chromatography (ISCO®; 40 g SepaFlash® Silica Flash Column, Eluent of 0˜10% Ethylacetate/Petroleum ethergradient @60 mL/min). Compound methyl 3-bromo-4-[3-[[tert-butyl (dimethyl)silyl]oxymethyl]cyclobutoxy]benzoate (1.2 g, 2.79 mmol, 75.59% yield) was obtained as a yellow oil.

Step 4: Preparation of methyl 3-bromo-4-[3-(hydroxymethyl)cyclobutoxy]benzoate. To a solution of methyl 3-bromo-4-[3-[[tert-butyl(dimethyl)silyl]oxymethyl]cyclobutoxy]benzoate (1.2 g, 2.79 mmol, 1 eq) in THE (20 mL) was added tetrabutylammonium fluoride;trihydrate (1 M, 3.36 mL, 1.2 eq). The mixture was stirred at 20° C. for 1 hour. The reaction mixture was quenched by addition HCl (2 M, 10 mL) at 0° C., and then extracted with EA 10 mL (5 mL*2). The combined organic layers were dried over Na₂SO₄, filtered and concentrated under reduced pressure to give a residue. Compound methyl 3-bromo-4-[3-(hydroxymethyl)cyclobutoxy]benzoate (850 mg, 2.70 mmol, 96.51% yield) was obtained as a white solid without purification.

Step 5: Preparation of methyl 3-bromo-4-(3-formylcyclobutoxy) benzoate. To a solution of methyl 3-bromo-4-[3-(hydroxymethyl)cyclobutoxy]benzoate (850 mg, 2.70 mmol, 1 eq) in DCM (10 mL) was added DMP (1.49 g, 3.51 mmol, 1.09 mL, 1.3 eq). The mixture was stirred at 20° C. for 1 hr. The reaction mixture was added saturated aqueous Na₂S₂O₃ (10 mL) and NaHCO₃ (10 mL) at 0° C., and the mixture was stirred at 0° C. for 0.5 hour. The mixture was extracted with DCM (10 mL*2). The combined organic phase was washed with brine (20 mL), dried over anhydrous Na₂SO₄ and then filtered. The filtration was concentrated in vacuum to give a residue. The residue was purified by flash silica gel chromatography (ISCO®; 12 g SepaFlash® Silica Flash Column, Eluent of 0˜25% Ethylacetate/Petroleum ethergradient @30 mL/min). Compound methyl 3-bromo-4-(3-formylcyclobutoxy) benzoate (320 mg, 1.02 mmol, 37.89% yield) was obtained as a white solid.

Step 6: Preparation of benzyl 4-[[1-[[3-(2-bromo-4-methoxycarbonyl-phenoxy)cyclobutyl]methyl]azetidin-3-yl]methyl]piperidine-1-carboxylate. To a solution of benzyl 4-(azetidin-3-ylmethyl)piperidine-1-carboxylate (500 mg, 1.73 mmol, 1 eq) and methyl 3-bromo-4-(3-formylcyclobutoxy)benzoate (500.00 mg, 1.60 mmol, 9.21e-1 eq) in MeOH (5 mL) was added AcOH (104.12 mg, 1.73 mmol, 99.25 μL, 1 eq). The mixture was stirred at 20° C. for 1 hour. Then added NaBH(OAc)3 (734.93 mg, 3.47 mmol, 2 eq) and stirred at 20° C. for 5 hr. The reaction mixture was concentrated under reduced pressure to remove solvent. The residue was purified by flash silica gel chromatography (ISCO®; 12 g SepaFlash® Silica Flash Column, Eluent of 0˜80% Ethyl acetate/Petroleum ethergradient @30 mL/min). Compound benzyl 4-[[1-[[3-(2-bromo-4-methoxycarbonyl-phenoxy)cyclobutyl]methyl]azetidin-3-yl]methyl]piperidine-1-carboxylate (850 mg, 1.45 mmol, 83.73% yield) was obtained as a colorless solid.

Step 7: Preparation of benzyl 4-[[1-[[3-[2-bromo-4-(1-hydroxy-1-methyl-ethyl)phenoxy]cyclobutyl]methyl]azetidin-3-yl]methyl]piperidine-1-carboxylate. To a solution of benzyl 4-[[1-[[3-(2-bromo-4-methoxycarbonyl-phenoxy)cyclobutyl]methyl]azetidin-3-yl]methyl]piperidine-1-carboxylate (850 mg, 1.45 mmol, 1 eq) in DCM (10 mL) was added MeMgBr (3 M, 2.50 mL, 5.17 eq) and the resulting mixture was stirred at 0° C. Then the mixture was wormed to 20° C. and stirred for 12 hours. The reaction mixture was quenched by addition NH₄Cl 4 mL at 0° C., and then diluted with H₂O 30 mL and extracted with EtOAc 40 mL (20 mL*2). The combined organic layers were washed with brine 20 mL, dried over anhydrous Na₂SO₄, filtered and concentrated under reduced pressure to give a residue. The residue was purified by flash silica gel chromatography (ISCO®; 12 g SepaFlash® Silica Flash Column, Eluent of 0-50% Ethyl acetate/Petroleum ethergradient @30 mL/min). Compound benzyl 4-[[1-[[3-[2-bromo-4-(1-hydroxy-1-methyl-ethyl)phenoxy]cyclobutyl]methyl]azetidin-3-yl]methyl]piperidine-1-carboxylate (580 mg, 990.49 mol, 68.23% yield) was obtained as a colorless solid.

Step 8: Preparation of 4-[[1-[[3-[4-(1-hydroxy-1-methyl-ethyl)-2-(6-methyl-7-oxo-1H-pyrrolo[2,3-c]pyridin-4-yl)phenoxy]cyclobutyl]methyl]azetidin-3-yl]methyl]piperidine-1-carboxylate. To a solution of benzyl 4-[[1-[[3-[2-bromo-4-(1-hydroxy-1-methyl-ethyl)phenoxy]cyclobutyl]methyl]azetidin-3-yl]methyl]piperidine-1-carboxylate (580 mg, 990.49 mol, 1 eq) and 6-methyl-4-(4,4,5,5-tetramethyl-1,3,2-dioxaborolan-2-yl)-1H-pyrrolo[2,3-c]pyridin-7-one (298.67 mg, 1.09 mmol, 1.1 eq) in THE (5 mL) and H₂O (0.5 mL) was added K₂CO₃ (410.69 mg, 2.97 mmol, 3 eq) and [2-(2-aminophenyl)phenyl]palladium(1+); bis(1-adamantyl)-butyl-phosphane;methanesulfonate (36.07 mg, 49.52 mol, 0.05 eq), then the mixture was stirred at 70° C. for 12 hours under N₂ atmosphere. The residue was diluted with H₂O 30 mL and extracted with EtOAc 60 mL (20 mL*3). The combined organic layers were washed with brine 40 mL, dried over anhydrous Na₂SO₄, filtered and concentrated under reduced pressure to give a residue. The residue was purified by prep-HPLC (FA condition; column: Phenomenex luna C18 150*25 mm*10 um; mobile phase: [water(FA)-ACN];gradient:20%-50% B over 10 min). Compound benzyl 4-[[1-[[3-[4-(1-hydroxy-1-methyl-ethyl)-2-(6-methyl-7-oxo-1H-pyrrolo[2,3-c]pyridin-4-yl)phenoxy]cyclobutyl]methyl]azetidin-3-yl]methyl]piperidine-1-carboxylate (300 mg, 459.54 mol, 46.40% yield) was obtained as a white solid.

Step 9: Preparation of 4-[5-(1-hydroxy-1-methyl-ethyl)-2-[3-[[3-(4-piperidylmethyl) azetidin-1-yl]methyl]cyclobutoxy]phenyl]-6-methyl-11H-pyrrolo[2,3-c]pyridin-7-one. To a solution of benzyl 4-[[1-[[3-[4-(1-hydroxy-1-methyl-ethyl)-2-(6-methyl-7-oxo-1H-pyrrolo[2,3-c]pyridin-4-yl)phenoxy]cyclobutyl]methyl]azetidin-3-yl]methyl]piperidine-1-carboxylate (300 mg, 459.54 mol, 1 eq) in MeOH (10 mL) was added Pd/C (75.00 mg, 459.54 mol, 10% purity, 1.00 eq) under N₂ atmosphere. The suspension was degassed and purged with H₂ for 3 times. The mixture was stirred under H₂ (50 Psi) at 20° C. for 12 hours. The reaction mixture was filtered and concentrated under reduced pressure to give a residue. The crude product 4-[5-(1-hydroxy-1-methyl-ethyl)-2-[3-[[3-(4-piperidylmethyl) azetidin-1-yl]methyl]cyclobutoxy]phenyl]-6-methyl-1H-pyrrolo[2,3-c]pyridin-7-one (200 mg, 385.59 mol, 83.91% yield) was obtained as a colorless solid and used into the next step without further purification.

Step 10: Preparation of 2-[[6-[[5-chloro-2-[4-[[1-[[3-[4-(1-hydroxy-1-methyl-ethyl)-2-(6-methyl-7-oxo-1H-pyrrolo[2,3-c]pyridin-4-yl)phenoxy]cyclobutyl]methyl]azetidin-3-yl]methyl]-1-piperidyl]pyrimidin-4-yl]amino]-1-methyl-2-oxo-3-quinolyl]oxy]-N-methyl-acetamide. To a solution of 4-[5-(1-hydroxy-1-methyl-ethyl)-2-[3-[[3-(4-piperidylmethyl) azetidin-1-yl]methyl]cyclobutoxy]phenyl]-6-methyl-1H-pyrrolo[2,3-c]pyridin-7-one (190 mg, 366.31 mol, 1 eq) and 2-[[6-[(2,5-dichloropyrimidin-4-yl)amino]-1-methyl-2-oxo-3-quinolyl]oxy]-N-methyl-acetamide (164.50 mg, 402.94 mol, 1.1 eq) in DMSO (2 mL) was added DIEA (95.00 mg, 735.05 mol, 128.03 μL, 2.01 eq). The mixture was stirred at 60° C. for 12 hours. The reaction was filtered. The residue was purified by prep-HPLC (FA condition; column: Phenomenex luna C18 150*25 mm*10 um;mobile phase: [water(FA)-ACN];gradient:12%-42% B over 10 min). Compound 2-[[6-[[5-chloro-2-[4-[[1-[[3-[4-(1-hydroxy-1-methyl-ethyl)-2-(6-methyl-7-oxo-1H-pyrrolo[2,3-c]pyridin-4-yl)phenoxy]cyclobutyl]methyl]azetidin-3-yl]methyl]-1-piperidyl]pyrimidin-4-yl]amino]-1-methyl-2-oxo-3-quinolyl]oxy]-N-methyl-acetamide (34.81 mg, 35.92 mol, 63.97% yield, 91.89% purity) was obtained as an off-white solid. ¹H NMR (400 MHz, CDCl₃): δ (ppm)=7.67 (d, J=2.4 Hz, 1H), 7.37-7.31 (m, 6H), 6.72-6.60 (m, 1H), 5.12 (s, 2H), 4.22-4.13 (m, 3H), 3.76-3.52 (m, 2H), 2.99-2.55 (m, 8H), 2.51-2.24 (m, 4H), 1.99-1.88 (m, 1H), 1.80-1.65 (m, 2H), 1.55 (s, 6H), 1.40-1.34 (m, 1H), 1.20-0.99 (m, 3H). LC-MS: MS (ES⁺): RT=1.657 min, m/z=890.4[M+H+]; LCMS Method: Method 10.

### Example 5—Synthesis of 2-[[6-[[5-chloro-2-[4-[2-[4-[2-[3-[4-(1-hydroxy-1-methyl-ethyl)-2-(6-methyl-7-oxo-1H-pyrrolo[2,3-c]pyridin-4-yl)phenoxy]phenoxy]ethyl]piperazin-1-yl]ethyl]piperazin-1-yl]pyrimidin-4-yl]amino]-1-methyl-2-oxo-3-quinolyl]oxy]-N-methyl-acetamide (I-23)

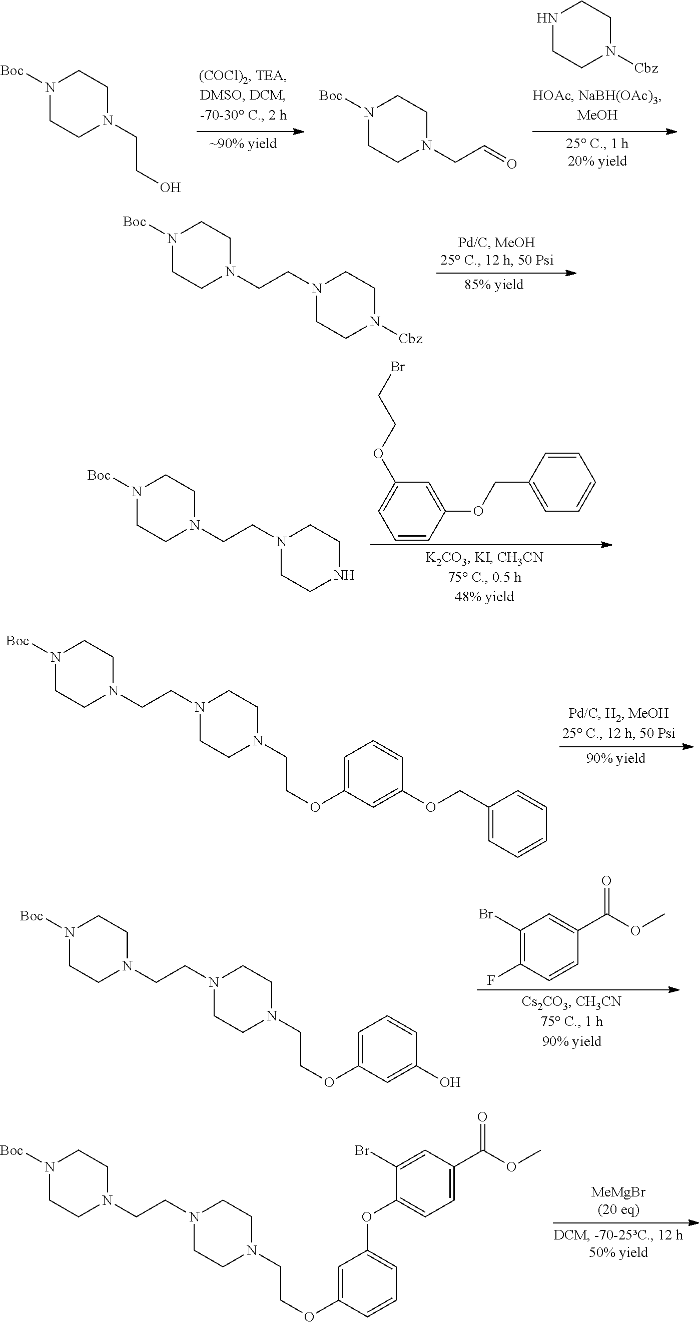

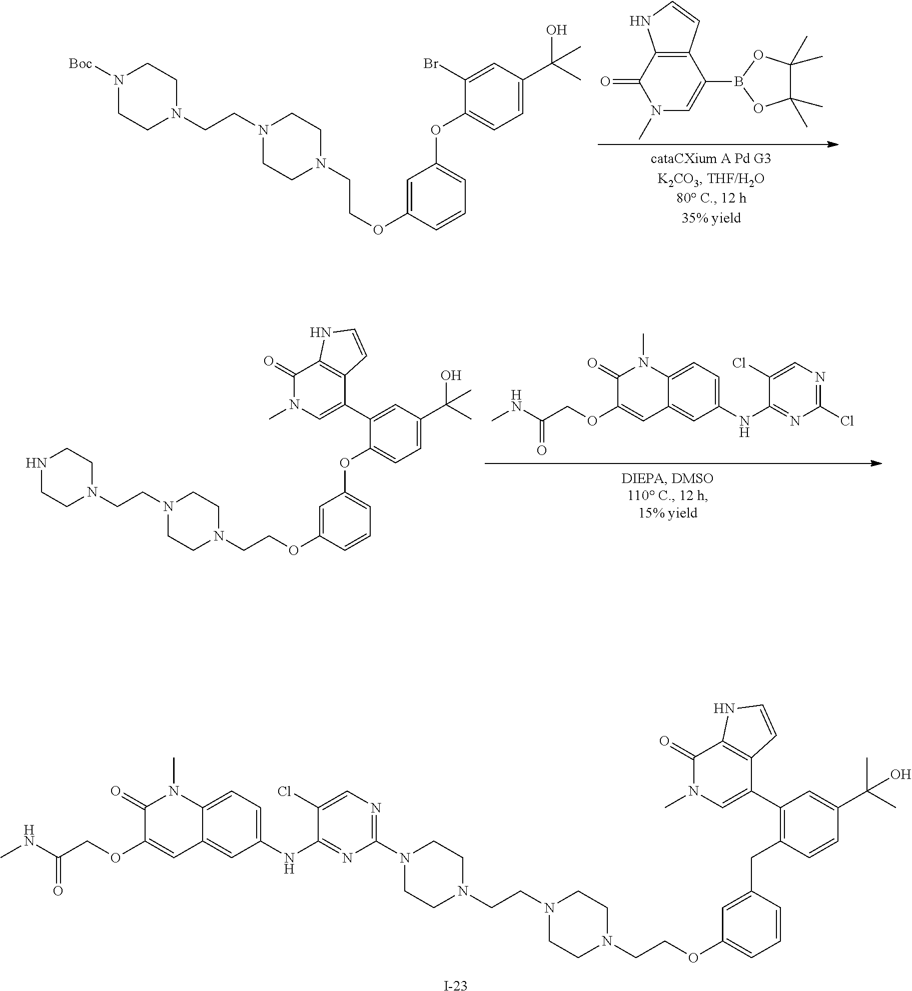

Step 1: Preparation of tert-butyl 4-(2-oxoethyl) piperazine-1-carboxylate. To a solution of (COCl)₂ (9.3 g, 73.11 mmol, 6.40 mL, 2.10 eq) in DCM (100.0 mL) was cooled to −70° C., then DMSO (11.2 g, 143.34 mmol, 11.20 mL, 4.13 eq) was added slowly at −70° C. The mixture was stirred at −70° C. 30 min, then tert-butyl 4-(2-hydroxyethyl) piperazine-1-carboxylate (8 g, 34.74 mmol, 1 eq) in DCM (20.0 mL) was added and the mixture was stirred at −70° C. for 1 hour, then TEA (29.1 g, 287.38 mmol, 40.00 mL, 8.27 eq) was added and then the mixture was warmed to 30° C., and the mixture was stirred at 30° C. for another 30 min. TLC (DCM/MeOH=10/1) showed the tert-butyl 4-(2-hydroxyethyl) piperazine-1-carboxylate was consumed completely and observed one new spot. The reaction mixture was quenched by addition H₂O (100.0 mL), and then extracted with DCM 150.0 mL (50.0 mL*3). The combined organic layers were dried over anhydrous Na₂SO₄, filtered and concentrated under reduced pressure to give a residue. Compound tert-butyl 4-(2-oxoethyl) piperazine-1-carboxylate (8.0 g, crude) was obtained as a yellow oil.

Step 2: Preparation of benzyl 4-[2-(4-tert-butoxycarbonylpiperazin-1-yl) ethyl]piperazine-1-carboxylate. To a solution of tert-butyl 4-(2-oxoethyl) piperazine-1-carboxylate (2.0 g, 8.76 mmol, 1 eq) and benzyl piperazine-1-carboxylate (2.0 g, 9.08 mmol, 1.75 mL, 1.04 eq) in MeOH (10.0 mL) was added HOAc (250.0 mg, 4.16 mmol, 238.32 μL, 4.75e-1 eq), the mixture was stirred at 25° C. for 0.5 hour. Then NaBH(OAc)3 (3.0 g, 14.15 mmol, 1.62 eq) was added, the resulting mixture was stirred at 25° C. for 0.5 hour. The reaction mixture was concentrated under reduced pressure to give a residue. The residue was purified by prep-HPLC (FA condition).(column: Phenomenex luna C18 150*40 mm*15 um;mobile phase: [water(FA)-ACN];B %: 13%-43%,15 min). Compound benzyl 4-[2-(4-tert-butoxycarbonylpiperazin-1-yl) ethyl]piperazine-1-carboxylate (0.9 g) was obtained as a yellow oil.

Step 3: Preparation of tert-butyl 4-(2-piperazin-1-ylethyl) piperazine-1-carboxylate. To a solution of Pd/C (90.0 mg, 10% purity) in MeOH (20.0 mL) was added benzyl 4-[2-(4-tertbutoxycarbonylpiperazin-1-yl) ethyl]piperazine-1-carboxylate (0.9 g, 2.08 mmol, 1 eq) under N₂ atmosphere. The mixture was stirred at 25° C. for 12 hours under H₂ atmosphere(50 Psi). TLC (DCM/MeOH=10/1) showed the benzyl 4-[2-(4-tert-butoxycarbonylpiperazin-1-yl) ethyl]piperazine-1-carboxylate was consumed completely and observed one new spot. The reaction mixture was filtered and concentrated under reduced pressure to give a residue. Compound tert-butyl 4-(2-piperazin-1-ylethyl) piperazine-1-carboxylate (0.55 g, 1.84 mmol, 88.58% yield) was obtained as a brown solid.

Step 4: Preparation of tert-butyl 4-[2-[4-[2-(3-benzyloxyphenoxy) ethyl]piperazin-1-yl]ethyl]piperazine-1-carboxylate. To a solution of tert-butyl 4-(2-piperazin-1-ylethyl) piperazine-1-carboxylate (0.35 g, 1.17 mmol, 1 eq) in CH₃CN (3.0 mL) were added 1-benzyloxy-3-(2-bromoethoxy) benzene (385.0 mg, 1.25 mmol, 1.07 eq), K₂CO₃ (350.0 mg, 2.53 mmol, 2.16 eq) and KI (175.0 mg, 1.05 mmol, 8.99e-1 eq). The mixture was stirred at 75° C. for 0.5 hour. The reaction mixture was filtered and concentrated under reduced pressure to give a residue. The residue was purified by prep-HPLC (TFA condition). (column: Phenomenex luna C18 150*40 mm*15 um;mobile phase: [water(TFA)-ACN];B %: 15%-45%,10 min). Compound tert-butyl 4-[2-[4-[2-(3-benzyloxyphenoxy) ethyl]piperazin-1-yl]ethyl]piperazine-1-carboxylate (0.3 g, 571.76 mol, 48.75% yield) was obtained as a colorless oil.

Step 5: Preparation of tert-butyl 4-[2-[4-[2-(3-hydroxyphenoxy) ethyl]piperazin-1-yl]ethyl]piperazine-1-carboxylate. To a solution of Pd/C (20.0 mg, 10% purity) in MeOH (5.0 mL) was added tertbutyl4-[2-[4-[2-(3-benzyloxyphenoxy) ethyl]piperazin-1-yl]ethyl]piperazine-1-carboxylate (0.2 g, 381.18 mol, 1 eq) under N₂ atmosphere. The mixture was stirred at 25° C. for 12 hours under H₂ atmosphere (50 Psi). The reaction mixture was filtered and concentrated under reduced pressure to give a residue. Compound tert-butyl 4-[2-[4-[2-(3-hydroxyphenoxy) ethyl]piperazin-1-yl]ethyl]piperazine-1-carboxylate (0.15 g, 345.17 mol, 90.55% yield) was obtained as a colorless oil.

Step 6: Preparation of tert-butyl 4-[2-[4-[2-[3-(2-bromo-4-methoxycarbonyl-phenoxy) phenoxy]ethyl]piperazin-1-yl]ethyl]piperazine-1-carboxylate. To a solution of tert-butyl 4-[2-[4-[2-(3-hydroxyphenoxy) ethyl]piperazin-1-yl]ethyl]piperazine-1-carboxylate (0.4 g, 920.45 mol, 1 eq) in CH₃CN (3.0 mL) were added Cs₂CO₃ (600.0 mg, 1.84 mmol, 2.00 eq) and methyl 3-bromo-4-fluoro-benzoate (0.35 g, 1.50 mmol, 1.63 eq). The mixture was stirred at 75° C. for 1 hour. The reaction mixture was filtered and concentrated under reduced pressure to give a residue. The residue was purified by prep-HPLC (TFA condition). (column: Phenomenex luna C18 150*40 mm*15 um;mobile phase: [water(TFA)-ACN]; B %: 20%-50%,10 min. Compound tert-butyl 4-[2-[4-[2-[3-(2-bromo-4-methoxycarbonyl-phenoxy) phenoxy]ethyl]piperazin-1-yl]ethyl]piperazine-1-carboxylate (0.55 g, 849.29 mol, 92.27% yield) was obtained as a yellow oil.

Step 7: Preparation of 2-[3-bromo-4-[3-[2-[4-(2-piperazin-1-ylethyl) piperazin-1-yl]ethoxy]phenoxy]phenyl]propan-2-ol. To a solution of tert-butyl 4-[2-[4-[2-[3-(2-bromo-4-methoxycarbonyl-phenoxy) phenoxy]ethyl]piperazin-1-yl]ethyl]piperazine-1-carboxylate (0.5 g, 772.08 mol, 1 eq) in DCM (10.0 mL) was added dropwise MeMgBr (3 M, 5.6 mL, 21.76 eq) at −78° C. The mixture was stirred at 25° C. for 12 hours. The reaction mixture was quenched by addition sat. NH₄Cl (40.0 mL), and then diluted with H₂O (20.0 mL) and extracted with DCM 90.0 mL (30.0 mL*3). The combined organic layers were dried over anhydrous Na₂SO₄, filtered and concentrated under reduced pressure to give a residue. Compound 2-[3-bromo-4-[3-[2-[4-(2-piperazin-1-ylethyl) piperazin-1-yl]ethoxy]phenoxy]phenyl]propan-2-ol (250 mg, crude) was obtained as a yellow oil.

Step 8: Preparation of 4-[5-(1-hydroxy-1-methyl-ethyl)-2-[3-[2-[4-(2-piperazin-1-ylethyl) piperazin-1-yl]ethoxy]phenoxy]phenyl]-6-methyl-1H-pyrrolo[2,3-c]pyridin-7-one. To a solution of 2-[3-bromo-4-[3-[2-[4-(2-piperazin-1-ylethyl) piperazin-1-yl]ethoxy]phenoxy]phenyl]propan-2-ol (250.0 mg, 456.60 mol, 1 eq) and 6-methyl-4-(4,4,5,5-tetramethyl-1,3,2-dioxaborolan-2-yl)-1H-pyrrolo[2,3-c]pyridin-7-one (141.7 mg, 516.80 mol, 1.13 eq) in THE (3.0 mL) and H₂O (1.0 mL) were added K₂CO₃ (250.0 mg, 1.81 mmol, 3.96 eq) and [2-(2-aminophenyl)phenyl]palladium(1+); bis(1-adamantyl)-butyl-phosphane; methanesulfonate (125.0 mg, 171.64 mol, 3.76e⁻¹ eq). The mixture was stirred at 80° C. for 12 hours under N₂ atmosphere. The reaction mixture was filtered and concentrated under reduced pressure to give a residue. The residue was purified by prep-HPLC (TFA condition). (column: Phenomenex luna C18 150*40 mm*15 um;mobile phase: [water(TFA)-ACN];gradient:10%-40% B over 10 min). Compound 4-[5-(1-hydroxy-1-methyl-ethyl)-2-[3-[2-[4-(2-piperazin-1-ylethyl) piperazin-1-yl]ethoxy]phenoxy]phenyl]-6-methyl-1H-pyrrolo[2,3-c]pyridin-7-one (0.1 g, 162.66 mol, 35.62% yield) was obtained as a yellow oil.

Step 9: Preparation of 2-[[6-[[5-chloro-2-[4-[2-[4-[2-[3-[4-(1-hydroxy-1-methyl-ethyl)-2-(6-methyl-7-oxo-1H-pyrrolo[2,3-c]pyridin-4-yl)phenoxy]phenoxy]ethyl]piperazin-1-yl]ethyl]piperazin-1-yl]pyrimidin-4-yl]amino]-1-methyl-2-oxo-3-quinolyl]oxy]-N-methyl-acetamide. To a solution of 4-[5-(1-hydroxy-1-methyl-ethyl)-2-[3-[2-[4-(2-piperazin-1-ylethyl) piperazin-1-yl]ethoxy]phenoxy]phenyl]-6-methyl-1H-pyrrolo[2,3-c]pyridin-7-one (0.1 g, 162.66 mol, 1 eq) in DMSO (5.0 mL) were added DIEA (74.2 mg, 574.11 mol, 0.1 mL, 3.53 eq) and 2-[[6-[(2,5-dichloropyrimidin-4-yl) amino]-1-methyl-2-oxo-3-quinolyl]oxy]-N-methyl-acetamide (85.0 mg, 208.21 mol, 1.28 eq). The mixture was stirred at 110° C. for 12 hours. The reaction mixture was filtered and concentrated under reduced pressure to give a residue. The residue was purified by prep-HPLC (basic condition). (column: Waters Xbridge C18 150*50 mm*10 um; mobile phase: [water (ammonia hydroxide v/v)-ACN]; gradient:34%-60% B over 9 min). Compound 2-[[6-[[5-chloro-2-[4-[2-[4-[2-[3-[4-(1-hydroxy-1-methyl-ethyl)-2-(6-methyl-7-oxo-1H-pyrrolo[2,3-c]pyridin-4-yl)phenoxy]phenoxy]ethyl]piperazin-1-yl]ethyl]piperazin-1-yl]pyrimidin-4-yl]amino]-1-methyl-2-oxo-3-quinolyl]oxy]-N-methyl-acetamide (24.3 mg, 24.48 mol, 15.05% yield, 99.4% purity) was obtained as an off-white solid. ¹H NMR (400 MHz, CDCl₃): δ (ppm) 10.12-10.04 (m, 1H), 8.03 (s, 1H), 7.78-7.72 (m, 2H), 7.67 (d, J=2.4 Hz, 1H), 7.62-7.57 (m, 1H), 7.48 (dd, J=8.8, 2.4 Hz, 1H), 7.36 (d, J=8.8 Hz, 1H), 7.23 (s, 1H), 7.12-7.01 (m, 4H), 6.93 (s, 1H), 6.52 (dd, J=8.0, 2.0 Hz, 1H), 6.46 (dd, J=8.0, 2.0 Hz, 1H), 6.40 (d, J=2.4 Hz, 1H), 6.37 (t, J=2.4 Hz, 1H), 4.57 (s, 2H), 3.99 (t, J=5.6 Hz, 2H), 3.82 (s, 3H), 3.80-3.73 (m, 4H), 3.60 (s, 3H), 2.92 (d, J=4.8 Hz, 3H), 2.77 (br t, J=5.6 Hz, 2H), 2.71-2.41 (m, 17H), 1.67 (s, 6H). LCMS (ES+): RT=1.953 min, m/z=986.7 [M+H+]; LCMS method: Method 01.

### Example 6—Synthesis of 2-[[6-[[5-chloro-2-[2-[4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenoxy]-7-azaspiro[3.5]nonan-7-yl]pyrimidin-4-yl]amino]-1-methyl-2-oxo-3-quinolyl]oxy]-N-methyl-acetamide (I-125)

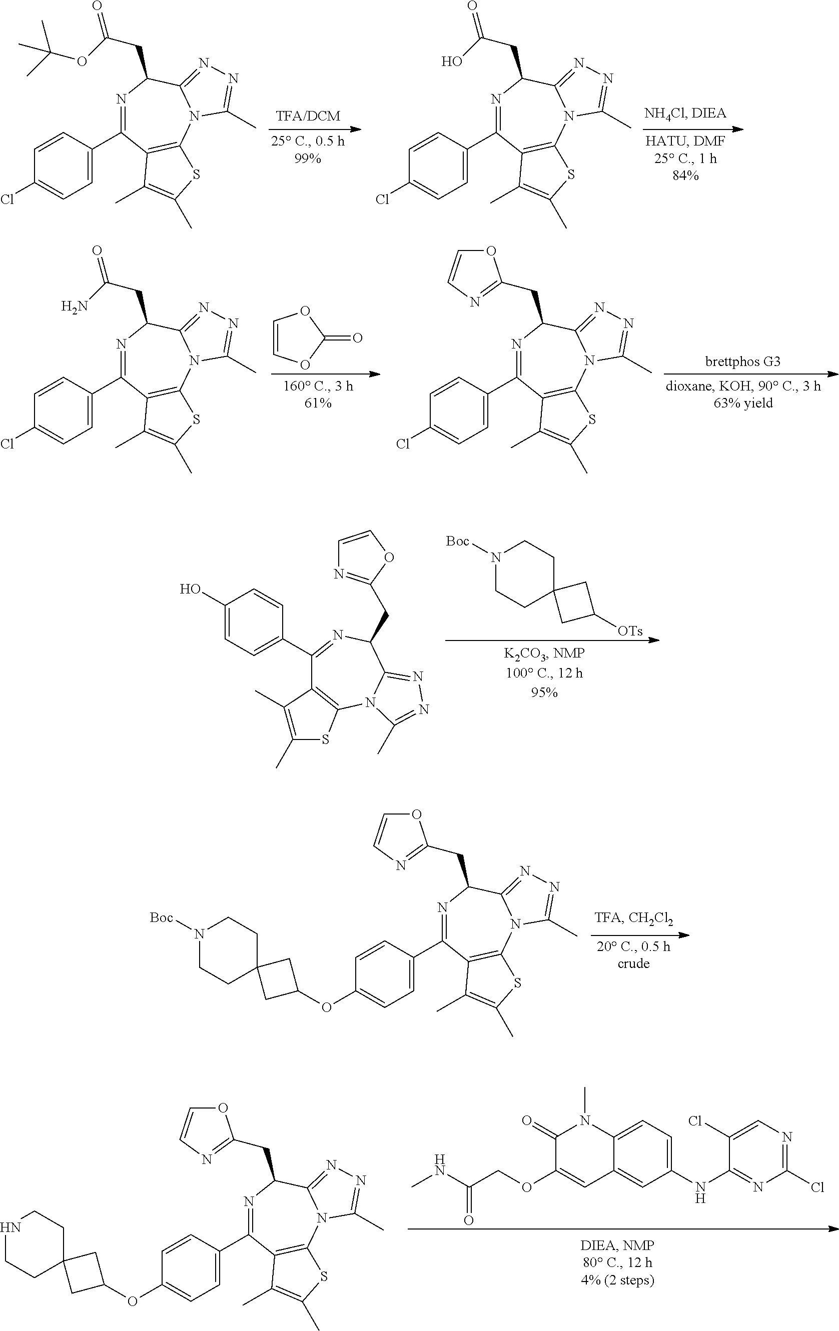

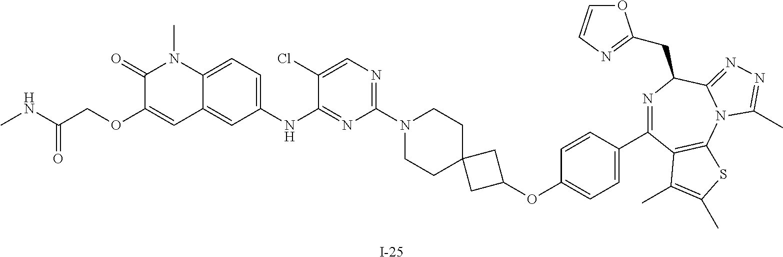

Step 1: Preparation of 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetic acid. To a solution of tert-butyl 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetate (1.0 g, 2.1 mmol, 1.0 equiv) in DCM (10 mL) was added TFA (7.7 g, 67 mmol, 5.0 mL, 30 equiv). The mixture was stirred at 25° C. for 0.5 h. The reaction mixture was filtered and concentrated under reduced pressure to give a residue. The residue was used for next step directly to give compound 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetic acid (877 mg, 99% yield).

Step 2: Preparation of 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetamide. To a solution of 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetic acid (877 mg, 2.19 mmol, 1.0 equiv) and NH₄Cl (351 mg, 6.56 mmol, 3.0 equiv) in DMF (8 mL) was added HATU (915 mg, 2.41 mmol, 1.1 equiv) and DIEA (848 mg, 6.56 mmol, 3.0 equiv). The mixture was stirred at 25° C. for 1 h. The mixture was filtered and concentrated. The residue was purified by prep-HPLC (column: Waters Xbridge C18 150*50 mm*10 um;mobile phase: [water(NH4HCO3)-ACN];B %: 24%-54%,10 min) to give 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetamide (740 mg, 84% yield).

Step 3: Preparation of 2-[[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]methyl]oxazole. To a solution of 1,3-dioxol-2-one (103 mg, 1.20 mmol, 1.2 equiv) and 2-[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]acetamide (400 mg, 1.00 mmol, 1.0 equiv) in PPA (4 mL). The mixture was stirred at 160° C. for 3 h. The mixture was quenched by addition H₂O 50 mL at 0° C., and then diluted with EA 200 mL. The mixture was extracted with H₂O (15 mL*2). The combined organic layers were washed with H₂O (10 mL*3), dried over Na₂SO₄, filtered and concentrated. The residue was purified by column chromatography (SiO₂, Petroleum ether/Ethyl acetate=2/1 to 0/1) to give compound 2-[[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]methyl]oxazole (260 mg, 61% yield).

Step 4: Preparation of 4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenol. A mixture of 2-[[(9S)-7-(4-chlorophenyl)-4,5,13-trimethyl-3-thia-1,8,11,12-tetrazatricyclo[8.3.0.02,6]trideca-2 (6),4,7,10,12-pentaen-9-yl]methyl]oxazole (5.8 g, 13.7 mmol, 1.0 equiv), BrettPhos Pd G3 (1.2 g, 1.4 mmol, 0.1 equiv), KOH (3.8 g, 68.4 mmol, 5.0 equiv) in dioxane (58 mL) and H₂O (11.6 mL) was degassed and purged with N₂ for 3 times, and then the mixture was stirred at 90° C. for 3 h under N₂ atmosphere. The reaction mixture was partitioned between Ethyl acetate (2000 mL) and water (500 mL). The organic phase was separated, washed with brine (500 mL×2), dried over Na₂SO₄, filtered and concentrated under reduced pressure to give a residue. The residue was purified by column chromatography (SiO₂, Dichloromethane. Methanol=40/1) to give the 4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenol (3.5 g, 8.6 mmol, 63% yield) as a yellow solid.

Step 5: Preparation of tert-butyl 2-[4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenoxy]-7-azaspiro[3.5]nonane-7-carboxylate. To a solution of tert-butyl 2-(p-tolylsulfonyloxy)-7-azaspiro[3.5]nonane-7-carboxylate (93.6 mg, 0.24 mmol, 1.2 equiv) in NMP (2 mL) was added K₂CO₃ (81.8 mg, 0.59 mmol, 3 equiv) and 4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenol (80.0 mg, 0.20 mmol, 1 equiv). The mixture was stirred at 100° C. for 12 h. The residue was poured into water (10 mL). The aqueous phase was extracted with ethyl acetate (5 mL*3). The combined organic phase was washed with brine (5 mL*3), dried with anhydrous Na₂SO₄, filtered and concentrated in vacuo. The residue was purified by prep-TLC on silica gel (CH₂Cl₂/MeOH=10/1) to give tert-butyl 2-[4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenoxy]-7-azaspiro[3.5]nonane-7-carboxylate (118 mg, 0.19 mmol, 95% yield) as a yellow oil.

Step 6: Preparation of 2-[[(9S)-7-[4-(7-azaspiro[3.5]nonan-2-yloxy)phenyl]-4,5,13-trimethyl-3-thia-1,8,11,12-tetrazatricyclo[8.3.0.02,6]trideca-2 (6),4,7,10,12-pentaen-9-yl]methyl]oxazole. To a solution of tert-butyl 2-[4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenoxy]-7-azaspiro[3.5]nonane-7-carboxylate (108 mg, 0.17 mmol, 1 equiv) in CH₂Cl₂ (2 mL) was added TFA (1 mL). The mixture was stirred at 20° C. for 0.5 h. The mixture was concentrated in vacuo to give 2-[[(9S)-7-[4-(7-azaspiro[3.5]nonan-2-yloxy)phenyl]-4,5,13-trimethyl-3-thia-1,8,11,12-tetrazatricyclo[8.3.0.02,6]trideca-2 (6),4,7,10,12-pentaen-9-yl]methyl]oxazole (110 mg, crude, TFA slat) as a yellow solid.

Step 7: Preparation of 2-[[6-[[5-chloro-2-[2-[4-[(9S)-4,5,13-trimethyl-9-(oxazol-2-ylmethyl)-3-thia-1,8,11,12-tetrazatricyclo[8.3.0.02,6]trideca-2 (6),4,7,10,12-pentaen-7-yl]phenoxy]-7-azaspiro[3.5]nonan-7-yl]pyrimidin-4-yl]amino]-1-methyl-2-oxo-3-quinolyl]oxy]-N-methyl-acetamide. To a solution of 2-[[(9S)-7-[4-(7-azaspiro[3.5]nonan-2-yloxy)phenyl]-4,5,13-trimethyl-3-thia-1,8,11,12-tetrazatricyclo[8.3.0.02,6]trideca-2 (6),4,7, 10,12-pentaen-9-yl]methyl]oxazole (100 mg, 0.16 mmol, 1 equiv, TFA) and 2-[[6-[(2,5-dichloropyrimidin-4-yl)amino]-1-methyl-2-oxo-3-quinolyl]oxy]-N-methyl-acetamide (38.1 mg, 0.09 mmol, 0.6 equiv) in NMP (2 mL) was added DIEA (60.3 mg, 0.47 mmol, 0.08 mL, 3 equiv). The mixture was stirred at 80° C. for 12 h. The residue was poured into water (10 mL). The aqueous phase was extracted with ethyl acetate (5 mL*3). The combined organic phase was washed with brine (3 mL*4), dried with anhydrous Na₂SO₄, filtered and concentrated in vacuo. The crude product was purified by prep-HPLC (column: Phenomenex luna C18 150*25 mm*10 um;mobile phase: [water(FA)-ACN];gradient:36%-66% B over 10 min) to afford 2-[[6-[[5-chloro-2-[2-[4-[(9S)-4,5,13-trimethyl-9-(oxazol-2-ylmethyl)-3-thia-1,8,11,12-tetrazatricyclo [8.3.0.02,6]trideca-2 (6),4,7,10,12-pentaen-7-yl]phenoxy]-7-azaspiro[3.5]nonan-7-yl]pyrimidin-4-yl]amino]-1-methyl-2-oxo-3-quinolyl]oxy]-N-methyl-acetamide (5.80 mg, 0.01 mmol, 4% yield for 2 steps) as a yellow solid. ¹H NMR (400 MHz, METHANOL-d₄) δ 7.91-7.88 (m, 3H), 7.79 (dd, 1H, J=8.8, 2.4 Hz), 7.51 (d, 1H, J=8.8 Hz), 7.30 (d, 2H, J=8.8 Hz), 7.20 (s, 1H), 7.12 (s, 1H), 6.81 (d, 2H, J=8.8 Hz), 4.80-4.76 (m, 1H), 4.74-4.70 (m, 1H), 4.57 (s, 2H), 4.00-3.92 (m, 2H), 3.80 (s, 3H), 3.72-3.66 (m, 2H), 3.66-3.60 (m, 2H), 2.86 (s, 3H), 2.69 (s, 3H), 2.46-2.52 (m, 2H), 2.45 (s, 3H), 2.00-1.90. (m, 2H), 1.69 (s, 3H), 1.68-1.64 (m, 2H), 1.63-1.59 (m, 2H). LC-MS: MS (ES⁺): RT=2.098 min, m/z=900.4 [M+H⁺]; LCMS method: Method 10.

### Example 7—Synthesis of 2-[[6-[[5-chloro-2-[2-[4-[(9S)-4,5,9,13-tetramethyl-3-thia-1,8,11,12-tetrazatricyclo[8.3.0.02,6]trideca-2 (6),4,7,10,12-pentaen-7-yl]phenoxy]-7-azaspiro[3.5]nonan-7-yl]pyrimidin-4-yl]amino]-1-methyl-2-oxo-3-quinolyl]oxy]-N-methyl-acetamide (I-28)

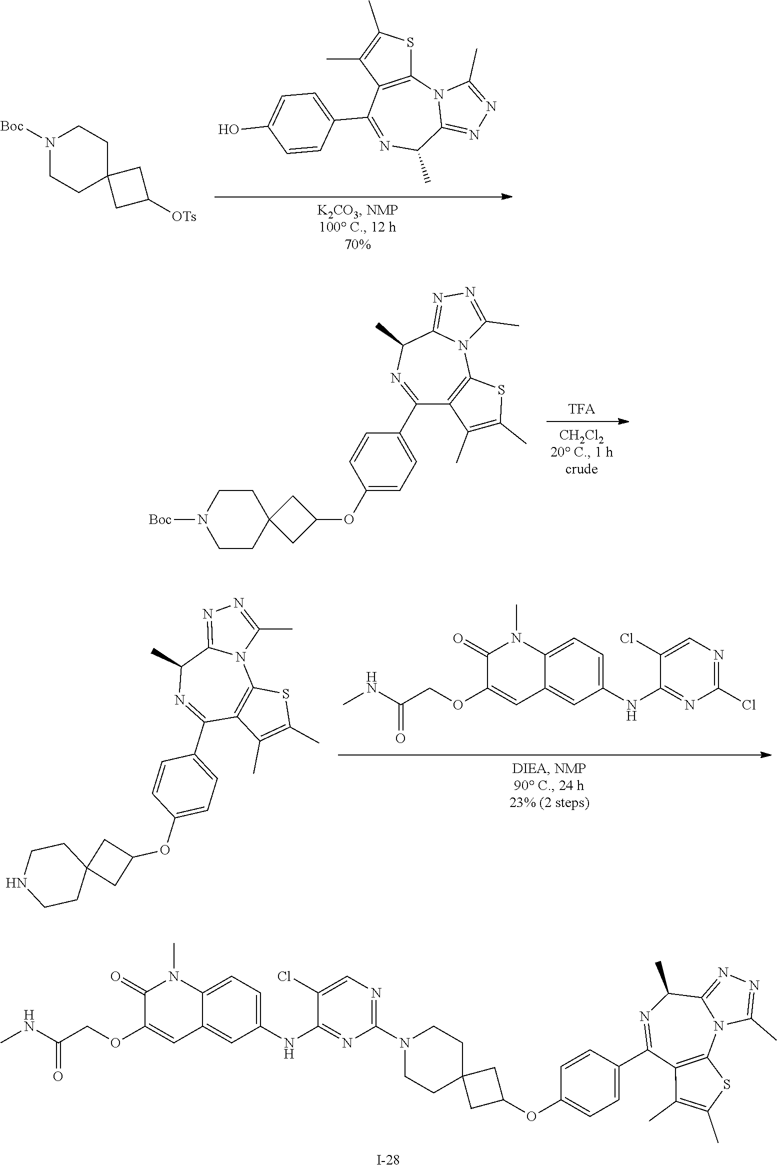

Step 1: Preparation of tert-butyl 2-[4-[(9S)-4,5,9,13-tetramethyl-3-thia-1,8,11,12-tetrazatricyclo[8.3.0.02,6]trideca-2 (6),4,7,10,12-pentaen-7-yl]phenoxy]-7-azaspiro[3.5]nonane-7-carboxylate. To a solution of tert-butyl 2-(p-tolylsulfonyloxy)-7-azaspiro[3.5]nonane-7-carboxylate (200 mg, 0.51 mmol, 1.0 equiv) in NMP (2 mL) was added K₂CO₃ (210 mg, 1.52 mmol, 3.0 equiv) and 4-[(9S)-4,5,9,13-tetramethyl-3-thia-1,8,11,12-tetrazatricyclo[8.3.0.02,6]trideca-2 (6),4,7,10,12-pentaen-7-yl]phenol (171 mg, 0.51 mmol, 1 equiv) at 20° C. The mixture was stirred at 100° C. for 12 h. Water (20 mL) was added to the reaction mixture and extracted with ethyl acetate (10 mL*3). The combined organic phase was washed with brine (5*6 mL), dried with Na₂SO₄, filtered and concentrated in vacuo. The residue was purified by prep-TLC (Dichloromethane/Methanol=10/1) to afford tert-butyl 2-[4-[(9S)-4,5,9,13-tetramethyl-3-thia-1,8,11,12-tetrazatricyclo[8.3.0.02,6]trideca-2 (6),4,7,10,12-pentaen-7-yl]phenoxy]-7-azaspiro[3.5]nonane-7-carboxylate (200 mg, 0.36 mmol, 70% yield) as a yellow solid.

Step 2: Preparation of (9S)-7-[4-(7-azaspiro[3.5]nonan-2-yloxy)phenyl]-4,5,9,13-tetramethyl-3-thia-1,8,11,12-tetrazatricyclo[8.3.0.02,6]trideca-2 (6),4,7,10,12-pentaene. To a solution of tert-butyl 2-[4-[(9S)-4,5,9,13-tetramethyl-3-thia-1,8,11,12-tetrazatricyclo[8.3.0.02,6]trideca-2 (6),4,7,10,12-pentaen-7-yl]phenoxy]-7-azaspiro[3.5]nonane-7-carboxylate (200 mg, 0.36 mmol, 1 equiv) in CH₂Cl₂ (3 mL) was added TFA (1 mL) at 20° C. The mixture was stirred at 20° C. for 1h to afford (9S)-7-[4-(7-azaspiro[3.5]nonan-2-yloxy)phenyl]-4,5,9,13-tetramethyl-3-thia-1,8,11,12-tetrazatricyclo[8.3.0.02,6]trideca-2 (6),4,7,10,12-pentaene (250 mg, crude, TFA) as a yellow oil.

Step 3: Preparation of 2-[[6-[[5-chloro-2-[2-[4-[(9S)-4,5,9,13-tetramethyl-3-thia-1,8,11,12-tetrazatricyclo[8.3.0.02,6]trideca-2 (6),4,7,10,12-pentaen-7-yl]phenoxy]-7-azaspiro[3.5]nonan-7-yl]pyrimidin-4-yl]amino]-1-methyl-2-oxo-3-quinolyl]oxy]-N-methyl-acetamide. To a solution of (9S)-7-[4-(7-azaspiro[3.5]nonan-2-yloxy)phenyl]-4,5,9,13-tetramethyl-3-thia-1,8,11,12-tetrazatricyclo[8.3.0.02,6]trideca-2 (6),4,7,10,12-pentaene (280 mg, 0.49 mmol, 1 equiv, TFA) in NMP (2.5 mL) was added DIEA (223 mg, 1.72 mmol 3.5 equiv) and 2-[[6-[(2,5-dichloropyrimidin-4-yl)amino]-1-methyl-2-oxo-3-quinolyl]oxy]-N-methyl-acetamide (120 mg, 0.29 mmol, 0.6 equiv) at 20° C. The mixture was stirred at 90° C. for 24 h. The reaction mixture was diluted with water (20 mL). The mixture was extracted with ethyl acetate (10 mL×2). The combined organic phase was washed with brine (20 mL), dried with Na₂SO₄, filtered and concentrated in vacuo. The residue was purified by prep-TLC (Dichloromethane/Methanol) and SFC (column: DAICEL CHIRALPAK AD (250 mm*30 mm,10 um);mobile phase: [CO₂-i-PrOH/ACN];B %:60%, isocratic elution mode) to afford 2-[[6-[[5-chloro-2-[2-[4-[(9S)-4,5,9,13-tetramethyl-3-thia-1,8,11,12-tetrazatricyclo[8.3.0.02,6]trideca-2 (6),4,7,10,12-pentaen-7-yl]phenoxy]-7-azaspiro[3.5]nonan-7-yl]pyrimidin-4-yl]amino]-1-methyl-2-oxo-3-quinolyl]oxy]-N-methyl-acetamide (96.0 mg, 0.11 mmol, 23% yield for 2 steps, 98% purity) as a white solid. ¹H NMR (400 MHz, CD₃OD) δ 7.95-7.84 (m, 2H), 7.75-7.70 (m, 1H), 7.60-7.50 (m, 1H), 7.45-7.35 (m, 2H), 7.26 (s, 1H), 6.87-6.84 (m, 2H), 4.75-7.65 (m, 1H), 4.60 (s, 2H), 4.29-4.24 (m, 1H), 3.83 (s, 3H), 3.71-3.64 (m, 4H), 2.89-2.80 (m, 3H), 2.70 (s, 3H), 2.65-2.50 (m, 2H), 2.43 (s, 3H), 2.02-1.85 (m, 5H), 1.68 (s, 3H), 1.70-1.55 (m, 2H), 1.47-1.35 (m, 1H), 1.32-1.31 (in, 1H). LC-MS: MS (ES⁺): RT=2.193 min m/z=833.2 [M+H⁺]; LCMS method: 01.

### Example 8—Synthesis of Additional Compounds

The following compounds were prepared based on procedures described herein: I-2, I-3, I-4, I-5, I-6, I-7, I-8, I-9, I-10, I-11, I-12, I-13, I-14, I-15, I-17, I-19, I-20, I-21, I-24, I-26, I-27, 1-38, I-39, I-40, I-41, I-42, I-43, I-44, I-45, 5-46, I-47, I-48, I-49, I-50, I-51, I-52, I-53, I-54, and I-55.

Exemplary compounds were characterized by LC-MS. Characterization data is provided in Table 2.

TABLE 2

| Compound | MW Exact | Observed Mass | LCMS Purity UV 220 |
|---|---|---|---|
| No. | (g/mol) | (g/mol) | (%) |
| I-2 | 937.3 | 469.6, 938.2 | 97.17 |
| I-3 | 1025.4 | 513.7, 1026.3 | 97.3 |
| I-4 | 1019.4 | 510.9, 1020.6 | 94.07 |
| I-5 | 962.33 | 482.1, 963.2 | 94.45 |
| I-6 | 1033.4 | 1034.4 | 98.52 |
| I-7 | 1019.4 | 1020.4 | 98.82 |
| I-8 | 1033.4 | 1034.4 | 99.79 |
| I-17 | 1003.4 | 1004.4 | 96.93 |
| I-9 | 1047.4 | 1048.4 | 98.18 |
| I-10 | 1047.4 | 1048.4 | 96 |
| I-11 | 1047.4 | 1048.4 | 98.97 |
| I-12 | 1047.4 | 1048.4 | 96.62 |
| I-13 | 1033.4 | 517.8, 1034.4 | 97.31 |
| I-14 | 1033.4 | 517.8, 1034.4 | 95.76 |
| I-19 | 989.5 | 990.6 | 98.92 |
| I-20 | 961.47 | 962.7 | 95.33 |
| I-21 | 975.49 | 976.6 | 99.24 |
| I-24 | 984.44 | 985.4 | 93.62 |
| I-15 | 1034.4 | 1035.7 | 95.84 |
| I-26 | 928.34 | 929.4 | 96.53 |
| I-27 | 899.31 | 900.4 | 96.65 |
| I-38 | 940.42 | 941.4 | 98.93 |
| I-39 | 940.42 | 941.4 | 96.53 |
| I-40 | 926.4 | 927.4 | 95.68 |
| I-41 | 861.37 | 862.5 | 90.87 |
| I-42 | 861.37 | 862.6 | 96.74 |
| I-43 | 958.46 | 959.6 | 98.99 |
| I-44 | 958.46 | 959.7 | 99.51 |
| I-45 | 932.45 | 933.6 | 96.85 |
| I-46 | 932.45 | 933.6 | 89.17 |
| I-47 | 955.43 | 956.4 | 99.17 |
| I-48 | 926.4 | 927.4 | 92.77 |
| I-49 | 889.4 | 890.4 | 94.45 |
| I-50 | 955.43 | 956.4 | 98.28 |
| I-51 | 889.4 | 890.4 | 95.95 |
| I-52 | 860.38 | 861.4 | 98.97 |
| I-53 | 957.47 | 958.4 | 91.79 |
| I-54 | 957.47 | 958.4 | 99.56 |
| I-55 | 890.4 | 891.4 | 98.17 |

### Example 9—Assay for Binding Affinity to BCL6

Exemplary compounds were tested for ability to bind to B-cell lymphoma 6 (BCL6) protein. Experimental procedures and results are provided below.

### Part I—Experimental Procedure

Binding studies by surface plasmon resonance (SPR) were performed using a Biacore 8K (Cytiva) instrument at 25° C. Briefly, biotinylated BCL6 (5-129) was diluted in 10 mM HEPES pH 7.4, 150 mM NaCl, 0.05% Surfactant P20, 0.5 mM TCEP, 3% DMSO to 1-5 μg/mL and was immobilized on an SA sensor chip (Cytiva, BR100032), resulting in surface densities at 1000-3000 resonance units (RUs). Test compounds were serially diluted five times in the same buffer and were injected to the immobilized surface. Kinetic determination experiments were performed using single-cycle kinetics with 110-120 s contact time, 1000-4000 s dissociation time, 30 μL/min flow rate. Binding data were analyzed by using Biacore insight evaluation software to determine K_{D} values for test compounds.

### Part II—Results

Results showing ability of exemplary compounds to bind to BCL6 are provided in Table 3. below. The symbol “++++” indicates a K_{D} less than 0.001 μM. The symbol “+++” indicates a K_{D} in the range of 0.001 μM to 0.01 μM. The symbol “++” indicates a K_{D} in the range of greater than 0.01 μM to 0.1 μM. The symbol “+” indicates a K_{D} greater than 0.1 μM.

TABLE 3

| Compound No. | K_{D} |
|---|---|
| I-4 | + |
| I-18 | +++ |
| I-19 | +++ |
| I-20 | +++ |
| I-21 | +++ |
| I-23 | +++ |
| I-24 | +++ |
| I-15 | +++ |
| I-25 | + |
| I-26 | ++ |
| I-22 | +++ |
| I-27 | + |
| I-38 | + |
| I-39 | ++ |
| I-40 | ++ |
| I-41 | +++ |
| I-42 | ++++ |
| I-43 | +++ |
| I-44 | +++ |
| I-45 | +++ |
| I-46 | +++ |
| I-47 | +++ |
| I-48 | ++ |

### Example 10—Assay for Binding Affinity to BRD4

Exemplary compounds were tested for ability to bind to bromodomain-containing protein 4 (BRD4). Experimental procedures and results are provided below.

### Part I—Experimental Procedure

Compounds were tested using a developed AlphaLISA competition assay. Briefly, His-tagged BRD4 BD1 (46-168) and biotinylated histone H4K5/8/12/16(Ac) peptide (ANASPEC, AS-64989-1) were incubated in 20 mM HEPES pH 7.5, 100 mM NaCl, 0.5 mM TCEP, 0.1% BSA, 0.02% CHAPS at a final concentration of 10 nM. Test compounds were serially diluted ten times in the same buffer and were added to the BRD4-peptide mixture for 1 h incubation at room temperature. Following incubation, Streptavidin AlphaLISA acceptor beads (PerkinElmer, AL125M) were added to BRD4-peptide-compound samples to a final concentration of 0.01 mg/mL for a 0.5 h incubation at room temperature in the dark. Following incubation, the same amount of Nickel Chelate Alpha donor beads (PerkinElmer, AS101M) were added to the samples for a final 1 h incubation. The AlphaLISA signals were measured using an EnVision plate reader (PerkinElmer). The data were uploaded to Screening Ultra (Dotmatics) to determine the half-maximal inhibitory concentration (IC₅₀) for each test compound.

The same assay was developed for His-tagged BRD4 BD2 (333-460) and biotinylated histone H4K5/8/12/16(Ac) peptide at a final concentration of 20 nM.

### Part II—Results

Results showing ability of exemplary compounds to bind to BRD4-BD1 and BRD4-BD2 are provided in Tables 4 and 5 respectively below. The symbol “++++” indicates an IC₅₀ less than 0.005 μM. The symbol “+++” indicates an IC₅₀ in the range of 0.005 μM to 0.05 μM. The symbol “++” indicates an IC₅₀ in the range of greater than 0.05 μM to 0.5 μM. The symbol “+” indicates an IC₅₀ greater than 0.5 μM.

TABLE 4

| Compound No. | IC₅₀ |
|---|---|
| I-4 | ++ |
| I-6 | +++ |
| I-16 | +++ |
| I-7 | +++ |
| I-8 | +++ |
| I-17 | +++ |
| I-9 | +++ |
| I-10 | +++ |
| I-11 | +++ |
| I-12 | +++ |
| I-13 | +++ |
| I-14 | +++ |
| I-18 | +++ |
| I-19 | +++ |
| I-20 | +++ |
| I-21 | ++++ |
| I-23 | ++++ |
| I-24 | ++ |
| I-15 | ++++ |
| I-25 | +++ |
| I-26 | ++++ |
| I-22 | ++++ |
| I-27 | +++ |
| I-38 | ++++ |
| I-39 | +++ |
| I-40 | ++++ |
| I-41 | ++++ |
| I-42 | ++++ |
| I-43 | ++++ |
| I-44 | ++++ |
| I-45 | ++++ |
| I-46 | ++++ |
| I-47 | ++++ |
| I-48 | ++++ |

TABLE 5

| Compound No. | IC₅₀ |
|---|---|
| I-4 | +++ |
| I-6 | +++ |
| I-16 | ++++ |
| I-7 | +++ |
| I-8 | +++ |
| I-17 | +++ |
| I-9 | +++ |
| I-10 | ++++ |
| I-11 | +++ |
| I-12 | +++ |
| I-13 | +++ |
| I-14 | ++++ |
| I-18 | ++++ |
| I-19 | ++++ |
| I-20 | ++++ |
| I-21 | ++++ |
| I-23 | ++++ |
| I-24 | ++++ |
| I-15 | ++++ |
| I-25 | ++++ |
| I-26 | ++++ |
| I-22 | ++++ |
| I-27 | ++++ |
| I-38 | ++++ |
| I-39 | ++++ |
| I-40 | ++++ |
| I-41 | + |
| I-42 | + |
| I-43 | ++++ |
| I-44 | ++++ |
| I-45 | ++++ |
| I-46 | ++++ |
| I-47 | ++++ |
| I-48 | ++++ |

### Example 11—Cellular Growth Inhibition Assay Using T-Rex 293 Cells

Exemplary compounds were tested for ability to inhibit the proliferation of the following types of cells: (i) a T-Rex 293 cell line having increased expression of BCL6 protein due to exposure of doxycycline and (ii) a T-Rex 293 cell line lacking increased expression of BCL6 protein. Experimental procedures and results are provided below.

### Part I—Experimental Procedure

The following types of cells were prepared for this experiment: (i) a T-Rex 293 cell line having increased expression of BCL6 protein due to exposure to doxycycline and (ii) a T-Rex 293 cell line lacking increased expression of BCL6 protein. Ability of the test compounds to inhibit proliferation of the foregoing cell types was evaluated according to the procedures set forth below.

The doxycycline-inducible BCL6 protein expressing cell line was established using the following protocol: T-Rex 293 cells were purchased from Invitrogen (Cat #R71007) and transfected using Lipofectamine 2000 with the wild-type BCL6 protein sequence cloned into the pcDNA4/TO vector. Transfected cells were selected using 400 μg/mL Zeocin (Invitrogen Cat #R25001). Following selection, single clones were raised and maintained in Dulbecco's Modified Eagle Medium (DMEM) supplemented with 10% w/w Tetracycline-free fetal bovine serum (FBS) and 250 μg/mL Zeocin. Clones were analyzed for expression of BCL6 protein in the presence and absence of 10 ng/mL doxycycline (Sigma Cat #D9891), and a single doxycycline-inducible clone (hereinafter “SC”) was selected for use in downstream assays.

The SC cells were seeded on poly-D-lysine coated, black clear-bottom 384-well plates at 2500/well, in 25 μL Dulbecco's Modified Eagle Medium (DMEM) supplemented with 10% w/w Tetracycline-free fetal bovine serum (FBS) and 1% w/w pen-strep, with or without 10 ng/mL doxycycline. Pen-Strep is a commercially available mixture of penicillin G and streptomycin, which is used in mammalian cell culture media to prevent bacterial contamination. Dulbecco's Modified Eagle Medium (DMEM) supplemented with 10% w/w Tetracycline-free fetal bovine serum (FBS) and 1% w/w pen-strep, with or without 10 ng/mL doxycycline is herein referred to as Treatment Medium. Following seeding of cells in the plates, the plates were spun at 300×g for 30 seconds, then equilibrated to room temperature for 30 minutes, and then deposited in a humidified tissue culture incubator maintained at 37° C. with 5% CO₂.

At 24 hours after seeding of the cells, dilutions of test compound were prepared in DMSO and dissolved in Treatment Medium, to achieve a final DMSO concentration of 0.5% w/w, thereby providing the Test Compound Solution. A 25 μL aliquot of the Test Compound Solution was added to cells in the well plates. An equal volume of a solution containing DMSO and Treatment Medium was used as a negative control. Following treatment of cells with Test Compound Solution or said equal volume of a solution containing DMSO and Treatment Medium, the plates were spun at 300×g for 30 seconds, and then left in an incubator for 72 hours.

At the end of the treatment duration, cell viability was quantified with CellTiter-Glo 2.0 reagent (Promega Cat #G9243). For this purpose, plates were equilibrated to room temperature for 30 minutes, and then 25 μL of CellTiter-Glo 2.0 reagent was added to cells in the plate wells. Plates were then agitated on a shaker for two minutes at 500 rpm and subsequently incubated at room temperature for 10 minutes. Following incubation, the plates were spun at 3000×g for 30 seconds, then sealed with an optical adhesive cover, and luminescence readings were measured with an EnVision Plate Reader (Perkin Elmer).

Data was normalized using zero luminescence for baseline. A four-parameter non-linear regression curve fit was applied to dose-response data in GraphPad Prism data analysis software to determine the half-maximal growth inhibitory concentration (GI₅₀) for each test compound.

### Part II—Results

The half-maximal growth inhibitory concentration (GI₅₀) results are provided in Tables 6 and 7 below for exemplary compounds. Table 6 provides results from the experiment analyzing ability of test compounds to inhibit proliferation of the T-Rex 293 cell line SC cells having increased expression of BCL6 protein due to exposure to doxycycline. The symbol “++++” indicates an GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM.

TABLE 6

| Compound No. | GI₅₀ |
|---|---|
| I-2 | ++++ |
| I-3 | ++ |
| I-4 | ++++ |
| I-5 | ++++ |
| I-6 | ++++ |
| I-16 | ++++ |
| I-7 | ++++ |
| I-8 | ++++ |
| I-17 | + |
| I-9 | ++++ |
| I-10 | ++++ |
| I-11 | ++++ |
| I-12 | ++++ |
| I-13 | ++++ |
| I-14 | ++++ |
| I-18 | ++++ |
| I-19 | ++++ |
| I-20 | ++++ |
| I-21 | ++++ |
| I-23 | ++++ |
| I-24 | ++++ |
| I-15 | ++++ |
| I-25 | ++++ |
| I-26 | ++++ |
| I-22 | ++++ |
| I-27 | ++++ |
| I-38 | ++++ |
| I-39 | ++++ |
| I-40 | ++++ |
| I-41 | ++++ |
| I-42 | ++++ |
| I-43 | ++++ |
| I-44 | ++++ |
| I-45 | ++++ |
| I-46 | ++++ |
| I-47 | ++++ |
| I-48 | ++++ |
| I-49 | ++++ |
| I-50 | ++++ |
| I-51 | ++++ |
| I-52 | ++++ |
| I-28 | ++++ |
| I-53 | ++++ |
| I-54 | ++++ |
| I-55 | ++++ |

Table 7 provides results from the experiment analyzing ability of test compounds to inhibit proliferation of the T-Rex 293 cell line SC cells lacking increased expression of BCL6 protein since such cells were not exposed to doxycycline. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM.

TABLE 7

| Compound No. | GI₅₀ |
|---|---|
| I-2 | + |
| I-3 | + |
| I-4 | ++++ |
| I-5 | ++++ |
| I-6 | ++++ |
| I-16 | ++++ |
| I-7 | ++++ |
| I-8 | ++++ |
| I-17 | ++++ |
| I-9 | ++++ |
| I-10 | ++++ |
| I-11 | ++++ |
| I-12 | ++++ |
| I-13 | ++++ |
| I-14 | +++ |
| I-18 | +++ |
| I-19 | ++ |
| I-20 | +++ |
| I-21 | ++ |
| I-23 | +++ |
| I-24 | ++++ |
| I-15 | +++ |
| I-25 | ++++ |
| I-26 | ++++ |
| I-22 | ++++ |
| I-27 | ++++ |
| I-38 | ++++ |
| I-39 | ++++ |
| I-40 | ++++ |
| I-41 | + |
| I-42 | + |
| I-43 | ++++ |
| I-44 | +++ |
| I-45 | ++++ |
| I-46 | ++++ |
| I-47 | ++++ |
| I-48 | ++++ |
| I-49 | ++++ |
| I-50 | ++++ |
| I-51 | ++++ |
| I-52 | ++++ |
| I-28 | ++++ |
| I-53 | ++++ |
| I-54 | ++++ |
| I-55 | ++++ |

### Example 12—Cellular Growth Inhibition Assay for Su-DHL-4 and Farage Cells

Exemplary compounds were tested for ability to inhibit the proliferation of Su-DHL-4 and Farage cells. Su-DHL-4 and Farage cells are commercially available diffuse large B cell lymphoma cell lines (DLBCL). Experimental procedures and results are provided below.

### Part I—Experimental Procedure

Su-DHL-4 and Farage cells were purchased from American Type Cell Culture (ATCC Cat #CRL-2957/CRL-2630) and then seeded on poly-D-lysine coated, black clear-bottom 384-well plates at 3000/well in 25 μL Roswell Park Memorial Institute media (RPMI-1640) supplemented with 10% w/w Fetal bovine serum (FBS) and 1% w/w Pen-Strep. Pen-Strep is a commercially available mixture of penicillin G and streptomycin, which is used in mammalian cell culture media to prevent bacterial contamination. Roswell Park Memorial Institute media (RPMI-1640) supplemented with 10% w/w fetal bovine serum (FBS), and 1% w/w Pen-Strep is herein referred to as Treatment Medium. Following seeding of cells in the plates, the plates were spun at 300×g for 30 seconds, then equilibrated to room temperature for 30 minutes, and then deposited in a humidified tissue culture incubator maintained at 37° C. with 5% CO₂.

At 24 hours after seeding of the cells, dilutions of test compound were prepared in DMSO and dissolved in Treatment Medium, to achieve a final DMSO concentration of 0.5% w/w, thereby providing the Test Compound Solution. A 25 μL aliquot of the Test Compound Solution was added to cells in the well plates. An equal volume of a solution containing DMSO and Treatment Medium was used as a negative control. Following treatment of cells with Test Compound Solution or said equal volume of a solution containing DMSO and Treatment Medium, the plates were spun at 300×g for 30 seconds, and then left in an incubator for 72 hours.

At the end of the treatment duration, cell viability was quantified with CellTiter-Glo 2.0 reagent (Promega Cat #G9243). For this purpose, plates were equilibrated to room temperature for 30 minutes, and then 25 μL of CellTiter-Glo 2.0 reagent was added to cells in the plate wells. Plates were then agitated on a shaker for two minutes at 500 rpm and subsequently incubated at room temperature for 10 minutes. Following incubation, the plates were spun at 3000×g for 30 seconds, then sealed with an optical adhesive cover, and luminescence readings were measured with an EnVision Plate Reader (Perkin Elmer).

Data was normalized using zero luminescence for baseline. A four-parameter non-linear regression curve fit was applied to dose-response data in GraphPad Prism data analysis software to determine the half-maximal growth inhibitory concentration (GI₅₀) for each test compound.

### Part II—Results

The half-maximal growth inhibitory concentration (GI₅₀) results are provided in Tables 8 and 9 below for exemplary compounds. Table 8 provides results from the experiment analyzing ability of test compounds to inhibit proliferation of the Su-DHL-4 cell line. The symbol “++++” indicates an GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM.

TABLE 8

| Compound No. | GI₅₀ |
|---|---|
| I-1 | ++++ |
| I-2 | ++++ |
| I-3 | +++ |
| I-4 | ++++ |
| I-5 | ++++ |
| I-18 | ++++ |
| I-19 | ++++ |
| I-20 | ++++ |
| I-21 | ++++ |
| I-23 | ++++ |
| I-24 | ++++ |
| I-15 | ++++ |
| I-25 | ++++ |
| I-26 | ++++ |
| I-22 | ++++ |
| I-27 | ++++ |

Table 9 provides results from the experiment analyzing ability of test compounds to inhibit proliferation of the Farage cell line. The symbol “++++” indicates a GI₅₀ less than 0.5 μM. The symbol “+++” indicates an GI₅₀ in the range of 0.5 μM to 1.5 μM. The symbol “++” indicates a GI₅₀ in the range of greater than 1.5 μM to 3 μM. The symbol “+” indicates a GI₅₀ greater than 3 μM.

TABLE 9

| Compound No. | GI₅₀ |
|---|---|
| I-18 | ++++ |
| I-19 | ++++ |
| I-20 | ++++ |
| I-21 | ++++ |
| I-23 | ++++ |
| I-24 | ++++ |
| I-15 | ++++ |
| I-25 | ++++ |
| I-22 | ++++ |
| I-27 | ++++ |
| I-38 | ++++ |
| I-39 | ++++ |
| I-40 | ++++ |
| I-41 | ++ |
| I-42 | ++ |
| I-43 | ++++ |
| I-44 | ++++ |
| I-45 | ++++ |
| I-46 | ++++ |
| I-47 | ++++ |
| I-48 | ++++ |
| I-49 | ++++ |
| I-50 | ++++ |
| I-51 | ++++ |
| I-52 | ++++ |
| I-28 | ++++ |
| I-53 | ++++ |
| I-54 | ++++ |
| I-55 | ++++ |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound represented by Formula I: 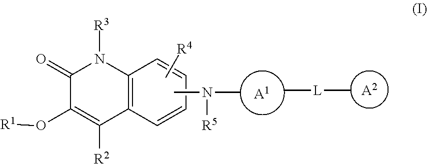
or a pharmaceutically acceptable salt thereof, wherein:
R¹ is —(C₁₋₃ alkylene)-C(O)N(R⁶)₂;
R², R³, R⁵, and R⁶ are independently hydrogen or C₁₋₄ alkyl;
R⁴ is hydrogen, C₁₋₄ alkyl, or halo;
R⁷ represents independently for each occurrence halo or C₁₋₄ alkyl;
A¹ is a pyrimidinylene, pyridazinylene, pyrazinylene, pyridinylene, or phenylene, each of which is substituted with n occurrences of R⁷;
L is a linker; and
A² is one of the following: 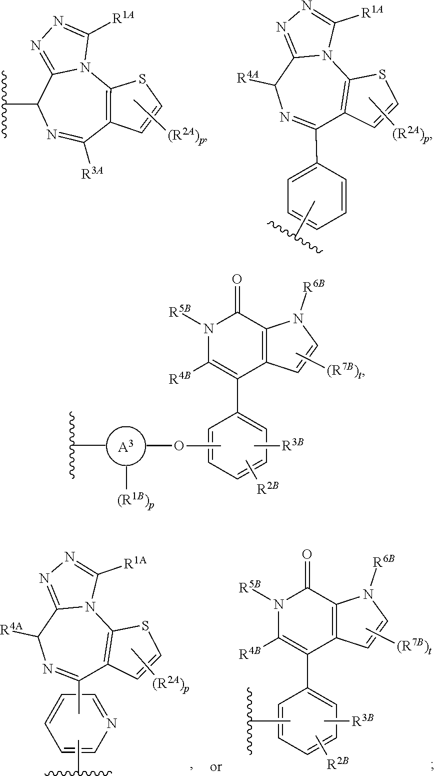
R^{1A} is C₁₋₄ alkyl or C₃₋₄ cycloalkyl;
R^{2A} represents independently for each occurrence C₁₋₄ alkyl or C₃₋₄ cycloalkyl;
R^{3A} is phenyl substituted with 1, 2, or 3 substituents independently selected from halo, C₁-C₄ alkyl, or C₁-C₄ haloalkyl;
R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, C₁₋₆ alkyl, or —(C₀₋₆ alkylene)-(5-6 membered heteroaryl containing 1, 2, or 3 heteroatoms independently selected from nitrogen, oxygen, and sulfur);
R^{5A} and R^{6A} are independently hydrogen, C₁₋₆ alkyl, or C₃₋₆ cycloalkyl; or R^{5A} and R^{6A} are taken together with the nitrogen atom to which they are attached to form a 3-7 membered ring containing 1 nitrogen atom;
R^{7A} is C₁₋₆ alkyl, —(C₁₋₆ alkylene)-(C₃₋₆ cycloalkyl), or C₃₋₆ cycloalkyl;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
A³ is phenylene, a 3-10 membered saturated monocyclic, bicyclic or spirocyclic carbocycylene, or C₁₋₆ alkylene; and
n, p, and t are independently 0, 1, or 2.

2. The compound of claim 1, wherein R¹ is —CH₂—C(O)N(R⁶)₂.

3. The compound of claim 1, wherein R¹ is —CH₂—C(O)N(H)CH₃.

4. The compound of any one of claims 1-3, wherein R² is hydrogen.

5. The compound of any one of claims 1-4, wherein R³ is C₁₋₄ alkyl.

6. The compound of any one of claims 1-4, wherein R³ is methyl.

7. The compound of any one of claims 1-5, wherein R⁴ and R⁵ are hydrogen.

8. The compound of any one of claims 1-7, wherein the compound is a compound of Formula I.

9. The compound of any one of claims 1-7, wherein the compound is a compound of Formula Ia or a pharmaceutically acceptable salt thereof:

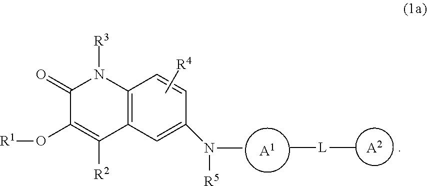

10. The compound of claim 1, wherein the compound is a compound of Formula Ib or a pharmaceutically acceptable salt thereof:

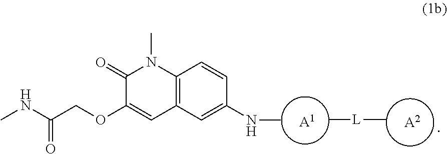

11. The compound of any one of claims 1-10, wherein A¹ is pyrimidinylene substituted with n occurrences of R⁵.

12. The compound of any one of claims 1-10, wherein A¹ is 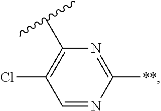
where ** is the point of attachment to L.

13. The compound of any one of claims 1-10, wherein A¹ is pyrazinylene substituted with n occurrences of R⁵.

14. The compound of any one of claims 1-10, wherein A¹ is pyridazinylene substituted with n occurrences of R⁵.

15. The compound of any one of claims 1-10, wherein A¹ is pyridinylene substituted with n occurrences of R⁵.

16. The compound of any one of claims 1-10, wherein A¹ is phenylene substituted with n occurrences of R⁵.

17. The compound of any one of claims 1-11 or 13-16, wherein n is 1.

18. The compound of any one of claims 1-11 or 13-17, wherein R⁷ is chloro or fluoro.

19. The compound of any one of claims 1-18, wherein A² is

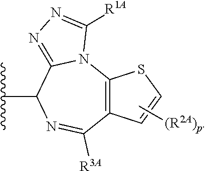

20. The compound of any one of claims 1-19, wherein R^{3A} is phenyl substituted with halo.

21. The compound of any one of claims 1-18, wherein A² is

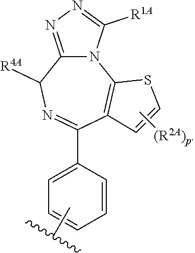

22. The compound of any one of claims 1-18 or 21, wherein R^{4A} is —(C₁₋₆ alkylene)-C(O)N(R^{5A})(R^{6A}).

23. The compound of any one of claims 1-18 or 21, wherein R^{4A} is —(C₁₋₆ alkylene)-CO₂R^{7A}.

24. The compound of any one of claims 1-23, wherein R^{1A} is C₁₋₄ alkyl.

25. The compound of any one of claims 1-23, wherein R^{1A} is methyl.

26. The compound of any one of claims 1-25, wherein R^{2A} is C₁₋₄ alkyl.

27. The compound of any one of claims 1-25, wherein R^{2A} is methyl.

28. The compound of any one of claims 1-27, wherein p is 2.

29. The compound of any one of claims 1-18, wherein A² is

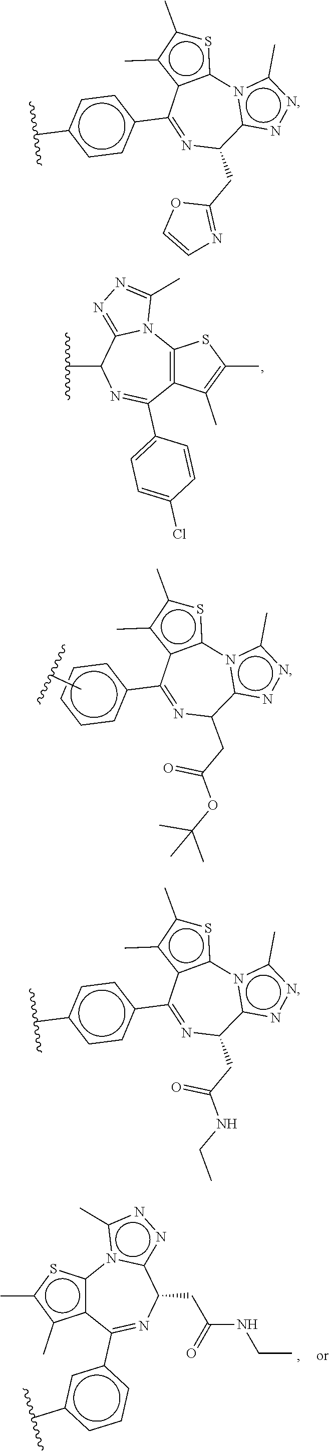

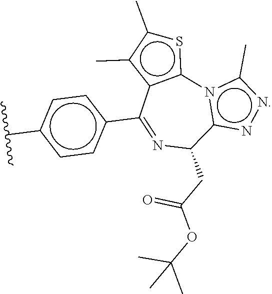

30. The compound of any one of claims 1-18, wherein A² is

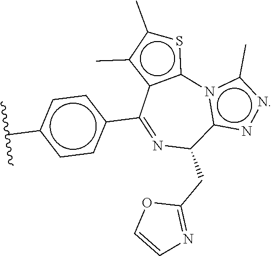

31. The compound of any one of claims 1-18, wherein A² is

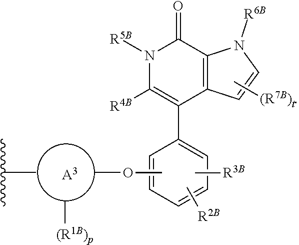

32. The compound of any one of claims 1-18 or 31, wherein A³ is a 3-10 membered saturated monocyclic, bicyclic or spirocyclic carbocycylene.

33. The compound of any one of claims 1-18, wherein A² is

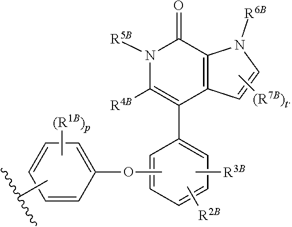

34. The compound of any one of claims 1-18, wherein A² is

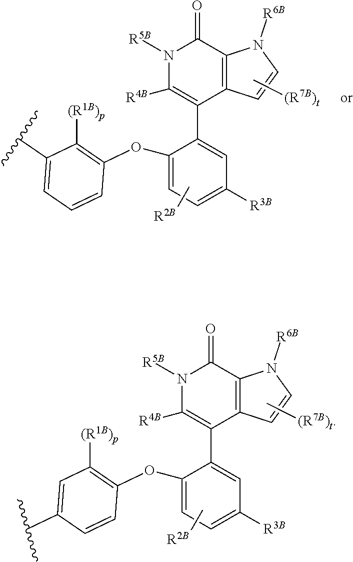

35. The compound of any one of claims 1-18 or 31-34, wherein R^{1B} is halo.

36. The compound of any one of claims 1-18 or 31-34, wherein R^{1B} is fluoro.

37. The compound of any one of claims 1-18 or 31-36, wherein p is 1.

38. The compound of any one of claims 1-18 or 31-34, wherein p is 0.

39. The compound of any one of claims 1-18, wherein A² is

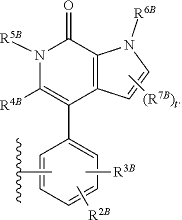

40. The compound of any one of claims 1-18, wherein A² is

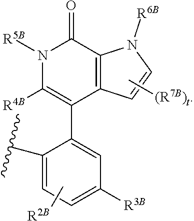

41. The compound of any one of claims 1-18 or 31-40, wherein R^{2B} is hydrogen.

42. The compound of any one of claims 1-18 or 31-41, wherein R^{3B} is —N(R^{8B})SO₂R^{9B}.

43. The compound of any one of claims 1-18 or 31-41, wherein R^{3B} is SO₂R^{9B}.

44. The compound of any one of claims 1-18 or 31-41, wherein R^{3B} is —(C₁₋₆ alkylene)-SO₂R^{9B}.

45. The compound of any one of claims 1-18 or 31-41, wherein R^{3B} is —SO₂N(R^{8B})₂.

46. The compound of any one of claims 1-18 or 31-41, wherein R^{3B} is C₁₋₆ hydroxyalkyl.

47. The compound of any one of claims 1-18 or 31-44, wherein R^{9B} is C₁₋₆ alkyl or C₁₋₆ haloalkyl.

48. The compound of any one of claims 1-18 or 31-47, wherein R^{4B} is hydrogen.

49. The compound of any one of claims 1-18 or 31-48, wherein R^{5B} is C₁₋₄ alkyl.

50. The compound of any one of claims 1-18 or 31-48, wherein R^{5B} is methyl.

51. The compound of any one of claims 1-18 or 31-50, wherein R^{6B} is hydrogen.

52. The compound of any one of claims 1-18 or 31-51, wherein R^{7B} is C₁₋₄ alkyl.

53. The compound of any one of claims 1-18 or 31-51, wherein R^{7B} is methyl.

54. The compound of any one of claims 1-18 or 31-53, wherein t is 2.

55. The compound of anyone of claims 1-18, wherein A² is

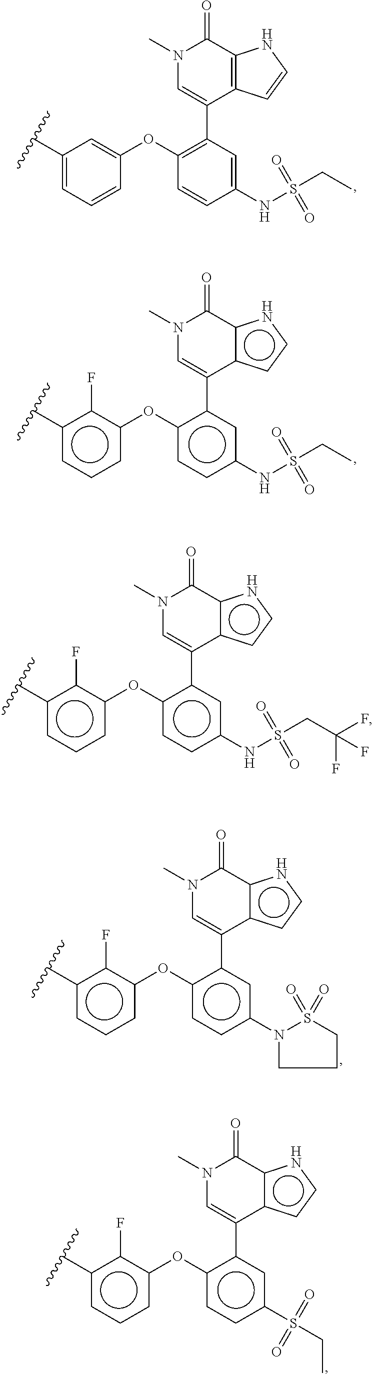

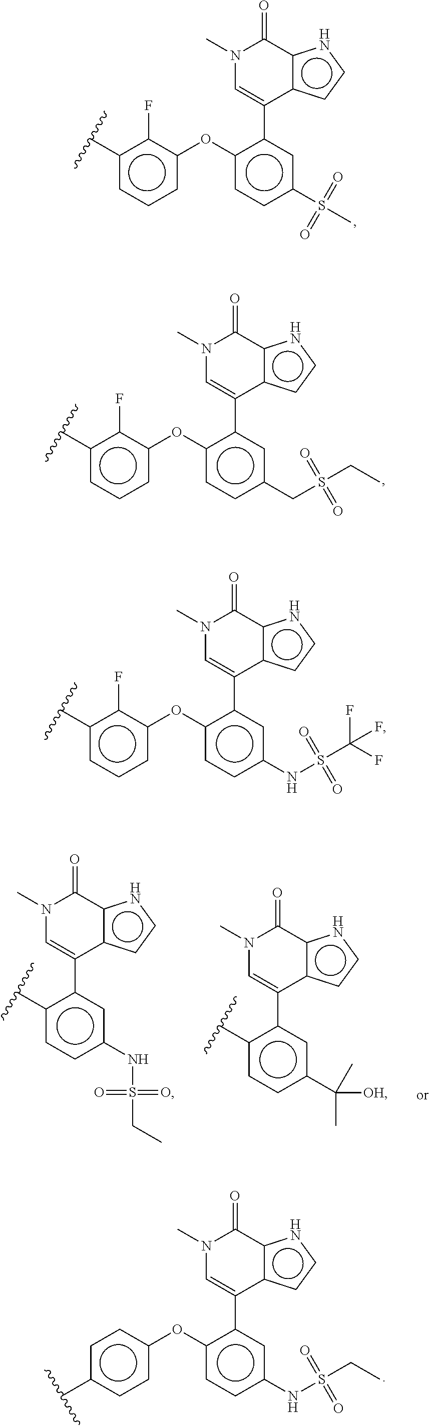

56. The compound of any one of claims 1-18, wherein A² is

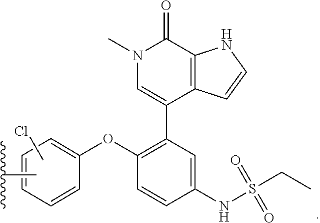

57. The compound of any one of claims 1-56, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-11 membered heterocyclyl containing 1, 2, 3, or 4 heteroatoms independently selected from nitrogen, oxygen, and sulfur.

58. The compound of any one of claims 1-56, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

59. The compound of any one of claims 1-56, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

60. The compound of any one of claims 1-56, wherein L is a bivalent, saturated or unsaturated, straight or branched C₅₋₄₀ hydrocarbon chain, wherein 1-20 methylene units of the hydrocarbon are independently replaced with —O—, —N(H)—, —N(C₁₋₆ alkyl)-, —N(H)C(O)—, —N(C₁₋₆ alkyl)C(O)—, —C(O)N(H)—, —C(O)N(C₁₋₆ alkyl)-, optionally substituted 3-10 membered carbocyclyl, or optionally substituted 3-10 membered heterocyclyl containing 1, 2, 3, or 4 heteroatoms independently selected from nitrogen, oxygen, and sulfur.

61. The compound of any one of claims 1-56, wherein L is -(3-7 membered, monocyclic, saturated heterocyclic ring containing 1 or 2 heteroatoms selected from nitrogen)-(OCH₂CH₂)₁₋₁₅—O—***, wherein *** is the point of attachment to A².

62. The compound of any one of claims 1-56, wherein L is -(3-7 membered, monocyclic, saturated heterocyclic ring containing 1 or 2 heteroatoms selected from nitrogen)-(OCH₂CH₂)₁₋₅—O—***, wherein *** is the point of attachment to A².

63. The compound of any one of claims 1-56, wherein L is -(3-7 membered, monocyclic, saturated heterocyclic ring containing 1 or 2 heteroatoms selected from nitrogen)-(OCH₂CH₂)₆₋₁₀—O—***, wherein *** is the point of attachment to A².

64. The compound of any one of claims 1-56, wherein L is -piperidinylene-(OCH₂CH₂)₁₋₁₅—O—***, wherein *** is the point of attachment to A².

65. The compound of any one of claims 1-56, wherein L is 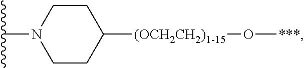
wherein *** is the point of attachment to A².

66. The compound of any one of claims 1-56, wherein L is 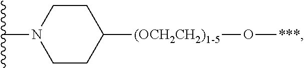
wherein *** is the point of attachment to A².

67. The compound of any one of claims 1-56, wherein L is 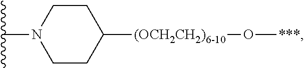
wherein *** is the point of attachment to A².

68. The compound of any one of claims 1-56, wherein L is -(3-7 membered, monocyclic, saturated heterocyclic ring containing 1 or 2 heteroatoms selected from nitrogen)-(OCH₂CH₂)₁₋₁₅—N(H)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—N(C₁₋₄ alkyl)C(O)—C₁₋₁₀ alkylene-***, -(3-7 membered, monocyclic, saturated heterocyclic ring containing 1 or 2 heteroatoms selected from nitrogen)-(OCH₂CH₂)₁₋₁₅—C(O)N(H)—C₁₋₁₀ alkylene-***, or -(3-7 membered, monocyclic, saturated heterocyclic ring containing 1 or 2 heteroatoms selected from nitrogen)-(OCH₂CH₂)₁₋₁₅—C(O)N(C₁₋₄ alkyl)-C₁₋₁₀ alkylene-***, wherein *** is the point of attachment to A².

69. The compound of any one of claims 1-56, wherein L is -piperidinylene-(OCH₂CH₂)₁₋₅—N(H)C(O)—C₁₋₅ alkylene-***, -piperidinylene-(OCH₂CH₂)₁₋₅—N(C₁₋₄ alkyl)C(O)—C₁₋₅ alkylene-***, -piperidinylene-(OCH₂CH₂)₁₋₅—C(O)N(H)—C₁₋₅ alkylene-***, or -piperidinylene-(OCH₂CH₂)₁₋₅—C(O)N(C₁₋₄ alkyl)-C₁₋₅ alkylene-***, wherein *** is the point of attachment to A².

70. The compound of any one of claims 1-56, wherein L is -(3-7 membered, monocyclic, saturated heterocyclic ring containing 1 or 2 heteroatoms selected from nitrogen)-(OCH₂CH₂)₁₋₁₀—***, -(3-7 membered, monocyclic, saturated heterocyclic ring containing 1 or 2 heteroatoms selected from nitrogen)-(C₀₋₁₀ alkylene)-O—***, or -(3-7 membered, monocyclic, saturated heterocyclic ring containing 1 or 2 heteroatoms selected from nitrogen)-C₁₋₁₀ alkylene, wherein *** is the point of attachment to A².

71. The compound of any one of claims 1-56, wherein L is -piperidinylene-(OCH₂CH₂)₁₋₅—***, -piperidinylene-(C₀₋₅ alkylene)-O—***, or -piperidinylene-(C₁₋₅ alkylene)-***, wherein *** is the point of attachment to A².

72. The compound of any one of claims 1-56, wherein L is -(3-7 membered monocyclic saturated heterocyclic ring containing 1 or 2 heteroatoms selected from nitrogen)-X¹—***, wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

73. The compound of any one of claims 1-56, wherein L is -(piperidinylene)-X¹—***, wherein *** is the point of attachment to A², and X¹ is (i) C₁₋₅ alkylene where 1 or 2 methylene groups are optionally replaced by —O—, —N(H)—, or —N(C₁₋₄ alkyl)-, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(Cis alkylene)-.

74. The compound of any one of claims 1-56, wherein L is 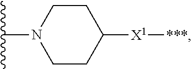
wherein *** is the point of attachment to A², and X¹ is (i) C₁₋₁₀ alkylene where 1 or 2 methylene groups are optionally replaced by —O—, —N(H)—, or —N(C₁₋₄ alkyl)-, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

75. The compound of any one of claims 1-56, wherein L is -(piperazinylene)-X¹—***, wherein *** is the point of attachment to A², and X¹ is (i) C₁₋₅ alkylene where 1 or 2 methylene groups are optionally replaced by —O—, (ii) a 3-4 membered monocyclic saturated heterocyclic ring containing 1 heteroatom selected from nitrogen, or (iii) -(3-4 membered monocyclic saturated heterocyclic ring containing 1 heteroatom selected from nitrogen)-(C₁₋₅ alkylene)-.

76. The compound of any one of claims 1-56, wherein L is 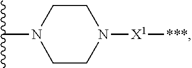
wherein *** is the point of attachment to A², and X¹ is (i) C₁₋₁₀ alkylene where 1 or 2 methylene groups are optionally replaced by —O—, (ii) a 3-7 membered monocyclic saturated heterocyclic ring containing 1 or 2 heteroatoms selected from nitrogen, or (iii) -(3-7 membered monocyclic saturated heterocyclic ring containing 1 or 2 heteroatoms selected from nitrogen)-(C₁₋₁₀ alkylene)-.

77. The compound of any one of claims 1-56, wherein L is -(3-7 membered monocyclic saturated heterocyclic ring containing 1 or 2 heteroatoms selected from nitrogen)-X²—(C₁₋₁₀ alkylene)-***, wherein *** is the point of attachment to A², and X² is —O—, —N(H)—, or —N(C₁₋₆ alkyl)-.

78. The compound of any one of claims 1-36, wherein L is -(piperidinylene)-X²—(C₁₋₁₀ alkylene)-***, wherein *** is the point of attachment to A², and X² is —O—, —N(H)—, or —N(C₁₋₆ alkyl)-.

79. The compound of any one of claims 1-56, wherein L is -(piperidinylene)-X²-(a 3-7 membered monocyclic saturated heterocyclic ring containing 1 or 2 heteroatoms selected from nitrogen)-***, wherein *** is the point of attachment to A², and X² is —O—, —N(H)—, or —N(C₁₋₆ alkyl)-.

80. The compound of any one of claims 1-56, wherein L is 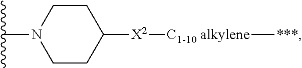
wherein *** is the point of attachment to A², and X² is —O—, —N(H)—, or —N(C₁₋₆ alkyl)-.

81. The compound of any one of claims 1-56, wherein L is 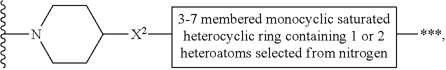
wherein *** is the point of attachment to A², and X² is —O—, —N(H)—, or —N(C₁₋₆ alkyl)-.

82. The compound of any one of claims 1-56, wherein L is -(3-7 membered monocyclic saturated heterocyclic ring containing 1 or 2 heteroatoms selected from nitrogen)-X¹—***, wherein *** is the point of attachment to A², and X¹ is —(OCH₂CH₂)₁₋₁₀ where 1 CH₂ group is optionally replaced with —C(H)(C₃₋₆ cycloalkyl)-.

83. The compound of any one of claims 1-56 wherein L is a 7-11 membered spirocyclic or fused bicyclic saturated heterocyclic ring containing 1, 2, or 3 heteroatoms selected from nitrogen and oxygen.

84. The compound of any one of claims 1-56, wherein L is a 7-8 membered spirocyclic or fused bicyclic saturated heterocyclic ring containing 2 heteroatoms selected from nitrogen.

85. The compound of any one of claims 1-56, wherein L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-, wherein X³ is C₁₋₁₀ alkylene, —O—, —N(H)—, —N(C₁₋₄ alkyl)-, or a bond.

86. The compound of any one of claims 1-56, wherein L is -(piperidinylene)-(C₁₋₅ alkylene)-(piperazinylene)-***, wherein *** is the point of attachment to A².

87. The compound of any one of claims 1-56, wherein L is -(piperazinylene)-(azetidinylene)-*** or (azetidinylene)-(piperazinylene)-***, wherein *** is the point of attachment to A².

88. The compound of any one of claims 1-56, wherein L is -(3-7 membered monocyclic saturated heterocyclic ring containing 1 or 2 heteroatoms selected from nitrogen)-X³-(3-7 membered monocyclic saturated heterocyclic ring containing 1 or 2 heteroatoms selected from nitrogen)-(C₀₋₆ alkylene)-O—***, wherein X³ is C₁₋₁₀ alkylene, —O—, —N(H)—, —N(C₁₋₄ alkyl)-, or a bond, and *** is the point of attachment to A².

89. The compound of any one of claims 1-56, wherein L is -(3-7 membered monocyclic saturated heterocyclic ring containing 1 or 2 heteroatoms selected from nitrogen)-(C₁₋₆ alkylene)-X³—(C₃₋₆ cycloalkylene)-(C₀₋₆ alkylene)-O—***, -(3-7 membered monocyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₁₋₆ alkylene)-X³—(C₃₋₆ cycloalkylene)-(C₀₋₆ alkylene)-O—***, X³—(C₃₋₆ cycloalkylene)-(C₀₋₆ alkylene)-O—***, -(3-7 membered monocyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₁₋₆ alkylene)-(C₃₋₆ cycloalkylene)-(C₀₋₆ alkylene)-O—***, or -(3-7 membered monocyclic saturated heterocyclic ring containing 1 or 2 heteroatoms selected from nitrogen)-(C₁₋₆ alkylene)-(3-7 membered monocyclic saturated heterocyclic ring containing 1 or 2 heteroatoms selected from nitrogen)-(C₀₋₆ alkylene)-O—***, wherein X³ is —N(H)— or —N(C₁₋₄ alkyl)-, and *** is the point of attachment to A².

90. The compound of any one of claims 1-56, wherein L is -(3-7 membered monocyclic saturated heterocyclic ring containing 1 or 2 heteroatoms selected from nitrogen)-X³—(C₃₋₆ cycloalkylene)-O—***, -(3-7 membered monocyclic saturated heterocyclic ring containing 1 or 2 heteroatoms selected from nitrogen)-X³—(C₃₋₆ cycloalkylene)-N(H)—***, or -(3-7 membered monocyclic saturated heterocyclic ring containing 1 or 2 heteroatoms selected from nitrogen)-X³—(C₃₋₆ cycloalkylene)-N(C₁₋₄ alkyl)-***, wherein *** is the point of attachment to A², and X³ is C₁₋₁₀ alkylene, —O—, —N(H)—, —N(C₁₋₄ alkyl)-, or a bond.

91. The compound of any one of claims 1-56, wherein L is -(piperidinylene)-X³—(C₃₋₆ cycloalkylene)-O—***, -(piperidinylene)-X³—(C₃₋₆ cycloalkylene)-N(H)—***, or -(piperidinylene)-X³—(C₃₋₆ cycloalkylene)-***, wherein *** is the point of attachment to A², and X³ is C₁₋₁₀ alkylene, —O—, —N(H)—, —N(C₁₋₄ alkyl)-, or a bond.

92. The compound of any one of claims 1-56, wherein L has the formula —(C₀₋₁₂ alkylene)-(optionally substituted 3-40 membered heteroalkylene)-(C₀₋₁₂ alkylene)-.

93. The compound of any one of claims 1-56, wherein L is one of the following: 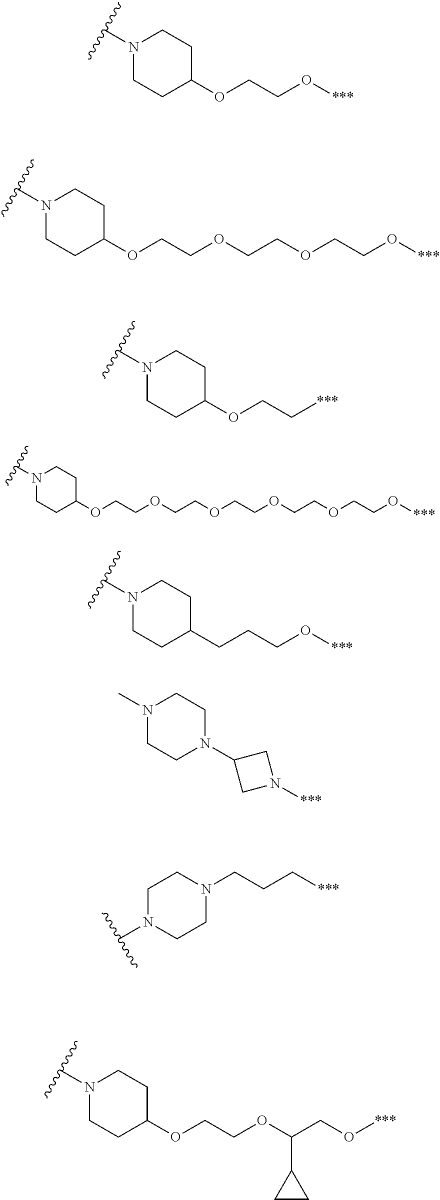
wherein *** is the point of attachment to A².

94. The compound of any one of claims 1-56, wherein L is one of the following: 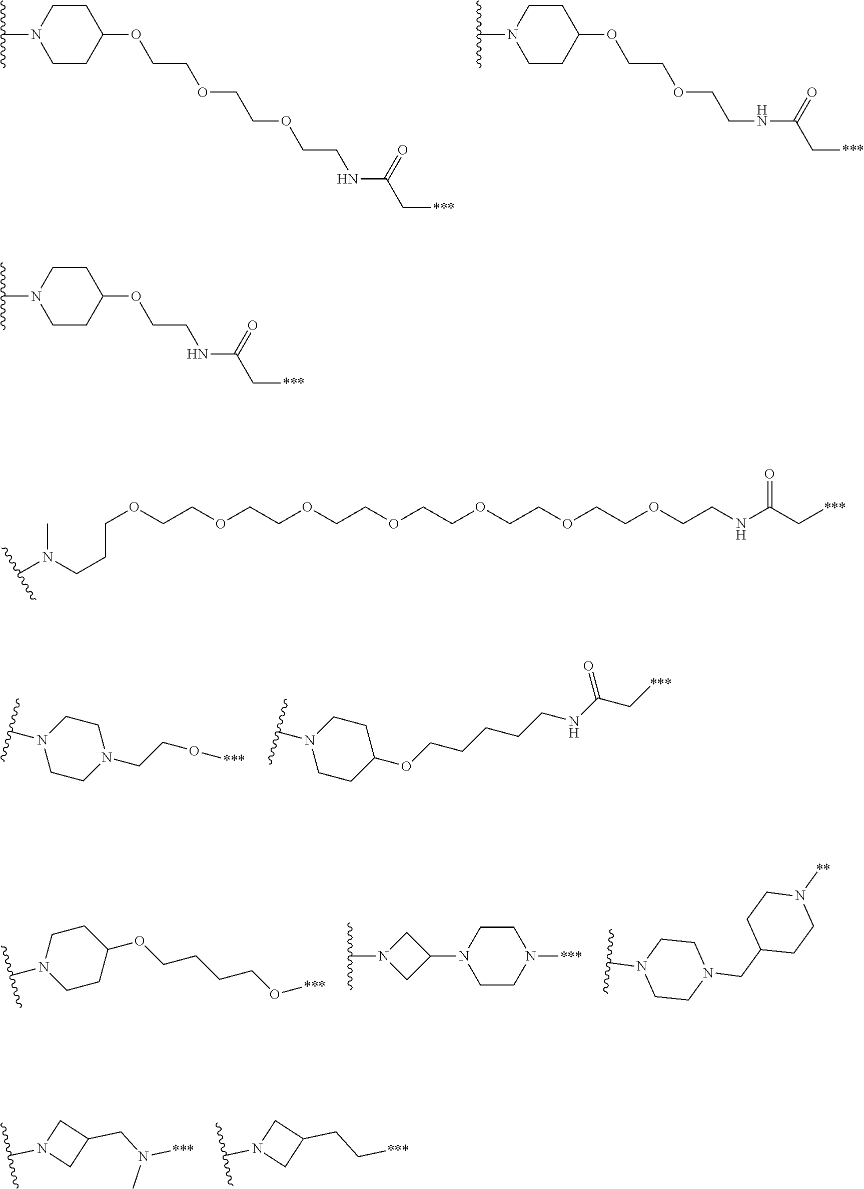
wherein *** is the point of attachment to A².

95. The compound of any one of claims 1-56, wherein L is one of the following: 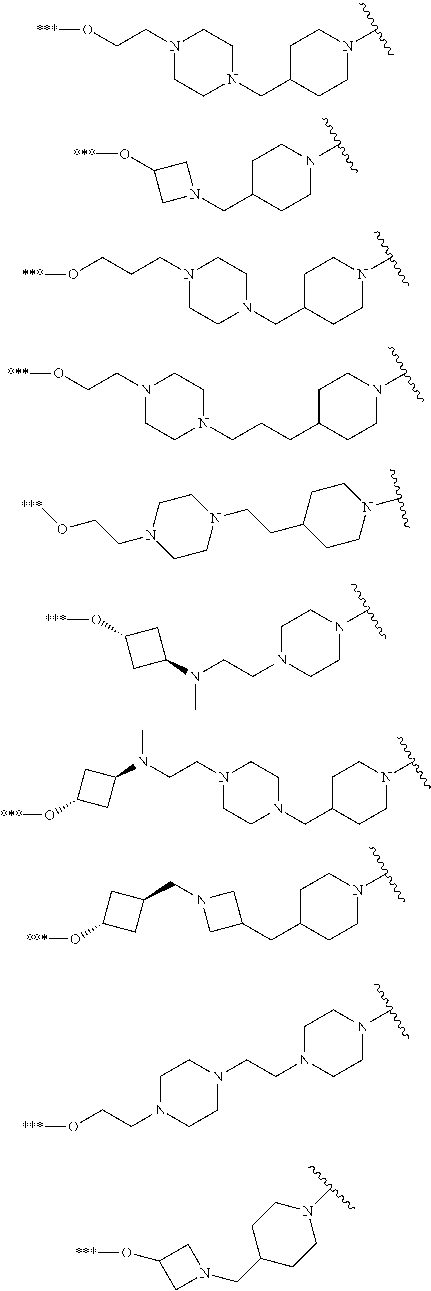
wherein *** is the point of attachment to A².

96. The compound of any one of claims 1-56, wherein L is one of the following: 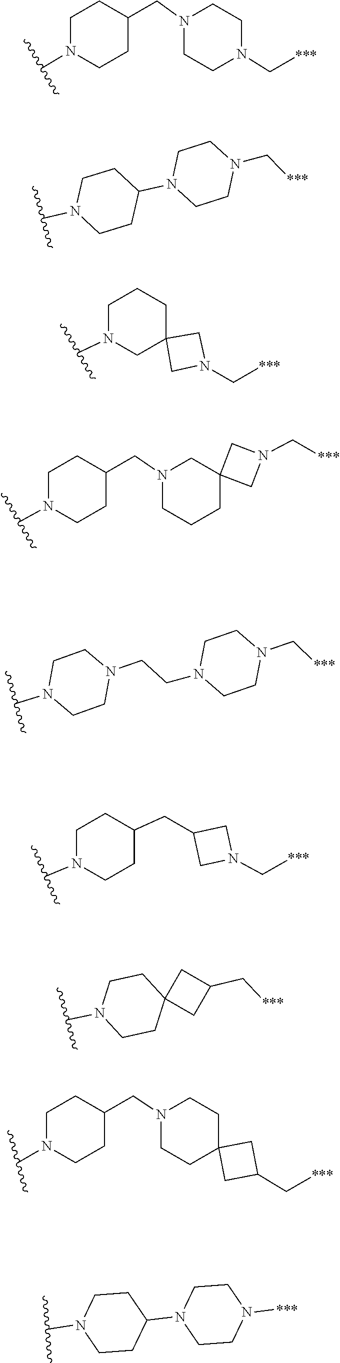
wherein *** is the point of attachment to A².

97. The compound of any one of claims 1-56, wherein L is -(3-7 membered monocyclic saturated heterocyclic ring containing 1 or 2 heteroatoms selected from nitrogen)-(C₁₋₄ alkylene)-(3-7 membered monocyclic saturated heterocyclic ring containing 1 or 2 heteroatoms selected from nitrogen)-(C₁₋₄ alkylene)-*** or -(3-7 membered monocyclic saturated heterocyclic ring containing 1 or 2 heteroatoms selected from nitrogen)-(C₁₋₄ alkylene)-(7-11 membered spirocyclic saturated heterocyclic ring containing 1 or 2 heteroatoms selected from nitrogen)-(C₁₋₄ alkylene)-***, wherein *** is the point of attachment to A².

98. A compound in Table 1, or a pharmaceutically acceptable salt thereof.

99. A compound represented by Formula II: 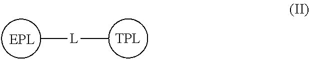
or a pharmaceutically acceptable salt thereof, wherein:
TPL is a group defined by Formula II-1 that is substituted by one occurrence of R^{II-1A}, wherein Formula II-1 is represented by: 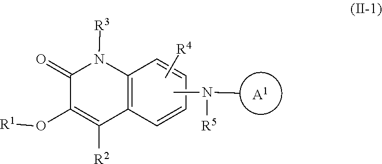
or a pharmaceutically acceptable salt thereof, wherein:
R^{II-1A} is a bond to L;
R¹ is —(C₁₋₃ alkylene)-C(O)N(R⁶)₂;
R², R³, R⁵, and R⁶ are independently hydrogen or C₁₋₄ alkyl;
R⁴ is hydrogen, C₁₋₄ alkyl, or halo;
R⁷ represents independently for each occurrence halo or C₁₋₄ alkyl;
A¹ is a pyrimidinyl, pyridazinyl, pyrazinyl, pyridinyl, or phenyl, each of which is substituted with n occurrences of R⁷;
L is a linker;
EPL is a moiety that binds to BRD4; and
n is 0, 1, or 2.

100. The compound of claim 99, wherein the TPL is 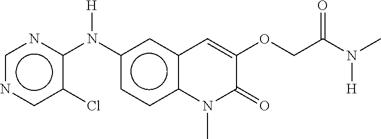
is substituted by one occurrence of R^{II-1A}.

101. The compound of claim 99, wherein the TPL is

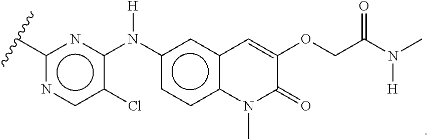

102. The compound of any one of claims 99-101, wherein the EPL is defined by Formula II-2 that is substituted by one occurrence of R^{II-2A}, wherein Formula II-2 is represented by: 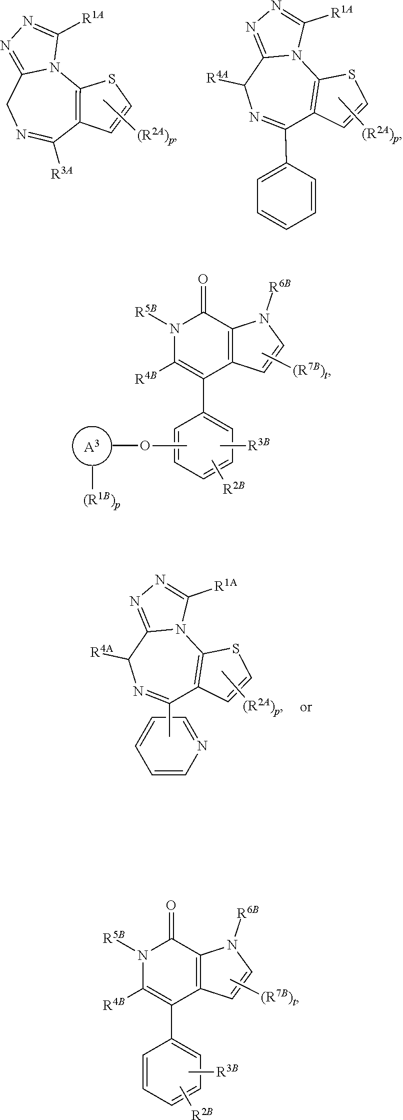
wherein
R^{II-2A} is a bond to L;
R^{1A} is C₁₋₄ alkyl or C₃₋₄ cycloalkyl;
R^{2A} represents independently for each occurrence C₁₋₄ alkyl or C₃₋₄ cycloalkyl;
R^{3A} is phenyl substituted with 1, 2, or 3 substituents independently selected from halo, C₁-C₄ alkyl, or C₁-C₄ haloalkyl;
R^{4A} is —(C₁₋₆ alkylene)-C(O)N(R^{5A})(R^{6A}), —(C₁₋₆ alkylene)-N(R^{5A})C(O)R^{7A}, —(C₁₋₆ alkylene)-CO₂R^{7A}, —(C₁₋₆ alkylene)-OC(O)R^{7A}, C₁₋₆ alkyl, or —(C₀₋₆ alkylene)-(5-6 membered heteroaryl containing 1, 2, or 3 heteroatoms independently selected from nitrogen, oxygen, and sulfur);
R^{5A} and R^{6A} are independently hydrogen, C₁₋₆ alkyl, or C₃₋₆ cycloalkyl; or R^{5A} and R^{6A} are taken together with the nitrogen atom to which they are attached to form a 3-7 membered ring containing 1 nitrogen atom;
R^{7A} is C₁₋₆ alkyl, —(C₁₋₆ alkylene)-(C₃₋₆ cycloalkyl), or C₃₋₆ cycloalkyl;
R^{1B} represents independently for each occurrence halo, C₁₋₄ alkyl, or C₁₋₄ haloalkyl;
R^{2B} is hydrogen, halo, C₁₋₄ alkyl, or C₁₋₄ haloalkyl;
R^{3B} is —N(R^{8B})SO₂R^{9B}, —SO₂N(R^{8B})₂, —SO₂R^{9B}, —(C₁₋₆ alkylene)-SO₂R^{9B}, C₁₋₆ hydroxyalkyl, or a 4-7 membered saturated carbocyclic ring in which one CH₂ is replaced with SO₂;
R^{4B} is hydrogen, halo, or C₁₋₄ alkyl;
R^{5B} is C₁₋₄ alkyl or C₃₋₄ cycloalkyl;
R^{6B} is hydrogen, C₁₋₄ alkyl, or C₃₋₄ cycloalkyl;
R^{7B} represents independently for each occurrence C₁₋₄ alkyl or C₃₋₄ cycloalkyl;
R^{8B} represents independently for each occurrence hydrogen or C₁₋₄ alkyl; or two occurrences of R^{8B} are taken together with the nitrogen atom to which they are attached to form a 3-7 membered heterocyclic ring containing 1 or 2 heteroatoms independently selected from nitrogen and oxygen; or R^{8B} and R^{9B} are taken together with their intervening atoms to a form a 5-7 membered ring containing 1, 2, or 3 heteroatoms independently selected from nitrogen, oxygen, and sulfur;
R^{9B} is C₁₋₆ alkyl, C₁₋₆ haloalkyl, —(C₁₋₆ alkylene)-(C₃₋₆ cycloalkyl), or C₃₋₆ cycloalkyl;
A³ is phenylene, a 3-10 membered saturated monocyclic, bicyclic or spirocyclic carbocycylene, or C₁₋₆ alkylene; and
p and t are independently 0, 1, or 2.

103. The compound of any one of claims 99-101, wherein the EPL is 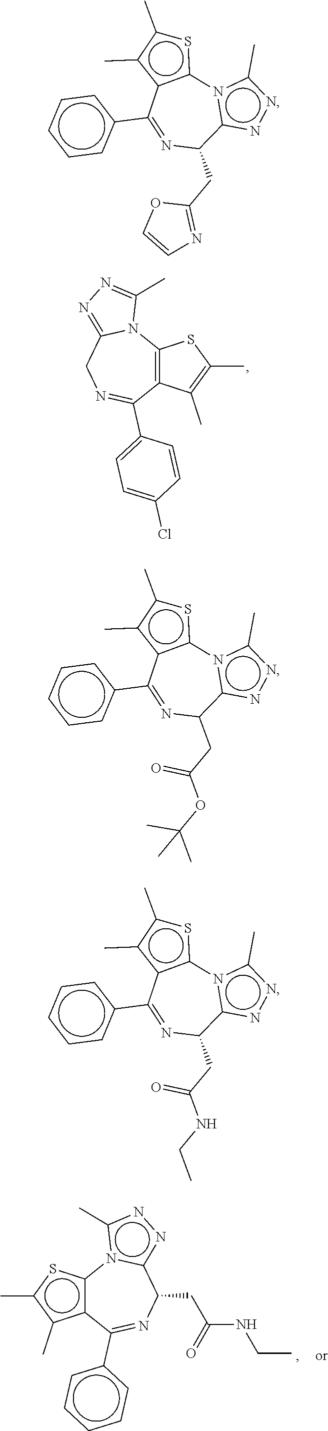 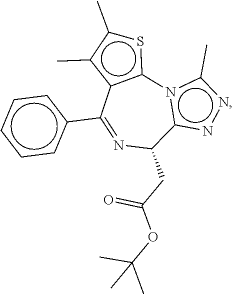
each of which is substituted by one occurrence of R^{II-2A}, wherein R^{II-2A} is a bond to L.

104. The compound of any one of claims 99-101, wherein the EPL is

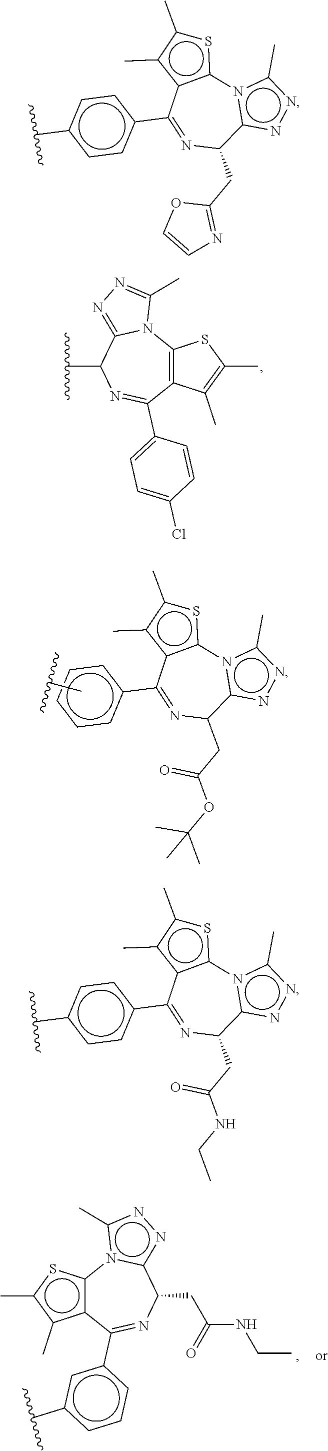

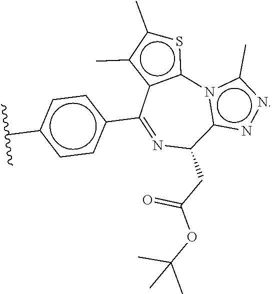

105. The compound of any one of claims 99-101, wherein the EPL is 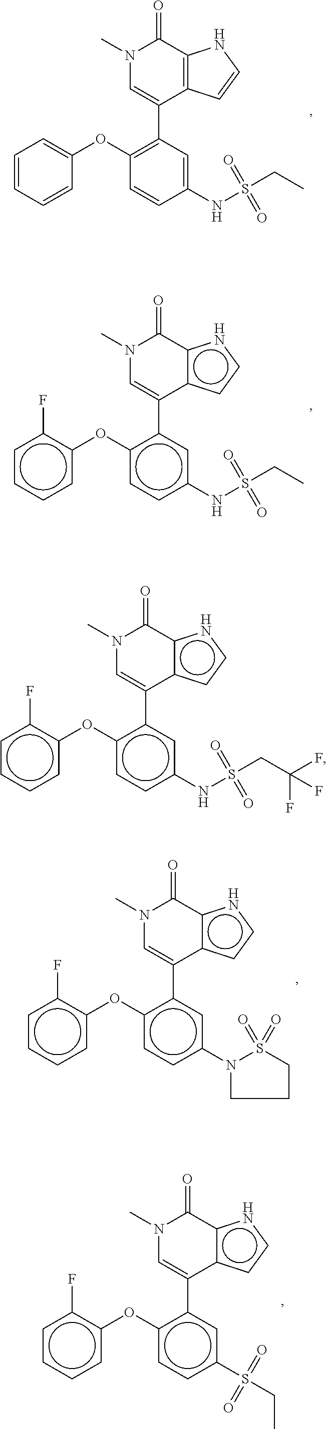 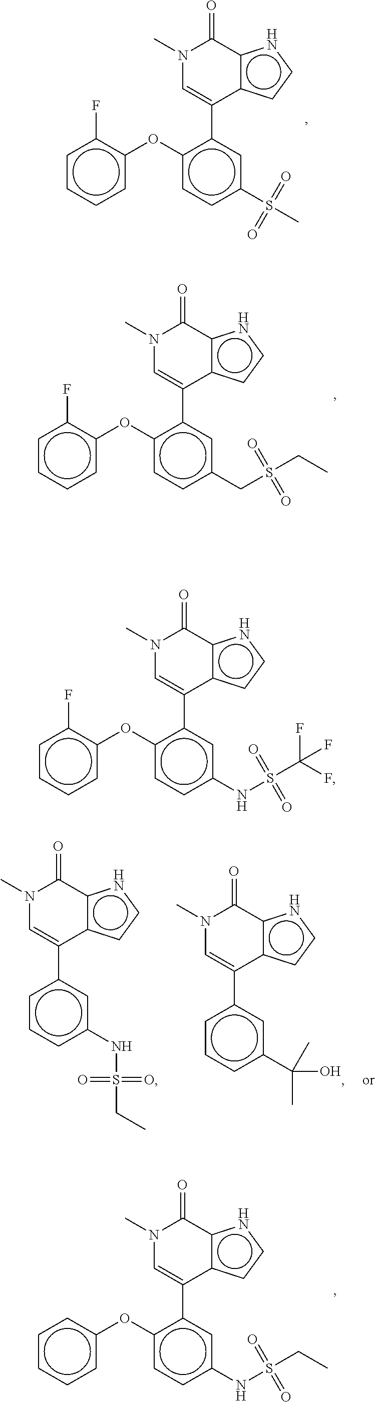
each of which is substituted by one occurrence of R^{II-2A}, wherein R^{II-2A} is a bond to L.

106. The compound of any one of claims 99-101, wherein the EPL is

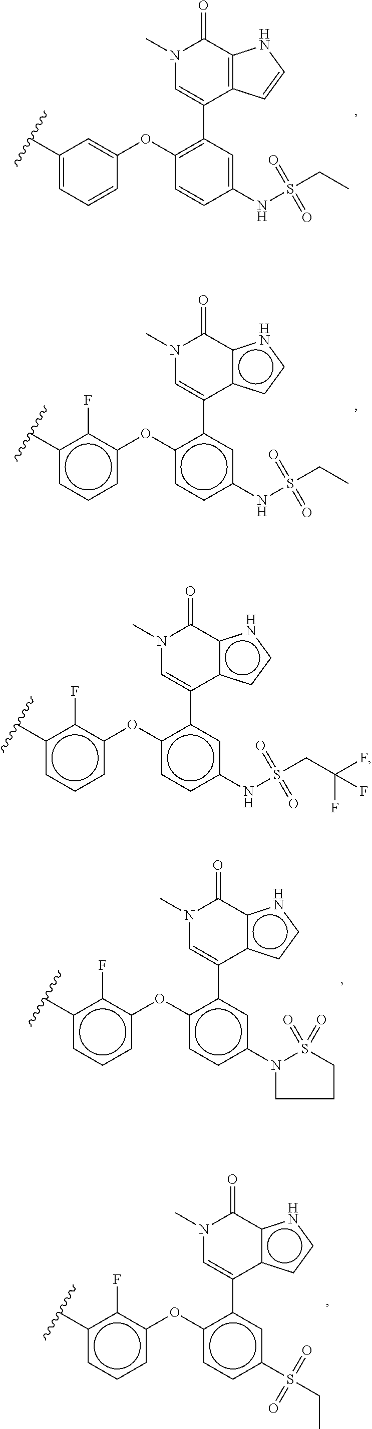

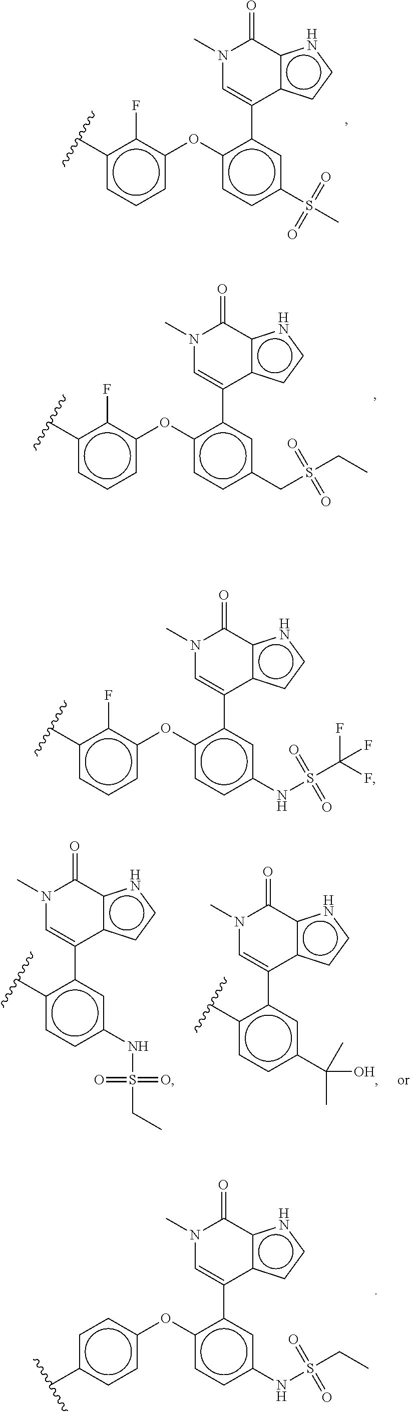

107. The compound of any one of claims 99-106, wherein L is a bivalent, saturated or unsaturated, straight or branched C₁₋₆₀ hydrocarbon chain, wherein 0-20 methylene units of the hydrocarbon are independently replaced with —O—, —S—, —N(H)—, —N(C₁₋₆ alkyl)-, —OC(O)—, —C(O)O—, —S(O)—, —S(O)₂—, —N(H)S(O)₂—, —N(C₁₋₆ alkyl)S(O)₂—, —S(O)₂N(H)—, —S(O)₂N(C₁₋₆ alkyl)-, —N(H)C(O)—, —N(C₁₋₆ alkyl)C(O)—, —C(O)N(H)—, —C(O)N(C₁₋₆ alkyl)-, —OC(O)N(H)—, —OC(O)N(C₁₋₆ alkyl)-, —N(H)C(O)O—, —N(C₁₋₆ alkyl)C(O)O—, optionally substituted 3-10 membered carbocyclyl, or optionally substituted 3-11 membered heterocyclyl containing 1, 2, 3, or 4 heteroatoms independently selected from nitrogen, oxygen, and sulfur.

108. The compound of any one of claims 99-106, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, —C(O)N(C₁₋₆ alkyl)-, 3-10 membered carbocyclyl, or 3-10 membered heterocyclyl containing 1, 2, or 3 heteroatoms independently selected from nitrogen, oxygen, and sulfur.

109. The compound of any one of claims 99-106, wherein L is a bivalent, saturated, straight or branched C₃₋₃₀ hydrocarbon chain, wherein 0-15 methylene units of the hydrocarbon are independently replaced with —O—, —N(H)—, —N(C₁₋₆ alkyl)-, —OC(O)—, —C(O)O—, —N(H)C(O)—, —N(C₁₋₆ alkyl)C(O)—, —C(O)N(H)—, or —C(O)N(C₁₋₆ alkyl)-.

110. A pharmaceutical composition comprising a compound of any one of claims 1-109 and a pharmaceutically acceptable carrier.

111. A method of treating cancer, comprising administering to a patient in need thereof a therapeutically effective amount of a compound of any one of claims 1-109 to treat the cancer.

112. The method of claim 111, wherein the cancer is ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia.

113. A method of causing death of a cancer cell, comprising contacting a cancer cell with an effective amount of a compound of any one of claims 1-109 to cause death of the cancer cell.

114. The method of claim 113, wherein the cancer cell is selected from an ovarian cancer, uterine cancer, endometrial cancer, cervical cancer, prostate cancer, testicular cancer, breast cancer, brain cancer, lung cancer, oral cancer, esophageal cancer, head and neck cancer, stomach cancer, colon cancer, rectal cancer, skin cancer, sebaceous gland carcinoma, bile duct cancer, gallbladder cancer, liver cancer, pancreatic cancer, bladder cancer, urinary tract cancer, kidney cancer, eye cancer, thyroid cancer, lymphoma, or leukemia cell.
